(19) United States
(12) Patent Application Publication
(10) Pub. No.: US 2022/0273767 A1
(43) Pub. Date: Sep. 1, 2022

(54) Interleukin 10 Conjugates and Uses Thereof

(71) Applicant: Synthorx, Inc., La Jolla, CA (US)

(72) Inventors: Carolina E. CAFFARO, San Diego, CA (US); Jerod PTACIN, San Diego, CA (US); Marcos MILLA, Carlsbad, CA (US)

(73) Assignee: Synthorx, Inc., La Jolla, CA (US)

(21) Appl. No.: 17/735,564

(22) Filed: May 3, 2022

Related U.S. Application Data
(63) Continuation of application No. PCT/US2020/058845, filed on Nov. 4, 2020.
(60) Provisional application No. 62/930,322, filed on Nov. 4, 2019.
(60) Provisional application No. 62/953,095, filed on Dec. 23, 2019.

(51) Int. Cl. A61K 38/20; A61K 47/54; C07K 1/107; A61P 35/00

(52) U.S. Cl. CPC A61K 38/2066; A61P 35/00; C07K 1/1077; A61K 47/545

(57) ABSTRACT

Disclosed herein are interleukin 10 (IL-10) conjugates and uses in the treatment of one or more indications. Also described herein are pharmaceutical compositions and kits comprising one or more of the IL-10 conjugates.

### CROSS-REFERENCE TO RELATED APPLICATIONS

This application is a continuation of International Application No. PCT/US2020/058845, filed on Nov. 4, 2020, which claims priority to U.S. Provisional Application No. 62/930,322, filed on Nov. 4, 2019, and U.S. Provisional Application No. 62/953,095, filed on Dec. 23, 2019, the disclosures of each of which are hereby incorporated by reference in their entireties.

### SEQUENCE LISTING

This application contains a Sequence Listing which has been submitted electronically in ASCII format and is hereby incorporated by reference in its entirety. Said ASCII copy, created on Nov. 2, 2020, is named 01183-0077-00PCT_sequence_listing.txt and is 135 KB in size.

### BACKGROUND

Distinct populations of T cells modulate the immune system to maintain immune homeostasis and tolerance. For example, regulatory T (Treg) cells prevent inappropriate responses by the immune system by preventing pathological self-reactivity while cytotoxic T cells target and destroy infected cells and/or cancerous cells. In some instances, modulation of the different populations of T cells provides an option for treatment of a disease or indication.

Cytokines comprise a family of cell signaling proteins such as chemokines, interferons, interleukins, lymphokines, tumor necrosis factors, and other growth factors playing roles in innate and adaptive immune cell homeostasis. Cytokines are produced by immune cells such as macrophages, B lymphocytes, T lymphocytes and mast cells, endothelial cells, fibroblasts, and different stromal cells. In some instances, cytokines modulate the balance between humoral and cell-based immune responses.

Interleukins are signaling proteins that modulate the development and differentiation of T and B lymphocytes, cells of the monocytic lineage, neutrophils, basophils, eosinophils, megakaryocytes, and hematopoietic cells. Interleukins are produced by helper CD4+ T and B lymphocytes, monocytes, macrophages, endothelial cells, and other tissue residents.

In some instances, interleukin 10 (IL-10) signaling is used to modulate T cell responses. Accordingly, in one aspect, provided herein are IL-10 conjugates and uses thereof.

### SUMMARY

Disclosed herein, in certain embodiments, are interleukin 10 (IL-10) conjugates and uses thereof in the treatment of one or more indications. In some embodiments, disclosed herein are IL-10 conjugates for the treatment of cancer. In additional cases, disclosed herein are pharmaceutical compositions and kits that comprise an IL-10 conjugate described herein.

The following embodiments are encompassed.

Embodiment A1. An IL-10 conjugate comprising the amino acid sequence of SEQ ID NO: 1 in which at least one amino acid residue in the IL-10 conjugate is replaced by the structure of Formula (I):

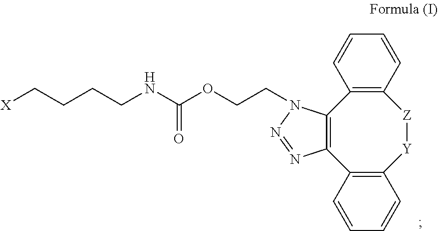

wherein:

### Z is CH₂ and Y is

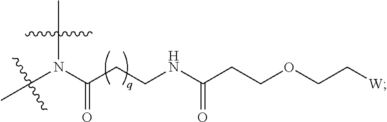

### Y is CH₂ and Z is

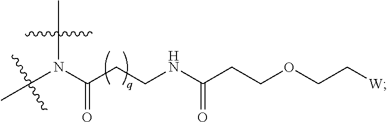

### Z is CH₂ and Y is

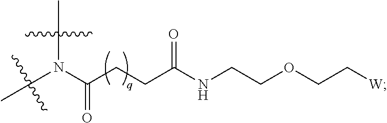

or

### Y is CH₂ and Z is

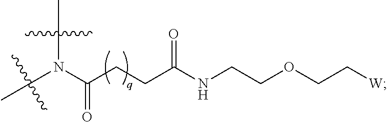

W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa;
q is 1, 2, or 3;
X has the structure:

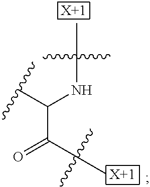

X−1 indicates the point of attachment to the preceding amino acid residue; and
X+1 indicates the point of attachment to the following amino acid residue.

Embodiment A2. The IL-10 conjugate of embodiment A1, wherein Z is CH₂ and Y is

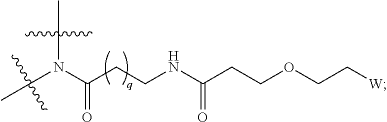

Embodiment A3. The IL-10 conjugate of embodiment A1, wherein Y is CH₂ and Z is

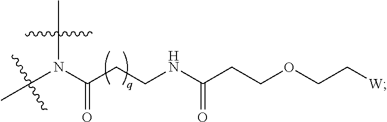

Embodiment A4. The IL-10 conjugate of embodiment A1, wherein Z is CH₂ and Y is

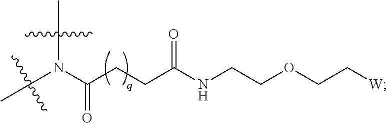

Embodiment A5. The IL-10 conjugate of embodiment 1, wherein Y is CH₂ and Z is

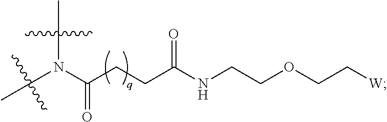

Embodiment A6. The IL-10 conjugate of any one of embodiments A1-5, wherein the PEG group has an average molecular weight selected from 5 kDa, 10 kDa, 20 kDa and 30 kDa.

Embodiment A7. The IL-10 conjugate of embodiment A6, wherein the PEG group has an average molecular weight selected from 10 kDa and 20 kDa.

Embodiment A8. The IL-10 conjugate of any one of embodiments A1-7, wherein the position of the structure of Formula (I) is selected from N82, K88, A89, K99, K125, N126, N129, and K130.

Embodiment A9. The IL-10 conjugate of embodiment A8, wherein the position of the structure of Formula (I) is selected from N82 and N129.

Embodiment A10. The IL-10 conjugate of embodiment A1, wherein the structure of Formula (I) has the structure of Formula (X) or Formula (XI), or is a mixture of Formula (X) and Formula (XI):

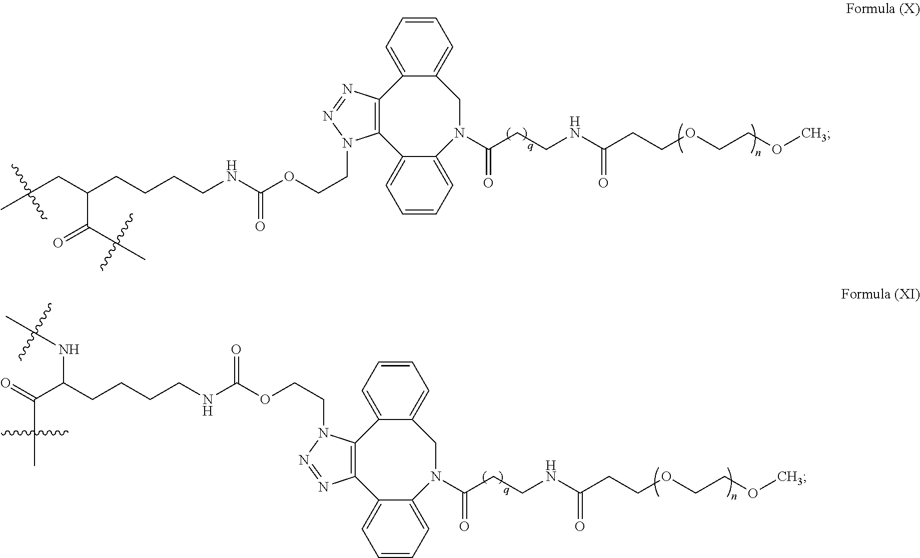

wherein:
q is 1, 2, or 3;
n is an integer in the range from about 2 to about 5000; and
the wavy lines indicate covalent bonds to amino acid residues within SEQ ID NO: 1 that are not replaced.

Embodiment A11. The IL-10 conjugate of embodiment A10, wherein the position of the structure of Formula (X) or Formula (XI) in SEQ ID NO: 1 is selected from N82, K88, A89, K99, K125, N126, N129, and K130.

Embodiment A12. The IL-10 conjugate of embodiment A11, wherein the position of the structure of Formula (X) or Formula (XI) in SEQ ID NO: 1 is selected from N82 and N129.

Embodiment A13. The IL-10 conjugate of any one of embodiments A10-12, wherein n is an integer such that —(OCH₂CH₂)ₙ—OCH₃ has a molecular weight of about 10 kDa or 20 kDa.

Embodiment A14. The IL-10 conjugate of embodiment A1, wherein the structure of Formula (I) has the structure of Formula (XII) or Formula (XIII), or is a mixture of Formula (XII) and Formula (XIII):

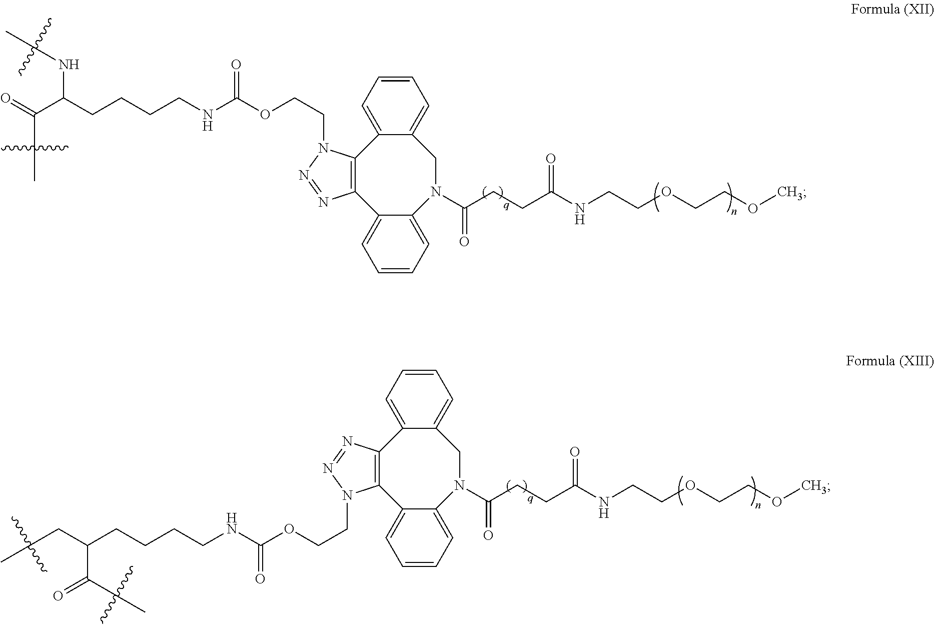

wherein:
q is 1, 2, or 3;
n is an integer in the range from about 2 to about 5000; and
the wavy lines indicate covalent bonds to amino acid residues within SEQ ID NO: 1 that are not replaced.

Embodiment A15. The IL-10 conjugate of embodiment A14, wherein the position of the structure of Formula (XII) or Formula (XIII) in SEQ ID NO: 1 is selected from N82, K88, A89, K99, K125, N126, N129, and K130.

Embodiment A16. The IL-10 conjugate of embodiment A14, wherein the position of the structure of Formula (XII) or Formula (XIII) in SEQ ID NO: 1 is selected from N82 and N129.

Embodiment A17. The IL-10 conjugate of any one of embodiments A14-16, wherein n is an integer such that —(OCH₂CH₂)ₙ—OCH₃ has a molecular weight of about 10 kDa or 20 kDa.

Embodiment A18. The IL-10 conjugate of any one of embodiments A1-17, wherein q is 1.

Embodiment A19. The IL-10 conjugate of any one of embodiments A1-17, wherein q is 2.

Embodiment A20. The IL-10 conjugate of any one of embodiments A1-17, wherein q is 3.

Embodiment A21. The IL-10 conjugate of any one of embodiments A1-20, wherein the IL-10 conjugate is a pharmaceutically acceptable salt, solvate, or hydrate.

Embodiment A22. A method of treating cancer in a subject in need thereof, comprising administering to the subject an effective amount of the IL-10 conjugate of any one of embodiments A1-21.

Embodiment A23. The method of embodiment A22, wherein the cancer is selected from renal cell carcinoma (RCC), non-small cell lung cancer (NSCLC), head and neck squamous cell cancer (HNSCC), classical Hodgkin lymphoma (cHL), primary mediastinal large B-cell lymphoma (PMBCL), urothelial carcinoma, microsatellite unstable cancer, microsatellite stable cancer, microsatellite-stable colorectal cancer, gastric cancer, cervical cancer, hepatocellular carcinoma (HCC), Merkel cell carcinoma (MCC), melanoma, small cell lung cancer (SCLC), esophageal, glioblastoma, mesothelioma, breast cancer, triple-negative breast cancer, prostate cancer, bladder cancer, ovarian cancer, tumors of moderate to low mutational burden, cutaneous squamous cell carcinoma (CSCC), squamous cell skin cancer (SCSC), tumors of low- to non-expressing PD-L1, tumors disseminated systemically to the liver and CNS beyond their primary anatomic originating site, and diffuse large B-cell lymphoma.

Embodiment A24. The method of embodiment A22 or A23, wherein the IL-10 conjugate is administered to the subject once per day, twice per day, three times per day, once per week, once every two weeks, once every three weeks, once every 4 weeks, once every 5 weeks, once every 6 weeks, once every 7 weeks, or once every 8 weeks.

Embodiment A25. The method of any one of embodiments A22-24, wherein the IL-10 conjugate is administered to the subject by intravenous administration.

Embodiment A26. A method of making an IL-10 conjugate, comprising:

reacting an IL-10 polypeptide comprising an unnatural amino acid of formula

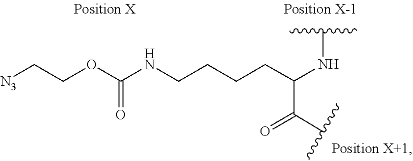 [SMILES: *NC(CCCCNC(=O)OCCN=[N+]=[N-])C(*)=O]

wherein the IL-10 polypeptide comprises the amino acid sequence of SEQ ID NO: 1 in which at least one amino acid residue in the IL-10 polypeptide is replaced by the unnatural amino acid, Position X−1 indicates the point of attachment to the preceding amino acid residue, Position X+1 indicates the point of attachment to the following amino acid residue, and Position X indicates the position of the amino acid for which the unnatural amino acid substitutes, with an mPEG-DBCO of formula

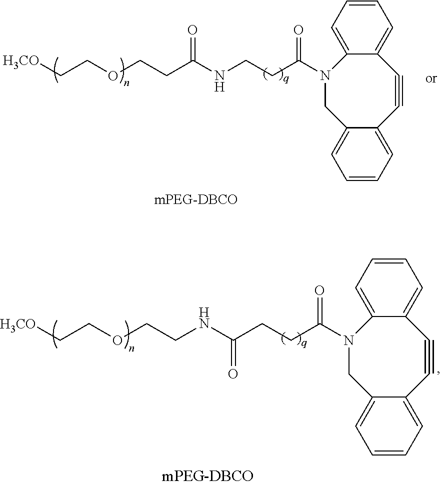

wherein q is 1, 2, or 3, and n is such that the mPEG-DBCO comprises a PEG having a molecular weight of about 5 kDa, 10 kDa, 15 kDa, 20 kDa, 25 kDa, 30 kDa, 35 kDa, 40 kDa, 45 kDa, 50 kDa, or 60 kDa,
thereby producing the IL-10 conjugate.

Embodiment A27. The method of embodiment A26, wherein q is 1.

Embodiment A28. The method of embodiment A26, wherein q is 2.

Embodiment A29. The method of embodiment A26, wherein q is 3.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a representative SDS-PAGE and Western Blot analysis of Compound A under reducing conditions, and shows homogeneous pegylation of IL-10 monomers as described in Example 2.

FIG. 2 illustrates a representative molar mass determination of Compound A as described in Example 2 by SEC-MALS.

FIG. 3 illustrates a representative analysis of dimer stability of Compound A as described in Example 2 at low concentrations by size exclusion chromatography (SEC).

FIG. 4 illustrates a trace concentration of Compound A (pg/mL) versus proliferation (OD₄₅₀) in the MC/9 proliferation assay from Example 3.

FIG. 5 illustrates a trace concentration of Compound D (pg/mL) versus proliferation (OD₄₅₀) in the MC/9 proliferation assay from Example 3.

FIG. 6 illustrates a trace concentration of Compound E (pg/mL) versus proliferation (OD₄₅₀) in the MC/9 proliferation assay from Example 3.

FIG. 7A illustrates a trace concentration of Compound F (pg/mL) versus proliferation (OD₄₅₀) in the MC/9 proliferation assay from Example 3.

FIG. 7B illustrates a trace concentration of Compound G and Compound H (pg/mL) versus proliferation (OD₄₅₀) in the MC/9 proliferation assay from Example 3.

FIG. 8 illustrates the measurement of bioactivity of wild-type IL-10 in the PathHunter assay from Example 3.

FIG. 9 illustrates the measurement of bioactivity of Compound A in the PathHunter assay from Example 3.

FIGS. 10A-C illustrate pSTAT3 profiling in Balb/c mouse splenocytes for wild-type IL-10 (closed circles), Compound A (open triangles), and Compound D (open squares) from Example 4 in CD8+ T cells, NK cells, and B cells, respectively.

FIGS. 11A-C illustrate pSTAT3 profiling in B57BL/6 mouse splenocytes for wild-type IL-10 (closed circles), Compound A (open triangles), and Compound D (open squares) from Example 4 in CD8+ T cells, NK cells, and B cells, respectively.

FIGS. 12A-C illustrate the concentration of wild-type His-IL-10, Compound A, and Compound D versus MFI of pSTAT3 from Example 5 in CD8+ T cells, NK cells, and B cells, respectively.

FIGS. 13A-B illustrate IFNγ release upon antigen-specific TCR activation by wild-type His-IL-10 or Compound A from Example 6. N.D.=not detected.

FIGS. 14A-B illustrate the upregulation of PD-1 following treatment with [His]-IL-10 or Compound A from Example 6 and demonstrates that such upregulation is independent of TCR activation.

### DETAILED DESCRIPTION

Cytokines comprise a family of cell signaling proteins such as chemokines, interferons, interleukins, lymphokines, tumor necrosis factors, and other growth factors playing roles in innate and adaptive immune cell homeostasis. Cytokines are produced by immune cells such as macrophages, B lymphocytes, T lymphocytes and mast cells, endothelial cells, fibroblasts, and different stromal cells. In some instances, cytokines modulate the balance between humoral and cell-based immune responses.

Interleukins are signaling proteins which modulate the development and differentiation of T and B lymphocytes, cells of the monocytic lineage, neutrophils, basophils, eosinophils, megakaryocytes, and hematopoietic cells. Interleukins are produced by helper CD4 T and B lymphocytes, monocytes, macrophages, endothelial cells, and other tissue residents. In some cases, there are about 15 interleukins, interleukins 1-13, interleukin 15, and interleukin 17.

IL-10 generates tumor immunity by activation of tumor-infiltrating CD8+ T cells, cellular proliferation of CD8+ T cells, induction of IFN-7 which increases MHC class I on tumor cells and MHC class II on macrophages, and induction of cytotoxic proteins mediating target cell lysis. Increased T cell receptor stimulation on CD8+ T cells provides antiapoptotic and proliferation signals. An unexpected role for IL-10 in the tumor microenvironment (TME) is the inhibition of pro-inflammatory Th17 cells and cytokines responsible for tumor associated inflammation leading to suppression of anti-tumor effector cell responses. Preclinical studies have shown that IL-10 deficiency increases tumor incidence and reduces immune surveillance. Additionally, treatment of Her2 transgenic mice with pegylated IL-10 has led to tumor rejection but requires expression of IFN-γ and granzyme-expressing CD8+ T cells, with a significant increase in CD8a/b+ T cells in the tumor.

IL-10 has a relatively short serum half-life in the body. Indeed, the half-life in mice as measured by in vitro bioassay or by efficacy in the septic shock model system (see Smith et al., Cellular Immunology 173:207-214 (1996), the disclosure of which is incorporated herein by reference) is about 2 to 6 hours.

Disclosed herein, in certain embodiments, is a modified IL-10 polypeptide which has an enhanced plasma half-life. In some embodiments, also described herein is a modified IL-10 polypeptide which, upon dimerization, enhances the exposure of a plurality of tumor cells to tumor infiltrating immune cells. In other embodiments, further described herein is a modified IL-10 polypeptide which forms a biologically active IL-10 dimer. In some embodiments, described herein is a modified IL-10 polypeptide which forms a biologically active modified IL-10 dimer.

Additionally described herein are IL-10 conjugates, where the IL-10 conjugates are IL-10 or modified IL-10 polypeptides conjugated with at least one conjugation moiety. Also described herein are pharmaceutical compositions comprising one or more of the modified IL-10 polypeptides or the IL-10 conjugates, and methods of treating a disease or indication.

### Definitions

Unless defined otherwise, all technical and scientific terms used herein have the same meaning as is commonly understood by one of skill in the art to which the claimed subject matter belongs. It is to be understood that the detailed descriptions are exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the present disclosure.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, such as for example, within 15%, 10%, or 5%.

The section headings used herein are for organizational purposes only and are not to be construed as limiting the subject matter described.

As used herein, the term “subject(s)” or “patient(s)” means any mammal. In some embodiments, the mammal is a human. In some embodiments, the mammal is a non-human. In some embodiments, the subject does not have a disease. In some embodiments, the subject is not diagnosed with a disease. In some embodiments, the subject is diagnosed with a disease. In some embodiments, the subject is diagnosed with at least one disease. In some cases, the subject is a patient. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the terms “significant” and “significantly” in reference to receptor binding means a change sufficient to impact binding of the IL-10 polypeptide to a target receptor. In some instances, the term refers to a change of at least 10%, 20%, 30%, 40%, 50%, 60%, 70%, 80%, 90%, 95%, or more. In some instances, the term means a change of at least 2-fold, 3-fold, 4-fold, 5-fold, 6-fold, 7-fold, 8-fold, 9-fold, 10-fold, 50-fold, 100-fold, 500-fold, 1000-fold, or more.

In some instances, the term “substantially” in reference to dimerization means a change sufficient to prevent formation of an IL-10 dimer.

As used herein, the term “tumor infiltrating immune cell(s)” refers to immune cells that have infiltrated into a region comprising tumor cells (e.g., in a tumor microenvironment). In some instances, the tumor infiltrating immune cells are associated with tumor cell destruction, a decrease in tumor cell proliferation, a reduction in tumor burden, or combinations thereof. In some instances, the tumor infiltrating immune cells comprise tumor infiltration lymphocytes (TILs). In some instances, the tumor infiltrating immune cells comprise T cells, B cells, natural killer cells, macrophages, neutrophils, dendritic cells, mast cells, eosinophils or basophils. In some instances, the tumor infiltrating immune cells comprise CD4+ or CD8+ T cells.

As used herein, the term “unnatural amino acid” refers to an amino acid other than one of the 20 naturally occurring amino acids. Exemplary unnatural amino acids are described in Young et al., “Beyond the canonical 20 amino acids: expanding the genetic lexicon,” J. of Biological Chemistry 285(15): 11039-11044 (2010), the disclosure of which is incorporated herein by reference.

As used herein, “nucleotide” refers to a compound comprising a nucleoside moiety and a phosphate moiety. Exemplary natural nucleotides include, without limitation, adenosine triphosphate (ATP), uridine triphosphate (UTP), cytidine triphosphate (CTP), guanosine triphosphate (GTP), adenosine diphosphate (ADP), uridine diphosphate (UDP), cytidine diphosphate (CDP), guanosine diphosphate (GDP), adenosine monophosphate (AMP), uridine monophosphate (UMP), cytidine monophosphate (CMP), and guanosine monophosphate (GMP), deoxyadenosine triphosphate (dATP), deoxythymidine triphosphate (dTTP), deoxycytidine triphosphate (dCTP), deoxyguanosine triphosphate (dGTP), deoxyadenosine diphosphate (dADP), thymidine diphosphate (dTDP), deoxycytidine diphosphate (dCDP), deoxyguanosine diphosphate (dGDP), deoxyadenosine monophosphate (dAMP), deoxythymidine monophosphate (dTMP), deoxycytidine monophosphate (dCMP), and deoxyguanosine monophosphate (dGMP). Exemplary natural deoxyribonucleotides, which comprise a deoxyribose as the sugar moiety, include dATP, dTTP, dCTP, dGTP, dADP, dTDP, dCDP, dGDP, dAMP, dTMP, dCMP, and dGMP. Exemplary natural ribonucleotides, which comprise a ribose as the sugar moiety, include ATP, UTP, CTP, GTP, ADP, UDP, CDP, GDP, AMP, UMP, CMP, and GMP.

As used herein, “base” or “nucleobase” refers to at least the nucleobase portion of a nucleoside or nucleotide (nucleoside and nucleotide encompass the ribo or deoxyribo variants), which may in some cases contain further modifications to the sugar portion of the nucleoside or nucleotide. In some cases, “base” is also used to represent the entire nucleoside or nucleotide (for example, a “base” may be incorporated by a DNA polymerase into DNA, or by an RNA polymerase into RNA). However, the term “base” should not be interpreted as necessarily representing the entire nucleoside or nucleotide unless required by the context. In the chemical structures provided herein of a base or nucleobase, only the base of the nucleoside or nucleotide is shown, with the sugar moiety and, optionally, any phosphate residues omitted for clarity. As used in the chemical structures provided herein of a base or nucleobase, the wavy line represents connection to a nucleoside or nucleotide, in which the sugar portion of the nucleoside or nucleotide may be further modified. In some embodiments, the wavy line represents attachment of the base or nucleobase to the sugar portion, such as a pentose, of the nucleoside or nucleotide. In some embodiments, the pentose is a ribose or a deoxyribose.

In some embodiments, a nucleobase is generally the heterocyclic base portion of a nucleoside. Nucleobases may be naturally occurring, may be modified, may bear no similarity to natural bases, and/or may be synthesized, e.g., by organic synthesis. In certain embodiments, a nucleobase comprises any atom or group of atoms in a nucleoside or nucleotide, where the atom or group of atoms is capable of interacting with a base of another nucleic acid with or without the use of hydrogen bonds. In certain embodiments, an unnatural nucleobase is not derived from a natural nucleobase. It should be noted that unnatural nucleobases do not necessarily possess basic properties, however, they are referred to as nucleobases for simplicity. In some embodiments, when referring to a nucleobase, a “(d)” indicates that the nucleobase can be attached to a deoxyribose or a ribose, while “d” without parentheses indicates that the nucleobase is attached to deoxyribose.

As used herein, a “nucleoside” is a compound comprising a nucleobase moiety and a sugar moiety. Nucleosides include, but are not limited to, naturally occurring nucleosides (as found in DNA and RNA), abasic nucleosides, modified nucleosides, and nucleosides having mimetic bases and/or sugar groups. Nucleosides include nucleosides comprising any variety of substituents. A nucleoside can be a glycoside compound formed through glycosidic linking between a nucleic acid base and a reducing group of a sugar.

An “analog” of a chemical structure, as the term is used herein, refers to a chemical structure that preserves substantial similarity with the parent structure, although it may not be readily derived synthetically from the parent structure. In some embodiments, a nucleotide analog is an unnatural nucleotide. In some embodiments, a nucleoside analog is an unnatural nucleoside. A related chemical structure that is readily derived synthetically from a parent chemical structure is referred to as a “derivative.”

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Modified IL-10 Polypeptides

Described herein, in some embodiments, are IL-10 polypeptides modified at an amino acid position. In some instances, the modification is to a natural amino acid. In some instances, the modification is to an unnatural amino acid. In some instances, described herein is an isolated and modified IL-10 polypeptide that comprises at least one unnatural amino acid. In some instances, the modified IL-10 polypeptide is an isolated and purified mammalian IL-10, for example, a rodent IL-10 protein, or a human IL-10 protein. In some cases, the modified IL-10 polypeptide is a human IL-10 protein. In some embodiments, the modified IL-polypeptide is modified from a parental IL-10 sequence. In some cases, the parental IL-10 sequence is a wild-type IL-10 sequence. In some cases, the parental IL-10 sequence is SEQ ID NO: 1. In some embodiments, the modified IL-10 polypeptides as described herein comprise an optional methionine at the N-terminus as depicted by (M) of SEQ ID NOS: 1 and 3-73. In some embodiments, the modified IL-10 polypeptides comprise a methionine at the N-terminus of the wild-type or parental IL-10 sequence followed by the serine. In some instances, the modified IL-10 polypeptides comprise the serine at the N-terminus of the wild-type or parental IL-10 sequence. In some embodiments, the modified IL-10 polypeptides comprise a methionine substituting and replacing the serine at the N-terminus of the wild-type or parental IL-10 sequence. In some embodiments, the modified IL-10 polypeptides comprise a methionine at the N-terminus followed by the serine as depicted by (M) of SEQ ID NO: 1. In some instances, the modified IL-10 polypeptides comprise the serine at the N-terminus of SEQ ID NO: 1. In some embodiments, the modified IL-10 polypeptides comprise a methionine substituting and replacing the serine at the N-terminus as depicted by (M) of SEQ ID NO: 1. In some cases, the parental IL-10 sequence is SEQ ID NO: 2.

In some cases, the modified IL-10 polypeptide comprises about 80%, 85%, 90%, 95%, 96%, 97%, 98%, or 99% sequence identity to SEQ ID NO: 1. In some cases, the modified IL-10 polypeptide comprises about 80% sequence identity to SEQ ID NO: 1. In some cases, the modified IL-10 polypeptide comprises about 85% sequence identity to SEQ ID NO: 1. In some cases, the modified IL-10 polypeptide comprises about 90% sequence identity to SEQ ID NO: 1. In some cases, the modified IL-10 polypeptide comprises about 95% sequence identity to SEQ ID NO: 1. In some cases, the modified IL-10 polypeptide comprises about 96% sequence identity to SEQ ID NO: 1. In some cases, the modified IL-10 polypeptide comprises about 97% sequence identity to SEQ ID NO: 1. In some cases, the modified IL-10 polypeptide comprises about 98% sequence identity to SEQ ID NO: 1. In some cases, the modified IL-10 polypeptide comprises about 99% sequence identity to SEQ ID NO: 1. In some cases, the modified IL-10 polypeptide comprises the sequence of SEQ ID NO: 1. In some cases, the modified IL-10 polypeptide consists of the sequence of SEQ ID NO: 1. In additional cases, the modified IL-10 polypeptide comprises about 80%, 85%, 90%, 95%, 96%, 97%, 98%, or 99% sequence identity to SEQ ID NO: 2. In additional cases, the modified IL-10 polypeptide comprises the sequence of SEQ ID NO: 2. In additional cases, the modified IL-10 polypeptide consists of the sequence of SEQ ID NO: 2.

In some instances, the modified IL-10 polypeptide is a truncated variant. In some instances, the truncation is an N-terminal deletion. In other instances, the truncation is a C-terminal deletion. In additional instances, the truncation comprises both N-terminal and C-terminal deletions. For example, the truncation can be a deletion of at least or about 1, 2, 3, 4, 5, 6, 7, 8, 9, 10, 11, 12, 13, 14, 15, 20, or more residues from either the N-terminus or the C-terminus, or both termini. In some cases, the modified IL-10 polypeptide comprises an N-terminal deletion of at least or about 1, 2, 3, 4, 5, 6, 7, 8, 9, 10, 11, 12, 13, 14, 15, 20, or more residues. In some cases, the modified IL-10 polypeptide comprises an N-terminal deletion of at least or about 1, 2, 3, 4, 5, 6, 7, 8, 9, or 10 residues. In some cases, the modified IL-10 polypeptide comprises an N-terminal deletion of at least or about 2 residues. In some cases, the modified IL-10 polypeptide comprises an N-terminal deletion of at least or about 3 residues. In some cases, the modified IL-10 polypeptide comprises an N-terminal deletion of at least or about 4 residues. In some cases, the modified IL-10 polypeptide comprises an N-terminal deletion of at least or about 5 residues. In some cases, the modified IL-10 polypeptide comprises an N-terminal deletion of at least or about 6 residues. In some cases, the modified IL-10 polypeptide comprises an N-terminal deletion of at least or about 7 residues. In some cases, the modified IL-10 polypeptide comprises an N-terminal deletion of at least or about 8 residues. In some cases, the modified IL-10 polypeptide comprises an N-terminal deletion of at least or about 9 residues. In some cases, the modified IL-10 polypeptide comprises an N-terminal deletion of at least or about 10 residues.

In some embodiments, the modified IL-10 polypeptide is a functionally active fragment. In some cases, the functionally active fragment comprises IL-10 region 5-160, 10-160, 15-160, 20-160, 1-155, 5-155, 10-155, 15-155, 20-155, 1-150, 5-150, 10-150, 15-150, or 20-150, wherein the residue positions are in reference to the positions in SEQ ID NO: 1. In some instances, the functionally active fragment comprises IL-10 region 5-160, wherein the residue positions are in reference to the positions in SEQ ID NO: 1. In some instances, the functionally active fragment comprises IL-10 region 10-160, wherein the residue positions are in reference to the positions in SEQ ID NO: 1. In some instances, the functionally active fragment comprises IL-10 region 15-160, wherein the residue positions are in reference to the positions in SEQ ID NO: 1. In some instances, the functionally active fragment comprises IL-10 region 20-160, wherein the residue positions are in reference to the positions in SEQ ID NO: 1. In some instances, the functionally active fragment comprises IL-10 region 1-155, wherein the residue positions are in reference to the positions in SEQ ID NO: 1. In some instances, the functionally active fragment comprises IL-10 region 5-155, wherein the residue positions are in reference to the positions in SEQ ID NO: 1. In some instances, the functionally active fragment comprises IL-10 region 10-155, wherein the residue positions are in reference to the positions in SEQ ID NO: 1. In some instances, the functionally active fragment comprises IL-10 region 15-155, wherein the residue positions are in reference to the positions in SEQ ID NO: 1. In some instances, the functionally active fragment comprises IL-10 region 20-155, wherein the residue positions are in reference to the positions in SEQ ID NO: 1. In some instances, the functionally active fragment comprises IL-10 region 1-150, wherein the residue positions are in reference to the positions in SEQ ID NO: 1. In some instances, the functionally active fragment comprises IL-10 region 5-150, wherein the residue positions are in reference to the positions in SEQ ID NO: 1. In some instances, the functionally active fragment comprises IL-10 region 10-150, wherein the residue positions are in reference to the positions in SEQ ID NO: 1. In some instances, the functionally active fragment comprises IL-10 region 15-150, wherein the residue positions are in reference to the positions in SEQ ID NO: 1. In some instances, the functionally active fragment comprises IL-10 region 20-150, wherein the residue positions are in reference to the positions in SEQ ID NO: 1.

In some embodiments, described herein is an IL-10 polypeptide which comprises at least one unnatural amino acid. In some instances, the at least one unnatural amino acid is located in helix C, D, or E. In some cases, helix C comprises residues L60-N82, in which the positions are in reference to the positions in SEQ ID NO: 1. In some cases, helix D comprises residues I87-C108, in which the positions are in reference to the positions in SEQ ID NO: 1. In some cases, helix E comprises residues S118-L131, in which the positions are in reference to the positions in SEQ ID NO: 1. In some cases, the at least one unnatural amino acid is located at a surface exposed location in helix C, D, or E.

In some embodiments, described herein is a modified IL-10 polypeptide which comprises at least one unnatural amino acid at a position selected from E67, Q70, E74, E75, Q79, N82, K88, A89, K99, K125, N126, N129, K130, or Q132, wherein the residue positions correspond to positions 67, 70, 74, 75, 79, 82, 88, 89, 99, 125, 126, 129, 130, and 132 as set forth in SEQ ID NO: 1. In some instances, the position of the at least one unnatural amino acid is selected from E67, Q70, E74, E75, Q79, or N82, wherein the residue positions correspond to positions 67, 70, 74, 75, 79, and 82 as set forth in SEQ ID NO: 1. In some instances, the position of the at least one unnatural amino acid is selected from K88, A89, K99, K125, N126, N129, K130, or Q132, wherein the residue positions correspond to positions 88, 89, 99, 125, 126, 129, 130, and 132 as set forth in SEQ ID NO: 1. In some instances, the position of the at least one unnatural amino acid is selected from K125, N126, N129, K130, or Q132, wherein the residue positions correspond to positions 125, 126, 129, 130, and 132 as set forth in SEQ ID NO: 1. In some instances, the position of the at least one unnatural amino acid is selected from E67, Q70, E74, E75, Q79, N82, K88, A89, K99, K125, N126, N129, K130, or Q132, wherein the residue positions correspond to positions 67, 70, 74, 75, 79, 82, 88, 89, 99, 125, 126, 129, 130, and 132 as set forth in SEQ ID NO: 1. In some instances, the position of the at least one unnatural amino acid is E67. In some instances, the position of the at least one unnatural amino acid is Q70. In some instances, the position of the at least one unnatural amino acid is E74. In some instances, the position of the at least one unnatural amino acid is E75. In some instances, the position of the at least one unnatural amino acid is Q79. In some instances, the position of the at least one unnatural amino acid is N82. In some instances, the position of the at least one unnatural amino acid is K88. In some instances, the position of the at least one unnatural amino acid is A89. In some instances, the position of the at least one unnatural amino acid is K99. In some instances, the position of the at least one unnatural amino acid is K125. In some instances, the position of the at least one unnatural amino acid is N126. In some instances, the position of the at least one unnatural amino acid is N129. In some instances, the position of the at least one unnatural amino acid is K130. In some instances, the position of the at least one unnatural amino acid is Q132.

In some embodiments, described herein are IL-10 polypeptides modified at an amino acid position. In some instances, the modification is to a natural amino acid. In some instances, the modification is to an unnatural amino acid. In some instances, described herein is an isolated and modified IL-10 polypeptide that comprises at least one unnatural amino acid. In some instances, the modified IL-10 polypeptide is an isolated and purified mammalian IL-10, for example, a rodent IL-10 protein, or a human IL-10 protein. In some cases, the modified IL-10 polypeptide is a human IL-10 protein.

In some cases, the modified IL-10 polypeptide comprises about 80%, 85%, 90%, 95%, 96%, 97%, 98%, or 99% sequence identity to SEQ ID NO: 1. In some cases, the modified IL-10 polypeptide comprises the sequence of SEQ ID NO: 1. In some cases, the modified IL-10 polypeptide consists of the sequence of SEQ ID NO: 1. In some cases, the modified IL-10 polypeptide comprises about 80%, 85%, 90%, 95%, 96%, 97%, 98%, or 99% sequence identity to SEQ ID NO: 3. In some cases, the modified IL-10 polypeptide comprises the sequence of SEQ ID NO: 3. In some cases, the modified IL-10 polypeptide consists of the sequence of SEQ ID NO: 3. In additional cases, the modified IL-10 polypeptide comprises about 80%, 85%, 90%, 95%, 96%, 97%, 98%, or 99% sequence identity to SEQ ID NO: 4. In additional cases, the modified IL-10 polypeptide comprises the sequence of SEQ ID NO: 4. In additional cases, the modified IL-10 polypeptide consists of the sequence of SEQ ID NO: 4. In some cases, the modified IL-10 polypeptide comprises about 80%, 85%, 90%, 95%, 96%, 97%, 98%, or 99% sequence identity to SEQ ID NO: 5. In some cases, the modified IL-10 polypeptide comprises the sequence of SEQ ID NO: 5. In some cases, the modified IL-10 polypeptide consists of the sequence of SEQ ID NO: 5. In additional cases, the modified IL-10 polypeptide comprises about 80%, 85%, 90%, 95%, 96%, 97%, 98%, or 99% sequence identity to SEQ ID NO: 6. In additional cases, the modified IL-10 polypeptide comprises the sequence of SEQ ID NO: 6. In additional cases, the modified IL-10 polypeptide consists of the sequence of SEQ ID NO: 6. In some cases, the modified IL-10 polypeptide comprises about 80%, 85%, 90%, 95%, 96%, 97%, 98%, or 99% sequence identity to SEQ ID NO: 7. In some cases, the modified IL-10 polypeptide comprises the sequence of SEQ ID NO: 7. In some cases, the modified IL-10 polypeptide consists of the sequence of SEQ ID NO: 7. In additional cases, the modified IL-10 polypeptide comprises about 80%, 85%, 90%, 95%, 96%, 97%, 98%, or 99% sequence identity to SEQ ID NO: 8. In additional cases, the modified IL-10 polypeptide comprises the sequence of SEQ ID NO: 8. In additional cases, the modified IL-10 polypeptide consists of the sequence of SEQ ID NO: 8. In some cases, the modified IL-10 polypeptide comprises about 80%, 85%, 90%, 95%, 96%, 97%, 98%, or 99% sequence identity to SEQ ID NO: 9. In some cases, the modified IL-10 polypeptide comprises the sequence of SEQ ID NO: 9. In some cases, the modified IL-10 polypeptide consists of the sequence of SEQ ID NO: 9. In some cases, the modified IL-10 polypeptide comprises about 80%, 85%, 90%, 95%, 96%, 97%, 98%, or 99% sequence identity to SEQ ID NO: 10. In some cases, the modified IL-10 polypeptide comprises the sequence of SEQ ID NO: 10. In some cases, the modified IL-10 polypeptide consists of the sequence of SEQ ID NO: 10. In some cases, the modified IL-10 polypeptide comprises about 80%, 85%, 90%, 95%, 96%, 97%, 98%, or 99% sequence identity to SEQ ID NO: 11. In some cases, the modified IL-10 polypeptide comprises the sequence of SEQ ID NO: 11. In some cases, the modified IL-10 polypeptide consists of the sequence of SEQ ID NO: 11. In some cases, the modified IL-10 polypeptide comprises about 80%, 85%, 90%, 95%, 96%, 97%, 98%, or 99% sequence identity to SEQ ID NO: 12. In some cases, the modified IL-10 polypeptide comprises the sequence of SEQ ID NO: 12. In some cases, the modified IL-10 polypeptide consists of the sequence of SEQ ID NO: 12. In some cases, the modified IL-10 polypeptide comprises about 80%, 85%, 90%, 95%, 96%, 97%, 98%, or 99% sequence identity to SEQ ID NO: 13. In some cases, the modified IL-10 polypeptide comprises the sequence of SEQ ID NO: 13. In some cases, the modified IL-10 polypeptide consists of the sequence of SEQ ID NO: 13. In some cases, the modified IL-10 polypeptide comprises about 80%, 85%, 90%, 95%, 96%, 97%, 98%, or 99% sequence identity to SEQ ID NO: 14. In some cases, the modified IL-10 polypeptide comprises the sequence of SEQ ID NO: 14. In some cases, the modified IL-10 polypeptide consists of the sequence of SEQ ID NO: 14. In some cases, the modified IL-10 polypeptide comprises about 80%, 85%, 90%, 95%, 96%, 97%, 98%, or 99% sequence identity to SEQ ID NO: 15. In some cases, the modified IL-10 polypeptide comprises the sequence of SEQ ID NO: 15. In some cases, the modified IL-10 polypeptide consists of the sequence of SEQ ID NO: 15. In some cases, the modified IL-10 polypeptide comprises about 80%, 85%, 90%, 95%, 96%, 97%, 98%, or 99% sequence identity to SEQ ID NO: 16. In some cases, the modified IL-10 polypeptide comprises the sequence of SEQ ID NO: 16. In some cases, the modified IL-10 polypeptide consists of the sequence of SEQ ID NO: 16. In some cases, the modified IL-10 polypeptide comprises about 80%, 85%, 90%, 95%, 96%, 97%, 98%, or 99% sequence identity to SEQ ID NO: 17. In some cases, the modified IL-10 polypeptide comprises the sequence of SEQ ID NO: 17. In some cases, the modified IL-10 polypeptide consists of the sequence of SEQ ID NO: 17. In some cases, the modified IL-10 polypeptide comprises about 80%, 85%, 90%, 95%, 96%, 97%, 98%, or 99% sequence identity to SEQ ID NO: 18. In some cases, the modified IL-10 polypeptide comprises the sequence of SEQ ID NO: 18. In some cases, the modified IL-10 polypeptide consists of the sequence of SEQ ID NO: 18.

In some instances, the at least one unnatural amino acid is located proximal to the N-terminus. As used herein, proximal refers to a residue located at least 1 residue away from the N-terminal residue and up to about 50 residues away from the N-terminal residue. In some cases, the at least one unnatural amino acid is located within the first 10, 20, 30, 40, or 50 residues from the N-terminal residue. In some cases, the at least one unnatural amino acid is located within the first 10 residues from the N-terminal residue. In some cases, the at least one unnatural amino acid is located within the first 20 residues from the N-terminal residue. In some cases, the at least one unnatural amino acid is located within the first 30 residues from the N-terminal residue. In some cases, the at least one unnatural amino acid is located within the first 40 residues from the N-terminal residue. In some cases, the at least one unnatural amino acid is located within the first 50 residues from the N-terminal residue.

In some instances, the at least one unnatural amino acid is the N-terminal residue.

In some instances, the at least one unnatural amino acid is located proximal to the C-terminus. As used herein, proximal refers to a residue located at least 1 residue away from the C-terminal residue and up to about 50 residues away from the C-terminal residue. In some cases, the at least one unnatural amino acid is located within the first 10, 20, 30, 40, or 50 residues from the C-terminal residue. In some cases, the at least one unnatural amino acid is located within the first 10 residues from the C-terminal residue. In some cases, the at least one unnatural amino acid is located within the first 20 residues from the C-terminal residue. In some cases, the at least one unnatural amino acid is located within the first 30 residues from the C-terminal residue. In some cases, the at least one unnatural amino acid is located within the first 40 residues from the C-terminal residue. In some cases, the at least one unnatural amino acid is located within the first 50 residues from the C-terminal residue.

In some instances, the at least one unnatural amino acid is the C-terminal residue.

In some embodiments, the modified IL-10 polypeptide is a functionally active monomer or a functionally active dimer that is capable of binding to the IL-10R and activates the signaling pathway. In some cases, the functionally active modified IL-10 monomer or dimer has an enhanced plasma half-life. In some cases, the enhanced plasma half-life is compared to a plasma half-life of a wild-type IL-10 protein. In some cases, the enhanced plasma half-life of the modified IL-10 polypeptide is at least 90 minutes, 2 hours, 3 hours, 4 hours, 5 hours, 6 hours, 7 hours, 8 hours, 9 hours, 10 hours, 11 hours, 12 hours, 18 hours, 24 hours, 36 hours, 48 hours, 3 days, 4 days, 5 days, 6 days, 7 days, 10 days, 12 days, 14 days, 21 days, 28 days, 30 days, or longer than the plasma half-life of the wild-type IL-10 protein. In some cases, the enhanced plasma half-life of the modified IL-10 polypeptide is about 90 minutes, 2 hours, 3 hours, 4 hours, 5 hours, 6 hours, 7 hours, 8 hours, 9 hours, 10 hours, 11 hours, 12 hours, 18 hours, 24 hours, 36 hours, 48 hours, 3 days, 4 days, 5 days, 6 days, 7 days, 10 days, 12 days, 14 days, 21 days, 28 days, or 30 days compared to the plasma half-life of the wild-type IL-10 protein.

In some embodiments, the modified IL-10 monomer or dimer has a plasma half-life that is capable of proliferating and/or expanding tumor infiltration lymphocytes (TILs), T cells, B cells, natural killer cells, macrophages, neutrophils, dendritic cells, mast cells, eosinophils basophils, or CD4+ or CD8+ T cells.

In some embodiments, the modified IL-10 monomer or dimer is administered to a subject. In some embodiments, the modified IL-10 monomer or dimer administered to the subject comprises a reduced toxicity compared to a toxicity of the wild-type IL-10 administered to the subject. In some embodiments, the modified IL-10 monomer or dimer comprises the reduced toxicity that is at least 1-fold, 2-fold, 3-fold, 4-fold, 5-fold, 6-fold, 7-fold, 8-fold, 9-fold, 10-fold, 20-fold, 30-fold, 50-fold, 100-fold, or more reduced relative to the wild type IL-10 dimer. In some cases, the reduced toxicity is at least 10%, 20%, 30%, 40%, 50%, 60%, 70%, 80%, 90%, 100%, 200%, 300%, 400%, 500%, or more reduced relative to the wild-type IL-10 protein.

In some embodiments, the modified IL-10 monomer or dimer is administered to a subject. In some embodiments, the modified IL-10 monomer or dimer administered to the subject does not cause grade 3 or grade 4 adverse events. In some embodiments, the modified IL-10 monomer or dimer administered to the subject comprises a reduced occurrence or severity of grade 3 or grade 4 adverse events compared to an occurrence or severity of grade 3 or grade 4 adverse events caused by the administering the wild-type IL-10 protein to the subject. Exemplary grade 3 and grade 4 adverse events include anemia, leukopenia, thrombocytopenia, increased ALT, anorexia, arthralgia, back pain, chills, diarrhea, dyslipidemia, fatigue, fever, flu-like symptoms, hypoalbuminemia, increased lipase, injection site reaction, myalgia, nausea, night sweats, pruritis, rash, erythematous rash, maculopapular rash, transaminitis, vomiting, and weakness.

In some embodiments, the modified IL-10 monomer or dimer decreases the occurrence of the grade 3 or grade 4 adverse events in the subject by about 10%, 20%, 30%, 40%, 50%, 60%, 70%, 80%, 90%, 95%, 99%, or about 100%, relative to administering the wild-type IL-10 protein to the subject. In some instances, the modified IL-10 monomer or dimer decreases the severity of grade 3 or grade 4 adverse events in the subject by about 10%, 20%, 30%, 40%, 50%, 60%, 70%, 80%, 90%, 95%, 99%, or about 100%, relative to administering the wild-type IL-10 protein to the subject.

In some embodiments, the modified IL-10 monomer or dimer as described herein comprises a decreased affinity to the IL-10R compared to an affinity of wild-type IL-10 protein to the IL-10R. In some embodiments, the affinity of the modified IL-10 monomer or dimer to IL-10R compared to the affinity of the wild-type IL-10 protein to IL-10R is decreased about 10%, 20%, 30%, 40%, 50%, 60%, 70%, 80%, 90%, 95%, or 99%, or greater than 99%. In some cases, the decreased affinity is about 10%. In some cases, the decreased affinity is about 20%. In some cases, the decreased affinity is about 30%. In some cases, the decreased affinity is about 40%. In some cases, the decreased affinity is about 50%. In some cases, the decreased affinity is about 60%. In some cases, the decreased affinity is about 70%. In some cases, the decreased affinity is about 80%. In some cases, the decreased affinity is about 90%. In some cases, the decreased affinity is about 95%. In some cases, the decreased affinity is about 99%. In some cases, the decreased affinity is about 100%.

In some embodiments, the decreased affinity of the modified IL-10 monomer or dimer compared to the wild-type IL-10 protein is about 1-fold, 2-fold, 3-fold, 4-fold, 5-fold, 6-fold, 7-fold, 8-fold, 9-fold, 10-fold, 30-fold, 50-fold, 100-fold, 200-fold, 300-fold, 400-fold, 500-fold, 1,000-fold, or more. In some cases, the decreased affinity is about 1-fold. In some cases, the decreased affinity is about 2-fold. In some cases, the decreased affinity is about 3-fold. In some cases, the decreased affinity is about 4-fold. In some cases, the decreased affinity is about 5-fold. In some cases, the decreased affinity is about 6-fold. In some cases, the decreased affinity is about 7-fold. In some cases, the decreased affinity is about 8-fold. In some cases, the decreased affinity is about 9-fold. In some cases, the decreased affinity is about 10-fold. In some cases, the decreased affinity is about 30-fold. In some cases, the decreased affinity is about 50-fold. In some cases, the decreased affinity is about 100-fold. In some cases, the decreased affinity is about 200-fold. In some cases, the decreased affinity is about 300-fold. In some cases, the decreased affinity is about 400-fold. In some cases, the decreased affinity is about 500-fold. In some cases, the decreased affinity is about 1000-fold. In some cases, the decreased affinity is more than 1,000-fold.

In some cases, the modified IL-10 monomer or dimer does not interact with IL-10R. In some cases, the modified IL-10 monomer or dimer has about the same affinity to IL-10R as the affinity of the wild-type IL-10 to IL-10R.

In some embodiments, the modified IL-10 monomer or dimer as described herein comprises an increased affinity to the IL-10R compared to an affinity of wild-type IL-10 protein to the IL-10R. In some embodiments, the affinity of the modified IL-10 monomer or dimer to the IL-10R compared to the affinity of the wild-type IL-10 protein to IL-10R is increased about 10%, 20%, 30%, 40%, 50%, 60%, 70%, 80%, 90%, 95%, or 99%, or greater than 99%. In some cases, the increased affinity is about 10%. In some cases, the increased affinity is about 20%. In some cases, the increased affinity is about 30%. In some cases, the increased affinity is about 40%. In some cases, the increased affinity is about 50%. In some cases, the increased affinity is about 60%. In some cases, the increased affinity is about 70%. In some cases, the increased affinity is about 80%. In some cases, the increased affinity is about 90%. In some cases, the increased affinity is about 95%. In some cases, the increased affinity is about 99%. In some cases, the increased affinity is about 100%.

In some embodiments, the increased affinity of the modified IL-10 monomer or dimer compared to the wild-type IL-10 protein is about 1-fold, 2-fold, 3-fold, 4-fold, 5-fold, 6-fold, 7-fold, 8-fold, 9-fold, 10-fold, 30-fold, 50-fold, 100-fold, 200-fold, 300-fold, 400-fold, 500-fold, 1,000-fold, or more. In some cases, the increased affinity is about 1-fold. In some cases, the increased affinity is about 2-fold. In some cases, the increased affinity is about 3-fold. In some cases, the increased affinity is about 4-fold. In some cases, the increased affinity is about 5-fold. In some cases, the increased affinity is about 6-fold. In some cases, the increased affinity is about 7-fold. In some cases, the increased affinity is about 8-fold. In some cases, the increased affinity is about 9-fold. In some cases, the increased affinity is about 10-fold. In some cases, the increased affinity is about 30-fold. In some cases, the increased affinity is about 50-fold. In some cases, the increased affinity is about 100-fold. In some cases, the increased affinity is about 200-fold. In some cases, the increased affinity is about 300-fold. In some cases, the increased affinity is about 400-fold. In some cases, the increased affinity is about 500-fold. In some cases, the increased affinity is about 1000-fold. In some cases, the increased affinity is more than 1,000-fold.

In some instances, IL-10R signaling potency as mediated by IL-10 is measured by a decreased half maximal effective concentration (EC50). In some embodiments, the EC50 of the modified IL-10 monomer or dimer is decreased compared to EC50 of the wild-type IL-10 protein. In some embodiments, the decreased EC50 of the modified IL-10 monomer or dimer is about 10%, 20%, 30%, 40%, 50%, 60%, 70%, 80%, 90%, 95%, or 99%, or greater than 99%. In some cases, the EC50 of the modified IL-10 monomer or dimer is decreased about 10%. In some cases, the EC50 of the modified IL-10 monomer or dimer is decreased about 20%. In some cases, the EC50 of the modified IL-10 monomer or dimer is decreased about 30%. In some cases, the EC50 of the modified IL-10 monomer or dimer is decreased about 40%. In some cases, the EC50 of the modified IL-10 monomer or dimer is decreased about 50%. In some cases, the EC50 of the modified IL-10 monomer or dimer is decreased about 60%. In some cases, the EC50 of the modified IL-10 monomer or dimer is decreased about 70%. In some cases, the EC50 of the modified IL-10 monomer or dimer is decreased about 80%. In some cases, the EC50 of the modified IL-10 monomer or dimer is decreased about 90%. In some cases, the EC50 of the modified IL-10 monomer or dimer is decreased about 95%. In some cases, the EC50 of the modified IL-10 monomer or dimer is decreased about 99%. In some cases, the EC50 of the modified IL-10 monomer or dimer is decreased about 100%.

In some embodiments, the decreased EC50 of the modified IL-10 monomer or dimer compared to the wild-type IL-10 protein is about 1-fold, 2-fold, 3-fold, 4-fold, 5-fold, 6-fold, 7-fold, 8-fold, 9-fold, 10-fold, 30-fold, 50-fold, 100-fold, 200-fold, 300-fold, 400-fold, 500-fold, 1,000-fold, or more. In some cases, the EC50 of the modified IL-10 monomer or dimer is decreased about 1-fold. In some cases, the EC50 of the modified IL-10 monomer or dimer is decreased about 2-fold. In some cases, the EC50 of the modified IL-10 monomer or dimer is decreased about 3-fold. In some cases, the EC50 of the modified IL-10 monomer or dimer is decreased about 4-fold. In some cases, the EC50 of the modified IL-10 monomer or dimer is decreased about 5-fold. In some cases, the EC50 of the modified IL-10 monomer or dimer is decreased about 6-fold. In some cases, the EC50 of the modified IL-10 monomer or dimer is decreased about 7-fold. In some cases, the EC50 of the modified IL-10 monomer or dimer is decreased about 8-fold. In some cases, the EC50 of the modified IL-10 monomer or dimer is decreased about 9-fold. In some cases, the EC50 of the modified IL-10 monomer or dimer is decreased about 10-fold. In some cases, the EC50 of the modified IL-10 monomer or dimer is decreased about 30-fold. In some cases, the EC50 of the modified IL-10 monomer or dimer is decreased about 50-fold. In some cases, the EC50 of the modified IL-10 monomer or dimer is decreased about 100-fold. In some cases, the EC50 of the modified IL-10 monomer or dimer is decreased about 200-fold. In some cases, the EC50 of the modified IL-10 monomer or dimer is decreased about 300-fold. In some cases, the EC50 of the modified IL-10 monomer or dimer is decreased about 400-fold. In some cases, the EC50 of the modified IL-10 monomer or dimer is decreased about 500-fold. In some cases, the EC50 of the modified IL-10 monomer or dimer is decreased about 1000-fold. In some cases, the EC50 of the modified IL-10 monomer or dimer is decreased more than 1,000-fold.

In some cases, the EC50 of the modified IL-10 monomer or dimer is about the same as the EC50 of the wild-type IL-10 protein.

In some instances, the modified IL-10 monomer or dimer as described herein has an increased EC50 compared to EC50 of the wild-type IL-10 protein in activating IL-10R signaling. In some embodiments, the increased EC50 of the modified IL-10 monomer or dimer is about 10%, 20%, 30%, 40%, 50%, 60%, 70%, 80%, 90%, 95%, or 99%, or greater than 99%. In some cases, the EC50 of the modified IL-10 monomer or dimer is increased about 10%. In some cases, the EC50 of the modified IL-10 monomer or dimer is increased about 20%. In some cases, the EC50 of the modified IL-10 monomer or dimer is increased about 30%. In some cases, the EC50 of the modified IL-10 monomer or dimer is increased about 40%. In some cases, the EC50 of the modified IL-10 monomer or dimer is increased about 50%. In some cases, the EC50 of the modified IL-10 monomer or dimer is increased about 60%. In some cases, the EC50 of the modified IL-10 monomer or dimer is increased about 70%. In some cases, the EC50 of the modified IL-10 monomer or dimer is increased about 80%. In some cases, the EC50 of the modified IL-10 monomer or dimer is increased about 90%. In some cases, the EC50 of the modified IL-10 monomer or dimer is increased about 95%. In some cases, the EC50 of the modified IL-10 monomer or dimer is increased about 99%. In some cases, the EC50 of the modified IL-10 monomer or dimer is increased about 100%.

In some embodiments, the increased EC50 of the modified IL-10 monomer or dimer compared to the EC50 of the wild-type IL-10 protein is about 1-fold, 2-fold, 3-fold, 4-fold, 5-fold, 6-fold, 7-fold, 8-fold, 9-fold, 10-fold, 30-fold, 50-fold, 100-fold, 200-fold, 300-fold, 400-fold, 500-fold, 1,000-fold, or more. In some cases, the EC50 of the modified IL-10 monomer or dimer is increased about 1-fold. In some cases, the EC50 of the modified IL-10 monomer or dimer is increased about 2-fold. In some cases, the EC50 of the modified IL-10 monomer or dimer is increased about 3-fold. In some cases, the EC50 of the modified IL-10 monomer or dimer is increased about 4-fold. In some cases, the EC50 of the modified IL-10 monomer or dimer is increased about 5-fold. In some cases, the EC50 of the modified IL-10 monomer or dimer is increased about 6-fold. In some cases, the EC50 of the modified IL-10 monomer or dimer is increased about 7-fold. In some cases, the EC50 of the modified IL-10 monomer or dimer is increased about 8-fold. In some cases, the EC50 of the modified IL-10 monomer or dimer is increased about 9-fold. In some cases, the EC50 of the modified IL-10 monomer or dimer is increased about 10-fold. In some cases, the EC50 of the modified IL-10 monomer or dimer is increased about 30-fold. In some cases, the EC50 of the modified IL-10 monomer or dimer is increased about 50-fold. In some cases, the EC50 of the modified IL-10 monomer or dimer is increased about 100-fold. In some cases, the EC50 of the modified IL-10 monomer or dimer is increased about 200-fold. In some cases, the EC50 of the modified IL-10 monomer or dimer is increased about 300-fold. In some cases, the EC50 of the modified IL-10 monomer or dimer is increased about 400-fold. In some cases, the EC50 of the modified IL-10 monomer or dimer is increased about 500-fold. In some cases, the EC50 of the modified IL-10 monomer or dimer is increased about 1000-fold. In some cases, the EC50 of the modified IL-10 monomer or dimer is increased more than 1,000-fold.

In some instances, IL-10R signaling potency as mediated by IL-10 is measured by a median effective dose (ED50). In some embodiments, the modified IL-10 monomer or dimer as described herein has a decreased ED50 compared to an ED50 of the wild-type IL-10 protein. In some embodiments, the decreased ED50 of the modified IL-10 monomer or dimer is about 10%, 20%, 30%, 40%, 50%, 60%, 70%, 80%, 90%, 95%, or 99%, or greater than 99%. In some cases, the ED50 of the modified IL-10 monomer or dimer is decreased about 10%. In some cases, the ED50 of the modified IL-10 monomer or dimer is decreased about 20%. In some cases, the ED50 of the modified IL-10 monomer or dimer is decreased about 30%. In some cases, the ED50 of the modified IL-10 monomer or dimer is decreased about 40%. In some cases, the ED50 of the modified IL-10 monomer or dimer is decreased about 50%. In some cases, the ED50 of the modified IL-10 monomer or dimer is decreased about 60%. In some cases, the ED50 of the modified IL-10 monomer or dimer is decreased about 70%. In some cases, the ED50 of the modified IL-10 monomer or dimer is decreased about 80%. In some cases, the ED50 of the modified IL-10 monomer or dimer is decreased about 90%. In some cases, the ED50 of the modified IL-10 monomer or dimer is decreased about 95%. In some cases, the ED50 of the modified IL-10 monomer or dimer is decreased about 99%. In some cases, the ED50 of the modified IL-10 monomer or dimer is decreased about 100%.

In some embodiments, the decreased ED50 of the modified IL-10 monomer or dimer compared to the ED50 of the wild-type IL-10 protein is about 1-fold, 2-fold, 3-fold, 4-fold, 5-fold, 6-fold, 7-fold, 8-fold, 9-fold, 10-fold, 30-fold, 50-fold, 100-fold, 200-fold, 300-fold, 400-fold, 500-fold, 1,000-fold, or more. In some cases, the ED50 of the modified IL-10 monomer or dimer is decreased about 1-fold. In some cases, the ED50 of the modified IL-10 monomer or dimer is decreased about 2-fold. In some cases, the ED50 of the modified IL-10 monomer or dimer is decreased about 3-fold. In some cases, the ED50 of the modified IL-10 monomer or dimer is decreased about 4-fold. In some cases, the ED50 of the modified IL-10 monomer or dimer is decreased about 5-fold. In some cases, the ED50 of the modified IL-10 monomer or dimer is decreased about 6-fold. In some cases, the ED50 of the modified IL-10 monomer or dimer is decreased about 7-fold. In some cases, the ED50 of the modified IL-10 monomer or dimer is decreased about 8-fold. In some cases, the ED50 of the modified IL-10 monomer or dimer is decreased about 9-fold. In some cases, the ED50 of the modified IL-10 monomer or dimer is decreased about 10-fold. In some cases, the ED50 of the modified IL-10 monomer or dimer is decreased about 30-fold. In some cases, the ED50 of the modified IL-10 monomer or dimer is decreased about 50-fold. In some cases, the ED50 of the modified IL-10 monomer or dimer is decreased about 100-fold. In some cases, the ED50 of the modified IL-10 monomer or dimer is decreased about 200-fold. In some cases, the ED50 of the modified IL-10 monomer or dimer is decreased about 300-fold. In some cases, the ED50 of the modified IL-10 monomer or dimer is decreased about 400-fold. In some cases, the ED50 of the modified IL-10 monomer or dimer is decreased about 500-fold. In some cases, the ED50 of the modified IL-10 monomer or dimer is decreased about 1000-fold. In some cases, the ED50 of the modified IL-10 monomer or dimer is decreased more than 1,000-fold.

In some cases, the ED50 of the modified IL-10 monomer or dimer is about the same as the ED50 of the wild-type IL-10 protein.

In some instances, the modified IL-10 monomer or dimer as described herein has an increased ED50 compared to ED50 of wild-type IL-10 protein. In some embodiments, the increased ED50 of the modified IL-10 monomer or dimer is about 10%, 20%, 30%, 40%, 50%, 60%, 70%, 80%, 90%, 95%, or 99%, or greater than 99%. In some cases, the ED50 of the modified IL-10 monomer or dimer is increased about 10%. In some cases, the ED50 of the modified IL-10 monomer or dimer is increased about 20%. In some cases, the ED50 of the modified IL-10 monomer or dimer is increased about 30%. In some cases, the ED50 of the modified IL-10 monomer or dimer is increased about 40%. In some cases, the ED50 of the modified IL-10 monomer or dimer is increased about 50%. In some cases, the ED50 of the modified IL-10 monomer or dimer is increased about 60%. In some cases, the ED50 of the modified IL-10 monomer or dimer is increased about 70%. In some cases, the ED50 of the modified IL-10 monomer or dimer is increased about 80%. In some cases, the ED50 of the modified IL-10 monomer or dimer is increased about 90%. In some cases, the ED50 of the modified IL-10 monomer or dimer is increased about 95%. In some cases, the ED50 of the modified IL-10 monomer or dimer is increased about 99%. In some cases, the ED50 of the modified IL-10 monomer or dimer is increased about 100%.

In some embodiments, the increased ED50 of the modified IL-10 monomer or dimer compared to the ED50 of the wild-type IL-10 protein is about 1-fold, 2-fold, 3-fold, 4-fold, 5-fold, 6-fold, 7-fold, 8-fold, 9-fold, 10-fold, 30-fold, 50-fold, 100-fold, 200-fold, 300-fold, 400-fold, 500-fold, 1,000-fold, or more. In some cases, the ED50 of the modified IL-10 monomer or dimer is increased about 1-fold. In some cases, the ED50 of the modified IL-10 monomer or dimer is increased about 2-fold. In some cases, the ED50 of the modified IL-10 monomer or dimer is increased about 3-fold. In some cases, the ED50 of the modified IL-10 monomer or dimer is increased about 4-fold. In some cases, the ED50 of the modified IL-10 monomer or dimer is increased about 5-fold. In some cases, the ED50 of the modified IL-10 monomer or dimer is increased about 6-fold. In some cases, the ED50 of the modified IL-10 monomer or dimer is increased about 7-fold. In some cases, the ED50 of the modified IL-10 monomer or dimer is increased about 8-fold. In some cases, the ED50 of the modified IL-10 monomer or dimer is increased about 9-fold. In some cases, the ED50 of the modified IL-10 monomer or dimer is increased about 10-fold. In some cases, the ED50 of the modified IL-10 monomer or dimer is increased about 30-fold. In some cases, the ED50 of the modified IL-10 monomer or dimer is increased about 50-fold. In some cases, the ED50 of the modified IL-10 monomer or dimer is increased about 100-fold. In some cases, the ED50 of the modified IL-10 monomer or dimer is increased about 200-fold. In some cases, the ED50 of the modified IL-10 monomer or dimer is increased about 300-fold. In some cases, the ED50 of the modified IL-10 monomer or dimer is increased about 400-fold. In some cases, the ED50 of the modified IL-10 monomer or dimer is increased about 500-fold. In some cases, the ED50 of the modified IL-10 monomer or dimer is increased about 1000-fold. In some cases, the ED50 of the modified IL-10 monomer or dimer is increased more than 1,000-fold.

### IL-10 Conjugates

Described herein, in certain embodiments, are IL-10 conjugates. In some embodiments, the modified IL-10 polypeptides as described herein are IL-10 conjugates. In some embodiments, the IL-10 conjugate comprises an IL-10 polypeptide comprising at least one unnatural amino acid and at least one conjugating moiety bound to the at least one unnatural amino acid. In some instances, the at least one conjugating moiety is directly bound to the at least one unnatural amino acid. In other instances, the at least one conjugating moiety is indirectly bound to the at least one unnatural amino acid via a linker described herein.

In some embodiments, the IL-10 conjugate comprises at least one mutation comprising at least one unnatural amino acid and at least one conjugating moiety bound to the at least one unnatural amino acid at least at one of any one of the positions of SEQ ID NO: 1-66 (Table 1). In some embodiments, the IL-10 conjugates as described herein comprise an optional methionine at the N-terminus as depicted by (M) of SEQ ID NOS: 1 and 3-73. In some embodiments, the IL-10 conjugates comprise a methionine at the N-terminus of the wild-type or parental IL-10 sequence followed by the serine. In some instances, the IL-10 conjugates as described herein comprise the serine at the N-terminus of the wild-type or parental IL-10 sequence. In some embodiments, the modified IL-10 conjugates comprise a methionine substituting and replacing the serine at the N-terminus of the wild-type or parental IL-10 sequence. In some embodiments, the IL-10 conjugates comprise a methionine at the N-terminus followed by the serine as depicted by (M) of SEQ ID NO: 1. In some instances, the IL-10 conjugates comprise the serine at the N-terminus of SEQ ID NO: 1. In some embodiments, the IL-10 conjugates comprise a methionine substituting and replacing the serine at the N-terminus as depicted by (M) of SEQ ID NO: 1.

TABLE 1
SEQ ID Listings for IL-10 Conjugates.

| SEQ ID | | |
|---|---|---|
| NO: | Name | Sequence |
| 1 | IL-10 | (M)SPGQGTQSENSCTHFPGNLPNMLRDLRDAFSRVKTF |
| | (homo sapiens) | FQMKDQLDNLLLKESLLEDFKGYLGCQALSEMIQFYLE |
| | (mature form) | EVMPQAENQDPDIKAHVNSLGENLKTLRLRLRRCHRFL |
| | | PCENKSKAVEQVKNAFNKLQEKGIYKAMSEFDIFINYIE |
| | | AYMTMKIRN |
| 2 | IL-10 | MHSSALLCCLVLLTGVRASPGQGTQSENSCTHFPGNLP |
| | (homo sapiens) | NMLRDLRDAFSRVKTFFQMKDQLDNLLLKESLLEDFKG |
| | (precursor) | YLGCQALSEMIQFYLEEVMPQAENQDPDIKAHVNSLGE |
| | NCBI Accession No.: | NLKTLRLRLRRCHRFLPCENKSKAVEQVKNAFNKLQEK |
| | NP_000563.1 | GIYKAMSEFDIFINYIEAYMTMKIRN |
| 3 | IL-10_N82X | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAEXQDPDIKAHVNSLGENLKTLRLRLRRCHRFL |
| | | PCENKSKAVEQVKNAFNKLQEKGIYKAMSEFDIFINYIE |
| | | AYMTMKIRN |
| 4 | IL-10_K88X | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIXAHVNSLGENLKTLRLRLRRCHRFL |
| | | PCENKSKAVEQVKNAFNKLQEKGIYKAMSEFDIFINYIE |
| | | AYMTMKIRN |
| 5 | IL-10_A89X | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKXHVNSLGENLKTLRLRLRRCHRFL |
| | | PCENKSKAVEQVKNAFNKLQEKGIYKAMSEFDIFINYIE |
| | | AYMTMKIRN |
| 6 | IL-10_K99X | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLXTLRLRLRRCHRFL |
| | | PCENKSKAVEQVKNAFNKLQEKGIYKAMSEFDIFINYIE |
| | | AYMTMKIRN |
| 7 | IL-10_K125X | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVXNAFNKLQEKGIYKAMSEEDIFINYIE |
| | | AYMTMKIRN |
| 8 | IL-10_N126X | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVKXAFNKLQEKGIYKAMSEFDIFINYIE |
| | | AYMTMKIRN |
| 9 | IL-10_N129X | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVKNAFXKLQEKGIYKAMSEFDIFINYIE |
| | | AYMTMKIRN |
| 10 | IL-10_K130X | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVKNAFNXLQEKGIYKAMSEFDIFINYIE |
| | | AYMTMKIRN |
| 11 | IL-10_N82[AzK] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAE[AzK]QDPDIKAHVNSLGENLKTLRLRLRRCH |
| | | RFLPCENKSKAVEQVKNAFNKLQEKGIYKAMSEFDIFIN |
| | | YIEAYMTMKIRN |
| 12 | IL-10_K88[AzK] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDI[AzK]AHVNSLGENLKTLRLRLRRCH |
| | | RFLPCENKSKAVEQVKNAFNKLQEKGIYKAMSEFDIFIN |
| | | YIEAYMTMKIRN |
| 13 | IL-10_A89[AzK] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIK[AzK]HVNSLGENLKTLRLRLRRCH |
| | | RFLPCENKSKAVEQVKNAFNKLQEKGIYKAMSEFDIFIN |
| | | YIEAYMTMKIRN |
| 14 | IL-10_K99[AzK] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENL[AzK]TLRLRLRRCH |
| | | RFLPCENKSKAVEQVKNAFNKLQEKGIYKAMSEFDIFIN |
| | | YIEAYMTMKIRN |
| 15 | IL-10_K125[AzK] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQV[AzK]NAFNKLQEKGIYKAMSEFDIFIN |
| | | YIEAYMTMKIRN |
| 16 | IL-10_N126[AzK] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVK[AzK]AFNKLQEKGIYKAMSEFDIFIN |
| | | YIEAYMTMKIRN |
| 17 | IL-10_N129[AzK] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVKNAF[AzK]KLQEKGIYKAMSEFDIFIN |
| | | YIEAYMTMKIRN |
| 18 | IL-10_K130[AzK] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVKNAFN[AzK]LQEKGIYKAMSEFDIFIN |
| | | YIEAYMTMKIRN |
| 19 | IL-10_N82[AzK_PEG] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAE[AzK_PEG]QDPDIKAHVNSLGENLKTLRLRL |
| | | RRCHRFLPCENKSKAVEQVKNAFNKLQEKGIYKAMSEF |
| | | DIFINYIEAYMTMKIRN |
| 20 | IL-10_K88[AzK_PEG] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDI[AzK_PEG]AHVNSLGENLKTLRLRL |
| | | RRCHRFLPCENKSKAVEQVKNAFNKLQEKGIYKAMSEF |
| | | DIFINYIEAYMTMKIRN |
| 21 | IL-10_A89[AzK_PEG] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIK[AzK_PEG]HVNSLGENLKTLRLRL |
| | | RRCHRFLPCENKSKAVEQVKNAFNKLQEKGIYKAMSEF |
| | | DIFINYIEAYMTMKIRN |
| 22 | IL-10_K99[AzK_PEG] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENL[AzK_PEG]TLRLRL |
| | | RRCHRFLPCENKSKAVEQVKNAFNKLQEKGIYKAMSEF |
| | | DIFINYIEAYMTMKIRN |
| 23 | IL-10_K125[AzK_PEG] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQV[AzK_PEG]NAFNKLQEKGIYKAMSEF |
| | | DIFINYIEAYMTMKIRN |
| 24 | IL-10_N126[AzK_PEG] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVK[AzK_PEG]AFNKLQEKGIYKAMSEF |
| | | DIFINYIEAYMTMKIRN |
| 25 | IL-10_N129[AzK_PEG] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVKNAF[AzK_PEG]KLQEKGIYKAMSEF |
| | | DIFINYIEAYMTMKIRN |
| 26 | IL-10_K130[AzK_PEG] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVKNAFN[AzK_PEG]LQEKGIYKAMSEF |
| | | DIFINYIEAYMTMKIRN |
| 27 | IL-10_N82[AzK_PEG20kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAE[AzK_PEG20kDa]QDPDIKAHVNSLGENLKTL |
| | | RLRLRRCHRFLPCENKSKAVEQVKNAFNKLQEKGIYKA |
| | | MSEFDIFINYIEAYMTMKIRN |
| 28 | IL-10_K88[AzK_PEG20kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDI[AzK_PEG20kDa]AHVNSLGENLKTL |
| | | RLRLRRCHRFLPCENKSKAVEQVKNAFNKLQEKGIYKA |
| | | MSEFDIFINYIEAYMTMKIRN |
| 29 | IL-10_A89[AzK_PEG20kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIK[AzK_PEG20kDa]HVNSLGENLKTL |
| | | RLRLRRCHRFLPCENKSKAVEQVKNAFNKLQEKGIYKA |
| | | MSEFDIFINYIEAYMTMKIRN |
| 30 | IL-10_K99[AzK_PEG20kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENL[AzK_PEG20kDa]TL |
| | | RLRLRRCHRFLPCENKSKAVEQVKNAFNKLQEKGIYKA |
| | | MSEFDIFINYIEAYMTMKIRN |
| 31 | IL-10_K125[AzK_PEG20kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQV[AzK_PEG20kDa]NAFNKLQEKGIYKA |
| | | MSEFDIFINYIEAYMTMKIRN |
| 32 | IL-10_N126[AzK_PEG20kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVK[AzK_PEG20kDa]AFNKLQEKGIYKA |
| | | MSEFDIFINYIEAYMTMKIRN |
| 33 | IL-10_N129[AzK_PEG20kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVKNAF[AzK_PEG20kDa]KLQEKGIYKA |
| | | MSEFDIFINYIEAYMTMKIRN |
| 34 | IL-10_K130[AzK_PEG20kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVKNAFN[AzK_PEG20kDa]LQEKGIYKA |
| | | MSEFDIFINYIEAYMTMKIRN |
| 35 | IL-10_N82[AzK_PEG30kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAE[AzK_PEG30kDa]QDPDIKAHVNSLGENLKTL |
| | | RLRLRRCHRFLPCENKSKAVEQVKNAFNKLQEKGIYKA |
| | | MSEFDIFINYIEAYMTMKIRN |
| 36 | IL-10_K88[AzK_PEG30kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDI[AzK_PEG30kDa]AHVNSLGENLKTL |
| | | RLRLRRCHRFLPCENKSKAVEQVKNAFNKLQEKGIYKA |
| | | MSEFDIFINYIEAYMTMKIRN |
| 37 | IL-10_A89[AzK_PEG30kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIK[AzK_PEG30kDa]HVNSLGENLKTL |
| | | RLRLRRCHRFLPCENKSKAVEQVKNAFNKLQEKGIYKA |
| | | MSEFDIFINYIEAYMTMKIRN |
| 38 | IL-10_K99[AzK_PEG30kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENL[AzK_PEG30kDa]TL |
| | | RLRLRRCHRFLPCENKSKAVEQVKNAFNKLQEKGIYKA |
| | | MSEFDIFINYIEAYMTMKIRN |
| 39 | IL-10_K125[AzK_PEG30kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQV[AzK_PEG30kDa]NAFNKLQEKGIYKA |
| | | MSEFDIFINYIEAYMTMKIRN |
| 40 | IL-10_N126[AzK_PEG30kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVK[AzK_PEG30kDa]AFNKLQEKGIYKA |
| | | MSEFDIFINYIEAYMTMKIRN |
| 41 | IL-10_N129[AzK_PEG30kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVKNAF[AzK_PEG30kDa]KLQEKGIYKA |
| | | MSEFDIFINYIEAYMTMKIRN |
| 42 | IL-10_K130[AzK_PEG30kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVKNAFN[AzK_PEG30kDa]LQEKGIYKA |
| | | MSEFDIFINYIEAYMTMKIRN |
| 43 | IL-10_N82[AzK_L1_PEG20kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAE[AzK_L1_PEG20kDa]QDPDIKAHVNSLGENL |
| | | KTLRLRLRRCHRFLPCENKSKAVEQVKNAFNKLQEKGI |
| | | YKAMSEFDIFINYIEAYMTMKIRN |
| 44 | IL-10_K88[AzK_L1_PEG20kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDI[AzK_L1_PEG20kDa]AHVNSLGENL |
| | | KTLRLRLRRCHRFLPCENKSKAVEQVKNAFNKLQEKGI |
| | | YKAMSEFDIFINYIEAYMTMKIRN |
| 45 | IL-10_A89[AzK_L1_PEG20kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIK[AzK_L1_PEG20kDa]HVNSLGENL |
| | | KTLRLRLRRCHRFLPCENKSKAVEQVKNAFNKLQEKGI |
| | | YKAMSEFDIFINYIEAYMTMKIRN |
| 46 | IL-10_K99[AzK_L1_PEG20kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENL[AzK_L1_PEG20kDa] |
| | | ITLRLRLRRCHRFLPCENKSKAVEQVKNAFNKLQEKGIY |
| | | KAMSEFDIFINYIEAYMTMKIRN |
| 47 | IL-10_K125[AzK_L1_PEG20kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQV[AzK_L1_PEG20kDa]NAFNKLQEKGI |
| | | YKAMSEFDIFINYIEAYMTMKIRN |
| 48 | IL-10_N126[AzK_L1_PEG20kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVK[AzK_L1_PEG20kDa]AFNKLQEKGI |
| | | YKAMSEFDIFINYIEAYMTMKIRN |
| 49 | IL-10_N129[AzK_L1_PEG20kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVKNAF[AzK_L1_PEG20kDa]KLQEKGI |
| | | YKAMSEFDIFINYIEAYMTMKIRN |
| 50 | IL-10_K130[AzK_L1_PEG20kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVKNAFN[AzK_L1_PEG20kDa]LQEKGI |
| | | YKAMSEFDIFINYIEAYMTMKIRN |
| 51 | IL-10_N82[AzK_L1_PEG30kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAE[AzK_L1_PEG30kDa]QDPDIKAHVNSLGENL |
| | | KTLRLRLRRCHRFLPCENKSKAVEQVKNAFNKLQEKGI |
| | | YKAMSEFDIFINYIEAYMTMKIRN |
| 52 | IL-10_K88[AzK_L1_PEG30kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDI[AzK_L1_PEG30kDa]AHVNSLGENL |
| | | KTLRLRLRRCHRFLPCENKSKAVEQVKNAFNKLQEKGI |
| | | YKAMSEFDIFINYIEAYMTMKIRN |
| 53 | IL-10_A89[AzK_L1_PEG30kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIK[AzK_L1_PEG30kDa]HVNSLGENL |
| | | KTLRLRLRRCHRFLPCENKSKAVEQVKNAFNKLQEKGI |
| | | YKAMSEFDIFINYIEAYMTMKIRN |
| 54 | IL-10_K99[AzK_L1_PEG30kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENL[AzK_L1_PEG30kDa] |
| | | TLRLRLRRCHRFLPCENKSKAVEQVKNAFNKLQEKGIY |
| | | KAMSEFDIFINYIEAYMTMKIRN |
| 55 | IL-10_K125[AzK_L1_PEG30kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQV[AzK_L1_PEG30kDa]NAFNKLQEKGI |
| | | YKAMSEFDIFINYIEAYMTMKIRN |
| 56 | IL-10_N126[AzK_L1_PEG30kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVK[AzK_L1_PEG30kDa]AFNKLQEKGI |
| | | YKAMSEFDIFINYIEAYMTMKIRN |
| 57 | IL-10_N129[AzK_L1_PEG30kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVKNAF[AzK_L1_PEG30kDa]KLQEKGI |
| | | YKAMSEFDIFINYIEAYMTMKIRN |
| 58 | IL-10_K130[AzK_L1_PEG30kDa] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVKNAFN[AzK_L1_PEG30kDa]LQEKGI |
| | | YKAMSEFDIFINYIEAYMTMKIRN |
| 59 | IL-10_N82[AzK_L1_PEG] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAE[AzK_L1_PEG]QDPDIKAHVNSLGENLKTLRL |
| | | RLRRCHRFLPCENKSKAVEQVKNAFNKLQEKGIYKAMS |
| | | EFDIFINYIEAYMTMKIRN |
| 60 | IL-10_K88[AzK_L1_PEG] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDI[AzK_L1_PEG]AHVNSLGENLKTLRL |
| | | RLRRCHRFLPCENKSKAVEQVKNAFNKLQEKGIYKAMS |
| | | EFDIFINYIEAYMTMKIRN |
| 61 | IL-10_A89[AzK_L1_PEG] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIK[AzK_L1_PEG]HVNSLGENLKTLRL |
| | | RLRRCHRFLPCENKSKAVEQVKNAFNKLQEKGIYKAMS |
| | | EFDIFINYIEAYMTMKIRN |
| 62 | IL-10_K99[AzK_L1_PEG] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENL[AzK_L1_PEG]TLRL |
| | | RLRRCHRFLPCENKSKAVEQVKNAFNKLQEKGIYKAMS |
| | | EFDIFINYIEAYMTMKIRN |
| 63 | IL-10_K125[AzK_L1_PEG] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQV[AzK_L1_PEG]NAFNKLQEKGIYKAM |
| | | SEFDIFINYIEAYMTMKIRN |
| 64 | IL-10_N126[AzK_L1_PEG] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVK[AzK_L1_PEG]AFNKLQEKGIYKAM |
| | | SEFDIFINYIEAYMTMKIRN |
| 65 | IL-10_N129[AzK_L1_PEG] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVKNAF[AzK_L1_PEG]KLQEKGIYKAM |
| | | SEFDIFINYIEAYMTMKIRN |
| 66 | IL-10_K130[AzK_L1_PEG] | (M)SPGQGTQSENSCTHFPGNLPNMLRDLRDAFSRVKTF |
| | | FQMKDQLDNLLLKESLLEDFKGYLGCQALSEMIQFYLE |
| | | EVMPQAENQDPDIKAHVNSLGENLKTLRLRLRRCHRFL |
| | | PCENKSKAVEQVKNAFN[AzK_L1_PEG]LQEKGIYKAM |
| | | SEFDIFINYIEAYMTMKIRN |
| 67 | His-IL-10 | (M)HHHHHHGSSENLYFQSPGQGTQSENSCTHFPGNLPN |
| | | MLRDLRDAFSRVKTFFQMKDQLDNLLLKESLLEDFKGY |
| | | LGCQALSEMIQFYLEEVMPQAENQDPDIKAHVNSLGEN |
| | | LKTLRLRLRRCHRFLPCENKSKAVEQVKNAFNKLQEKG |
| | | IYKAMSEFDIFINYIEAYMTMKIRN |
| 68 | His-IL-10_N82[AzK_PEG20kDa] | (M)HHHHHHGSSENLYFQSPGQGTQSENSCTHFPGNLPN |
| | | MLRDLRDAFSRVKTFFQMKDQLDNLLLKESLLEDFKGY |
| | | LGCQALSEMIQFYLEEVMPQAE[AzK_PEG20kDa]QDPD |
| | | IKAHVNSLGENLKTLRLRLRRCHRFLPCENKSKAVEQV |
| | | KNAFNKLQEKGIYKAMSEFDIFINYIEAYMTMKIRN |
| 69 | His-IL-10_K99[AzK_PEG20kDa] | (M)HHHHHHGSSENLYFQSPGQGTQSENSCTHFPGNLPN |
| | | MLRDLRDAFSRVKTFFQMKDQLDNLLLKESLLEDFKGY |
| | | LGCQALSEMIQFYLEEVMPQAENQDPDIKAHVNSLGEN |
| | | L[AzK_PEG20kDa]TLRLRLRRCHRFLPCENKSKAVEQV |
| | | KNAFNKLQEKGIYKAMSEFDIFINYIEAYMTMKIRN |
| 70 | His-IL-10_K125[AzK_PEG20kDa] | (M)HHHHHHGSSENLYFQSPGQGTQSENSCTHFPGNLPN |
| | | MLRDLRDAFSRVKTFFQMKDQLDNLLLKESLLEDFKGY |
| | | LGCQALSEMIQFYLEEVMPQAENQDPDIKAHVNSLGEN |
| | | LKTLRLRLRRCHRFLPCENKSKAVEQV[AzK_PEG20kDa] |
| | | NAFNKLQEKGIYKAMSEFDIFINYIEAYMTMKIRN |
| 71 | His-IL-10_N129[AzK_PEG20kDa] | (M)HHHHHHGSSENLYFQSPGQGTQSENSCTHFPGNLPNM |
| | | LRDLRDAFSRVKTFFQMKDQLDNLLLKESLLEDFKGYL |
| | | GCQALSEMIQFYLEEVMPQAENQDPDIKAHVNSLGENL |
| | | KTLRLRLRRCHRFLPCENKSKAVEQVKNAF[AzK_PEG20kDa] |
| | | KLQEKGIYKAMSEFDIFINYIEAYMTMKIRN |
| 72 | His-IL-10_K130[AzK_PEG20kDa] | (M)HHHHHHGSSENLYFQSPGQGTQSENSCTHFPGNLPNM |
| | | LRDLRDAFSRVKTFFQMKDQLDNLLLKESLLEDFKGYL |
| | | GCQALSEMIQFYLEEVMPQAENQDPDIKAHVNSLGENL |
| | | KTLRLRLRRCHRFLPCENKSKAVEQVKNAFN[AzK_PEG20kDa] |
| | | LQEKGIYKAMSEFDIFINYIEAYMTMKIRN |
| 73 | His-IL-10_N82[AzK_PEG10kDa] | (M)HHHHHHGSSENLYFQSPGQGTQSENSCTHFPGNLPNM |
| | | LRDLRDAFSRVKTFFQMKDQLDNLLLKESLLEDFKGYL |
| | | GCQALSEMIQFYLEEVMPQAE[AzK_PEG10kDa]QDPDIK |
| | | AHVNSLGENLKTLRLRLRRCHRFLPCENKSKAVEQVKN |
| | | AFNKLQEKGIYKAMSEFDIFINYIEAYMTMKIRN |

(M)=A methionine residue can be optionally added to the N-terminus of the modified IL-10 polypeptides and H_-10 conjugates as depicted in SEQ TD NO: 1 and 3-73. Alternatively, the methionine residue can substitute and replace the serine at the N terminus.
X=site comprising an unnatural amino acid.
[AzK]=N6-((2-azidoethoxy)-carbonyl)-L-lysine. The compound has Chemical Abstracts Registry No. 1167421-25-1.
[AzK_PEG]=N6-((2-azidoethoxy)-carbonyl)-L-lysine stably-conjugated to PEG via DBCO-mediated click chemistry, to form a compound comprising a structure of Formula (II) or Formula (III), or Formula (X) or Formula (XI). In some examples, the compound has a structure of Formula (II), Formula (III), Formula (X), or Formula (XI) wherein substituent q is present, and q is 1. In some examples, the compound has a structure of Formula (II), Formula (III), Formula (X), or Formula (XI) wherein substituent q is present, and q is 2. In some examples, the compound has a structure of Formula (II), Formula (III), Formula (X), or Formula (XI) wherein substituent q is present, and q is 3. For example, if specified, PEG20 kDa indicates, in the case of the compound comprising a structure of Formula (II) or Formula (III), a linear polyethylene glycol chain with an average molecular weight of 20 kiloDaltons, capped with a methoxy group. In another example, if specified, PEG20 kDa indicates, in the case of the compound comprising a structure of Formula (X) or Formula (XI), a compound wherein n is a value providing a PEG group having a weight of 20 kiloDaltons. The ratio of regioisomers generated from the click reaction is about 1:1 or greater than 1:1. The term “DBCO” means a chemical moiety comprising a dibenzocyclooctyne group, such as comprising the mPEG-DBCO compound.
[AzK_L1_PEG]=N6-((2-azidoethoxy)-carbonyl)-L-lysine stably-conjugated to PEG via DBCO-mediated click chemistry to form a compound comprising a structure of Formula (IV) or Formula (V), or Formula (XII) or Formula (XIII). In some examples, the compound has a structure of Formula (IV), Formula (V), Formula (XII), or Formula (XIII) wherein substituent q is present, and q is 1. In some examples, the compound has a structure of Formula (IV), Formula (V), Formula (XII), or Formula (XIII) wherein substituent q is present, and q is 2. In some examples, the compound has a structure of Formula (IV), Formula (V), Formula (XII), or Formula (XIII) wherein substituent q is present, and q is 3. For example, if specified, PEG20 kDa indicates, in the case of the compound comprising a structure of Formula (IV) or Formula (V), a linear polyethylene glycol chain with an average molecular weight of 20 kiloDaltons, capped with a methoxy group. In another example, if specified, PEG20 kDa indicates, in the case of the compound comprising a structure of Formula (XII) or Formula (XIII), a compound wherein n is a value providing a PEG group having a weight of 20 kiloDaltons. The ratio of regioisomers generated from the click reaction is about 1:1 or greater than 1:1. The term “DBCO” means a chemical moiety comprising a dibenzocyclooctyne group, such as comprising the mPEG-DBCO compound.
[His]=The amino acid sequence containing a histidine tag and a TEV recognition site, having the sequence HHHHHHGSSENLYFQ (residues 1-15 of SEQ ID NOS: 67-73). This sequence may be cleaved from the expressed IL-10 conjugate by methods described herein and those known to one having ordinary skill in the art to provide the IL-10 conjugate lacking the amino acid sequence HHHHHHGSSENLYFQ (residues 1-15 of SEQ ID NOS: 67-73). For example, the histidine tag and a TEV recognition site comprising the IL-10 conjugate of SEQ ID NO: 68 may be cleaved to afford the IL-10 conjugate having SEQ ID NO: 27. More generally, “[His]-SEQ ID NO: X” indicates that the sequence containing a histidine tag and a TEV recognition site shown above is present at the N-terminus of the indicated sequence, immediately following the initial methionine if present.

As described herein, the at least one unnatural amino acid is optionally located in helix C, D, or E, e.g., a surface accessible residue. In some cases, the residues include E67, Q70, E74, E75, Q79, N82, K88, A89, K99, K125, N126, N129, K130, or Q132, wherein the residue positions correspond to positions 67, 70, 74, 75, 79, 82, 88, 89, 99, 125, 126, 129, 130, and 132 as set forth in SEQ ID NO: 1. In some cases, the residues include E67, Q70, E74, E75, Q79, or N82, wherein the residue positions correspond to positions 67, 70, 74, 75, 79, and 82 as set forth in SEQ ID NO: 1. In some cases, the residue include K88, K125, N126, N129, K130, or Q132, wherein the residue positions correspond to positions 88, 125, 126, 129, 130, and 132 as set forth in SEQ ID NO: 1. In some cases, the residue include K125, N126, N129, K130, or Q132, wherein the residue positions correspond to positions 125, 126, 129, 130, and 132 as set forth in SEQ ID NO: 1. In some cases, the residue include Q70, E74, N82, K88, N126, K130, or Q132, wherein the residue positions correspond to positions 70, 74, 82, 88, 126, 130, and 132 as set forth in SEQ ID NO: 1. In some cases, the residue include A89 and K99, wherein the residue positions correspond to positions 89 and 99 as set forth in SEQ ID NO: 1.

In some instances, the position of the at least one unnatural amino acid is E67 of SEQ ID NO: 1. In some instances, the position of the at least one unnatural amino acid is Q70 of SEQ ID NO: 1. In some instances, the position of the at least one unnatural amino acid is E74 of SEQ ID NO: 1. In some instances, the position of the at least one unnatural amino acid is E75 of SEQ ID NO: 1. In some instances, the position of the at least one unnatural amino acid is Q79 of SEQ ID NO: 1. In some instances, the position of the at least one unnatural amino acid is N82 of SEQ ID NO: 1. In some instances, the position of the at least one unnatural amino acid is K88 of SEQ ID NO: 1. In some instances, the position of the at least one unnatural amino acid is A89 of SEQ ID NO: 1. In some instances, the position of the at least one unnatural amino acid is K99 of SEQ ID NO: 1. In some instances, the position of the at least one unnatural amino acid is K125 of SEQ ID NO: 1. In some instances, the position of the at least one unnatural amino acid is N126 of SEQ ID NO: 1. In some instances, the position of the at least one unnatural amino acid is N129 of SEQ ID NO: 1. In some instances, the position of the at least one unnatural amino acid is K130 of SEQ ID NO: 1. In some instances, the position of the at least one unnatural amino acid is Q132 of SEQ ID NO: 1.

In some cases, the at least one unnatural amino acid residue is selected from E85, Q88, E92, E93, Q97, N100, K106, A107, K117, K143, N144, N147, K148, or Q150, wherein the residue positions correspond to positions 85, 88, 92, 93, 97, 100, 106, 107, 117, 143, 144, 147, 148, and 150 as set forth in an IL-10 precursor of SEQ ID NO: 2. In some instances, the position of the at least one unnatural amino acid is E85 of SEQ ID NO: 2. In some instances, the position of the at least one unnatural amino acid is Q88 of SEQ ID NO: 2. In some instances, the position of the at least one unnatural amino acid is E92 of SEQ ID NO: 2. In some instances, the position of the at least one unnatural amino acid is E93 of SEQ ID NO: 2. In some instances, the position of the at least one unnatural amino acid is Q97 of SEQ ID NO: 2. In some instances, the position of the at least one unnatural amino acid is N100 of SEQ ID NO: 2. In some instances, the position of the at least one unnatural amino acid is K106 of SEQ ID NO: 2. In some instances, the position of the at least one unnatural amino acid is A107 of SEQ ID NO: 2. In some instances, the position of the at least one unnatural amino acid is K117 of SEQ ID NO: 2. In some instances, the position of the at least one unnatural amino acid is K143 of SEQ ID NO: 2. In some instances, the position of the at least one unnatural amino acid is N144 of SEQ ID NO: 2. In some instances, the position of the at least one unnatural amino acid is N147 of SEQ ID NO: 2. In some instances, the position of the at least one unnatural amino acid is K148 of SEQ ID NO: 2. In some instances, the position of the at least one unnatural amino acid is Q150 of SEQ ID NO: 2.

Described herein, in some embodiments, is an IL-10 conjugate comprising the amino acid sequence of SEQ ID NO: 1 in which at least one amino acid residue in the IL-10 conjugate is replaced by the structure of Formula (I):

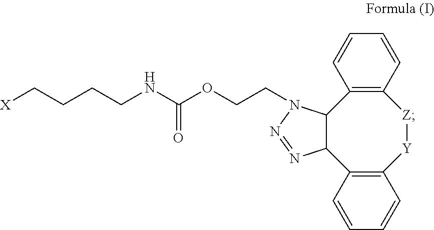

wherein:

### Z is CH₂ and Y is

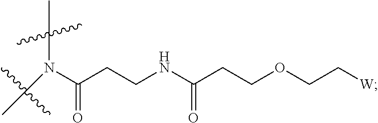

### Y is CH₂ and Z is

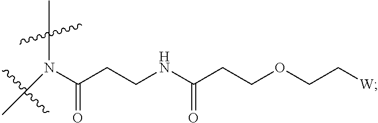

### Z is CH₂ and Y is

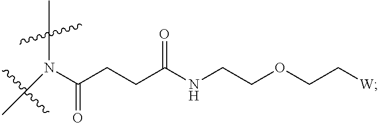

or

### Y is CH₂ and Z is

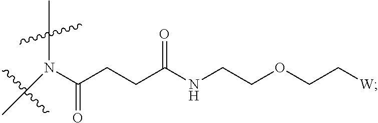

W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

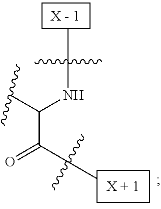 [SMILES: *NC(*)C(*)=O]

X−1 indicates the point of attachment to the preceding amino acid residue; and
X+1 indicates the point of attachment to the following amino acid residue.

In other embodiments, described herein is an IL-10 conjugate comprising the amino acid sequence of SEQ ID NO: 1 in which at least one amino acid residue in the IL-10 conjugate is replaced by the structure of Formula (I):

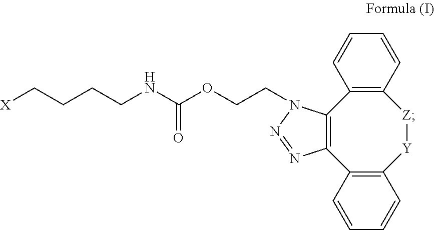

wherein:

### Z is CH₂ and Y is

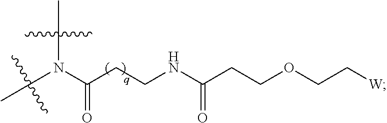

### Y is CH₂ and Z is

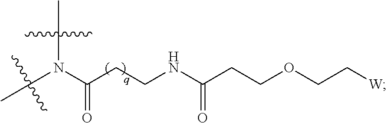

### Z is CH₂ and Y is or

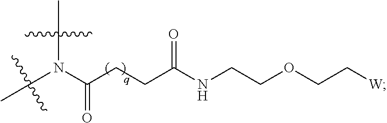

### Y is CH₂ and Z is

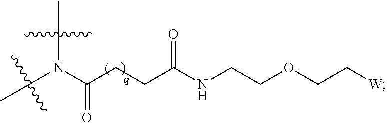

q is 1, 2, or 3;
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure.

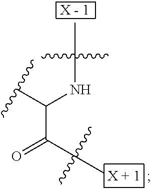

X−1 indicates the point of attachment to the preceding amino acid residue; and
X+1 indicates the point of attachment to the following amino acid residue.

Here and throughout, the term “IL-10 conjugate” encompasses pharmaceutically acceptable salts, solvates, and hydrates of the indicated structure.

Here and throughout, the structure of Formula (I) encompasses pharmaceutically acceptable salts, solvates, or hydrates thereof. In some embodiments, the structure of Formula (I), or any embodiment or variation thereof, is provided as a pharmaceutically acceptable salt thereof. In some embodiments, the structure of Formula (I), or any embodiment or variation thereof, is provided as a solvate thereof. In some embodiments, the structure of Formula (I), or any embodiment or variation thereof, is provided as a hydrate thereof. In some embodiments, the structure of Formula (I), or any embodiment or variation thereof, is provided as the free base.

In some embodiments of the IL-10 conjugate comprising Formula (I), Z is CH₂ and Y is

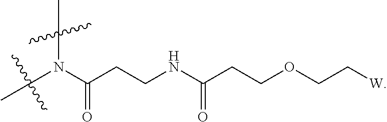

In some embodiments of the IL-10 conjugate comprising Formula (I), Y is CH₂ and Z is

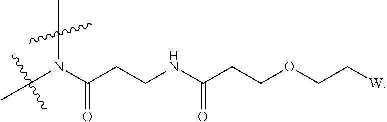

In some embodiments of the IL-10 conjugate comprising Formula (I), Z is CH₂ and Y is

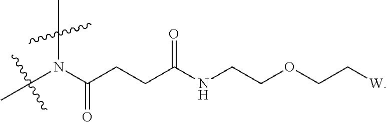

In some embodiments of the IL-10 conjugate comprising Formula (I), Y is CH₂ and Z is

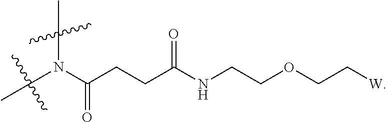

In some embodiments of the IL-10 conjugate comprising Formula (I), Z is CH₂ and Y is

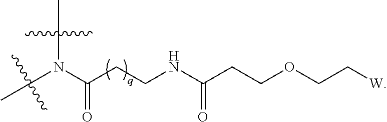

In some embodiments of the IL-10 conjugate comprising Formula (I), Y is CH₂ and Z is

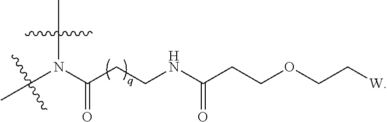

In some embodiments of the IL-10 conjugate comprising Formula (I), Z is CH₂ and Y is

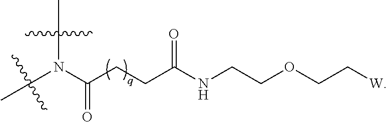

In some embodiments of the IL-10 conjugate comprising Formula (I), Y is CH₂ and Z is

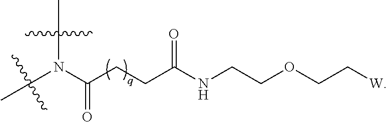

Here and throughout, embodiments of Z and Y also encompass a pharmaceutically acceptable salt, solvate, or hydrate thereof.

In some embodiments of the IL-10 conjugate comprising Formula (I), q is 1. In some embodiments of the IL-10 conjugate comprising Formula (I), q is 2. In some embodiments of the IL-10 conjugate comprising Formula (I), q is 3.

In some embodiments of the IL-10 conjugate comprising Formula (I), the PEG group has an average molecular weight selected from 500 Daltons, 1 kDa, 2 kDa, 3 kDa, 4 kDa, 5 kDa, 10 kDa, 15 kDa, 20 kDa, 25 kDa, 30 kDa, 35 kDa, 40 kDa, 45 kDa, 50 kDa, and 100 kDa. In some embodiments, the PEG group has an average molecule weight selected from 5 kDa, 10 kDa, 20 kDa and 30 kDa. In some embodiments of the IL-10 conjugate comprising Formula (I), the PEG group has an average molecular weight of 20 kDa. In some embodiments of the IL-10 conjugate comprising Formula (I), the PEG group has an average molecular weight of 30 kDa.

In some embodiments of the IL-10 conjugate comprising Formula (I), the position of the structure of Formula (I) in the amino acid sequence of the IL-10 conjugate is selected from E67, Q70, E74, E75, Q79, N82, K88, A89, K99, K125, N126, N129, K130, and Q132, wherein the position of the structure of Formula (I) in the amino acid sequence of the IL-10 conjugate is in reference to the positions in SEQ ID NO: 1. In some embodiments of the IL-10 conjugate comprising Formula (I), the position of the structure of Formula (I) in the amino acid sequence of the IL-10 conjugate is selected from N82, K88, A89, K99, K125, N126, N129, and K130, wherein the position of the structure of Formula (I) in the amino acid sequence of the IL-10 conjugate is in reference to the positions in SEQ ID NO: 1. In some embodiments of the IL-10 conjugate comprising Formula (I), the position of the structure of Formula (I) in the amino acid sequence of the IL-10 conjugate is E67, wherein the position of the structure of Formula (I) in the amino acid sequence of the IL-10 conjugate is in reference to the positions in SEQ ID NO: 1. In some embodiments of the IL-10 conjugate comprising Formula (I), the position of the structure of Formula (I) in the amino acid sequence of the IL-10 conjugate is Q70, wherein the position of the structure of Formula (I) in the amino acid sequence of the IL-10 conjugate is in reference to the positions in SEQ ID NO: 1. In some embodiments of the IL-10 conjugate comprising Formula (I), the position of the structure of Formula (I) in the amino acid sequence of the IL-10 conjugate is E74, wherein the position of the structure of Formula (I) in the amino acid sequence of the IL-10 conjugate is in reference to the positions in SEQ ID NO: 1. In some embodiments of the IL-10 conjugate comprising Formula (I), the position of the structure of Formula (I) in the amino acid sequence of the IL-10 conjugate is E75, wherein the position of the structure of Formula (I) in the amino acid sequence of the IL-10 conjugate is in reference to the positions in SEQ ID NO: 1. In some embodiments of the IL-10 conjugate comprising Formula (I), the position of the structure of Formula (I) in the amino acid sequence of the IL-10 conjugate is Q79, wherein the position of the structure of Formula (I) in the amino acid sequence of the IL-10 conjugate is in reference to the positions in SEQ ID NO: 1. In some embodiments of the IL-10 conjugate comprising Formula (I), the position of the structure of Formula (I) in the amino acid sequence of the IL-10 conjugate is N82, wherein the position of the structure of Formula (I) in the amino acid sequence of the IL-10 conjugate is in reference to the positions in SEQ ID NO: 1 and in SEQ ID NO: 3. In some embodiments of the IL-10 conjugate comprising Formula (I), the position of the structure of Formula (I) in the amino acid sequence of the IL-10 conjugate is K88, wherein the position of the structure of Formula (I) in the amino acid sequence of the IL-10 conjugate is in reference to the positions in SEQ ID NO: 1 and in SEQ ID NO: 4. In some embodiments of the IL-10 conjugate comprising Formula (I), the position of the structure of Formula (I) in the amino acid sequence of the IL-10 conjugate is A89, wherein the position of the structure of Formula (I) in the amino acid sequence of the IL-10 conjugate is in reference to the positions in SEQ ID NO: 1 and in SEQ ID NO: 5. In some embodiments of the IL-10 conjugate comprising Formula (I), the position of the structure of Formula (I) in the amino acid sequence of the IL-10 conjugate is K99, wherein the position of the structure of Formula (I) in the amino acid sequence of the IL-10 conjugate is in reference to the positions in SEQ ID NO: 1 and in SEQ ID NO: 6. In some embodiments of the IL-10 conjugate comprising Formula (I), the position of the structure of Formula (I) in the amino acid sequence of the IL-10 conjugate is K125, wherein the position of the structure of Formula (I) in the amino acid sequence of the IL-10 conjugate is in reference to the positions in SEQ ID NO: 1 and in SEQ ID NO: 7. In some embodiments of the IL-10 conjugate comprising Formula (I), the position of the structure of Formula (I) in the amino acid sequence of the IL-10 conjugate is N126, wherein the position of the structure of Formula (I) in the amino acid sequence of the IL-10 conjugate is in reference to the positions in SEQ ID NO: 1 and in SEQ ID NO: 8. In some embodiments of the IL-10 conjugate comprising Formula (I), the position of the structure of Formula (I) in the amino acid sequence of the IL-10 conjugate is N129, wherein the position of the structure of Formula (I) in the amino acid sequence of the IL-10 conjugate is in reference to the positions in SEQ ID NO: 1 and in SEQ ID NO: 9. In some embodiments of the IL-10 conjugate comprising Formula (I), the position of the structure of Formula (I) in the amino acid sequence of the IL-10 conjugate is K130, wherein the position of the structure of Formula (I) in the amino acid sequence of the IL-10 conjugate is in reference to the positions in SEQ ID NO: 1 and in SEQ ID NO: 10. In some embodiments of the IL-10 conjugate comprising Formula (I), the position of the structure of Formula (I) in the amino acid sequence of the IL-10 conjugate is Q132, wherein the position of the structure of Formula (I) in the amino acid sequence of the IL-10 conjugate is in reference to the positions in SEQ ID NO: 1.

Described herein, in some embodiments, is an IL-10 conjugate comprising the amino acid sequence of any one of SEQ ID NOS: 19 to 26, wherein [AzK_PEG] has the structure of Formula (II), Formula (III), or a mixture of Formula (II) and Formula (III):

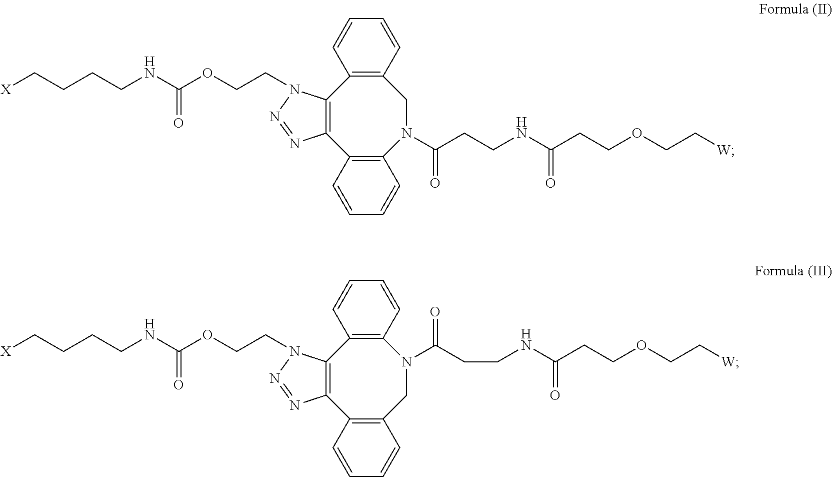

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

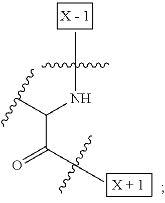

X−1 indicates the point of attachment to the preceding amino acid residue; and
X+1 indicates the point of attachment to the following amino acid residue.

Described herein, in some embodiments, is an IL-10 conjugate comprising the amino acid sequence of any one of SEQ ID NOS: 19 to 26, wherein [AzK_PEG] has the structure of Formula (II), Formula (III), or a mixture of Formula (II) and Formula (III):

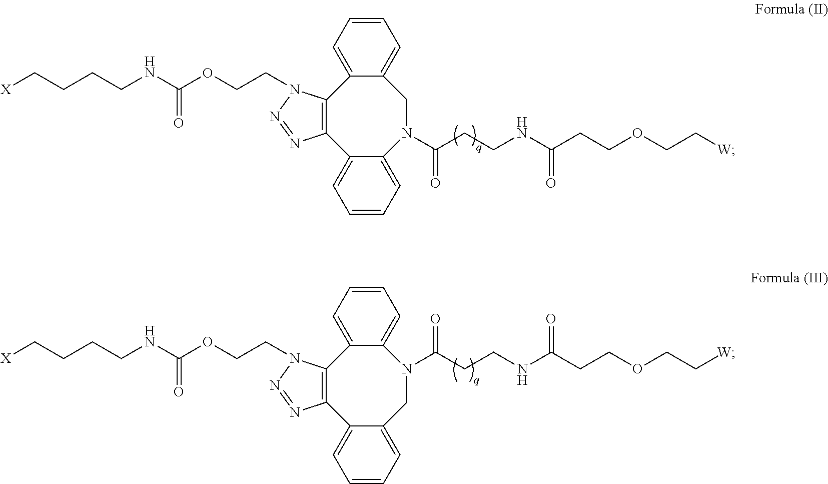

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa;
q is 1, 2, or 3; and
X has the structure:

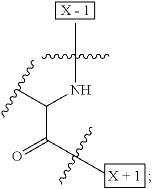

X−1 indicates the point of attachment to the preceding amino acid residue; and
X+1 indicates the point of attachment to the following amino acid residue.

Here and throughout, the structure of Formula (II) encompasses pharmaceutically acceptable salts, solvates, or hydrates thereof. Here and throughout, the structure of Formula (III) encompasses pharmaceutically acceptable salts, solvates, or hydrates thereof.

In some embodiments, the [AzK_PEG] has the structure of Formula (II). In some embodiments, the [AzK_PEG] has the structure of Formula (III). In some embodiments, the [AzK_PEG] is a mixture of Formula (II) and Formula (III).

In some embodiments of the IL-10 conjugate comprising Formula (II), the IL-10 conjugate has the amino acid sequence of SEQ ID NO: 19. In some embodiments of the IL-10 conjugate comprising Formula (II) and having an amino acid sequence of SEQ ID NO: 19, W is a PEG group having an average molecular weight selected from 5 kDa, 10 kDa, 15 kDa, 20 kDa, 25 kDa, and 30 kDa. In some embodiments of the IL-10 conjugate comprising Formula (II) and having an amino acid sequence of SEQ ID NO: 19, W is a PEG group having an average molecular weight selected from 20 kDa and 30 kDa. In some embodiments of the IL-10 conjugate comprising Formula (II) and having an amino acid sequence of SEQ ID NO: 19, W is a PEG group having an average molecular weight of 20 kDa. In some embodiments of the IL-10 conjugate comprising Formula (II) and having an amino acid sequence of SEQ ID NO: 19, W is a PEG group having an average molecular weight of 30 kDa.

In some embodiments of the IL-10 conjugate comprising Formula (II), the IL-10 conjugate has the amino acid sequence of SEQ ID NO: 20. In some embodiments of the IL-10 conjugate comprising Formula (II) and having an amino acid sequence of SEQ ID NO: 20, W is a PEG group having an average molecular weight selected from 5 kDa, 10 kDa, 15 kDa, 20 kDa, 25 kDa, and 30 kDa. In some embodiments of the IL-10 conjugate comprising Formula (II) and having an amino acid sequence of SEQ ID NO: 20, W is a PEG group having an average molecular weight selected from 20 kDa and 30 kDa. In some embodiments of the IL-10 conjugate comprising Formula (II) and having an amino acid sequence of SEQ ID NO: 20, W is a PEG group having an average molecular weight of 20 kDa. In some embodiments of the IL-10 conjugate comprising Formula (II) and having an amino acid sequence of SEQ ID NO: 20, W is a PEG group having an average molecular weight of 30 kDa.

In some embodiments of the IL-10 conjugate comprising Formula (II), the IL-10 conjugate has the amino acid sequence of SEQ ID NO: 21. In some embodiments of the IL-10 conjugate comprising Formula (II) and having an amino acid sequence of SEQ ID NO: 21, W is a PEG group having an average molecular weight selected from 5 kDa, 10 kDa, 15 kDa, 20 kDa, 25 kDa, and 30 kDa. In some embodiments of the IL-10 conjugate comprising Formula (II) and having an amino acid sequence of SEQ ID NO: 10, W is a PEG group having an average molecular weight selected from 20 kDa and 30 kDa. In some embodiments of the IL-10 conjugate comprising Formula (II) and having an amino acid sequence of SEQ ID NO: 21, W is a PEG group having an average molecular weight of 20 kDa. In some embodiments of the IL-10 conjugate comprising Formula (II) and having an amino acid sequence of SEQ ID NO: 21, W is a PEG group having an average molecular weight of 30 kDa.

In some embodiments of the IL-10 conjugate comprising Formula (II), the IL-10 conjugate has the amino acid sequence of SEQ ID NO: 22. In some embodiments of the IL-10 conjugate comprising Formula (II) and having an amino acid sequence of SEQ ID NO: 22, W is a PEG group having an average molecular weight selected from 5 kDa, 10 kDa, 15 kDa, 20 kDa, 25 kDa, and 30 kDa. In some embodiments of the IL-10 conjugate comprising Formula (II) and having an amino acid sequence of SEQ ID NO: 22, W is a PEG group having an average molecular weight selected from 20 kDa and 30 kDa. In some embodiments of the IL-10 conjugate comprising Formula (II) and having an amino acid sequence of SEQ ID NO: 22, W is a PEG group having an average molecular weight of 20 kDa. In some embodiments of the IL-10 conjugate comprising Formula (II) and having an amino acid sequence of SEQ ID NO: 22, W is a PEG group having an average molecular weight of 30 kDa.

In some embodiments of the IL-10 conjugate comprising Formula (II), the IL-10 conjugate has the amino acid sequence of SEQ ID NO: 23. In some embodiments of the IL-10 conjugate comprising Formula (II) and having an amino acid sequence of SEQ ID NO: 23, W is a PEG group having an average molecular weight selected from 5 kDa, 10 kDa, 15 kDa, 20 kDa, 25 kDa, and 30 kDa. In some embodiments of the IL-10 conjugate comprising Formula (II) and having an amino acid sequence of SEQ ID NO: 23, W is a PEG group having an average molecular weight selected from 20 kDa and 30 kDa. In some embodiments of the IL-10 conjugate comprising Formula (II) and having an amino acid sequence of SEQ ID NO: 23, W is a PEG group having an average molecular weight of 20 kDa. In some embodiments of the IL-10 conjugate comprising Formula (II) and having an amino acid sequence of SEQ ID NO: 23, W is a PEG group having an average molecular weight of 30 kDa.

In some embodiments of the IL-10 conjugate comprising Formula (II), the IL-10 conjugate has the amino acid sequence of SEQ ID NO: 24. In some embodiments of the IL-10 conjugate comprising Formula (II) and having an amino acid sequence of SEQ ID NO: 24, W is a PEG group having an average molecular weight selected from 5 kDa, 10 kDa, 15 kDa, 20 kDa, 25 kDa, and 30 kDa. In some embodiments of the IL-10 conjugate comprising Formula (II) and having an amino acid sequence of SEQ ID NO: 24, W is a PEG group having an average molecular weight selected from 20 kDa and 30 kDa. In some embodiments of the IL-10 conjugate comprising Formula (II) and having an amino acid sequence of SEQ ID NO: 24, W is a PEG group having an average molecular weight of 20 kDa. In some embodiments of the IL-10 conjugate comprising Formula (II) and having an amino acid sequence of SEQ ID NO: 24, W is a PEG group having an average molecular weight of 30 kDa.

In some embodiments of the IL-10 conjugate comprising Formula (II), the IL-10 conjugate has the amino acid sequence of SEQ ID NO: 25. In some embodiments of the IL-10 conjugate comprising Formula (II) and having an amino acid sequence of SEQ ID NO: 25, W is a PEG group having an average molecular weight selected from 5 kDa, 10 kDa, 15 kDa, 20 kDa, 25 kDa, and 30 kDa. In some embodiments of the IL-10 conjugate comprising Formula (II) and having an amino acid sequence of SEQ ID NO: 25, W is a PEG group having an average molecular weight selected from 20 kDa and 30 kDa. In some embodiments of the IL-10 conjugate comprising Formula (II) and having an amino acid sequence of SEQ ID NO: 25, W is a PEG group having an average molecular weight of 20 kDa. In some embodiments of the IL-10 conjugate comprising Formula (II) and having an amino acid sequence of SEQ ID NO: 25, W is a PEG group having an average molecular weight of 30 kDa.

In some embodiments of the IL-10 conjugate comprising Formula (II), the IL-10 conjugate has the amino acid sequence of SEQ ID NO: 26. In some embodiments of the IL-10 conjugate comprising Formula (II) and having an amino acid sequence of SEQ ID NO: 26, W is a PEG group having an average molecular weight selected from 5 kDa, 10 kDa, 15 kDa, 20 kDa, 25 kDa, and 30 kDa. In some embodiments of the IL-10 conjugate comprising Formula (II) and having an amino acid sequence of SEQ ID NO: 26, W is a PEG group having an average molecular weight selected from 20 kDa and 30 kDa. In some embodiments of the IL-10 conjugate comprising Formula (II) and having an amino acid sequence of SEQ ID NO: 26, W is a PEG group having an average molecular weight of 20 kDa. In some embodiments of the IL-10 conjugate comprising Formula (II) and having an amino acid sequence of SEQ ID NO: 26, W is a PEG group having an average molecular weight of 30 kDa.

In some embodiments of the IL-10 conjugate comprising Formula (III), the IL-10 conjugate has the amino acid sequence of SEQ ID NO: 19. In some embodiments of the IL-10 conjugate comprising Formula (III) and having an amino acid sequence of SEQ ID NO: 19, W is a PEG group having an average molecular weight selected from 5 kDa, 10 kDa, 15 kDa, 20 kDa, 25 kDa, and 30 kDa. In some embodiments of the IL-10 conjugate comprising Formula (III) and having an amino acid sequence of SEQ ID NO: 19, W is a PEG group having an average molecular weight selected from 20 kDa and 30 kDa. In some embodiments of the IL-10 conjugate comprising Formula (III) and having an amino acid sequence of SEQ ID NO: 19, W is a PEG group having an average molecular weight of 20 kDa. In some embodiments of the IL-10 conjugate comprising Formula (III) and having an amino acid sequence of SEQ ID NO: 19, W is a PEG group having an average molecular weight of 30 kDa.

In some embodiments of the IL-10 conjugate comprising Formula (III), the IL-10 conjugate has the amino acid sequence of SEQ ID NO: 20. In some embodiments of the IL-10 conjugate comprising Formula (III) and having an amino acid sequence of SEQ ID NO: 20, W is a PEG group having an average molecular weight selected from 5 kDa, 10 kDa, 15 kDa, 20 kDa, 25 kDa, and 30 kDa. In some embodiments of the IL-10 conjugate comprising Formula (III) and having an amino acid sequence of SEQ ID NO: 20, W is a PEG group having an average molecular weight selected from 20 kDa and 30 kDa. In some embodiments of the IL-10 conjugate comprising Formula (III) and having an amino acid sequence of SEQ ID NO: 20, W is a PEG group having an average molecular weight of 20 kDa. In some embodiments of the IL-10 conjugate comprising Formula (III) and having an amino acid sequence of SEQ ID NO: 20, W is a PEG group having an average molecular weight of 30 kDa.

In some embodiments of the IL-10 conjugate comprising Formula (III), the IL-10 conjugate has the amino acid sequence of SEQ ID NO: 21. In some embodiments of the IL-10 conjugate comprising Formula (III) and having an amino acid sequence of SEQ ID NO: 21, W is a PEG group having an average molecular weight selected from 5 kDa, 10 kDa, 15 kDa, 20 kDa, 25 kDa, and 30 kDa. In some embodiments of the IL-10 conjugate comprising Formula (III) and having an amino acid sequence of SEQ ID NO: 10, W is a PEG group having an average molecular weight selected from 20 kDa and 30 kDa. In some embodiments of the IL-10 conjugate comprising Formula (III) and having an amino acid sequence of SEQ ID NO: 21, W is a PEG group having an average molecular weight of 20 kDa. In some embodiments of the IL-10 conjugate comprising Formula (III) and having an amino acid sequence of SEQ ID NO: 21, W is a PEG group having an average molecular weight of 30 kDa.

In some embodiments of the IL-10 conjugate comprising Formula (III), the IL-10 conjugate has the amino acid sequence of SEQ ID NO: 22. In some embodiments of the IL-10 conjugate comprising Formula (III) and having an amino acid sequence of SEQ ID NO: 22, W is a PEG group having an average molecular weight selected from 5 kDa, 10 kDa, 15 kDa, 20 kDa, 25 kDa, and 30 kDa. In some embodiments of the IL-10 conjugate comprising Formula (III) and having an amino acid sequence of SEQ ID NO: 22, W is a PEG group having an average molecular weight selected from 20 kDa and 30 kDa. In some embodiments of the IL-10 conjugate comprising Formula (III) and having an amino acid sequence of SEQ ID NO: 22, W is a PEG group having an average molecular weight of 20 kDa. In some embodiments of the IL-10 conjugate comprising Formula (III) and having an amino acid sequence of SEQ ID NO: 22, W is a PEG group having an average molecular weight of 30 kDa.

In some embodiments of the IL-10 conjugate comprising Formula (III), the IL-10 conjugate has the amino acid sequence of SEQ ID NO: 23. In some embodiments of the IL-10 conjugate comprising Formula (III) and having an amino acid sequence of SEQ ID NO: 23, W is a PEG group having an average molecular weight selected from 5 kDa, 10 kDa, 15 kDa, 20 kDa, 25 kDa, and 30 kDa. In some embodiments of the IL-10 conjugate comprising Formula (III) and having an amino acid sequence of SEQ ID NO: 23, W is a PEG group having an average molecular weight selected from 20 kDa and 30 kDa. In some embodiments of the IL-10 conjugate comprising Formula (III) and having an amino acid sequence of SEQ ID NO: 23, W is a PEG group having an average molecular weight of 20 kDa. In some embodiments of the IL-10 conjugate comprising Formula (III) and having an amino acid sequence of SEQ ID NO: 23, W is a PEG group having an average molecular weight of 30 kDa.

In some embodiments of the IL-10 conjugate comprising Formula (III), the IL-10 conjugate has the amino acid sequence of SEQ ID NO: 24. In some embodiments of the IL-10 conjugate comprising Formula (III) and having an amino acid sequence of SEQ ID NO: 24, W is a PEG group having an average molecular weight selected from 5 kDa, 10 kDa, 15 kDa, 20 kDa, 25 kDa, and 30 kDa. In some embodiments of the IL-10 conjugate comprising Formula (III) and having an amino acid sequence of SEQ ID NO: 24, W is a PEG group having an average molecular weight selected from 20 kDa and 30 kDa. In some embodiments of the IL-10 conjugate comprising Formula (III) and having an amino acid sequence of SEQ ID NO: 24, W is a PEG group having an average molecular weight of 20 kDa. In some embodiments of the IL-10 conjugate comprising Formula (III) and having an amino acid sequence of SEQ ID NO: 24, W is a PEG group having an average molecular weight of 30 kDa.

In some embodiments of the IL-10 conjugate comprising Formula (III), the IL-10 conjugate has the amino acid sequence of SEQ ID NO: 25. In some embodiments of the IL-10 conjugate comprising Formula (III) and having an amino acid sequence of SEQ ID NO: 25, W is a PEG group having an average molecular weight selected from 5 kDa, 10 kDa, 15 kDa, 20 kDa, 25 kDa, and 30 kDa. In some embodiments of the IL-10 conjugate comprising Formula (III) and having an amino acid sequence of SEQ ID NO: 25, W is a PEG group having an average molecular weight selected from 20 kDa and 30 kDa. In some embodiments of the IL-10 conjugate comprising Formula (III) and having an amino acid sequence of SEQ ID NO: 25, W is a PEG group having an average molecular weight of 20 kDa. In some embodiments of the IL-10 conjugate comprising Formula (III) and having an amino acid sequence of SEQ ID NO: 25, W is a PEG group having an average molecular weight of 30 kDa.

In some embodiments of the IL-10 conjugate comprising Formula (III), the IL-10 conjugate has the amino acid sequence of SEQ ID NO: 26. In some embodiments of the IL-10 conjugate comprising Formula (III) and having an amino acid sequence of SEQ ID NO: 26, W is a PEG group having an average molecular weight selected from 5 kDa, 10 kDa, 15 kDa, 20 kDa, 25 kDa, and 30 kDa. In some embodiments of the IL-10 conjugate comprising Formula (III) and having an amino acid sequence of SEQ ID NO: 26, W is a PEG group having an average molecular weight selected from 20 kDa and 30 kDa. In some embodiments of the IL-10 conjugate comprising Formula (III) and having an amino acid sequence of SEQ ID NO: 26, W is a PEG group having an average molecular weight of 20 kDa. In some embodiments of the IL-10 conjugate comprising Formula (III) and having an amino acid sequence of SEQ ID NO: 26, W is a PEG group having an average molecular weight of 30 kDa.

In some embodiments of the IL-10 conjugate comprising Formula (II), Formula (III), or a mixture of Formula (II) and Formula (III), the IL-10 conjugate has the amino acid sequence of SEQ ID NO: 19. In some embodiments of the IL-10 conjugate comprising Formula (II), Formula (III), or a mixture of Formula (II) and Formula (III) and having an amino acid sequence of SEQ ID NO: 19, W is a PEG group having an average molecular weight selected from 5 kDa, 10 kDa, 15 kDa, 20 kDa, 25 kDa, and 30 kDa. In some embodiments of the IL-10 conjugate comprising Formula (II), Formula (III), or a mixture of Formula (II) and Formula (III) and having an amino acid sequence of SEQ ID NO: 19, W is a PEG group having an average molecular weight selected from 20 kDa and 30 kDa. In some embodiments of the IL-10 conjugate comprising Formula (II), Formula (III), or a mixture of Formula (II) and Formula (III) and having an amino acid sequence of SEQ ID NO: 19, W is a PEG group having an average molecular weight of 20 kDa. In some embodiments of the IL-10 conjugate comprising Formula (II), Formula (III), or a mixture of Formula (II) and Formula (III) and having an amino acid sequence of SEQ ID NO: 19, W is a PEG group having an average molecular weight of 30 kDa.

In some embodiments of the IL-10 conjugate comprising Formula (II), Formula (III), or a mixture of Formula (II) and Formula (III), the IL-10 conjugate has the amino acid sequence of SEQ ID NO: 20. In some embodiments of the IL-10 conjugate comprising Formula (II), Formula (III), or a mixture of Formula (II) and Formula (III) and having an amino acid sequence of SEQ ID NO: 20, W is a PEG group having an average molecular weight selected from 5 kDa, 10 kDa, 15 kDa, 20 kDa, 25 kDa, and 30 kDa. In some embodiments of the IL-10 conjugate comprising Formula (II), Formula (III), or a mixture of Formula (II) and Formula (III) and having an amino acid sequence of SEQ ID NO: 20, W is a PEG group having an average molecular weight selected from 20 kDa and 30 kDa. In some embodiments of the IL-10 conjugate comprising Formula (II), Formula (III), or a mixture of Formula (II) and Formula (III) and having an amino acid sequence of SEQ ID NO: 20, W is a PEG group having an average molecular weight of 20 kDa. In some embodiments of the IL-10 conjugate comprising Formula (II), Formula (III), or a mixture of Formula (II) and Formula (III) and having an amino acid sequence of SEQ ID NO: 20, W is a PEG group having an average molecular weight of 30 kDa.

In some embodiments of the IL-10 conjugate comprising Formula (II), Formula (III), or a mixture of Formula (II) and Formula (III), the IL-10 conjugate has the amino acid sequence of SEQ ID NO: 21. In some embodiments of the IL-10 conjugate comprising Formula (II), Formula (III), or a mixture of Formula (II) and Formula (III) and having an amino acid sequence of SEQ ID NO: 21, W is a PEG group having an average molecular weight selected from 5 kDa, 10 kDa, 15 kDa, 20 kDa, 25 kDa, and 30 kDa. In some embodiments of the IL-10 conjugate comprising Formula (II), Formula (III), or a mixture of Formula (II) and Formula (III) and having an amino acid sequence of SEQ ID NO: 10, W is a PEG group having an average molecular weight selected from 20 kDa and 30 kDa. In some embodiments of the IL-10 conjugate comprising Formula (II), Formula (III), or a mixture of Formula (II) and Formula (III) and having an amino acid sequence of SEQ ID NO: 21, W is a PEG group having an average molecular weight of 20 kDa. In some embodiments of the IL-10 conjugate comprising Formula (II), Formula (III), or a mixture of Formula (II) and Formula (III) and having an amino acid sequence of SEQ ID NO: 21, W is a PEG group having an average molecular weight of 30 kDa.

In some embodiments of the IL-10 conjugate comprising Formula (II), Formula (III), or a mixture of Formula (II) and Formula (III), the IL-10 conjugate has the amino acid sequence of SEQ ID NO: 22. In some embodiments of the IL-10 conjugate comprising Formula (II), Formula (III), or a mixture of Formula (II) and Formula (III) and having an amino acid sequence of SEQ ID NO: 22, W is a PEG group having an average molecular weight selected from 5 kDa, 10 kDa, 15 kDa, 20 kDa, 25 kDa, and 30 kDa. In some embodiments of the IL-10 conjugate comprising Formula (II), Formula (III), or a mixture of Formula (II) and Formula (III) and having an amino acid sequence of SEQ ID NO: 22, W is a PEG group having an average molecular weight selected from 20 kDa and 30 kDa. In some embodiments of the IL-10 conjugate comprising Formula (II), Formula (III), or a mixture of Formula (II) and Formula (III) and having an amino acid sequence of SEQ ID NO: 22, W is a PEG group having an average molecular weight of 20 kDa. In some embodiments of the IL-10 conjugate comprising Formula (II), Formula (III), or a mixture of Formula (II) and Formula (III) and having an amino acid sequence of SEQ ID NO: 22, W is a PEG group having an average molecular weight of 30 kDa.

In some embodiments of the IL-10 conjugate comprising Formula (II), Formula (III), or a mixture of Formula (II) and Formula (III), the IL-10 conjugate has the amino acid sequence of SEQ ID NO: 23. In some embodiments of the IL-10 conjugate comprising Formula (II), Formula (III), or a mixture of Formula (II) and Formula (III) and having an amino acid sequence of SEQ ID NO: 23, W is a PEG group having an average molecular weight selected from 5 kDa, 10 kDa, 15 kDa, 20 kDa, 25 kDa, and 30 kDa. In some embodiments of the IL-10 conjugate comprising Formula (II), Formula (III), or a mixture of Formula (II) and Formula (III) and having an amino acid sequence of SEQ ID NO: 23, W is a PEG group having an average molecular weight selected from 20 kDa and 30 kDa. In some embodiments of the IL-10 conjugate comprising Formula (II), Formula (III), or a mixture of Formula (II) and Formula (III) and having an amino acid sequence of SEQ ID NO: 23, W is a PEG group having an average molecular weight of 20 kDa. In some embodiments of the IL-10 conjugate comprising Formula (II), Formula (III), or a mixture of Formula (II) and Formula (III) and having an amino acid sequence of SEQ ID NO: 23, W is a PEG group having an average molecular weight of 30 kDa.

In some embodiments of the IL-10 conjugate comprising Formula (II), Formula (III), or a mixture of Formula (II) and Formula (III), the IL-10 conjugate has the amino acid sequence of SEQ ID NO: 24. In some embodiments of the IL-10 conjugate comprising Formula (II), Formula (III), or a mixture of Formula (II) and Formula (III) and having an amino acid sequence of SEQ ID NO: 24, W is a PEG group having an average molecular weight selected from 5 kDa, 10 kDa, 15 kDa, 20 kDa, 25 kDa, and 30 kDa. In some embodiments of the IL-10 conjugate comprising Formula (II), Formula (III), or a mixture of Formula (II) and Formula (III) and having an amino acid sequence of SEQ ID NO: 24, W is a PEG group having an average molecular weight selected from 20 kDa and 30 kDa. In some embodiments of the IL-10 conjugate comprising Formula (II), Formula (III), or a mixture of Formula (II) and Formula (III) and having an amino acid sequence of SEQ ID NO: 24, W is a PEG group having an average molecular weight of 20 kDa. In some embodiments of the IL-10 conjugate comprising Formula (II), Formula (III), or a mixture of Formula (II) and Formula (III) and having an amino acid sequence of SEQ ID NO: 24, W is a PEG group having an average molecular weight of 30 kDa.

In some embodiments of the IL-10 conjugate comprising Formula (II), Formula (III), or a mixture of Formula (II) and Formula (III), the IL-10 conjugate has the amino acid sequence of SEQ ID NO: 25. In some embodiments of the IL-10 conjugate comprising Formula (II), Formula (III), or a mixture of Formula (II) and Formula (III) and having an amino acid sequence of SEQ ID NO: 25, W is a PEG group having an average molecular weight selected from 5 kDa, 10 kDa, 15 kDa, 20 kDa, 25 kDa, and 30 kDa. In some embodiments of the IL-10 conjugate comprising Formula (II), Formula (III), or a mixture of Formula (II) and Formula (III) and having an amino acid sequence of SEQ ID NO: 25, W is a PEG group having an average molecular weight selected from 20 kDa and 30 kDa. In some embodiments of the IL-10 conjugate comprising Formula (II), Formula (III), or a mixture of Formula (II) and Formula (III) and having an amino acid sequence of SEQ ID NO: 25, W is a PEG group having an average molecular weight of 20 kDa. In some embodiments of the IL-10 conjugate comprising Formula (II), Formula (III), or a mixture of Formula (II) and Formula (III) and having an amino acid sequence of SEQ ID NO: 25, W is a PEG group having an average molecular weight of 30 kDa.

In some embodiments of the IL-10 conjugate comprising Formula (II), Formula (III), or a mixture of Formula (II) and Formula (III), the IL-10 conjugate has the amino acid sequence of SEQ ID NO: 26. In some embodiments of the IL-10 conjugate comprising Formula (II), Formula (III), or a mixture of Formula (II) and Formula (III) and having an amino acid sequence of SEQ ID NO: 26, W is a PEG group having an average molecular weight selected from 5 kDa, 10 kDa, 15 kDa, 20 kDa, 25 kDa, and 30 kDa. In some embodiments of the IL-10 conjugate comprising Formula (II), Formula (III), or a mixture of Formula (II) and Formula (III) and having an amino acid sequence of SEQ ID NO: 26, W is a PEG group having an average molecular weight selected from 20 kDa and 30 kDa. In some embodiments of the IL-10 conjugate comprising Formula (II), Formula (III), or a mixture of Formula (II) and Formula (III) and having an amino acid sequence of SEQ ID NO: 26, W is a PEG group having an average molecular weight of 20 kDa. In some embodiments of the IL-10 conjugate comprising Formula (II), Formula (III), or a mixture of Formula (II) and Formula (III) and having an amino acid sequence of SEQ ID NO: 26, W is a PEG group having an average molecular weight of 30 kDa.

In some embodiments of the IL-10 conjugate comprising Formula (II), Formula (III), or a mixture of Formula (II) and Formula (III) and having an amino acid sequence of one or more SEQ ID NO: 19-26, W is a linear or branched PEG group. In some embodiments of the IL-10 conjugate comprising Formula (II), Formula (III), or a mixture of Formula (II) and Formula (III) and having an amino acid sequence of one or more SEQ ID NO: 19-26, W is a linear PEG group. In some embodiments of the IL-10 conjugate comprising Formula (II), Formula (III), or a mixture of Formula (II) and Formula (III) and having an amino acid sequence of one or more SEQ ID NO: 19-26, W is a branched PEG group. In some embodiments of the IL-10 conjugate comprising Formula (II), Formula (III), or a mixture of Formula (II) and Formula (III) and having an amino acid sequence of one or more SEQ ID NO: 19-26, W is a methoxy PEG group. In some embodiments of the IL-10 conjugate comprising Formula (II), Formula (III), or a mixture of Formula (II) and Formula (III) and having an amino acid sequence of one or more SEQ ID NO: 19-26, the methoxy PEG group is linear or branched. In some embodiments of the IL-10 conjugate comprising Formula (II), Formula (III), or a mixture of Formula (II) and Formula (III) and having an amino acid sequence of one or more SEQ ID NO: 19-26, the methoxy PEG group is linear. In some embodiments of the IL-10 conjugate comprising Formula (II), Formula (III), or a mixture of Formula (II) and Formula (III) and having an amino acid sequence of one or more SEQ ID NO: 19-26, the methoxy PEG group is branched.

Described herein, in some embodiments, is an IL-10 conjugate comprising the amino acid sequence of any one of SEQ ID NOS: 27 to 34, wherein [AzK_PEG20 kDa] has the structure of Formula (II), Formula (III), or a mixture of Formula (II) and Formula (III):

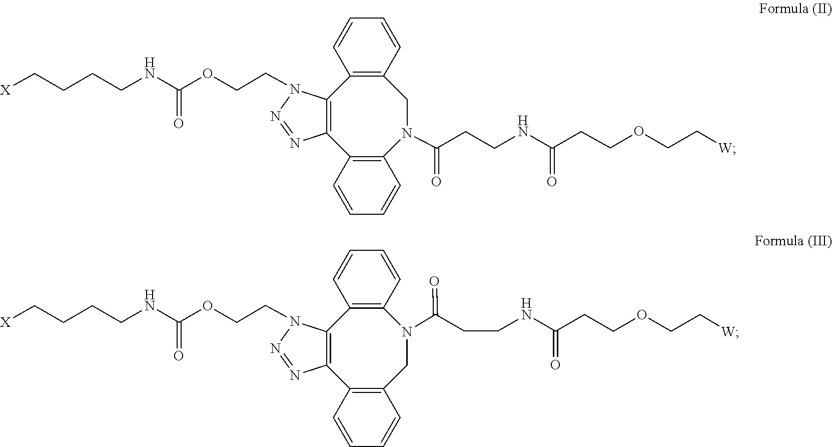

wherein:
W is a PEG group having an average molecular weight of 20 kDa; and
X has the structure:

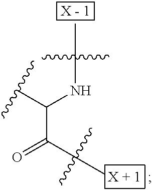

X−1 indicates the point of attachment to the preceding amino acid residue; and
X+1 indicates the point of attachment to the following amino acid residue.

Described herein, in some embodiments, is an IL-10 conjugate comprising the amino acid sequence of any one of SEQ ID NOS: 27 to 34, wherein [AzK_PEG20 kDa] has the structure of Formula (II), Formula (III), or a mixture of Formula (II) and Formula (III):

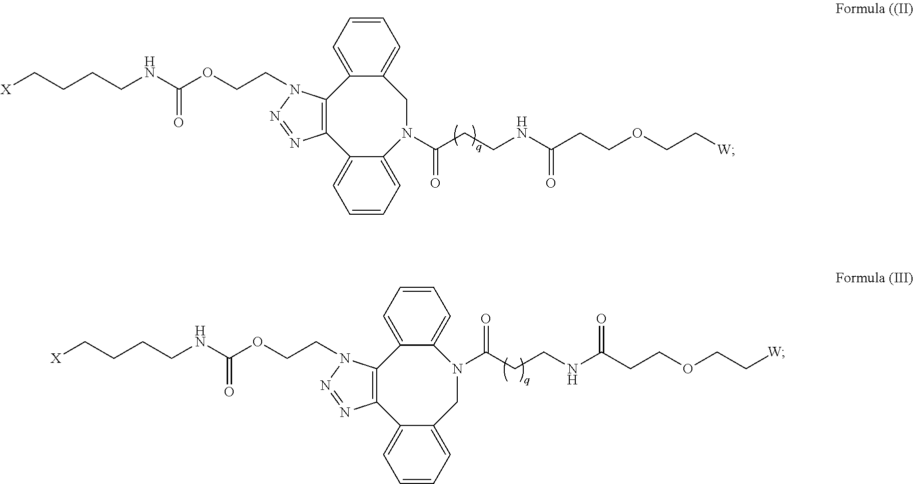

wherein:
W is a PEG group having an average molecular weight of 20 kDa;
q is 1, 2, or 3; and
X has the structure:

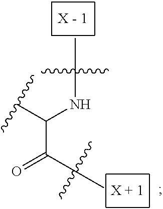 [SMILES: "X - 1" ; *NC(*)C(*)=O ; "X + 1"]

X−1 indicates the point of attachment to the preceding amino acid residue; and
X+1 indicates the point of attachment to the following amino acid residue. In some embodiments, q is 1. In some embodiments, q is 2. In some embodiments, q is 3.

In some embodiments of the IL-10 conjugate comprising [AzK_PEG20 kDa] and having the structure of Formula (II), Formula (III), or a mixture of Formula (II) and Formula (III), the IL-10 conjugate has the amino acid sequence of SEQ ID NO: 27. In some embodiments of the IL-10 conjugate comprising [AzK_PEG20 kDa] and having the structure of Formula (II), Formula (III), or a mixture of Formula (II) and Formula (III), the IL-10 conjugate has the amino acid sequence of SEQ ID NO: 28. In some embodiments of the IL-10 conjugate comprising [AzK_PEG20 kDa] and having the structure of Formula (II), Formula (III), or a mixture of Formula (II) and Formula (III), the IL-10 conjugate has the amino acid sequence of SEQ ID NO: 29. In some embodiments of the IL-10 conjugate comprising [AzK_PEG20 kDa] and having the structure of Formula (II), Formula (III), or a mixture of Formula (II) and Formula (III), the IL-10 conjugate has the amino acid sequence of SEQ ID NO: 30. In some embodiments of the IL-10 conjugate comprising [AzK_PEG20 kDa] and having the structure of Formula (II), Formula (III), or a mixture of Formula (II) and Formula (III), the IL-10 conjugate has the amino acid sequence of SEQ ID NO: 31. In some embodiments of the IL-10 conjugate comprising [AzK_PEG20 kDa] and having the structure of Formula (II), Formula (III), or a mixture of Formula (II) and Formula (III), the IL-10 conjugate has the amino acid sequence of SEQ ID NO: 32. In some embodiments of the IL-10 conjugate comprising [AzK_PEG20 kDa] and having the structure of Formula (II), Formula (III), or a mixture of Formula (II) and Formula (III), the IL-10 conjugate has the amino acid sequence of SEQ ID NO: 33. In some embodiments of the IL-10 conjugate comprising [AzK_PEG20 kDa] and having the structure of Formula (II), Formula (III), or a mixture of Formula (II) and Formula (III), the IL-10 conjugate has the amino acid sequence of SEQ ID NO: 34.

In some embodiments of the IL-10 conjugate comprising [AzK_PEG20 kDa] and having the structure of Formula (II), the IL-10 conjugate has the amino acid sequence of SEQ ID NO: 27. In some embodiments of the IL-10 conjugate comprising [AzK_PEG20 kDa] and having the structure of Formula (II), the IL-10 conjugate has the amino acid sequence of SEQ ID NO: 28. In some embodiments of the IL-10 conjugate comprising [AzK_PEG20 kDa] and having the structure of Formula (II), the IL-10 conjugate has the amino acid sequence of SEQ ID NO: 29. In some embodiments of the IL-10 conjugate comprising [AzK_PEG20 kDa] and having the structure of Formula (II), the IL-10 conjugate has the amino acid sequence of SEQ ID NO: 30. In some embodiments of the IL-10 conjugate comprising [AzK_PEG20 kDa] and having the structure of Formula (II), the IL-10 conjugate has the amino acid sequence of SEQ ID NO: 31. In some embodiments of the IL-10 conjugate comprising [AzK_PEG20 kDa] and having the structure of Formula (II), the IL-10 conjugate has the amino acid sequence of SEQ ID NO: 32. In some embodiments of the IL-10 conjugate comprising [AzK_PEG20 kDa] and having the structure of Formula (II), the IL-10 conjugate has the amino acid sequence of SEQ ID NO: 33. In some embodiments of the IL-10 conjugate comprising [AzK_PEG20 kDa] and having the structure of Formula (II), the IL-10 conjugate has the amino acid sequence of SEQ ID NO: 34.

In some embodiments of the IL-10 conjugate comprising [AzK_PEG20 kDa] and having the structure of Formula (III), the IL-10 conjugate has the amino acid sequence of SEQ ID NO: 27. In some embodiments of the IL-10 conjugate comprising [AzK_PEG20 kDa] and having the structure of Formula (III), the IL-10 conjugate has the amino acid sequence of SEQ ID NO: 28. In some embodiments of the IL-10 conjugate comprising [AzK_PEG20 kDa] and having the structure of Formula (III), the IL-10 conjugate has the amino acid sequence of SEQ ID NO: 29. In some embodiments of the IL-10 conjugate comprising [AzK_PEG20 kDa] and having the structure of Formula (III), the IL-10 conjugate has the amino acid sequence of SEQ ID NO: 30. In some embodiments of the IL-10 conjugate comprising [AzK_PEG20 kDa] and having the structure of Formula (III), the IL-10 conjugate has the amino acid sequence of SEQ ID NO: 31. In some embodiments of the IL-10 conjugate comprising [AzK_PEG20 kDa] and having the structure of Formula (III), the IL-10 conjugate has the amino acid sequence of SEQ ID NO: 32. In some embodiments of the IL-10 conjugate comprising [AzK_PEG20 kDa] and having the structure of Formula (III), the IL-10 conjugate has the amino acid sequence of SEQ ID NO: 33. In some embodiments of the IL-10 conjugate comprising [AzK_PEG20 kDa] and having the structure of Formula (III), the IL-10 conjugate has the amino acid sequence of SEQ ID NO: 34.

Described herein, in some embodiments, is an IL-10 conjugate comprising the amino acid sequence of any one of SEQ ID NOS: 35 to 42, wherein [AzK_PEG30 kDa] has the structure of Formula (II), Formula (III), or a mixture of Formula (II) and Formula (III):

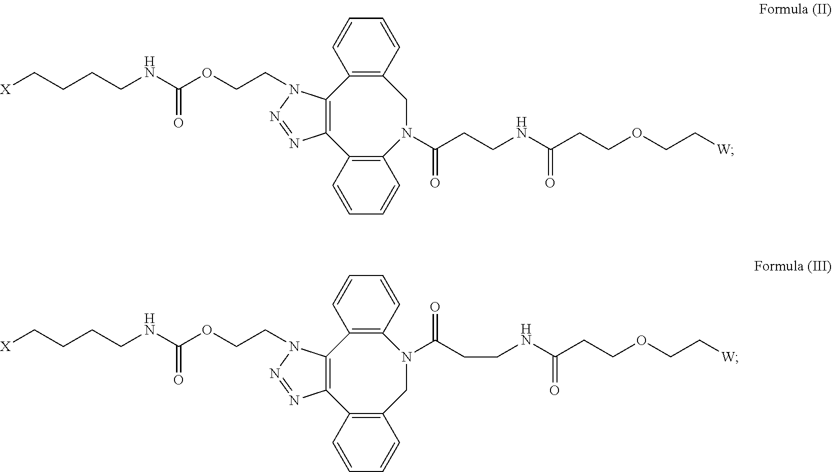

wherein:
W is a PEG group having an average molecular weight of 30 kDa; and
X has the structure:

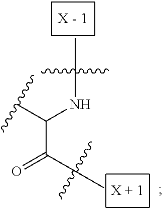 [SMILES: "X - 1" ; *NC(*)C(*)=O ; "X + 1"]

X−1 indicates the point of attachment to the preceding amino acid residue; and X+1 indicates the point of attachment to the following amino acid residue.

Described herein, in some embodiments, is an IL-10 conjugate comprising the amino acid sequence of any one of SEQ ID NOS: 35 to 42, wherein [AzK_PEG30 kDa] has the structure of Formula (II), Formula (III), or a mixture of Formula (II) and Formula (III):

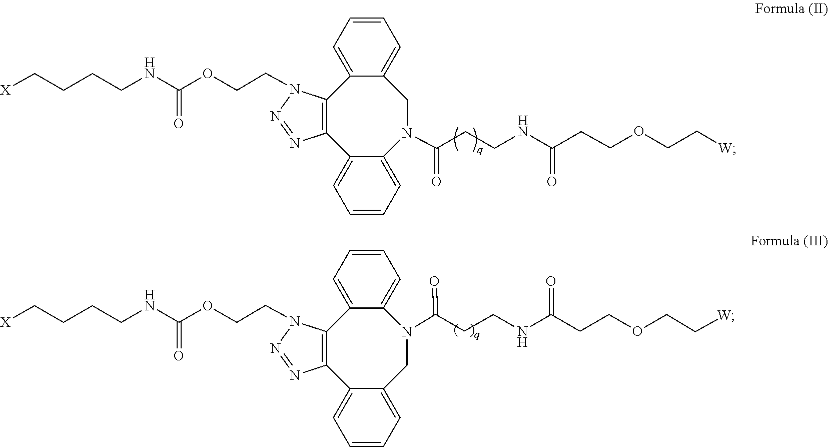

wherein:
W is a PEG group having an average molecular weight of 30 kDa;
q is 1, 2, or 3; and
X has the structure:

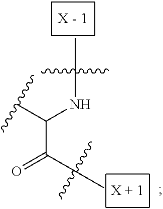 [SMILES: "X - 1" ; *NC(*)C(*)=O ; "X + 1"]

X−1 indicates the point of attachment to the preceding amino acid residue; and
X+1 indicates the point of attachment to the following amino acid residue. In some embodiments, q is 1. In some embodiments, q is 2. In some embodiments, q is 3.

In some embodiments of the IL-10 conjugate comprising [AzK_PEG30 kDa] and having the structure of Formula (II), Formula (III), or a mixture of Formula (II) and Formula (III), the IL-10 conjugate has the amino acid sequence of SEQ ID NO: 35. In some embodiments of the IL-10 conjugate comprising [AzK_PEG30 kDa] and having the structure of Formula (II), Formula (III), or a mixture of Formula (II) and Formula (III), the IL-10 conjugate has the amino acid sequence of SEQ ID NO: 36. In some embodiments of the IL-10 conjugate comprising [AzK_PEG30 kDa] and having the structure of Formula (II), Formula (III), or a mixture of Formula (II) and Formula (III), the IL-10 conjugate has the amino acid sequence of SEQ ID NO: 37. In some embodiments of the IL-10 conjugate comprising [AzK_PEG30 kDa] and having the structure of Formula (II), Formula (III), or a mixture of Formula (II) and Formula (III), the IL-10 conjugate has the amino acid sequence of SEQ ID NO: 38. In some embodiments of the IL-10 conjugate comprising [AzK_PEG30 kDa] and having the structure of Formula (II), Formula (III), or a mixture of Formula (II) and Formula (III), the IL-10 conjugate has the amino acid sequence of SEQ ID NO: 39. In some embodiments of the IL-10 conjugate comprising [AzK_PEG30 kDa] and having the structure of Formula (II), Formula (III), or a mixture of Formula (II) and Formula (III), the IL-10 conjugate has the amino acid sequence of SEQ ID NO: 40. In some embodiments of the IL-10 conjugate comprising [AzK_PEG30 kDa] and having the structure of Formula (II), Formula (III), or a mixture of Formula (II) and Formula (III), the IL-10 conjugate has the amino acid sequence of SEQ ID NO: 41. In some embodiments of the IL-10 conjugate comprising [AzK_PEG30 kDa] and having the structure of Formula (II), Formula (III), or a mixture of Formula (II) and Formula (III), the IL-10 conjugate has the amino acid sequence of SEQ ID NO: 42.

In some embodiments of the IL-10 conjugate comprising [AzK_PEG30 kDa] and having the structure of Formula (II), the IL-10 conjugate has the amino acid sequence of SEQ ID NO: 35. In some embodiments of the IL-10 conjugate comprising [AzK_PEG30 kDa] and having the structure of Formula (II), the IL-10 conjugate has the amino acid sequence of SEQ ID NO: 36. In some embodiments of the IL-10 conjugate comprising [AzK_PEG30 kDa] and having the structure of Formula (II), the IL-10 conjugate has the amino acid sequence of SEQ ID NO: 37. In some embodiments of the IL-10 conjugate comprising [AzK_PEG30 kDa] and having the structure of Formula (II), the IL-10 conjugate has the amino acid sequence of SEQ ID NO: 38. In some embodiments of the IL-10 conjugate comprising [AzK_PEG30 kDa] and having the structure of Formula (II), the IL-10 conjugate has the amino acid sequence of SEQ ID NO: 39. In some embodiments of the IL-10 conjugate comprising [AzK_PEG30 kDa] and having the structure of Formula (II), the IL-10 conjugate has the amino acid sequence of SEQ ID NO: 40. In some embodiments of the IL-10 conjugate comprising [AzK_PEG30 kDa] and having the structure of Formula (II), the IL-10 conjugate has the amino acid sequence of SEQ ID NO: 41. In some embodiments of the IL-10 conjugate comprising [AzK_PEG30 kDa] and having the structure of Formula (II), the IL-10 conjugate has the amino acid sequence of SEQ ID NO: 42.

In some embodiments of the IL-10 conjugate comprising [AzK_PEG30 kDa] and having the structure of Formula (III), the IL-10 conjugate has the amino acid sequence of SEQ ID NO: 35. In some embodiments of the IL-10 conjugate comprising [AzK_PEG30 kDa] and having the structure of Formula (III), the IL-10 conjugate has the amino acid sequence of SEQ ID NO: 36. In some embodiments of the IL-10 conjugate comprising [AzK_PEG30 kDa] and having the structure of Formula (III), the IL-10 conjugate has the amino acid sequence of SEQ ID NO: 37. In some embodiments of the IL-10 conjugate comprising [AzK_PEG30 kDa] and having the structure of Formula (III), the IL-10 conjugate has the amino acid sequence of SEQ ID NO: 38. In some embodiments of the IL-10 conjugate comprising [AzK_PEG30 kDa] and having the structure of Formula (III), the IL-10 conjugate has the amino acid sequence of SEQ ID NO: 39. In some embodiments of the IL-10 conjugate comprising [AzK_PEG30 kDa] and having the structure of Formula (III), the IL-10 conjugate has the amino acid sequence of SEQ ID NO: 40. In some embodiments of the IL-10 conjugate comprising [AzK_PEG30 kDa] and having the structure of Formula (III), the IL-10 conjugate has the amino acid sequence of SEQ ID NO: 41. In some embodiments of the IL-10 conjugate comprising [AzK_PEG30 kDa] and having the structure of Formula (III), the IL-10 conjugate has the amino acid sequence of SEQ ID NO: 42.

Described herein, in some embodiments, is an IL-10 conjugate comprising the amino acid sequence of any one of SEQ ID NOS: 19 to 26, wherein [AzK_PEG] is a mixture of the structures of Formula (II) and Formula (III):

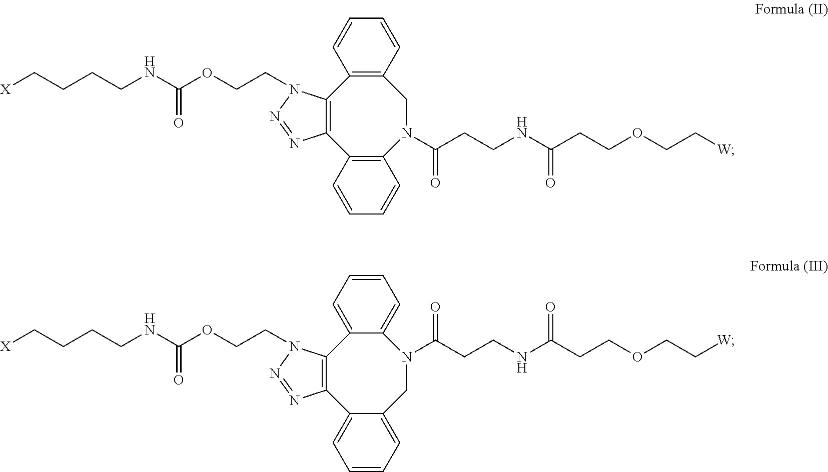

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

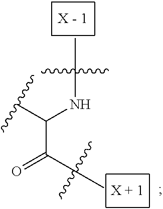 [SMILES: "X - 1" ; *NC(*)C(*)=O ; "X + 1"]

X−1 indicates the point of attachment to the preceding amino acid residue; and
X+1 indicates the point of attachment to the following amino acid residue.

Described herein, in some embodiments, is an IL-10 conjugate comprising the amino acid sequence of any one of SEQ ID NOS: 19 to 26, wherein [AzK_PEG] is a mixture of the structures of Formula (II) and Formula (III):

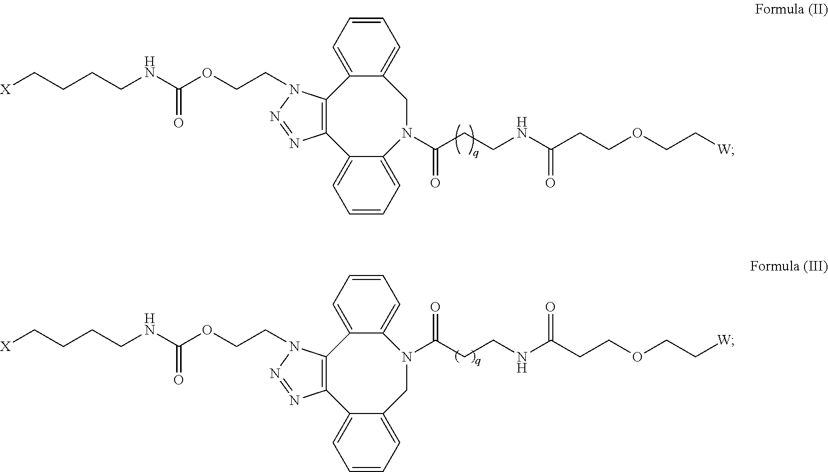

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa;
q is 1, 2, or 3; and
X has the structure:

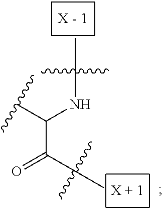 [SMILES: "X - 1" ; *NC(*)C(*)=O ; "X + 1"]

X−1 indicates the point of attachment to the preceding amino acid residue; and
X+1 indicates the point of attachment to the following amino acid residue. In some embodiments, q is 1. In some embodiments, q is 2. In some embodiments, q is 3.

In some embodiments, the IL-10 conjugate comprises the amino acid sequence of one or more SEQ ID NOS: 19-26, wherein [AzK_PEG] is a mixture of the structures of Formula (II) and Formula (III). In some embodiments of the IL-10 conjugate comprising the amino acid sequence of one or more SEQ ID NOS: 19-26 and having [AzK_PEG] as a mixture of the structures of Formula (II) and Formula (III), the ratio of the amount of the structure of Formula (II) to the amount of the structure of Formula (III) comprising the total amount of [AzK_PEG] in the IL-10 conjugate is about 1:1. In some embodiments of the IL-10 conjugate comprising the amino acid sequence of one or more SEQ ID NOS: 19-26 and having [AzK_PEG] as a mixture of the structures of Formula (II) and Formula (III), the ratio of the amount of the structure of Formula (II) to the amount of the structure of Formula (III) comprising the total amount of [AzK_PEG] in the IL-10 conjugate is greater than 1:1. In some embodiments of the IL-10 conjugate comprising the amino acid sequence of one or more SEQ ID NOS: 19-26 and having [AzK_PEG] as a mixture of the structures of Formula (II) and Formula (III), the ratio of the amount of the structure of Formula (II) to the amount of the structure of Formula (III) comprising the total amount of [AzK_PEG] in the IL-10 conjugate is less than 1:1.

In some embodiments of the IL-10 conjugate comprising the amino acid sequence of any one of SEQ ID NOS: 19 to 26 and having [AzK_PEG] as a mixture of the structures of Formula (II) and Formula (III), W is a linear or branched PEG group. In some embodiments of the IL-10 conjugate comprising the amino acid sequence of any one of SEQ ID NOS: 19 to 26 and having [AzK_PEG] as a mixture of the structures of Formula (II) and Formula (III), W is a linear PEG group. In some embodiments of the IL-10 conjugate comprising the amino acid sequence of any one of SEQ ID NOS: 19 to 26 and having [AzK_PEG] as a mixture of the structures of Formula (II) and Formula (III), W is a branched PEG group. In some embodiments of the IL-10 conjugate comprising the amino acid sequence of any one of SEQ ID NOS: 19 to 26 and having [AzK_PEG] as a mixture of the structures of Formula (II) and Formula (III), W is a methoxy PEG group. In some embodiments of the IL-10 conjugate comprising the amino acid sequence of any one of SEQ ID NOS: 19 to 26 and having [AzK_PEG] as a mixture of the structures of Formula (II) and Formula (III), the methoxy PEG group is linear or branched. In some embodiments of the IL-10 conjugate comprising the amino acid sequence of any one of SEQ ID NOS: 19 to 26 and having [AzK_PEG] as a mixture of the structures of Formula (II) and Formula (III), the methoxy PEG group is linear. In some embodiments of the IL-10 conjugate comprising the amino acid sequence of any one of SEQ ID NOS: 19 to 26 and having [AzK_PEG] as a mixture of the structures of Formula (II) and Formula (III), the methoxy PEG group is branched.

Described herein, in some embodiments, is an IL-10 conjugate comprising the amino acid sequence of any one of SEQ ID NOS: 27 to 34, wherein [AzK_PEG20 kDa] is a mixture of the structures of Formula (II) and Formula (III):

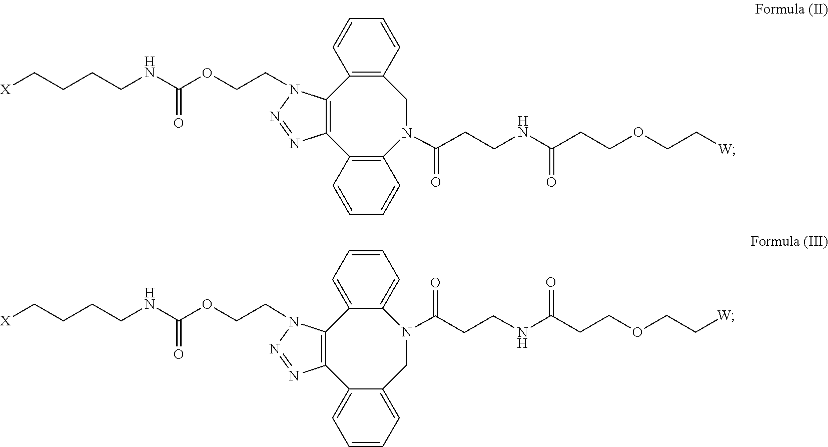

wherein:
W is a PEG group having an average molecular weight of 20 kDa; and
X has the structure:

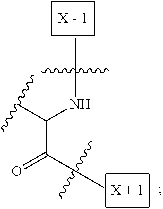 [SMILES: "X - 1" ; *NC(*)C(*)=O ; "X + 1"]

X−1 indicates the point of attachment to the preceding amino acid residue; and
X+1 indicates the point of attachment to the following amino acid residue.

Described herein, in some embodiments, is an IL-10 conjugate comprising the amino acid sequence of any one of SEQ ID NOS: 27 to 34, wherein [AzK_PEG20 kDa] is a mixture of the structures of Formula (II) and Formula (III):

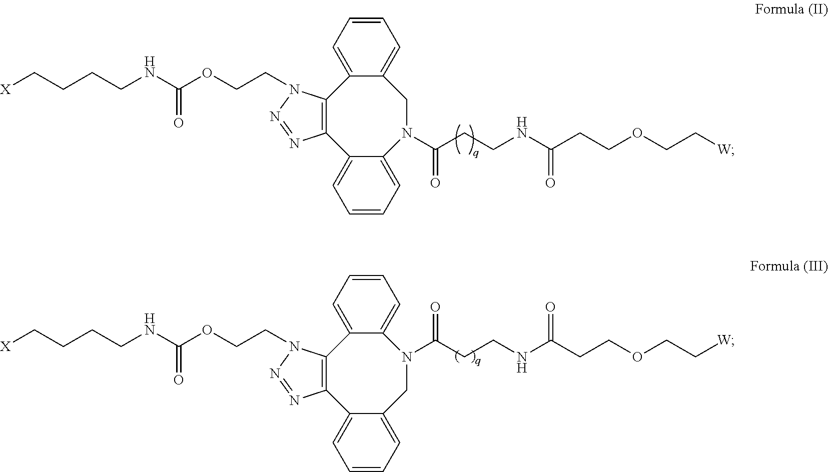

wherein:
W is a PEG group having an average molecular weight of 20 kDa;
q is 1, 2, or 3; and
X has the structure:

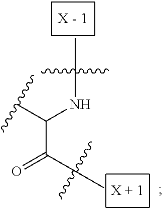 [SMILES: "X - 1" ; *NC(*)C(*)=O ; "X + 1"]

X−1 indicates the point of attachment to the preceding amino acid residue; and
X+1 indicates the point of attachment to the following amino acid residue. In some embodiments, q is 1. In some embodiments, q is 2. In some embodiments, q is 3.

In some embodiments, the IL-10 conjugate comprises the amino acid sequence of one or more of SEQ ID NOS: 27-34, wherein [AzK_PEG20 kDa] is a mixture of the structures of Formula (II) and Formula (III). In some embodiments of the IL-10 conjugate comprising the amino acid sequence one or more of SEQ ID NOS: 27-34 and having [AzK_PEG20 kDa] as a mixture of the structures of Formula (II) and Formula (III), the ratio of the amount of the structure of Formula (II) to the amount of the structure of Formula (III) comprising the total amount of [AzK_PEG20 kDa] in the IL-10 conjugate is about 1:1. In some embodiments of the IL-10 conjugate comprising the amino acid sequence one or more of SEQ ID NOS: 27-34 and having [AzK_PEG20 kDa] as a mixture of the structures of Formula (II) and Formula (III), the ratio of the amount of the structure of Formula (II) to the amount of the structure of Formula (III) comprising the total amount of [AzK_PEG20 kDa] in the IL-10 conjugate is greater than 1:1. In some embodiments of the IL-10 conjugate comprising the amino acid sequence of one or more of SEQ ID NOS: 27-34 and having [AzK_PEG20 kDa] as a mixture of the structures of Formula (II) and Formula (III), the ratio of the amount of the structure of Formula (II) to the amount of the structure of Formula (III) comprising the total amount of [AzK_PEG20 kDa] in the IL-10 conjugate less than about 1:1.

Described herein, in some embodiments, is an IL-10 conjugate comprising the amino acid sequence of any one of SEQ ID NOS: 35 to 42, wherein [AzK_PEG30 kDa] is a mixture of the structures of Formula (II) and Formula (III):

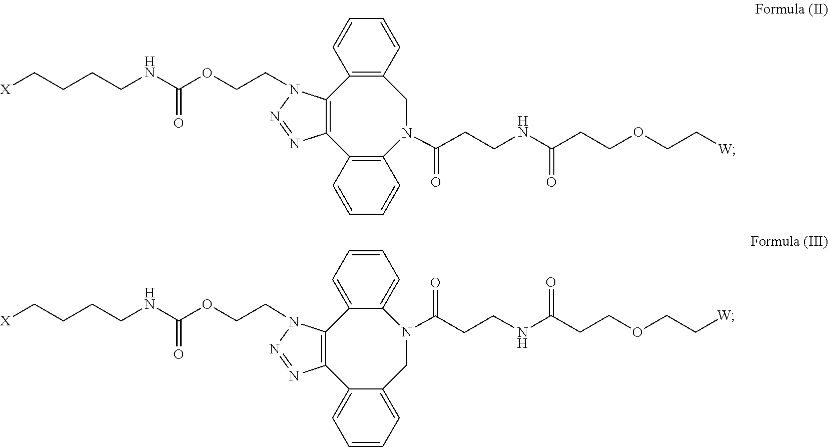

wherein:
W is a PEG group having an average molecular weight of 30 kDa; and
X has the structure:

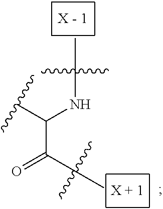 [SMILES: "X - 1" ; *NC(*)C(*)=O ; "X + 1"]

X−1 indicates the point of attachment to the preceding amino acid residue; and
X+1 indicates the point of attachment to the following amino acid residue.

Described herein, in some embodiments, is an IL-10 conjugate comprising the amino acid sequence of any one of SEQ ID NOS: 35 to 42, wherein [AzK_PEG30 kDa] is a mixture of the structures of Formula (II) and Formula (III):

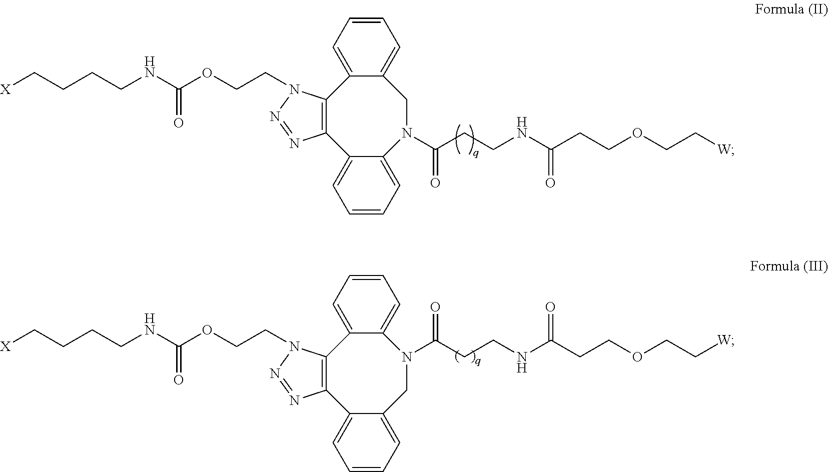

wherein:
W is a PEG group having an average molecular weight of 30 kDa;
q is 1, 2, or 3; and
X has the structure:

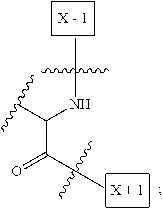 [SMILES: "X - 1" ; *NC(*)C(*)=O ; "X + 1"]

X−1 indicates the point of attachment to the preceding amino acid residue; and
X+1 indicates the point of attachment to the following amino acid residue. In some embodiments, q is 1. In some embodiments, q is 2. In some embodiments, q is 3.

In some embodiments, the IL-10 conjugate comprises the amino acid sequence of one or more of SEQ ID NOS: 35-42, wherein [AzK_PEG30 kDa] is a mixture of the structures of Formula (II) and Formula (III). In some embodiments of the IL-10 conjugate comprising the amino acid sequence of one or more of SEQ ID NOS: 35-42 and having [AzK_PEG30 kDa] as a mixture of the structures of Formula (II) and Formula (III), the ratio of the amount of the structure of Formula (II) to the amount of the structure of Formula (III) comprising the total amount of [AzK_PEG30 kDa] in the IL-10 conjugate is about 1:1. In some embodiments of the IL-10 conjugate comprising the amino acid sequence of one or more of SEQ ID NOS: 35-42 and having [AzK_PEG30 kDa] as a mixture of the structures of Formula (II) and Formula (III), the ratio of the amount of the structure of Formula (II) to the amount of the structure of Formula (III) comprising the total amount of [AzK_PEG30 kDa] in the IL-10 conjugate is greater than 1:1. In some embodiments of the IL-10 conjugate comprising the amino acid sequence of one or more of SEQ ID NOS: 35-42 and having [AzK_PEG30 kDa] as a mixture of the structures of Formula (II) and Formula (III), the ratio of the amount of the structure of Formula (II) to the amount of the structure of Formula (III) comprising the total amount of [AzK_PEG30 kDa] in the IL-10 conjugate less than about 1:1.

In some embodiments described herein of Formula (II), Formula (III), or a mixture of Formula (II) and Formula (III), q is 1. In some embodiments described herein of Formula (II), Formula (III), or a mixture of Formula (II) and Formula (III), q is 2. In some embodiments described herein of Formula (II), Formula (III), or a mixture of Formula (II) and Formula (III), q is 3. In some embodiments, the IL-10 conjugate comprises Formula (II) and q is 1. In some embodiments, the IL-10 conjugate comprises Formula (II) and q is 2. In some embodiments, the IL-10 conjugate comprises Formula (II) and q is 3. In some embodiments, the IL-10 conjugate comprises Formula (III) and q is 1. In some embodiments, the IL-10 conjugate comprises Formula (III) and q is 2. In some embodiments, the IL-10 conjugate comprises Formula (III) and q is 3. In some embodiments, the IL-10 conjugate comprises a mixture of Formula (II) and Formula (III) and q is 1. In some embodiments, the IL-10 conjugate comprises a mixture of Formula (II) and Formula (III) and q is 2. In some embodiments, the IL-10 conjugate comprises a mixture of Formula (II) and Formula (III) and q is 3.

Described herein, in some embodiments, is an IL-10 conjugate comprising the amino acid sequence of any one of SEQ ID NOS: 59 to 66, wherein [AzK_L1_PEG] has the structure of Formula (IV), Formula (V), or a mixture of Formula (IV) and Formula (V):

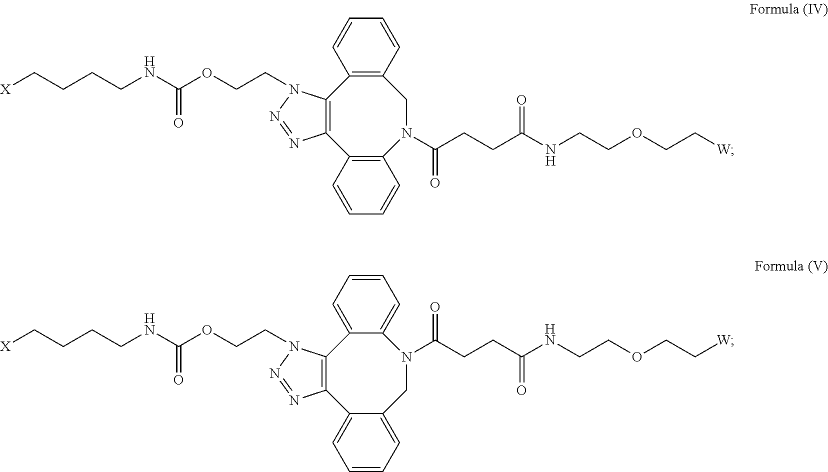

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

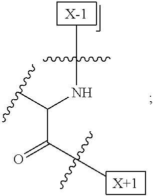

X−1 indicates the point of attachment to the preceding amino acid residue; and
X+1 indicates the point of attachment to the following amino acid residue.

Described herein, in some embodiments, is an IL-10 conjugate comprising the amino acid sequence of any one of SEQ ID NOS: 59 to 66, wherein [AzK_L1_PEG] has the structure of Formula (IV), Formula (V), or a mixture of Formula (IV) and Formula (V):

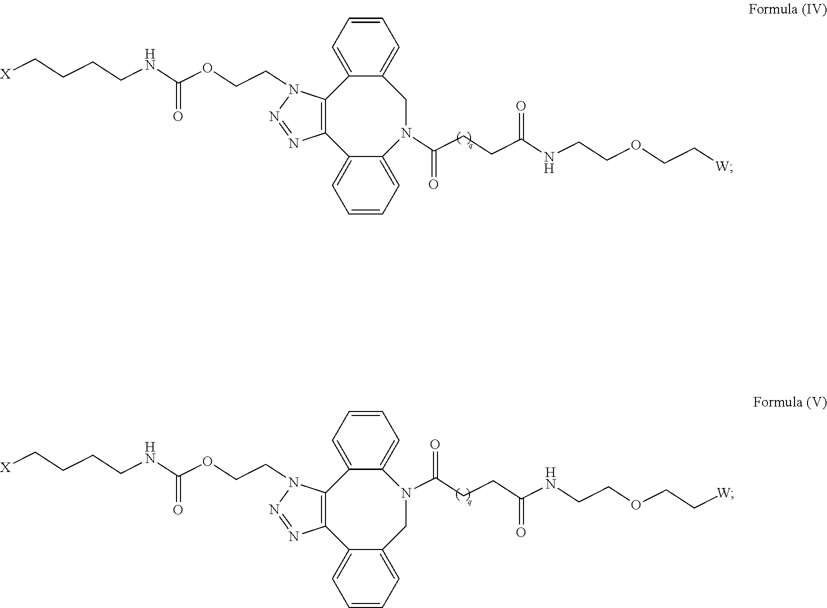

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa;
q is 1, 2, or 3; and
X has the structure:

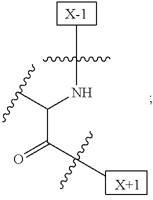

X−1 indicates the point of attachment to the preceding amino acid residue; and
X+1 indicates the point of attachment to the following amino acid residue. In some embodiments, q is 1. In some embodiments, q is 2. In some embodiments, q is 3.

Here and throughout, the structure of Formula (IV) encompasses pharmaceutically acceptable salts, solvates, or hydrates thereof. Here and throughout, the structure of Formula (V) encompasses pharmaceutically acceptable salts, solvates, or hydrates thereof.

In some embodiments, the methods use an IL-10 conjugate in which the [AzK_L1_PEG] is of Formula (IV). In some embodiments, the methods use an IL-10 conjugate in which the [AzK_L1_PEG] is of Formula (V). In some embodiments, the methods use an IL-10 conjugate in which the [AzK_L1_PEG] is a mixture of Formula (IV) and Formula (V).

In some embodiments of the IL-10 conjugate comprising the amino acid sequence of SEQ ID NO: 59 and [AzK_L1_PEG] having the structure of Formula (IV), Formula (V), or a mixture of Formula (IV) and Formula (V), W is a PEG group having an average molecular weight selected from 500 Daltons, 1 kDa, 2 kDa, 3 Da, 4 kDa, 5 kDa, 10 kDa, 15 kDa, 20 kDa, 25 kDa, 30 kDa, 35 kDa, 40 kDa, 45 kDa, 50 kDa, and 100 kDa. In some embodiments of the IL-10 conjugate comprising the amino acid sequence of SEQ ID NO: 59 and [AzK_L1_PEG] having the structure of Formula (IV), Formula (V), or a mixture of Formula (IV) and Formula (V), W is a PEG group having an average molecular weight selected from 5 kDa, 10 kDa, 15 kDa, 20 kDa, 25 kDa, and 30 kDa. In some embodiments of the IL-10 conjugate comprising the amino acid sequence of SEQ ID NO: 59 and [AzK_L1_PEG] having the structure of Formula (IV), Formula (V), or a mixture of Formula (IV) and Formula (V), W is a PEG group having an average molecular weight selected from 20 kDa and 30 kDa. In some embodiments of the IL-10 conjugate comprising the amino acid sequence of SEQ ID NO: 59 and [AzK_L1_PEG] having the structure of Formula (IV), Formula (V), or a mixture of Formula (IV) and Formula (V), W is a PEG group having an average molecular weight of 20 kDa. In some embodiments of the IL-10 conjugate comprising the amino acid sequence of SEQ ID NO: 59 and [AzK_L1_PEG] having the structure of Formula (IV), Formula (V), or a mixture of Formula (IV) and Formula (V), W is a PEG group having an average molecular weight of 30 kDa. In some embodiments of the IL-10 conjugate comprising the amino acid sequence of SEQ ID NO: 60 and [AzK_L1_PEG] having the structure of Formula (IV) Formula (V), or a mixture of Formula (IV) and Formula (V), W is a PEG group having an average molecular weight selected from 500 Daltons, 1 kDa, 2 kDa, 3 kDa, 4 kDa, 5 kDa, 10 kDa, 15 kDa, 20 kDa, 25 kDa, 30 kDa, 35 kDa, 40 kDa, 45 kDa, 50 kDa, and 100 kDa. In some embodiments of the IL-10 conjugate comprising the amino acid sequence of SEQ ID NO: 60 and [AzK_L1_PEG] having the structure of Formula (IV), Formula (V), or a mixture of Formula (IV) and Formula (V), W is a PEG group having an average molecular weight selected from 5 kDa, 10 kDa, 15 kDa, 20 kDa, 25 kDa, and 30 kDa. In some embodiments of the IL-10 conjugate comprising the amino acid sequence of SEQ ID NO: 60 and [AzK_L1_PEG] having the structure of Formula (IV), Formula (V), or a mixture of Formula (IV) and Formula (V), W is a PEG group having an average molecular weight selected from 20 kDa and 30 kDa. In some embodiments of the IL-10 conjugate comprising the amino acid sequence of SEQ ID NO: 60 and [AzK_L1_PEG] having the structure of Formula (IV), Formula (V), or a mixture of Formula (IV) and Formula (V), W is a PEG group having an average molecular weight of 20 kDa. In some embodiments of the IL-10 conjugate comprising the amino acid sequence of SEQ ID NO: 60 and [AzK_L1_PEG] having the structure of Formula (IV), Formula (V), or a mixture of Formula (IV) and Formula (V), W is a PEG group having an average molecular weight of 30 kDa.

In some embodiments of the IL-10 conjugate comprising the amino acid sequence of SEQ ID NO: 61 and [AzK_L1_PEG] having the structure of Formula (IV), Formula (V), or a mixture of Formula (IV) and Formula (V), W is a PEG group having an average molecular weight selected from 500 Daltons, 1 kDa, 2 kDa, 3 kDa, 4 kDa, 5 kDa, 10 kDa, 15 kDa, 20 kDa, 25 kDa, 30 kDa, 35 kDa, 40 kDa, 45 kDa, 50 kDa, and 100 kDa. In some embodiments of the IL-10 conjugate comprising the amino acid sequence of SEQ ID NO: 61 and [AzK_L1_PEG] having the structure of Formula (IV), Formula (V), or a mixture of Formula (IV) and Formula (V), W is a PEG group having an average molecular weight selected from 5 kDa, 10 kDa, 15 kDa, 20 kDa, 25 kDa, and 30 kDa. In some embodiments of the IL-10 conjugate comprising the amino acid sequence of SEQ ID NO: 61 and [AzK_L1_PEG] having the structure of Formula (IV), Formula (V), or a mixture of Formula (IV) and Formula (V), W is a PEG group having an average molecular weight selected from 20 kDa and 30 kDa, or a pharmaceutically acceptable salt, solvate, or hydrate thereof. In some embodiments of the IL-10 conjugate comprising the amino acid sequence of SEQ ID NO: 61 and [AzK_L1_PEG] having the structure of Formula (IV), Formula (V), or a mixture of Formula (IV) and Formula (V), W is a PEG group having an average molecular weight of 20 kDa. In some embodiments of the IL-10 conjugate comprising the amino acid sequence of SEQ ID NO: 61 and [AzK_L1_PEG] having the structure of Formula (IV), Formula (V), or a mixture of Formula (IV) and Formula (V), W is a PEG group having an average molecular weight of 30 kDa.

In some embodiments of the IL-10 conjugate comprising the amino acid sequence of SEQ ID NO: 62 and [AzK_L1_PEG] having the structure of Formula (IV), Formula (V), or a mixture of Formula (IV) and Formula (V), W is a PEG group having an average molecular weight selected from 500 Daltons, 1 kDa, 2 kDa, 3 kDa, 4 kDa, 5 kDa, 10 kDa, 15 kDa, 20 kDa, 25 kDa, 30 kDa, 35 kDa, 40 kDa, 45 kDa, 50 kDa, and 100 kDa. In some embodiments of the IL-10 conjugate comprising the amino acid sequence of SEQ ID NO: 62 and [AzK_L1_PEG] having the structure of Formula (IV), Formula (V), or a mixture of Formula (IV) and Formula (V), W is a PEG group having an average molecular weight selected from 5 kDa, 10 kDa, 15 kDa, 20 kDa, 25 kDa, and 30 kDa. In some embodiments of the IL-10 conjugate comprising the amino acid sequence of SEQ ID NO: 62 and [AzK_L1_PEG] having the structure of Formula (IV), Formula (V), or a mixture of Formula (IV) and Formula (V), W is a PEG group having an average molecular weight selected from 20 kDa and 30 kDa. In some embodiments of the IL-10 conjugate comprising the amino acid sequence of SEQ ID NO: 62 and [AzK_L1_PEG] having the structure of Formula (IV), Formula (V), or a mixture of Formula (IV) and Formula (V), W is a PEG group having an average molecular weight of 20 kDa. In some embodiments of the IL-10 conjugate comprising the amino acid sequence of SEQ ID NO: 62 and [AzK_L1_PEG] having the structure of Formula (IV), Formula (V), or a mixture of Formula (IV) and Formula (V), W is a PEG group having an average molecular weight of 30 kDa.

In some embodiments of the IL-10 conjugate comprising the amino acid sequence of SEQ ID NO: 63 and [AzK_L1_PEG] having the structure of Formula (IV), Formula (V), or a mixture of Formula (IV) and Formula (V), W is a PEG group having an average molecular weight selected from 500 Daltons, 1 kDa, 2 kDa, 3 kDa, 4 kDa, 5 kDa, 10 kDa, 15 kDa, 20 kDa, 25 kDa, 30 kDa, 35 kDa, 40 kDa, 45 kDa, 50 kDa, and 100 kDa. In some embodiments of the IL-10 conjugate comprising the amino acid sequence of SEQ ID NO: 63 and [AzK_L1_PEG] having the structure of Formula (IV), Formula (V), or a mixture of Formula (IV) and Formula (V), W is a PEG group having an average molecular weight selected from 5 kDa, 10 kDa, 15 kDa, 20 kDa, 25 kDa, and 30 kDa. In some embodiments of the IL-10 conjugate comprising the amino acid sequence of SEQ ID NO: 63 and [AzK_L1_PEG] having the structure of Formula (IV), Formula (V), or a mixture of Formula (IV) and Formula (V), W is a PEG group having an average molecular weight selected from 20 kDa and 30 kDa. In some embodiments of the IL-10 conjugate comprising the amino acid sequence of SEQ ID NO: 63 and [AzK_L1_PEG] having the structure of Formula (IV), Formula (V), or a mixture of Formula (IV) and Formula (V), W is a PEG group having an average molecular weight of 20 kDa. In some embodiments of the IL-10 conjugate comprising the amino acid sequence of SEQ ID NO: 63 and [AzK_L1_PEG] having the structure of Formula (IV), Formula (V), or a mixture of Formula (IV) and Formula (V), W is a PEG group having an average molecular weight of 30 kDa.

In some embodiments of the IL-10 conjugate comprising the amino acid sequence of SEQ ID NO: 64 and [AzK_L1_PEG] having the structure of Formula (IV), Formula (V), or a mixture of Formula (IV) and Formula (V), W is a PEG group having an average molecular weight selected from 500 Daltons, 1 kDa, 2 kDa, 3 kDa, 4 kDa, 5 kDa, 10 kDa, 15 kDa, 20 kDa, 25 kDa, 30 kDa, 35 kDa, 40 kDa, 45 kDa, 50 kDa, and 100 kDa. In some embodiments of the IL-10 conjugate comprising the amino acid sequence of SEQ ID NO: 64 and [AzK_L1_PEG] having the structure of Formula (IV), Formula (V), or a mixture of Formula (IV) and Formula (V), W is a PEG group having an average molecular weight selected from 5 kDa, 10 kDa, 15 kDa, 20 kDa, 25 kDa, and 30 kDa. In some embodiments of the IL-10 conjugate comprising the amino acid sequence of SEQ ID NO: 64 and [AzK_L1_PEG] having the structure of Formula (IV), Formula (V), or a mixture of Formula (IV) and Formula (V), W is a PEG group having an average molecular weight selected from 20 kDa and 30 kDa. In some embodiments of the IL-10 conjugate comprising the amino acid sequence of SEQ ID NO: 64 and [AzK_L1_PEG] having the structure of Formula (IV), Formula (V), or a mixture of Formula (IV) and Formula (V), W is a PEG group having an average molecular weight of 20 kDa. In some embodiments of the IL-10 conjugate comprising the amino acid sequence of SEQ ID NO: 64 and [AzK_L1_PEG] having the structure of Formula (IV), Formula (V), or a mixture of Formula (IV) and Formula (V), W is a PEG group having an average molecular weight of 30 kDa.

In some embodiments of the IL-10 conjugate comprising the amino acid sequence of SEQ ID NO: 65 and [AzK_L1_PEG] having the structure of Formula (IV), Formula (V), or a mixture of Formula (IV) and Formula (V), W is a PEG group having an average molecular weight selected from 500 Daltons, 1 kDa, 2 kDa, 3 kDa, 4 kDa, 5 kDa, 10 kDa, 15 kDa, 20 kDa, 25 kDa, 30 kDa, 35 kDa, 40 kDa, 45 kDa, 50 kDa, and 100 kDa. In some embodiments of the IL-10 conjugate comprising the amino acid sequence of SEQ ID NO: 65 and [AzK_L1_PEG] having the structure of Formula (IV), Formula (V), or a mixture of Formula (IV) and Formula (V), W is a PEG group having an average molecular weight selected from 5 kDa, 10 kDa, 15 kDa, 20 kDa, 25 kDa, and 30 kDa. In some embodiments of the IL-10 conjugate comprising the amino acid sequence of SEQ ID NO: 65 and [AzK_L1_PEG] having the structure of Formula (IV), Formula (V), or a mixture of Formula (IV) and Formula (V), W is a PEG group having an average molecular weight selected from 20 kDa and 30 kDa. In some embodiments of the IL-10 conjugate comprising the amino acid sequence of SEQ ID NO: 65 and [AzK_L1_PEG] having the structure of Formula (IV), Formula (V), or a mixture of Formula (IV) and Formula (V), W is a PEG group having an average molecular weight of 20 kDa. In some embodiments of the IL-10 conjugate comprising the amino acid sequence of SEQ ID NO: 65 and [AzK_L1_PEG] having the structure of Formula (IV), Formula (V), or a mixture of Formula (IV) and Formula (V), W is a PEG group having an average molecular weight of 30 kDa.

In some embodiments of the IL-10 conjugate comprising the amino acid sequence of SEQ ID NO: 66 and [AzK_L1_PEG] having the structure of Formula (IV), Formula (V), or a mixture of Formula (IV) and Formula (V), W is a PEG group having an average molecular weight selected from 500 Daltons, 1 kDa, 2 kDa, 3 kDa, 4 kDa, 5 kDa, 10 kDa, 15 kDa, 20 kDa, 25 kDa, 30 kDa, 35 kDa, 40 kDa, 45 kDa, 50 kDa, and 100 kDa. In some embodiments of the IL-10 conjugate comprising the amino acid sequence of SEQ ID NO: 66 and [AzK_L1_PEG] having the structure of Formula (IV), Formula (V), or a mixture of Formula (IV) and Formula (V), W is a PEG group having an average molecular weight selected from 5 kDa, 10 kDa, 15 kDa, 20 kDa, 25 kDa, and 30 kDa. In some embodiments of the IL-10 conjugate comprising the amino acid sequence of SEQ ID NO: 66 and [AzK_L1_PEG] having the structure of Formula (IV), Formula (V), or a mixture of Formula (IV) and Formula (V), W is a PEG group having an average molecular weight selected from 20 kDa and 30 kDa. In some embodiments of the IL-10 conjugate comprising the amino acid sequence of SEQ ID NO: 66 and [AzK_L1_PEG] having the structure of Formula (IV), Formula (V), or a mixture of Formula (IV) and Formula (V), W is a PEG group having an average molecular weight of 20 kDa. In some embodiments of the IL-10 conjugate comprising the amino acid sequence of SEQ ID NO: 66 and [AzK_L1_PEG] having the structure of Formula (IV), Formula (V), or a mixture of Formula (IV) and Formula (V), W is a PEG group having an average molecular weight of 30 kDa.

Described herein, in some embodiments, is an IL-10 conjugate comprising the amino acid sequence of any one of SEQ ID NOS: 43 to 50, wherein [AzK_L1_PEG20 kDa] has the structure of Formula (IV), Formula (V), or a mixture of Formula (IV) and Formula (V):

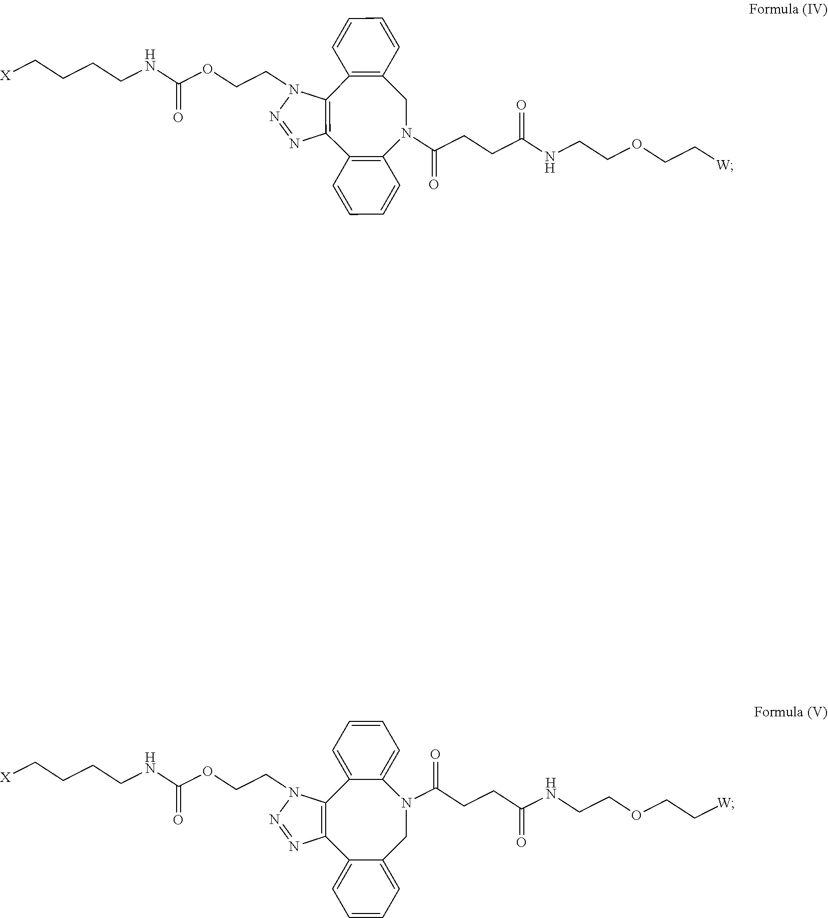

wherein:
W is a PEG group having an average molecular weight of 20 kDa; and
X has the structure:

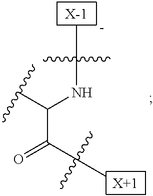

X−1 indicates the point of attachment to the preceding amino acid residue; and
X+1 indicates the point of attachment to the following amino acid residue.

Described herein, in some embodiments, is an IL-10 conjugate comprising the amino acid sequence of any one of SEQ ID NOS: 43 to 50, wherein [AzK_L1_PEG20 kDa] has the structure of Formula (IV), Formula (V), or a mixture of Formula (IV) and Formula (V):

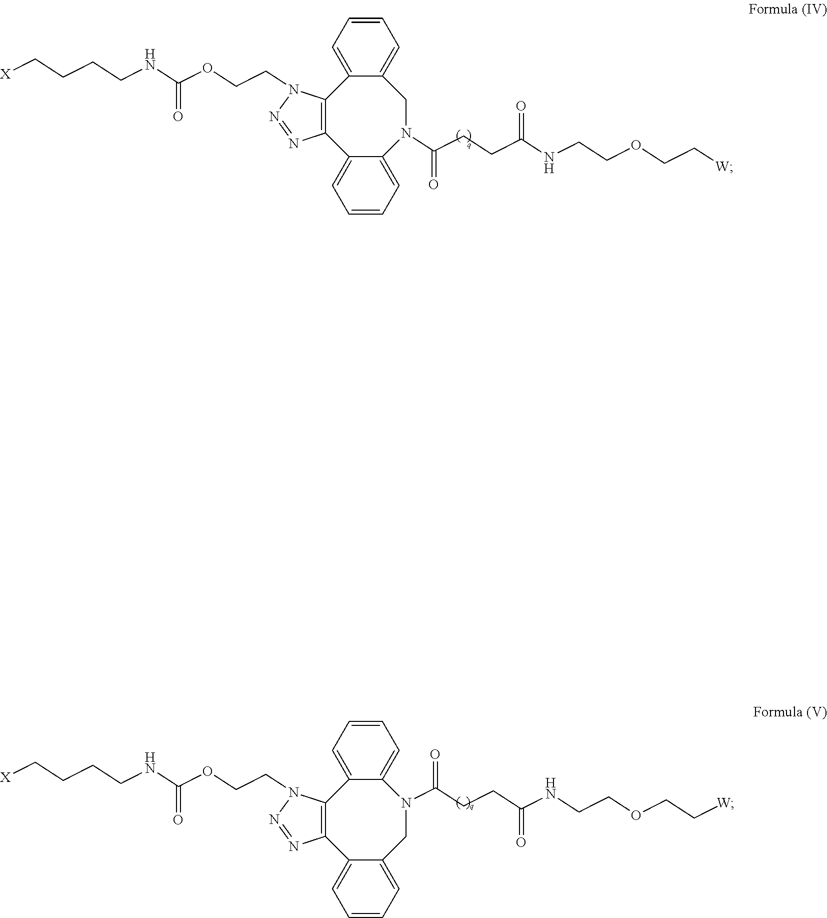

wherein:
W is a PEG group having an average molecular weight of 20 kDa;
q is 1, 2, or 3; and
X has the structure:

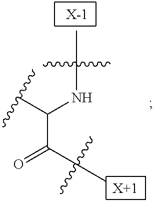

X−1 indicates the point of attachment to the preceding amino acid residue; and
X+1 indicates the point of attachment to the following amino acid residue. In some embodiments, q is 1. In some embodiments, q is 2. In some embodiments, q is 3.

In some embodiments, the IL-10 conjugate comprises the amino acid sequence of SEQ ID NO: 43, wherein [AzK_L1_PEG20 kDa] has the structure of Formula (IV), Formula (V), or a mixture of Formula (IV) and Formula (V).

In some embodiments, the IL-10 conjugate comprises the amino acid sequence of SEQ ID NO: 44, wherein [AzK_L1_PEG20 kDa] has the structure of Formula (IV), Formula (V), or a mixture of Formula (IV) and Formula (V). In some embodiments, the IL-10 conjugate comprises the amino acid sequence of SEQ ID NO: 45, wherein [AzK_L1_PEG20 kDa] has the structure of Formula (IV), Formula (V), or a mixture of Formula (IV) and Formula (V). In some embodiments, the IL-10 conjugate comprises the amino acid sequence of SEQ ID NO: 46, wherein [AzK_L1_PEG20 kDa] has the structure of Formula (IV), Formula (V), or a mixture of Formula (IV) and Formula (V). In some embodiments, the IL-10 conjugate comprises the amino acid sequence of SEQ ID NO: 47, wherein [AzK_L1_PEG20 kDa] has the structure of Formula (IV), Formula (V), or a mixture of Formula (IV) and Formula (V). In some embodiments, the IL-10 conjugate comprises the amino acid sequence of SEQ ID NO: 48, wherein [AzK_L1_PEG20 kDa] has the structure of Formula (IV), Formula (V), or a mixture of Formula (IV) and Formula (V). In some embodiments, the IL-10 conjugate comprises the amino acid sequence of SEQ ID NO: 49, wherein [AzK_L1_PEG20 kDa] has the structure of Formula (IV), Formula (V), or a mixture of Formula (IV) and Formula (V). In some embodiments, the IL-10 conjugate comprises the amino acid sequence of SEQ ID NO: 50, wherein [AzK_L1_PEG20 kDa] has the structure of Formula (IV), Formula (V), or a mixture of Formula (IV) and Formula (V).

In some embodiments, the IL-10 conjugate comprises the amino acid sequence of one or more of SEQ ID NOS: 43-50, wherein [AzK_L1_PEG20 kDa] has the structure of a mixture of Formula (IV) and Formula (V). In some embodiments of the IL-10 conjugate comprising the amino acid sequence of one or more of SEQ ID NOS: 43-50 and [AzK_L1_PEG20 kDa] having the structure of a mixture of Formula (IV) and Formula (V), the ratio of the amount of the structure of Formula (IV) to the amount of the structure of Formula (V) comprising the total amount of [AzK_L1_PEG20 kDa] in the IL-10 conjugate is about 1:1. In some embodiments of the IL-10 conjugate comprising the amino acid sequence of one or more of SEQ ID NOS: 43-50 and [AzK_L1_PEG20 kDa] having the structure of a mixture of Formula (IV) and Formula (V), the ratio of the amount of the structure of Formula (IV) to the amount of the structure of Formula (V) comprising the total amount of [AzK_L1_PEG20 kDa] in the IL-10 conjugate is greater than 1:1. In some embodiments of the IL-10 conjugate comprising the amino acid sequence of one or more of SEQ ID NOS: 43-50 and [AzK_L1_PEG20 kDa] having the structure of a mixture of Formula (IV) and Formula (V), the ratio of the amount of the structure of Formula (IV) to the amount of the structure of Formula (V) comprising the total amount of [AzK_L1_PEG20 kDa] in the IL-10 conjugate is less than 1:1.

Described herein, in some embodiments, is an IL-10 conjugate comprising the amino acid sequence of any one of SEQ ID NOS: 51 to 58, wherein [AzK_L1_PEG30 kDa] has the structure of Formula (IV), Formula (V), or a mixture of the structures of Formula (IV) and Formula (V):

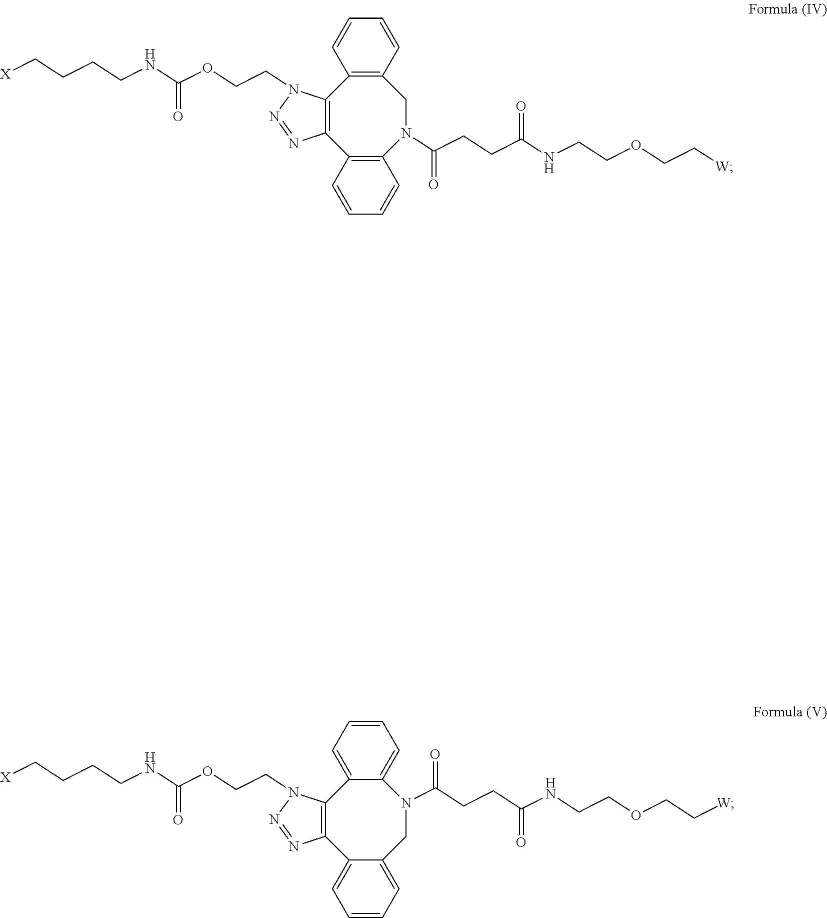

wherein:
W is a PEG group having an average molecular weight of 30 kDa; and
X has the structure:

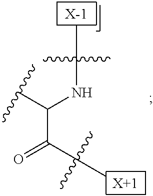

X−1 indicates the point of attachment to the preceding amino acid residue; and
X+1 indicates the point of attachment to the following amino acid residue.

Described herein, in some embodiments, is an IL-10 conjugate comprising the amino acid sequence of any one of SEQ ID NOS: 51 to 58, wherein [AzK_L1_PEG30 kDa] has the structure of Formula (IV), Formula (V), or a mixture of the structures of Formula (IV) and Formula (V):

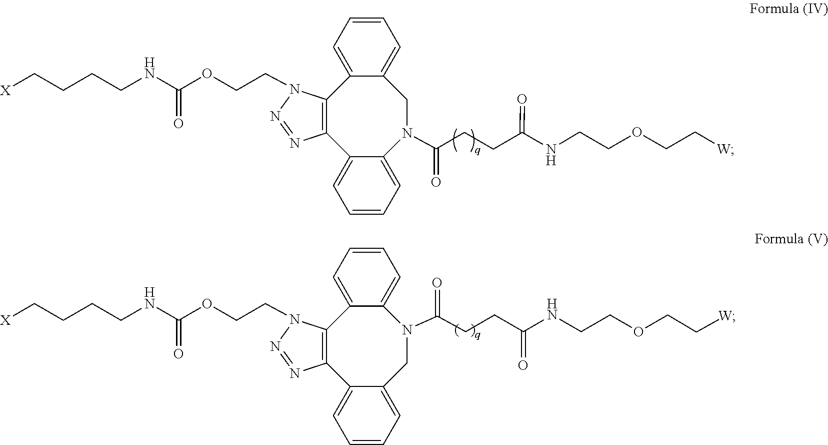

wherein:
W is a PEG group having an average molecular weight of 30 kDa;
q is 1, 2, or 3; and
X has the structure:

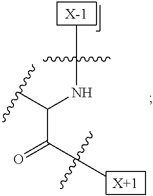

X−1 indicates the point of attachment to the preceding amino acid residue; and
X+1 indicates the point of attachment to the following amino acid residue. In some embodiments, q is 1. In some embodiments, q is 2. In some embodiments, q is 3.

In some embodiments, the IL-10 conjugate comprises the amino acid sequence of SEQ ID NO: 51, wherein [AzK_L1_PEG30 kDa] has the structure of Formula (IV), Formula (V), or a mixture of Formula (IV) and Formula (V). In some embodiments, the IL-10 conjugate comprises the amino acid sequence of SEQ ID NO: 52, wherein [AzK_L1_PEG30 kDa] has the structure of Formula (IV), Formula (V), or a mixture of Formula (IV) and Formula (V). In some embodiments, the IL-10 conjugate comprises the amino acid sequence of SEQ ID NO: 53, wherein [AzK_L1_PEG30 kDa] has the structure of Formula (IV), Formula (V), or a mixture of Formula (IV) and Formula (V). In some embodiments, the IL-10 conjugate comprises the amino acid sequence of SEQ ID NO: 54, wherein [AzK_L1_PEG30 kDa] has the structure of Formula (IV), Formula (V), or a mixture of Formula (IV) and Formula (V). In some embodiments, the IL-10 conjugate comprises the amino acid sequence of SEQ ID NO: 55, wherein [AzK_L1_PEG30 kDa] has the structure of Formula (IV), Formula (V), or a mixture of Formula (IV) and Formula (V). In some embodiments, the IL-10 conjugate comprises the amino acid sequence of SEQ ID NO: 56, wherein [AzK_L1_PEG30 kDa] has the structure of Formula (IV), Formula (V), or a mixture of Formula (IV) and Formula (V). In some embodiments, the IL-10 conjugate comprises the amino acid sequence of SEQ ID NO: 57, wherein [AzK_L1_PEG30 kDa] has the structure of Formula (IV), Formula (V), or a mixture of Formula (IV) and Formula (V). In some embodiments, the IL-10 conjugate comprises the amino acid sequence of SEQ ID NO: 58, wherein [AzK_L1_PEG30 kDa] has the structure of Formula (IV), Formula (V), or a mixture of Formula (IV) and Formula (V).

In some embodiments, the IL-10 conjugate comprises the amino acid sequence of one or more of SEQ ID NOS: 51-58, wherein [AzK_L1_PEG30 kDa] has the structure of a mixture of Formula (IV) and Formula (V). In some embodiments of the IL-10 conjugate comprising the amino acid sequence of one or more of SEQ ID NOS: 51-58 and [AzK_L1_PEG30 kDa] having the structure of a mixture of Formula (IV) and Formula (V), the ratio of the amount of the structure of Formula (IV) to the amount of the structure of Formula (V) comprising the total amount of [AzK_L1_PEG30 kDa] in the IL-10 conjugate is about 1:1. In some embodiments of the IL-10 conjugate comprising the amino acid sequence of one or more of SEQ ID NOS: 51-58 and [AzK_L1_PEG30 kDa] having the structure of a mixture of Formula (IV) and Formula (V), the ratio of the amount of the structure of Formula (IV) to the amount of the structure of Formula (V) comprising the total amount of [AzK_L1_PEG30 kDa] in the IL-10 conjugate is greater than 1:1. In some embodiments of the IL-10 conjugate comprising the amino acid sequence of one or more of SEQ ID NOS: 51-58 and [AzK_L1_PEG30 kDa] having the structure of a mixture of Formula (IV) and Formula (V), the ratio of the amount of the structure of Formula (IV) to the amount of the structure of Formula (V) comprising the total amount of [AzK_L1_PEG30 kDa] in the IL-10 conjugate is less than 1:1.

In some embodiments described herein of Formula (IV), Formula (V), or a mixture of Formula (IV) and Formula (V), q is 1. In some embodiments described herein of Formula (IV), Formula (V), or a mixture of Formula (IV) and Formula (V), q is 2. In some embodiments described herein of Formula (IV), Formula (V), or a mixture of Formula (IV) and Formula (V), q is 3. In some embodiments, the IL-10 conjugate comprises Formula (IV) and q is 1. In some embodiments, the IL-10 conjugate comprises Formula (IV) and q is 2. In some embodiments, the IL-10 conjugate comprises Formula (IV) and q is 3. In some embodiments, the IL-10 conjugate comprises Formula (V) and q is 1. In some embodiments, the IL-10 conjugate comprises Formula (V) and q is 2. In some embodiments, the IL-10 conjugate comprises Formula (V) and q is 3. In some embodiments, the IL-10 conjugate comprises a mixture of Formula (IV) and Formula (V) and q is 1. In some embodiments, the IL-10 conjugate comprises a mixture of Formula (IV) and Formula (V) and q is 2. In some embodiments, the IL-10 conjugate comprises a mixture of Formula (IV) and Formula (V) and q is 3.

Described herein, in some embodiments, is an IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII):

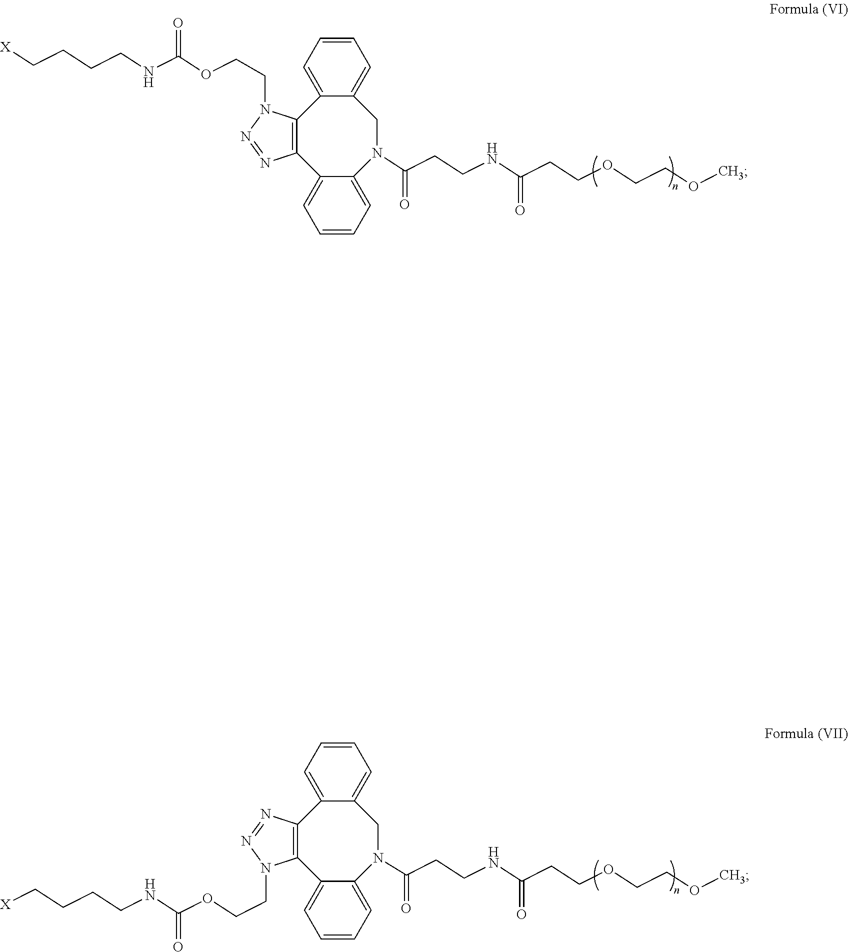

wherein:
n is an integer such that the molecular weight of the PEG group is from about 5,000 Daltons to about 60,000 Daltons; and
X has the structure:

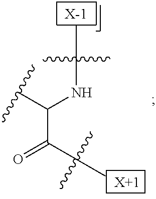

X−1 indicates the point of attachment to the preceding amino acid residue; and
X+1 indicates the point of attachment to the following amino acid residue.

Described herein, in some embodiments, is an IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII):

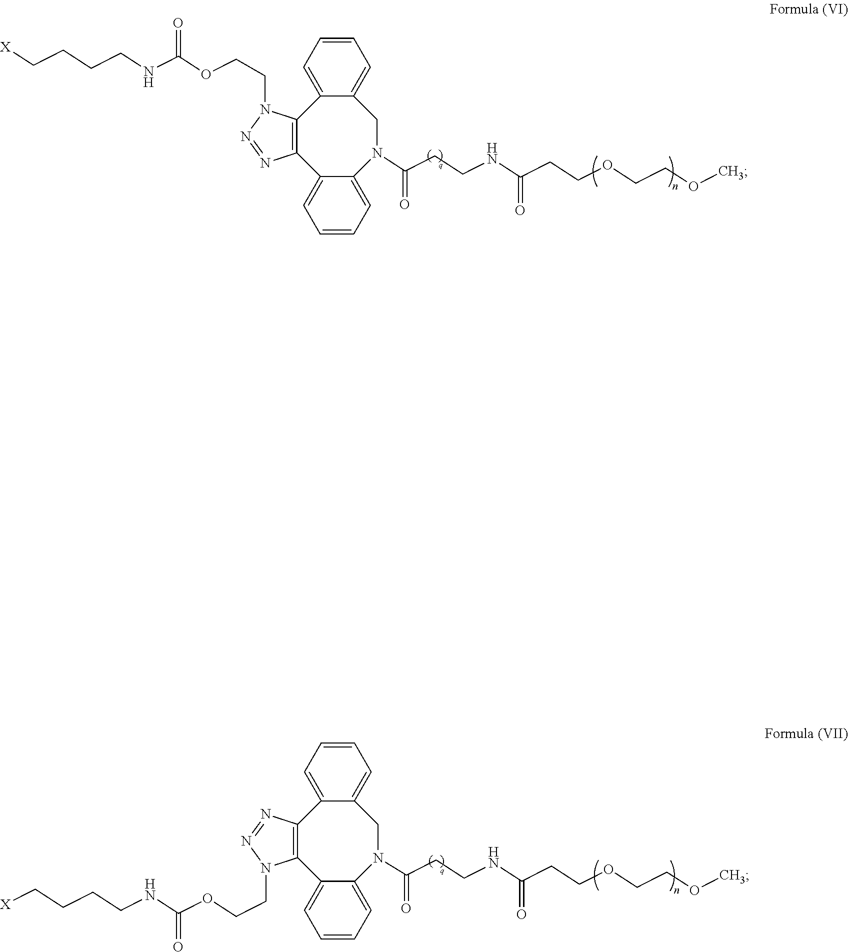

wherein:
n is an integer such that the molecular weight of the PEG group is from about 5,000 Daltons to about 60,000 Daltons;
q is 1, 2, or 3; and
X has the structure:

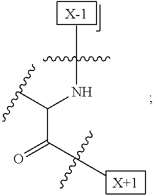

X−1 indicates the point of attachment to the preceding amino acid residue; and
X+1 indicates the point of attachment to the following amino acid residue. In some embodiments, q is 1. In some embodiments, q is 2. In some embodiments, q is 3.

Here and throughout, the structure of Formula (VI) encompasses pharmaceutically acceptable salts, solvates, or hydrates thereof. Here and throughout, the structure of Formula (VII) encompasses pharmaceutically acceptable salts, solvates, or hydrates thereof.

In some embodiments of the IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII), the position of the structure Formula (VI), Formula (VII), or a mixture of Formula (VI) and Formula (VII), in the amino acid sequence of the IL-10 conjugate is selected from E67, Q70, E74, E75, Q79, N82, K88, A89, K99, K125, N126, N129, K130, and Q132.

In some embodiments of the IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII), the position of the structure Formula (VI), Formula (VII), or a mixture of Formula (VI) and Formula (VII), in the amino acid sequence of the IL-10 conjugate is selected from N82, K88, A89, K99, K125, N126, N129, and K130. In some embodiments of the IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII), the position of the structure Formula (VI), Formula (VII), or a mixture of Formula (VI) and Formula (VII), in the amino acid sequence of the IL-10 conjugate is E67. In some embodiments of the IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII), the position of the structure Formula (VI), Formula (VII), or a mixture of Formula (VI) and Formula (VII), in the amino acid sequence of the IL-10 conjugate is Q70. In some embodiments of the IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII), the position of the structure Formula (VI), Formula (VII), or a mixture of Formula (VI) and Formula (VII), in the amino acid sequence of the IL-10 conjugate is E74. In some embodiments of the IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII), the position of the structure Formula (VI), Formula (VII), or a mixture of Formula (VI) and Formula (VII), in the amino acid sequence of the IL-10 conjugate is E75. In some embodiments of the IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII), the position of the structure Formula (VI), Formula (VII), or a mixture of Formula (VI) and Formula (VII), in the amino acid sequence of the IL-10 conjugate is Q79. In some embodiments of the IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII), the position of the structure Formula (VI), Formula (VII), or a mixture of Formula (VI) and Formula (VII), in the amino acid sequence of the IL-10 conjugate is N82. In some embodiments of the IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII), the position of the structure Formula (VI), Formula (VII), or a mixture of Formula (VI) and Formula (VII), in the amino acid sequence of the IL-10 conjugate is K88. In some embodiments of the IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII), the position of the structure Formula (VI), Formula (VII), or a mixture of Formula (VI) and Formula (VII), in the amino acid sequence of the IL-10 conjugate is A89. In some embodiments of the IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII), the position of the structure Formula (VI), Formula (VII), or a mixture of Formula (VI) and Formula (VII), in the amino acid sequence of the IL-10 conjugate is K99. In some embodiments of the IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII), the position of the structure Formula (VI), Formula (VII), or a mixture of Formula (VI) and Formula (VII), in the amino acid sequence of the IL-10 conjugate is K125. In some embodiments of the IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII), the position of the structure Formula (VI), Formula (VII), or a mixture of Formula (VI) and Formula (VII), in the amino acid sequence of the IL-10 conjugate is N126. In some embodiments of the IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII), the position of the structure Formula (VI), Formula (VII), or a mixture of Formula (VI) and Formula (VII), in the amino acid sequence of the IL-10 conjugate is N129.

In some embodiments of the IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII), the position of the structure Formula (VI), Formula (VII), or a mixture of Formula (VI) and Formula (VII), in the amino acid sequence of the IL-10 conjugate is K130. In some embodiments of the IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII), the position of the structure Formula (VI), Formula (VII), or a mixture of Formula (VI) and Formula (VII), in the amino acid sequence of the IL-10 conjugate is Q132.

In some embodiments of the IL-10 conjugate comprising the amino acid sequence of SEQ ID NO: 1 in which at least one of E67, Q70, E74, E75, Q79, N82, K88, A89, K99, K125, N126, N129, K130, and Q132 in the IL-10 conjugate is replaced by the structure of a mixture of Formula (VI) and Formula (VII), the ratio of the amount of the structure of Formula (VI) to the amount of the structure of Formula (VII) comprising the total amount of the IL-10 conjugate is about 1:1. In some embodiments of the IL-10 conjugate comprising the amino acid sequence of SEQ ID NO: 1 in which at least one of E67, Q70, E74, E75, Q79, N82, K88, A89, K99, K125, N126, N129, K130, and Q132 in the IL-10 conjugate is replaced by the structure of a mixture of Formula (VI) and Formula (VII), the ratio of the amount of the structure of Formula (VI) to the amount of the structure of Formula (VII) comprising the total amount of the IL-10 conjugate is greater than 1:1. In some embodiments of the IL-10 conjugate comprising the amino acid sequence of SEQ ID NO: 1 in which at least one of E67, Q70, E74, E75, Q79, N82, K88, A89, K99, K125, N126, N129, K130, and Q132 in the IL-10 conjugate is replaced by the structure of a mixture of Formula (VI) and Formula (VII), the ratio of the amount of the structure of Formula (VI) to the amount of the structure of Formula (VII) comprising the total amount of the IL-10 conjugate is less than 1:1.

In some embodiments of the IL-10 conjugate comprising the amino acid sequence of SEQ ID NO: 1 in which at least one amino acid residue in the IL-10 conjugate being replaced by the structure of Formula (VI), Formula (VII), or a mixture of Formula (VI) and Formula (VII), the position of the structure Formula (VI), Formula (VII), or a mixture of Formula (VI) and Formula (VII), in the amino acid sequence of the IL-10 conjugate being selected from E67, Q70, E74, E75, Q79, N82, K88, A89, K99, K125, N126, N129, K130, and Q132, n is an integer such that the molecular weight of the PEG group is from about 1,000 Daltons to about 100,000 Daltons, about 5,000 Daltons to about 50,000 Daltons, about 5,000 Daltons to about 40,000 Daltons, about 5,000 Daltons to about 30,000 Daltons, about 5,000 Daltons to about 25,000 Daltons, about 5,000 Daltons to about 20,000 Daltons about 5,000 Daltons to about 15,000 Daltons, or about 5,000 Daltons to about 10,000 Daltons.

In some embodiments, n is an integer such that the molecular weight of the PEG group is about 1,000 Daltons. In some embodiments, n is an integer such that the molecular weight of the PEG group is about 5,000 Daltons. In some embodiments, n is an integer such that the molecular weight of the PEG group is about 10,000 Daltons. In some embodiments, n is an integer such that the molecular weight of the PEG group is about 15,000 Daltons. In some embodiments, n is an integer such that the molecular weight of the PEG group is about 20,000 Daltons. In some embodiments, n is an integer such that the molecular weight of the PEG group is about 25,000 Daltons. In some embodiments, n is an integer such that the molecular weight of the PEG group is about 30,000 Daltons. In some embodiments, n is an integer such that the molecular weight of the PEG group is about 40,000 Daltons. In some embodiments, n is an integer such that the molecular weight of the PEG group is about 50,000 Daltons. In some embodiments, n is an integer such that the molecular weight of the PEG group is about 100,00 Daltons.

In some embodiments described herein of Formula (VI), Formula (VII), or a mixture of Formula (VI) and Formula (VII), q is 1. In some embodiments described herein of Formula (VI), Formula (VII), or a mixture of Formula (VI) and Formula (VII), q is 2. In some embodiments described herein of Formula (VI), Formula (VII), or a mixture of Formula (VI) and Formula (VII), q is 3. In some embodiments, the IL-10 conjugate comprises Formula (VI) and q is 1. In some embodiments, the IL-10 conjugate comprises Formula (VI) and q is 2. In some embodiments, the IL-10 conjugate comprises Formula (VI) and q is 3. In some embodiments, the IL-10 conjugate comprises Formula (VII) and q is 1. In some embodiments, the IL-10 conjugate comprises Formula (VII) and q is 2. In some embodiments, the IL-10 conjugate comprises Formula (VII) and q is 3. In some embodiments, the IL-10 conjugate comprises a mixture of Formula (VI) and Formula (VII) and q is 1. In some embodiments, the IL-10 conjugate comprises a mixture of Formula (VI) and Formula (VII) and q is 2. In some embodiments, the IL-10 conjugate comprises a mixture of Formula (VI) and Formula (VII) and q is 3.

Described herein, in some embodiments, is an IL-10 conjugate comprising the amino acid sequence of SEQ ID NO: 1 in which at least one amino acid residue in the IL-10 conjugate is replaced by the structure of Formula (VIII) or Formula (IX), or a mixture of Formula (VIII) and Formula (IX):

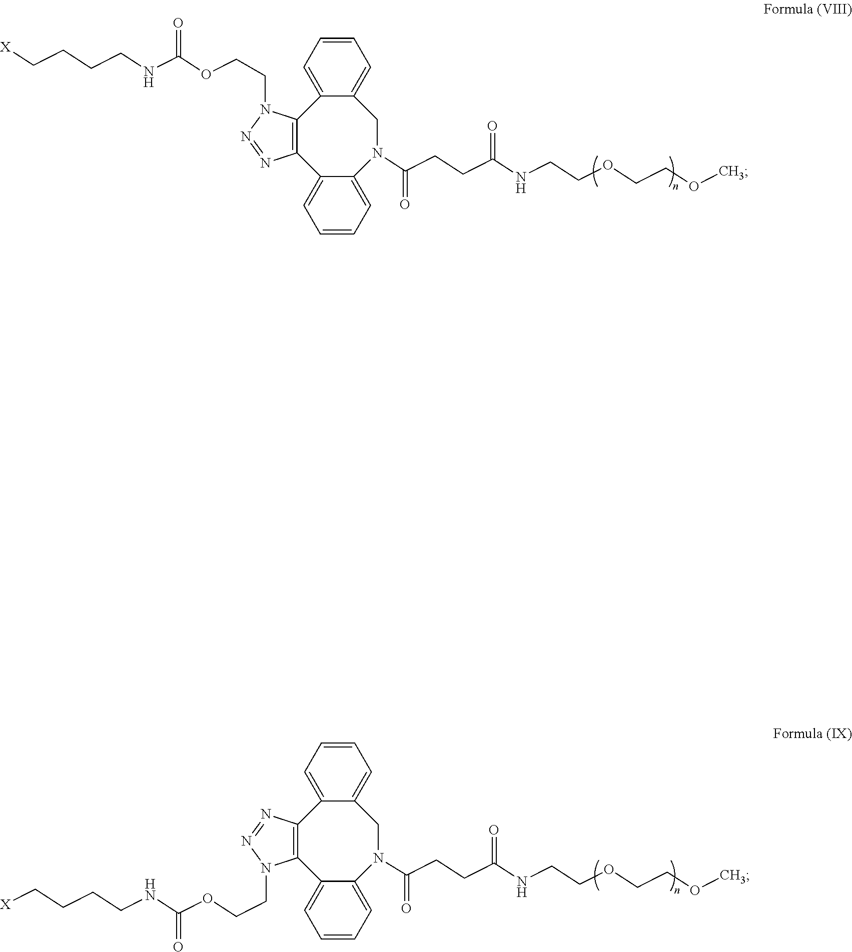

wherein:
n is an integer such that the molecular weight of the PEG group is from about 5,000 Daltons to about 60,000 Daltons; and
X has the structure:

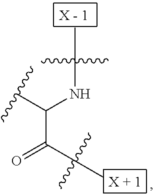 [SMILES: *NC(*)C(*)=O]

X−1 indicates the point of attachment to the preceding amino acid residue; and
X+1 indicates the point of attachment to the following amino acid residue.

Described herein, in some embodiments, is an IL-10 conjugate comprising the amino acid sequence of SEQ ID NO: 1 in which at least one amino acid residue in the IL-10 conjugate is replaced by the structure of Formula (VIII) or Formula (IX), or a mixture of Formula (VIII) and Formula (IX):

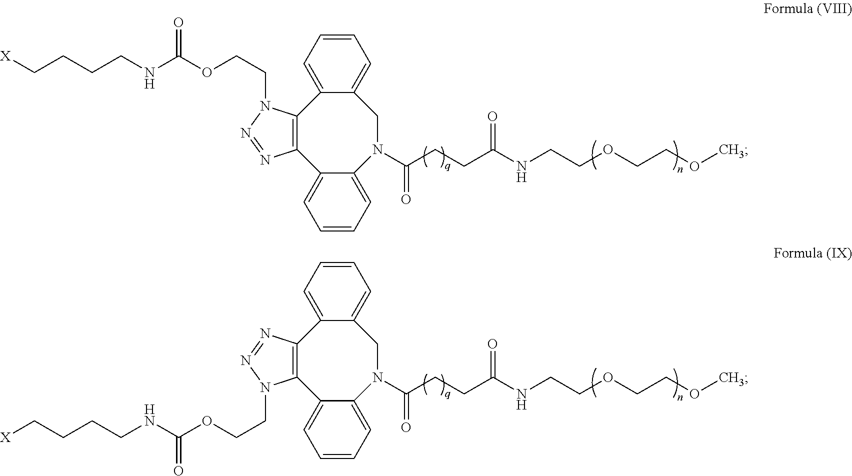

wherein:
n is an integer such that the molecular weight of the PEG group is from about 5,000 Daltons to about 60,000 Daltons;
q is 1, 2, or 3; and
X has the structure:

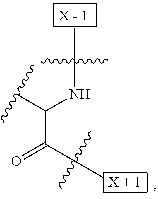 [SMILES: *NC(*)C(*)=O]

X−1 indicates the point of attachment to the preceding amino acid residue; and
X+1 indicates the point of attachment to the following amino acid residue. In some embodiments, q is 1. In some embodiments, q is 2. In some embodiments, q is 3.

Here and throughout, the structure of Formula (VIII) encompasses pharmaceutically acceptable salts, solvates, or hydrates thereof. Here and throughout, the structure of Formula (IX) encompasses pharmaceutically acceptable salts, solvates, or hydrates thereof.

In some embodiments of the IL-10 conjugate comprising the amino acid sequence of SEQ ID NO: 1 in which at least one amino acid residue in the IL-10 conjugate is replaced by the structure of Formula (VIII), Formula (IX), or a mixture of Formula (VIII) and Formula (IX), the position of the structure Formula (VIII), Formula (IX), or a mixture of Formula (VIII) and Formula (IX), in the amino acid sequence of the IL-10 conjugate is selected from E67, Q70, E74, E75, Q79, N82, K88, A89, K99, K125, N126, N129, K130, and Q132. In some embodiments of the IL-10 conjugate comprising the amino acid sequence of SEQ ID NO: 1 in which at least one amino acid residue in the IL-10 conjugate is replaced by the structure of Formula (VIII), Formula (IX), or a mixture of Formula (VIII) and Formula (IX), the position of the structure Formula (VIII), Formula (IX), or a mixture of Formula (VIII) and Formula (IX), in the amino acid sequence of the IL-10 conjugate is selected from N82, K88, A89, K99, K125, N126, N129, and K130.

In some embodiments of the IL-10 conjugate comprising the amino acid sequence of SEQ ID NO: 1 in which at least one amino acid residue in the IL-10 conjugate is replaced by the structure of Formula (VIII), Formula (IX), or a mixture of Formula (VIII) and Formula (IX), the position of the structure Formula (VIII), Formula (IX), or a mixture of Formula (VIII) and Formula (IX), in the amino acid sequence of the IL-10 conjugate is E67. In some embodiments of the IL-10 conjugate comprising the amino acid sequence of SEQ ID NO: 1 in which at least one amino acid residue in the IL-10 conjugate is replaced by the structure of Formula (VIII), Formula (IX), or a mixture of Formula (VIII) and Formula (IX), the position of the structure Formula (VIII), Formula (IX), or a mixture of Formula (VIII) and Formula (IX), in the amino acid sequence of the IL-10 conjugate is Q70. In some embodiments of the IL-10 conjugate comprising the amino acid sequence of SEQ ID NO: 1 in which at least one amino acid residue in the IL-10 conjugate is replaced by the structure of Formula (VIII), Formula (IX), or a mixture of Formula (VIII) and Formula (IX), the position of the structure Formula (VIII), Formula (IX), or a mixture of Formula (VIII) and Formula (IX), in the amino acid sequence of the IL-10 conjugate is E74. In some embodiments of the IL-10 conjugate comprising the amino acid sequence of SEQ ID NO: 1 in which at least one amino acid residue in the IL-10 conjugate is replaced by the structure of Formula (VIII), Formula (IX), or a mixture of Formula (VIII) and Formula (IX), the position of the structure Formula (VIII), Formula (IX), or a mixture of Formula (VIII) and Formula (IX), in the amino acid sequence of the IL-10 conjugate is E75. In some embodiments of the IL-10 conjugate comprising the amino acid sequence of SEQ ID NO: 1 in which at least one amino acid residue in the IL-10 conjugate is replaced by the structure of Formula (VIII), Formula (IX), or a mixture of Formula (VIII) and Formula (IX), the position of the structure Formula (VIII), Formula (IX), or a mixture of Formula (VIII) and Formula (IX), in the amino acid sequence of the IL-10 conjugate is Q79. In some embodiments of the IL-10 conjugate comprising the amino acid sequence of SEQ ID NO: 1 in which at least one amino acid residue in the IL-10 conjugate is replaced by the structure of Formula (VIII), Formula (IX), or a mixture of Formula (VIII) and Formula (IX), the position of the structure Formula (VIII), Formula (IX), or a mixture of Formula (VIII) and Formula (IX), in the amino acid sequence of the IL-10 conjugate is N82. In some embodiments of the IL-10 conjugate comprising the amino acid sequence of SEQ ID NO: 1 in which at least one amino acid residue in the IL-10 conjugate is replaced by the structure of Formula (VIII), Formula (IX), or a mixture of Formula (VIII) and Formula (IX), the position of the structure Formula (VIII), Formula (IX), or a mixture of Formula (VIII) and Formula (IX), in the amino acid sequence of the IL-10 conjugate is K88. In some embodiments of the IL-10 conjugate comprising the amino acid sequence of SEQ ID NO: 1 in which at least one amino acid residue in the IL-10 conjugate is replaced by the structure of Formula (VIII), Formula (IX), or a mixture of Formula (VIII) and Formula (IX), the position of the structure Formula (VIII), Formula (IX), or a mixture of Formula (VIII) and Formula (IX), in the amino acid sequence of the IL-10 conjugate is A89. In some embodiments of the IL-10 conjugate comprising the amino acid sequence of SEQ ID NO: 1 in which at least one amino acid residue in the IL-10 conjugate is replaced by the structure of Formula (VIII), Formula (IX), or a mixture of Formula (VIII) and Formula (IX), the position of the structure Formula (VIII), Formula (IX), or a mixture of Formula (VIII) and Formula (IX), in the amino acid sequence of the IL-10 conjugate is K99. In some embodiments of the IL-10 conjugate comprising the amino acid sequence of SEQ ID NO: 1 in which at least one amino acid residue in the IL-10 conjugate is replaced by the structure of Formula (VIII), Formula (IX), or a mixture of Formula (VIII) and Formula (IX), the position of the structure Formula (VIII), Formula (IX), or a mixture of Formula (VIII) and Formula (IX), in the amino acid sequence of the IL-10 conjugate is K125. In some embodiments of the IL-10 conjugate comprising the amino acid sequence of SEQ ID NO: 1 in which at least one amino acid residue in the IL-10 conjugate is replaced by the structure of Formula (VIII), Formula (IX), or a mixture of Formula (VIII) and Formula (IX), the position of the structure Formula (VIII), Formula (IX), or a mixture of Formula (VIII) and Formula (IX), in the amino acid sequence of the IL-10 conjugate is N126. In some embodiments of the IL-10 conjugate comprising the amino acid sequence of SEQ ID NO: 1 in which at least one amino acid residue in the IL-10 conjugate is replaced by the structure of Formula (VIII), Formula (IX), or a mixture of Formula (VIII) and Formula (IX), the position of the structure Formula (VIII), Formula (IX), or a mixture of Formula (VIII) and Formula (IX), in the amino acid sequence of the IL-10 conjugate is N129. In some embodiments of the IL-10 conjugate comprising the amino acid sequence of SEQ ID NO: 1 in which at least one amino acid residue in the IL-10 conjugate is replaced by the structure of Formula (VIII), Formula (IX), or a mixture of Formula (VIII) and Formula (IX), the position of the structure Formula (VIII), Formula (IX), or a mixture of Formula (VIII) and Formula (IX), in the amino acid sequence of the IL-10 conjugate is K130. In some embodiments of the IL-10 conjugate comprising the amino acid sequence of SEQ ID NO: 1 in which at least one amino acid residue in the IL-10 conjugate is replaced by the structure of Formula (VIII), Formula (IX), or a mixture of Formula (VIII) and Formula (IX), the position of the structure Formula (VIII), Formula (IX), or a mixture of Formula (VIII) and Formula (IX), in the amino acid sequence of the IL-10 conjugate is Q132.

In some embodiments of the IL-10 conjugate comprising the amino acid sequence of SEQ ID NO: 1 in which at least one of E67, Q70, E74, E75, Q79, N82, K88, A89, K99, K125, N126, N129, K130, and Q132 in the IL-10 conjugate is replaced by the structure of a mixture of Formula (VIII) and Formula (IX), the ratio of the amount of the structure of Formula (VIII) to the amount of the structure of Formula (IX) comprising the total amount of the IL-10 conjugate is about 1:1. In some embodiments of the IL-10 conjugate comprising the amino acid sequence of SEQ ID NO: 1 in which at least one of E67, Q70, E74, E75, Q79, N82, K88, A89, K99, K125, N126, N129, K130, and Q132 in the IL-10 conjugate is replaced by the structure of a mixture of Formula (VIII) and Formula (IX), the ratio of the amount of the structure of Formula (VIII) to the amount of the structure of Formula (IX) comprising the total amount of the IL-10 conjugate is greater than 1:1. In some embodiments of the IL-10 conjugate comprising the amino acid sequence of SEQ ID NO: 1 in which at least one of E67, Q70, E74, E75, Q79, N82, K88, A89, K99, K125, N126, N129, K130, and Q132 in the IL-10 conjugate is replaced by the structure of a mixture of Formula (VIII) and Formula (IX), the ratio of the amount of the structure of Formula (VIII) to the amount of the structure of Formula (IX) comprising the total amount of the IL-10 conjugate is less than 1:1.

In some embodiments of the IL-10 conjugate comprising the amino acid sequence of SEQ ID NO: 1 in which at least one amino acid residue in the IL-10 conjugate being replaced by the structure of Formula (VIII), Formula (IX), or a mixture of Formula (VIII) and Formula (IX), the position of the structure Formula (VIII), Formula (IX), or a mixture of Formula (VIII) and Formula (IX), in the amino acid sequence of the IL-10 conjugate being selected from E67, Q70, E74, E75, Q79, N82, K88, A89, K99, K125, N126, N129, K130, and Q132, n is an integer such that the molecular weight of the PEG group is from about 1,000 Daltons to about 100,000 Daltons, about 5,000 Daltons to about 50,000 Daltons, about 5,000 Daltons to about 40,000 Daltons, about 5,000 Daltons to about 30,000 Daltons, about 5,000 Daltons to about 25,000 Daltons, about 5,000 Daltons to about 20,000 Daltons, about 5,000 Daltons to about 15,000 Daltons, or about 5,000 Daltons to about 10,000 Daltons. In some embodiments, n is an integer such that the molecular weight of the PEG group is about 1,000 Daltons. In some embodiments, n is an integer such that the molecular weight of the PEG group is about 5,000 Daltons. In some embodiments, n is an integer such that the molecular weight of the PEG group is about 10,000 Daltons. In some embodiments, n is an integer such that the molecular weight of the PEG group is about 15,000 Daltons. In some embodiments, n is an integer such that the molecular weight of the PEG group is about 20,000 Daltons. In some embodiments, n is an integer such that the molecular weight of the PEG group is about 25,000 Daltons. In some embodiments, n is an integer such that the molecular weight of the PEG group is about 30,000 Daltons. In some embodiments, n is an integer such that the molecular weight of the PEG group is about 40,000 Daltons. In some embodiments, n is an integer such that the molecular weight of the PEG group is about 50,000 Daltons. In some embodiments, n is an integer such that the molecular weight of the PEG group is about 100,00 Daltons.

In some embodiments described herein of Formula (VIII), Formula (IX), or a mixture of Formula (VIII) and Formula (IX), q is 1. In some embodiments described herein of Formula (VIII), Formula (IX), or a mixture of Formula (VIII) and Formula (IX), q is 2. In some embodiments described herein of Formula (VIII), Formula (IX), or a mixture of Formula (VIII) and Formula (IX), q is 3. In some embodiments, the IL-10 conjugate comprises Formula (VIII) and q is 1. In some embodiments, the IL-10 conjugate comprises Formula (VIII) and q is 2. In some embodiments, the IL-10 conjugate comprises Formula (VIII) and q is 3. In some embodiments, the IL-10 conjugate comprises Formula (IX) and q is 1. In some embodiments, the IL-10 conjugate comprises Formula (IX) and q is 2. In some embodiments, the IL-10 conjugate comprises Formula (IX) and q is 3. In some embodiments, the IL-10 conjugate comprises a mixture of Formula (VIII) and Formula (IX) and q is 1. In some embodiments, the IL-10 conjugate comprises a mixture of Formula (VIII) and Formula (IX) and q is 2. In some embodiments, the IL-10 conjugate comprises a mixture of Formula (VIII) and Formula (IX) and q is 3.

Described herein, in some embodiments, is an IL-10 conjugate comprising the amino acid sequence of SEQ ID NO: 1 in which at least one amino acid residue in the IL-10 conjugate is replaced by the structure of Formula (X) or Formula (XI), or a mixture of Formula (X) and Formula (XI):

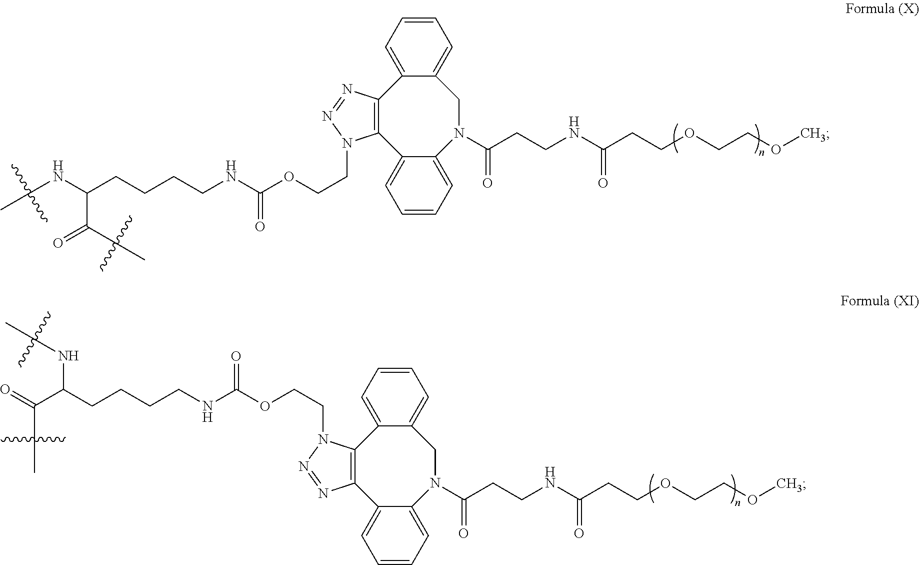

wherein:
n is an integer in the range from about 2 to about 5000; and
the wavy lines indicate covalent bonds to amino acid residues within SEQ ID NO: 1 that are not replaced.

Described herein, in some embodiments, is an IL-10 conjugate comprising the amino acid sequence of SEQ ID NO: 1 in which at least one amino acid residue in the IL-10 conjugate is replaced by the structure of Formula (X) or Formula (XI), or a mixture of Formula (X) and Formula (XI):

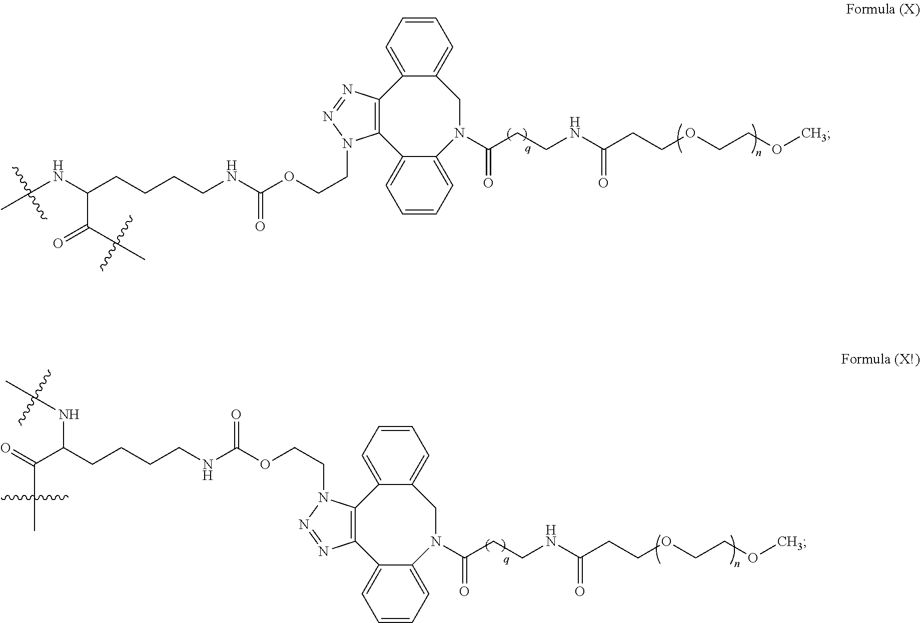

wherein:
n is an integer in the range from about 2 to about 5000;
q is 1, 2, or 3; and
the wavy lines indicate covalent bonds to amino acid residues within SEQ ID NO: 1 that are not replaced. In some embodiments, q is 1. In some embodiments, q is 2. In some embodiments, q is 3.

Here and throughout, the structure of Formula (X) encompasses pharmaceutically acceptable salts, solvates, or hydrates thereof. Here and throughout, the structure of Formula (XI) encompasses pharmaceutically acceptable salts, solvates, or hydrates thereof. In some embodiments, the IL-10 conjugate is a pharmaceutically acceptable salt, solvate, or hydrate.

In some embodiments, the stereochemistry of the chiral center within Formula (X) and Formula (XI) is racemic, is enriched in (R), is enriched in (S), is substantially (R), is substantially (S), is (R) or is (S). In some embodiments, the stereochemistry of the chiral center within Formula (X) and Formula (XI) is racemic. In some embodiments, the stereochemistry of the chiral center within Formula (X) and Formula (XI) is enriched in (R). In some embodiments, the stereochemistry of the chiral center within Formula (X) and Formula (XI) is enriched in (S). In some embodiments, the stereochemistry of the chiral center within Formula (X) and Formula (XI) is substantially (R). In some embodiments, the stereochemistry of the chiral center within Formula (X) and Formula (XI) is substantially (S). In some embodiments, the stereochemistry of the chiral center within Formula (X) and Formula (XI) is (R). In some embodiments, the stereochemistry of the chiral center within Formula (X) and Formula (XI) is (S).

In some embodiments of an IL-10 conjugate described herein, n in the compounds of Formula (X) or Formula (XI), or a mixture of Formula (X) and Formula (XI), is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments of an IL-10 conjugate described herein, n in the compounds of Formula (X) or Formula (XI), or a mixture of Formula (X) and Formula (XI), is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546. In some embodiments of an IL-10 conjugate described herein, the position of the structure of Formula (X) or Formula (XI), or a mixture of Formula (X) and Formula (XI), in the amino acid sequence of the IL-10 conjugate is selected from N82, K88, A89, K99, K125, N126, N129, and K130, wherein the position of the structure of Formula (I) in the amino acid sequence of the IL-10 conjugate is in reference to the positions in SEQ ID NO: 1. In some embodiments of an IL-10 conjugate described herein, the position of the structure of Formula (X) or Formula (XI), or a mixture of Formula (X) and Formula (XI), in the amino acid sequence of the IL-10 conjugate of SEQ ID NO: 1 is selected from N82, K88, A89, K99, K125, N126, N129, and K130. In some embodiments of an IL-10 conjugate described herein, the position of the structure of Formula (X) or Formula (XI) or a mixture of Formula (X) and Formula (XI), in the amino acid sequence of the IL-10 conjugate of SEQ ID NO: 1 is at position N82. In some embodiments of an IL-10 conjugate described herein, the position of the structure of Formula (X) or Formula (XI), or a mixture of Formula (X) and Formula (XI), in the amino acid sequence of the IL-10 conjugate of SEQ ID NO: 1 is at position K88. In some embodiments of an IL-10 conjugate described herein, the position of the structure of Formula (X) or Formula (XI), or a mixture of Formula (X) and Formula (XI), in the amino acid sequence of the IL-10 conjugate of SEQ ID NO: 1 is at position A89. In some embodiments of an IL-10 conjugate described herein, the position of the structure of Formula (X) or Formula (XI), or a mixture of Formula (X) and Formula (XI), in the amino acid sequence of the IL-10 conjugate of SEQ ID NO: 1 is at position K99. In some embodiments of an IL-10 conjugate described herein, the position of the structure of Formula (X) or Formula (XI), or a mixture of Formula (X) and Formula (XI), in the amino acid sequence of the IL-10 conjugate of SEQ ID NO: 1 is at position K125. In some embodiments of an IL-10 conjugate described herein, the position of the structure of Formula (X) or Formula (XI), or a mixture of Formula (X) and Formula (XI), in the amino acid sequence of the IL-10 conjugate of SEQ ID NO: 1 is at position N126. In some embodiments of an IL-10 conjugate described herein, the position of the structure of Formula (X) or Formula (XI), or a mixture of Formula (X) and Formula (XI), in the amino acid sequence of the IL-10 conjugate of SEQ ID NO: 1 is at position N129. In some embodiments of an IL-10 conjugate described herein, the position of the structure of Formula (X) or Formula (XI), or a mixture of Formula (X) and Formula (XI), in the amino acid sequence of the IL-10 conjugate of SEQ ID NO: 1 is at position K130. In some embodiments of an IL-10 conjugate described herein, the ratio of the amount of the structure of Formula (X) to the amount of the structure of Formula (XI) comprising the total amount of the IL-10 conjugate is about 1:1. In some embodiments of an IL-10 conjugate described herein, the ratio of the amount of the structure of Formula (X) to the amount of the structure of Formula (XI) comprising the total amount of the IL-10 conjugate is greater than 1:1. In some embodiments of an IL-10 conjugate described herein, the ratio of the amount of the structure of Formula (X) to the amount of the structure of Formula (XI) comprising the total amount of the IL-10 conjugate is less than 1:1.

In some embodiments described herein are IL-10 conjugates comprising the amino acid sequence of SEQ ID NO: 1 in which at least one amino acid residue in the IL-10 conjugate is replaced by the structure of Formula (X) or Formula (XI), or a mixture of Formula (X) and Formula (XI), wherein the amino acid residue in SEQ ID NO: 1 that is replaced is selected from N82, K88, A89, K99, K125, N126, N129, and K130, and wherein n is an integer from 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments of an IL-10 conjugate described herein, n in the compounds of formula (X) or Formula (XI), or a mixture of Formula (X) and Formula (XI), is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments described herein are IL-10 conjugates comprising the amino acid sequence of SEQ ID NO: 1 in which at least one amino acid residue in the IL-10 conjugate is replaced by the structure of Formula (X) or Formula (XI), or a mixture of Formula (X) and Formula (XI), wherein the amino acid residue in SEQ ID NO: 1 that is replaced is selected from N82, K88, A89, K99, K125, N126, N129, and K130, and wherein n is an integer from about 450 to about 800, or from about 454 to about 796, or from about 454 to about 682, or from about 568 to about 909. In some embodiments of an IL-10 conjugate described herein, n in the compounds of Formula (X) or Formula (XI), or a mixture of Formula (X) and Formula (XI), is an integer selected from 454, 455, 568, 569, 680, 681, 682, 794, 795, 796, 908, 909, 910, 1021, 1022, 1023, 1135, 1136, 1137, and 1249.

In some embodiments described herein are IL-10 conjugates comprising the amino acid sequence of SEQ ID NO: 1 in which at least one amino acid residue in the IL-10 conjugate is replaced by the structure of Formula (X) or Formula (XI), or a mixture of Formula (X) and Formula (XI), wherein the amino acid residue in SEQ ID NO: 1 that is replaced is selected from N82, K88, A89, K99, K125, N126, N129, and K130, and wherein n is an integer from about 450 to about 800, or from about 454 to about 796, or from about 454 to about 682, or from about 568 to about 909. In some embodiments of an IL-10 conjugate described herein, n in the compounds of Formula (X) or Formula (XI), or a mixture of Formula (X) and Formula (XI), is an integer selected from 454, 455, 568, 569, 680, 681, 682, 794, 795, 796, 908, 909, and 910.

In some embodiments described herein are IL-10 conjugates comprising the amino acid sequence of SEQ ID NO: 1 in which at least one amino acid residue in the IL-10 conjugate is replaced by the structure of Formula (X) or Formula (XI), or a mixture of Formula (X) and Formula (XI), wherein the amino acid residue in SEQ ID NO: 1 that is replaced is N82, and wherein n is an integer from about 450 to about 800, or from about 454 to about 796, or from about 454 to about 682, or from about 568 to about 909. In some embodiments of an IL-10 conjugate described herein, n in the compounds of Formula (X) or Formula (XI), or a mixture of Formula (X) and Formula (XI), is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 113, 227, 340, 454, 568, or 681.

In some embodiments described herein are IL-10 conjugates comprising the amino acid sequence of SEQ ID NO: 1 in which at least one amino acid residue in the IL-10 conjugate is replaced by the structure of Formula (X) or Formula (XI), or a mixture of Formula (X) and Formula (XI), wherein the amino acid residue in SEQ ID NO: 1 that is replaced is K88, and wherein n is an integer from about 450 to about 800, or from about 454 to about 796, or from about 454 to about 682, or from about 568 to about 909. In some embodiments of an IL-10 conjugate described herein, n in the compounds of Formula (X) or Formula (XI), or a mixture of Formula (X) and Formula (XI), is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is from about 550 to about 800. In some embodiments, n is about 113, 227, 340, 454, 568, or 681.

In some embodiments described herein are IL-10 conjugates comprising the amino acid sequence of SEQ ID NO: 1 in which at least one amino acid residue in the IL-10 conjugate is replaced by the structure of Formula (X) or Formula (XI), or a mixture of Formula (X) and Formula (XI), wherein the amino acid residue in SEQ ID NO: 1 that is replaced is A89, and wherein n is an integer from about 450 to about 800, or from about 454 to about 796, or from about 454 to about 682, or from about 568 to about 909. In some embodiments of an IL-10 conjugate described herein, n in the compounds of Formula (X) or Formula (XI), or a mixture of Formula (X) and Formula (XI), is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is from about 550 to about 800. In some embodiments, n is about 113, 227, 340, 454, 568, or 681.

In some embodiments described herein are IL-10 conjugates comprising the amino acid sequence of SEQ ID NO: 1 in which at least one amino acid residue in the IL-10 conjugate is replaced by the structure of Formula (X) or Formula (XI), or a mixture of Formula (X) and Formula (XI), wherein the amino acid residue in SEQ ID NO: 1 that is replaced is K99, and wherein n is an integer from about 450 to about 800, or from about 454 to about 796, or from about 454 to about 682, or from about 568 to about 909. In some embodiments of an IL-10 conjugate described herein, n in the compounds of Formula (X) or Formula (XI), or a mixture of Formula (X) and Formula (XI), is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is from about 550 to about 800. In some embodiments, n is about 113, 227, 340, 454, 568, or 681.

In some embodiments described herein are IL-10 conjugates comprising the amino acid sequence of SEQ ID NO: 1 in which at least one amino acid residue in the IL-10 conjugate is replaced by the structure of Formula (X) or Formula (XI), or a mixture of Formula (X) and Formula (XI), wherein the amino acid residue in SEQ ID NO: 1 that is replaced is K125, and wherein n is an integer from about 450 to about 800, or from about 454 to about 796, or from about 454 to about 682, or from about 568 to about 909. In some embodiments of an IL-10 conjugate described herein, n in the compounds of Formula (X) and Formula (XI), or a mixture of Formula (X) and Formula (XI), is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is from about 550 to about 800. In some embodiments, n is about 113, 227, 340, 454, 568, or 681.

In some embodiments described herein are IL-10 conjugates comprising the amino acid sequence of SEQ ID NO: 1 in which at least one amino acid residue in the IL-10 conjugate is replaced by the structure of Formula (X) or Formula (XI), or a mixture of Formula (X) and Formula (XI), wherein the amino acid residue in SEQ ID NO: 1 that is replaced is N126, and wherein n is an integer from about 450 to about 800, or from about 454 to about 796, or from about 454 to about 682, or from about 568 to about 909. In some embodiments of an IL-10 conjugate described herein, n in the compounds of Formula (X) and Formula (XI), or a mixture of Formula (X) and Formula (XI), is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is from about 550 to about 800. In some embodiments, n is about 113, 227, 340, 454, 568, or 681.

In some embodiments described herein are IL-10 conjugates comprising the amino acid sequence of SEQ ID NO: 1 in which at least one amino acid residue in the IL-10 conjugate is replaced by the structure of Formula (X) or Formula (XI), or a mixture of Formula (X) and Formula (XI), wherein the amino acid residue in SEQ ID NO: 1 that is replaced is N129, and wherein n is an integer from about 450 to about 800, or from about 454 to about 796, or from about 454 to about 682, or from about 568 to about 909. In some embodiments of an IL-10 conjugate described herein, n in the compounds of Formula (X) or Formula (XI), or a mixture of Formula (X) and Formula (XI), is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is from about 550 to about 800. In some embodiments, n is about 113, 227, 340, 454, 568, or 681.

In some embodiments described herein are IL-10 conjugates comprising the amino acid sequence of SEQ ID NO: 1 in which at least one amino acid residue in the IL-10 conjugate is replaced by the structure of Formula (X) or Formula (XI), or a mixture of Formula (X) and Formula (XI), wherein the amino acid residue in SEQ ID NO: 1 that is replaced is K130, and wherein n is an integer from about 450 to about 800, or from about 454 to about 796, or from about 454 to about 682, or from about 568 to about 909. In some embodiments of an IL-10 conjugate described herein, n in the compounds of Formula (X) or Formula (XI), or a mixture of Formula (X) and Formula (XI), is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is from about 550 to about 800. In some embodiments, n is about 113, 227, 340, 454, 568, or 681.

Described herein are IL-10 conjugates comprising the amino acid sequence of SEQ ID NO: 1 in which at least one amino acid residue in the IL-10 conjugate is replaced by the structure of Formula (X) or Formula (XI), or a mixture of Formula (X) and Formula (XI), wherein n is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons.

Described herein are IL-10 conjugates comprising the amino acid sequence of SEQ ID NO: 1 in which at least one amino acid residue in the IL-10 conjugate is replaced by the structure of Formula (X) or Formula (XI), or a mixture of Formula (X) and Formula (XI), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons. Described herein are IL-10 conjugates comprising the amino acid sequence of SEQ ID NO: 1 in which at least one amino acid residue in the IL-10 conjugate is replaced by the structure of Formula (X) or Formula (XI), or a mixture of Formula (X) and Formula (XI), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

In some embodiments described herein of Formula (X), Formula (XI), or a mixture of Formula (X) and Formula (XI), q is 1. In some embodiments described herein of Formula (X), Formula (XI), or a mixture of Formula (X) and Formula (XI), q is 2. In some embodiments described herein of Formula (X), Formula (XI), or a mixture of Formula (X) and Formula (XI), q is 3. In some embodiments, the IL-10 conjugate comprises Formula (X) and q is 1. In some embodiments, the IL-10 conjugate comprises Formula (X) and q is 2. In some embodiments, the IL-10 conjugate comprises Formula (X) and q is 3. In some embodiments, the IL-10 conjugate comprises Formula (XI) and q is 1. In some embodiments, the IL-10 conjugate comprises Formula (XI) and q is 2. In some embodiments, the IL-10 conjugate comprises Formula (XI) and q is 3. In some embodiments, the IL-10 conjugate comprises a mixture of Formula (X) and Formula (XI) and q is 1. In some embodiments, the IL-10 conjugate comprises a mixture of Formula (X) and Formula (XI) and q is 2. In some embodiments, the IL-10 conjugate comprises a mixture of Formula (X) and Formula (XI) and q is 3.

Described herein are IL-10 conjugates comprising the amino acid sequence of SEQ ID NO: 1 in which at least one amino acid residue in the IL-10 conjugate is replaced by the structure of Formula (XII) or Formula (XIII), or a mixture of Formula (XII) and Formula (XIII):

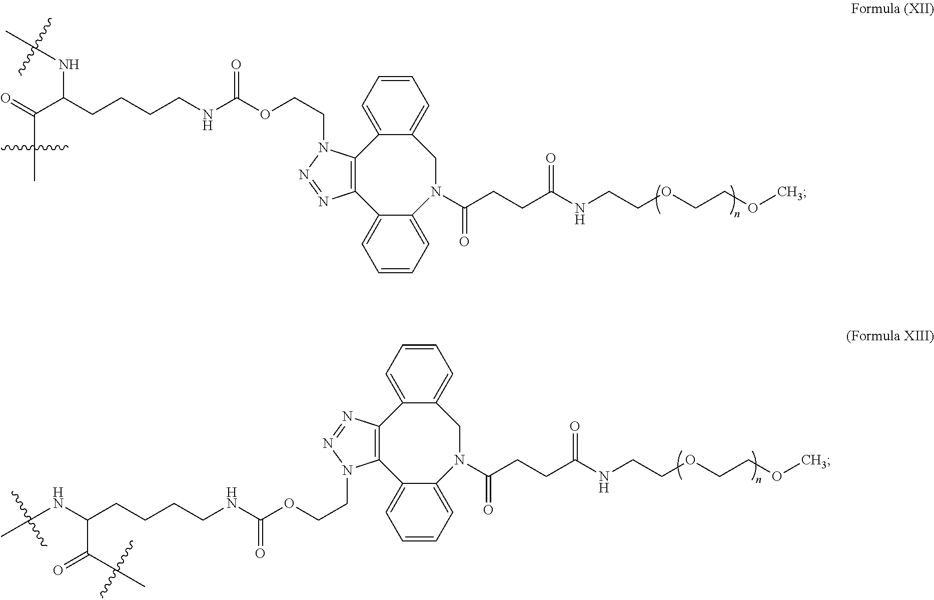

wherein:
n is an integer in the range from about 2 to about 5000; and the wavy lines indicate covalent bonds to amino acid residues within SEQ ID NO: 1 that are not replaced.

Described herein are IL-10 conjugates comprising the amino acid sequence of SEQ ID NO: 1 in which at least one amino acid residue in the IL-10 conjugate is replaced by the structure of Formula (XII) or Formula (XIII), or a mixture of Formula (XII) and Formula (XIII):

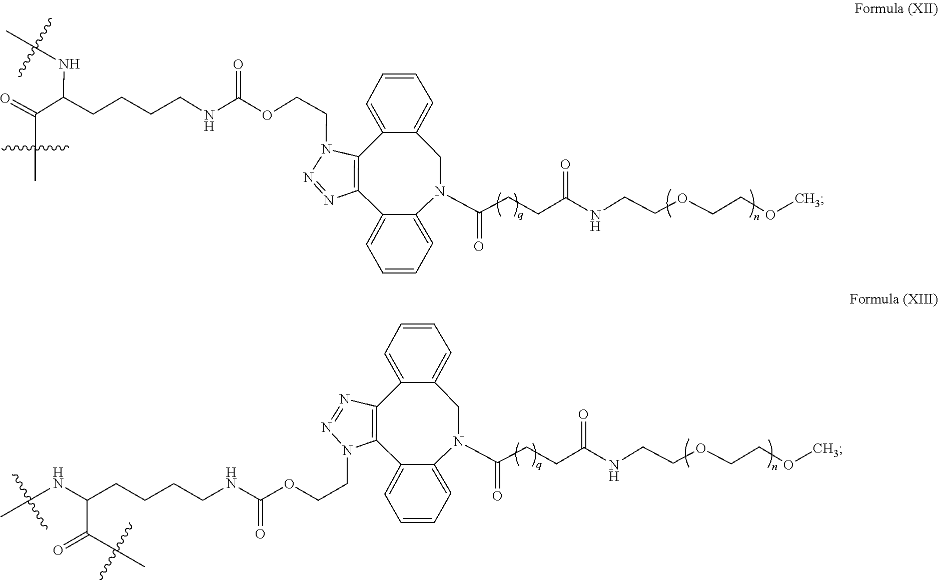

wherein:
n is an integer in the range from about 2 to about 5000;
q is 1, 2, or 3; and
the wavy lines indicate covalent bonds to amino acid residues within SEQ ID NO: 1 that are not replaced. In some embodiments, q is 1. In some embodiments, q is 2. In some embodiments, q is 3.

Here and throughout, the structure of Formula (XII) encompasses pharmaceutically acceptable salts, solvates, or hydrates thereof. Here and throughout, the structure of Formula (XIII) encompasses pharmaceutically acceptable salts, solvates, or hydrates thereof. In some embodiments, the IL-10 conjugate is a pharmaceutically acceptable salt, solvate, or hydrate.

In some embodiments, the stereochemistry of the chiral center within Formula (XII) and Formula (XIII) is racemic, is enriched in (R), is enriched in (S), is substantially (R), is substantially (S), is (R) or is (S). In some embodiments, the stereochemistry of the chiral center within Formula (XII) and Formula (XIII) is racemic. In some embodiments, the stereochemistry of the chiral center within Formula (XII) and Formula (XIII) is enriched in (R). In some embodiments, the stereochemistry of the chiral center within Formula (XII) and Formula (XIII) is enriched in (S). In some embodiments, the stereochemistry of the chiral center within Formula (XII) and Formula (XIII) is substantially (R). In some embodiments, the stereochemistry of the chiral center within Formula (XII) and Formula (XIII) is substantially (S). In some embodiments, the stereochemistry of the chiral center within Formula (XII) and Formula (XIII) is (R). In some embodiments, the stereochemistry of the chiral center within Formula (XII) and Formula (XIII) is (S).

In some embodiments of an IL-10 conjugate described herein, n in the compounds of Formula (XII) or Formula (XIII), or a mixture of Formula (XII) and Formula (XIII), is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments of an IL-10 conjugate described herein, n in the compounds of Formula (XII) or Formula (XIII), or a mixture of Formula (XII) and Formula (XIII), is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546. In some embodiments of an IL-10 conjugate described herein, the position of the structure of Formula (XII) or Formula (XIII), or a mixture of Formula (XII) and Formula (XIII), in the amino acid sequence of the IL-10 conjugate is selected from N82, K88, A89, K99, K125, N126, N129, and K130, wherein the position of the structure of Formula (I) in the amino acid sequence of the IL-10 conjugate is in reference to the positions in SEQ ID NO: 1. In some embodiments of an IL-10 conjugate described herein, the position of the structure of Formula (XII) or Formula (XIII), or a mixture of Formula (XII) and Formula (XIII), in the amino acid sequence of the IL-10 conjugate of SEQ ID NO: 1 is selected from N82, K88, A89, K99, K125, N126, N129, and K130. In some embodiments of an IL-10 conjugate described herein, the position of the structure of Formula (XII) or Formula (XIII), or a mixture of Formula (XII) and Formula (XIII), in the amino acid sequence of the IL-10 conjugate of SEQ ID NO: 1 is at position N82. In some embodiments of an IL-10 conjugate described herein, the position of the structure of Formula (XII) or Formula (XIII), or a mixture of Formula (XII) and Formula (XIII) in the amino acid sequence of the IL-10 conjugate of SEQ ID NO: 1 is at position K88. In some embodiments of an IL-10 conjugate described herein, the position of the structure of Formula (XII) or Formula (XIII), or a mixture of Formula (XII) and Formula (XIII), in the amino acid sequence of the IL-10 conjugate of SEQ ID NO: 1 is at position A89. In some embodiments of an IL-10 conjugate described herein, the position of the structure of Formula (XII) or Formula (XIII), or a mixture of Formula (XII) and Formula (XIII), in the amino acid sequence of the IL-10 conjugate of SEQ ID NO: 1 is at position K99. In some embodiments of an IL-10 conjugate described herein, the position of the structure of Formula (XII) or Formula (XIII), or a mixture of Formula (XII) and Formula (XIII), in the amino acid sequence of the IL-10 conjugate of SEQ ID NO: 1 is at position K125. In some embodiments of an IL-10 conjugate described herein, the position of the structure of Formula (XII) or Formula (XIII), or a mixture of Formula (XII) and Formula (XIII) in the amino acid sequence of the IL-10 conjugate of SEQ ID NO: 1 is at position N126. In some embodiments of an IL-10 conjugate described herein, the position of the structure of Formula (XII) or Formula (XIII), or a mixture of Formula (XII) and Formula (XIII), in the amino acid sequence of the IL-10 conjugate of SEQ ID NO: 1 is at position N129. In some embodiments of an IL-10 conjugate described herein, the position of the structure of Formula (XII) or Formula (XIII), or a mixture of Formula (XII) and Formula (XIII), in the amino acid sequence of the IL-10 conjugate of SEQ ID NO: 1 is at position K130.

In some embodiments of an IL-10 conjugate described herein, the ratio of the amount of the structure of Formula (XII) to the amount of the structure of Formula (XIII) comprising the total amount of the IL-10 conjugate is about 1:1. In some embodiments of an IL-10 conjugate described herein, the ratio of the amount of the structure of Formula (XII) to the amount of the structure of Formula (XIII) comprising the total amount of the IL-10 conjugate is greater than 1:1. In some embodiments of an IL-10 conjugate described herein, the ratio of the amount of the structure of Formula (XII) to the amount of the structure of Formula (XIII) comprising the total amount of the IL-10 conjugate is less than 1:1.

In some embodiments described herein are IL-10 conjugates comprising the amino acid sequence of SEQ ID NO: 1 in which at least one amino acid residue in the IL-10 conjugate is replaced by the structure of Formula (XII) or Formula (XIII), or a mixture of Formula (XII) and Formula (XIII), wherein the amino acid residue in SEQ ID NO: 1 that is replaced is selected from N82, K88, A89, K99, K125, N126, N129, and K130, and wherein n is an integer from 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments of an IL-10 conjugate described herein, n in the compounds of Formula (XII) or Formula (XIII), or a mixture of Formula (XII) and Formula (XIII), is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments described herein are IL-10 conjugates comprising the amino acid sequence of SEQ ID NO: 1 in which at least one amino acid residue in the IL-10 conjugate is replaced by the structure of Formula (XII) or Formula (XIII), or a mixture of Formula (XII) and Formula (XIII), wherein the amino acid residue in SEQ ID NO: 1 that is replaced is selected from N82, K88, A89, K99, K125, N126, N129, and K130, and wherein n is an integer from about 450 to about 800, or from about 454 to about 796, or from about 454 to about 682, or from about 568 to about 909. In some embodiments of an IL-10 conjugate described herein, n in the compounds of Formula (XII) or Formula (XIII), or a mixture of Formula (XII) and Formula (XIII), is an integer selected from 454, 455, 568, 569, 680, 681, 682, 794, 795, 796, 908, 909, 910, 1021, 1022, 1023, 1135, 1136, 1137, and 1249.

In some embodiments described herein are IL-10 conjugates comprising the amino acid sequence of SEQ ID NO: 1 in which at least one amino acid residue in the IL-10 conjugate is replaced by the structure of Formula (XII) or Formula (XIII), or a mixture of Formula (XII) and Formula (XIII), wherein the amino acid residue in SEQ ID NO: 1 that is replaced is selected from N82, K88, A89, K99, K125, N126, N129, and K130, and wherein n is an integer from about 450 to about 800, or from about 454 to about 796, or from about 454 to about 682, or from about 568 to about 909. In some embodiments of an IL-10 conjugate described herein, n in the compounds of Formula (XII) or Formula (XIII), or a mixture of Formula (XII) and Formula (XIII), is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 113, 227, 340, 454, 568, or 681.

Described herein are IL-10 conjugates comprising the amino acid sequence of SEQ ID NO: 1 in which at least one amino acid residue in the IL-10 conjugate is replaced by the structure of Formula (XII) or Formula (XIII), or a mixture of Formula (XII) and Formula (XIII), wherein n is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons.

Described herein are IL-10 conjugates comprising the amino acid sequence of SEQ ID NO: 1 in which at least one amino acid residue in the IL-10 conjugate is replaced by the structure of Formula (XII) or Formula (XIII), or a mixture of Formula (XII) and Formula (XIII), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons. Described herein are IL-10 conjugates comprising the amino acid sequence of SEQ ID NO: 1 in which at least one amino acid residue in the IL-10 conjugate is replaced by the structure of Formula (XII) or Formula (XIII), or a mixture of Formula (XII) and Formula (XIII), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

In some embodiments described herein of Formula (XII), Formula (XIII), or a mixture of Formula (XII) and Formula (XIII), q is 1. In some embodiments described herein of Formula (XII), Formula (XIII), or a mixture of Formula (XII) and Formula (XIII), q is 2. In some embodiments described herein of Formula (XII), Formula (XIII), or a mixture of Formula (XII) and Formula (XIII), q is 3. In some embodiments, the IL-10 conjugate comprises Formula (XII) and q is 1. In some embodiments, the IL-10 conjugate comprises Formula (XII) and q is 2. In some embodiments, the IL-10 conjugate comprises Formula (XII) and q is 3. In some embodiments, the IL-10 conjugate comprises Formula (XIII) and q is 1. In some embodiments, the IL-10 conjugate comprises Formula (XIII) and q is 2. In some embodiments, the IL-10 conjugate comprises Formula (XIII) and q is 3. In some embodiments, the IL-10 conjugate comprises a mixture of Formula (XII) and Formula (XIII) and q is 1. In some embodiments, the IL-10 conjugate comprises a mixture of Formula (XII) and Formula (XIII) and q is 2. In some embodiments, the IL-10 conjugate comprises a mixture of Formula (XII) and Formula (XIII) and q is 3.

Described herein are IL-10 conjugates comprising the amino acid sequence of SEQ ID NO: 1 in which at least one amino acid residue in the IL-10 conjugate is replaced by the structure of Formula (XIV) or Formula (XV), or a mixture of Formula (XIV) and Formula (XV):

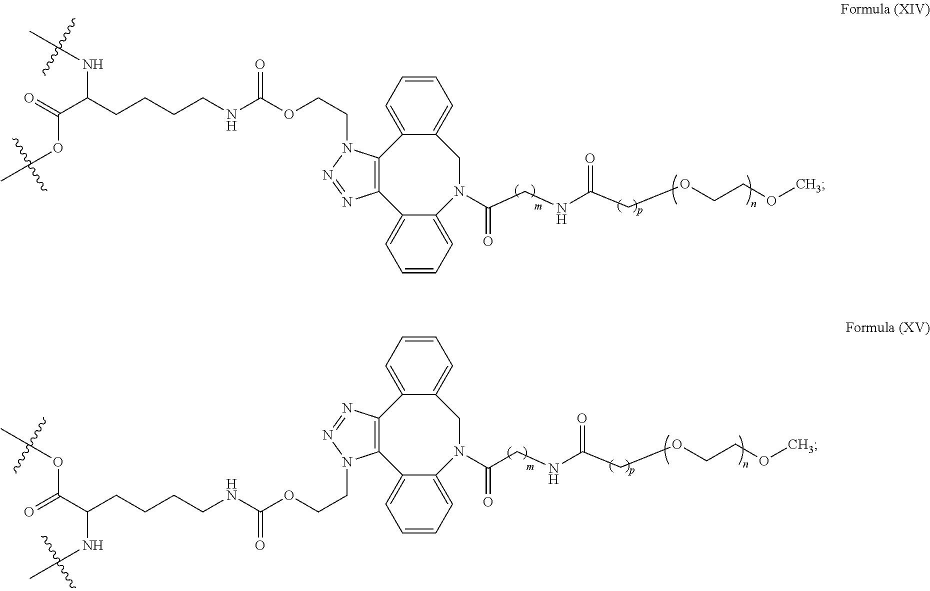

wherein:
m is an integer from 0 to 20;
p is an integer from 0 to 20;
n is an integer in the range from about 2 to about 5000; and
the wavy lines indicate covalent bonds to amino acid residues within SEQ ID NO: 1 that are not replaced.

Here and throughout, the structure of Formula (XIV) encompasses pharmaceutically acceptable salts, solvates, or hydrates thereof. Here and throughout, the structure of Formula (XV) encompasses pharmaceutically acceptable salts, solvates, or hydrates thereof. In some embodiments, the IL-10 conjugate is a pharmaceutically acceptable salt, solvate, or hydrate.

In some embodiments, the stereochemistry of the chiral center within Formula (XIV) and Formula (XV) is racemic, is enriched in (R), is enriched in (S), is substantially (R), is substantially (S), is (R) or is (S). In some embodiments, the stereochemistry of the chiral center within Formula (XIV) and Formula (XV) is racemic. In some embodiments, the stereochemistry of the chiral center within Formula (XIV) and Formula (XV) is enriched in (R). In some embodiments, the stereochemistry of the chiral center within Formula (XIV) and Formula (XV) is enriched in (S). In some embodiments, the stereochemistry of the chiral center within Formula (XIV) and Formula (XV) is substantially (R). In some embodiments, the stereochemistry of the chiral center within Formula (XIV) and Formula (XV) is substantially (S). In some embodiments, the stereochemistry of the chiral center within Formula (XIV) and Formula (XV) is (R). In some embodiments, the stereochemistry of the chiral center within Formula (XIV) and Formula (XV) is (S).

In some embodiments of an IL-10 conjugate described herein, m in the compounds of Formula (XIV) or Formula (XV), or a mixture of Formula (XIV) and Formula (XV), is from 0 to 20, or from 1 to 18, or from 1 to 16, or from 1 to 14, or from 1 to 12, or from 1 to 10, or from 1 to 9, or from 1 to 8, or from 1 to 7, or from 1 to 6, or from 1 to 5, or from 1 to 4, or from 1 to 3, or from 1 to 2. In some embodiments of an IL-10 conjugate described herein, m in the compounds of Formula (XIV) or Formula (XV), or a mixture of Formula (XIV) and Formula (XV), is 1. In some embodiments of an IL-10 conjugate described herein, m in the compounds of Formula (XIV) or Formula (XV), or a mixture of Formula (XIV) and Formula (XV), is 2. In some embodiments of an IL-10 conjugate described herein, m in the compounds of Formula (XIV) or Formula (XV), or a mixture of Formula (XIV) and Formula (XV), is 3. In some embodiments of an IL-10 conjugate described herein, m in the compounds of Formula (XIV) or Formula (XV), or a mixture of Formula (XIV) and Formula (XV), is 4. In some embodiments of an IL-10 conjugate described herein, m in the compounds of Formula (XIV) or Formula (XV), or a mixture of Formula (XIV) and Formula (XV), is 5. In some embodiments of an IL-10 conjugate described herein, m in the compounds of Formula (XIV) or Formula (XV), or a mixture of Formula (XIV) and Formula (XV), is 6. In some embodiments of an IL-10 conjugate described herein, m in the compounds of Formula (XIV) or Formula (XV), or a mixture of Formula (XIV) and Formula (XV), is 7. In some embodiments of an IL-10 conjugate described herein, m in the compounds of Formula (XIV) or Formula (XV), or a mixture of Formula (XIV) and Formula (XV), is 8. In some embodiments of an IL-10 conjugate described herein, m in the compounds of Formula (XIV) or Formula (XV), or a mixture of Formula (XIV) and Formula (XV), is 9. In some embodiments of an IL-10 conjugate described herein, m in the compounds of Formula (XIV) or Formula (XV), or a mixture of Formula (XIV) and Formula (XV), is 10. In some embodiments of an IL-10 conjugate described herein, m in the compounds of Formula (XIV) or Formula (XV), or a mixture of Formula (XIV) and Formula (XV), is 11. In some embodiments of an IL-10 conjugate described herein, m in the compounds of Formula (XIV) or Formula (XV), or a mixture of Formula (XIV) and Formula (XV), is 12. In some embodiments of an IL-10 conjugate described herein, m in the compounds of Formula (XIV) or Formula (XV), or a mixture of Formula (XIV) and Formula (XV), is 13. In some embodiments of an IL-10 conjugate described herein, m in the compounds of Formula (XIV) or Formula (XV), or a mixture of Formula (XIV) and Formula (XV), is 14. In some embodiments of an IL-10 conjugate described herein, m in the compounds of Formula (XIV) or Formula (XV), or a mixture of Formula (XIV) and Formula (XV), is 15. In some embodiments of an IL-10 conjugate described herein, m in the compounds of Formula (XIV) or Formula (XV), or a mixture of Formula (XIV) and Formula (XV), is 16. In some embodiments of an IL-10 conjugate described herein, m in the compounds of Formula (XIV) or Formula (XV), or a mixture of Formula (XIV) and Formula (XV), is 17. In some embodiments of an IL-10 conjugate described herein, m in the compounds of Formula (XIV) or Formula (XV), or a mixture of Formula (XIV) and Formula (XV), is 18. In some embodiments of an IL-10 conjugate described herein, m in the compounds of Formula (XIV) or Formula (XV), or a mixture of Formula (XIV) and Formula (XV), is 19. In some embodiments of an IL-10 conjugate described herein, m in the compounds of Formula (XIV) or Formula (XV), or a mixture of Formula (XIV) and Formula (XV), is 20.

In some embodiments of an IL-10 conjugate described herein, p in the compounds of Formula (XIV) or Formula (XV), or a mixture of Formula (XIV) and Formula (XV), is from 1 to 20, or from 1 to 18, or from 1 to 16, or from 1 to 14, or from 1 to 12, or from 1 to 10, or from 1 to 9, or from 1 to 8, or from 1 to 7, or from 1 to 6, or from 1 to 5, or from 1 to 4, or from 1 to 3, or from 1 to 2. In some embodiments of an IL-10 conjugate described herein, p in the compounds of Formula (XIV) or Formula (XV), or a mixture of Formula (XIV) and Formula (XV), is 1. In some embodiments of an IL-10 conjugate described herein, p in the compounds of Formula (XIV) or Formula (XV), or a mixture of Formula (XIV) and Formula (XV), is 2. In some embodiments of an IL-10 conjugate described herein, p in the compounds of Formula (XIV) or Formula (XV), or a mixture of Formula (XIV) and Formula (XV), is 3. In some embodiments of an IL-10 conjugate described herein, p in the compounds of Formula (XIV) or Formula (XV), or a mixture of Formula (XIV) and Formula (XV), is 4. In some embodiments of an IL-10 conjugate described herein, p in the compounds of Formula (XIV) or Formula (XV), or a mixture of Formula (XIV) and Formula (XV), is 5. In some embodiments of an IL-10 conjugate described herein, p in the compounds of Formula (XIV) or Formula (XV), or a mixture of Formula (XIV) and Formula (XV), is 6. In some embodiments of an IL-10 conjugate described herein, p in the compounds of Formula (XIV) or Formula (XV), or a mixture of Formula (XIV) and Formula (XV), is 7. In some embodiments of an IL-10 conjugate described herein, p in the compounds of Formula (XIV) or Formula (XV), or a mixture of Formula (XIV) and Formula (XV), is 8. In some embodiments of an IL-10 conjugate described herein, p in the compounds of Formula (XIV) or Formula (XV), or a mixture of Formula (XIV) and Formula (XV), is 9. In some embodiments of an IL-10 conjugate described herein, p in the compounds of Formula (XIV) or Formula (XV), or a mixture of Formula (XIV) and Formula (XV), is 10. In some embodiments of an IL-10 conjugate described herein, p in the compounds of Formula (XIV) or Formula (XV), or a mixture of Formula (XIV) and Formula (XV), is 11. In some embodiments of an IL-10 conjugate described herein, p in the compounds of Formula (XIV) or Formula (XV), or a mixture of Formula (XIV) and Formula (XV), is 12. In some embodiments of an IL-10 conjugate described herein, p in the compounds of Formula (XIV) or Formula (XV), or a mixture of Formula (XIV) and Formula (XV), is 13. In some embodiments of an IL-10 conjugate described herein, p in the compounds of Formula (XIV) or Formula (XV), or a mixture of Formula (XIV) and Formula (XV), is 14. In some embodiments of an IL-10 conjugate described herein, p in the compounds of Formula (XIV) or Formula (XV), or a mixture of Formula (XIV) and Formula (XV), is 15. In some embodiments of an IL-10 conjugate described herein, m in the compounds of Formula (XIV) or Formula (XV), or a mixture of Formula (XIV) and Formula (XV), is 16. In some embodiments of an IL-10 conjugate described herein, p in the compounds of Formula (XIV) or Formula (XV), or a mixture of Formula (XIV) and Formula (XV), is 17. In some embodiments of an IL-10 conjugate described herein, p in the compounds of Formula (XIV) or Formula (XV), or a mixture of Formula (XIV) and Formula (XV), is 18. In some embodiments of an IL-10 conjugate described herein, p in the compounds of Formula (XIV) or Formula (XV), or a mixture of Formula (XIV) and Formula (XV), is 19. In some embodiments of an IL-10 conjugate described herein, p in the compounds of Formula (XIV) or Formula (XV), or a mixture of Formula (XIV) and Formula (XV), is 20.

In some embodiments of an IL-10 conjugate described herein, n in the compounds of Formula (XIV) or Formula (XV), or a mixture of Formula (XIV) and Formula (XV), is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575.

In some embodiments of an IL-10 conjugate described herein in the compounds of Formula (XIV) or Formula (XV), or a mixture of Formula (XIV) and Formula (XV), m is an integer from 1 to 6, p is an integer from 1 to 6, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XIV) or Formula (XV), or a mixture of Formula (XIV) and Formula (XV), m is an integer from 2 to 6, p is an integer from 2 to 6, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XIV) or Formula (XV), or a mixture of Formula (XIV) and Formula (XV), m is an integer from 2 to 4, p is an integer from 2 to 4, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XIV) or Formula (XV), or a mixture of Formula (XIV) and Formula (XV), m is 1, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XIV) or Formula (XV), or a mixture of Formula (XIV) and Formula (XV), m is 2, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XIV) or Formula (XV), or a mixture of Formula (XIV) and Formula (XV), m is 3, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XIV) or Formula (XV), or a mixture of Formula (XIV) and Formula (XV), m is 4, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XIV) or Formula (XV), or a mixture of Formula (XIV) and Formula (XV), m is 5, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XIV) or Formula (XV), or a mixture of Formula (XIV) and Formula (XV), m is 6, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XIV) or Formula (XV), or a mixture of Formula (XIV) and Formula (XV), m is 7, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XIV) or Formula (XV), or a mixture of Formula (XIV) and Formula (XV), m is 8, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XIV) or Formula (XV), or a mixture of Formula (XIV) and Formula (XV), m is 9, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XIV) or Formula (XV), or a mixture of Formula (XIV) and Formula (XV), m is 10, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XIV) or Formula (XV), or a mixture of Formula (XIV) and Formula (XV), m is 11, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XIV) or Formula (XV), or a mixture of Formula (XIV) and Formula (XV), m is 11, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XIV) or Formula (XV), or a mixture of Formula (XIV) and Formula (XV), m is 2, p is 2, and n is an integer selected from 680, 681, 682, 794, 795, 796, 908, 909, 910, 1021, 1022, 1023, 1135, 1136, and 1137.

In some embodiments of an IL-10 conjugate described herein, n in the compounds of Formula (XIV) or Formula (XV), or a mixture of Formula (XIV) and Formula (XV), is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546. In some embodiments of an IL-10 conjugate described herein, the position of the structure of Formula (XIV) or Formula (XV), or a mixture of Formula (XIV) and Formula (XV), in the amino acid sequence of the IL-10 conjugate is selected from N82, K88, A89, K99, K125, N126, N129, and K130. In some embodiments of an IL-10 conjugate described herein, the position of the structure of Formula (XIV) or Formula (XV), or a mixture of Formula (XIV) and Formula (XV), in the amino acid sequence of the IL-10 conjugate of SEQ ID NO: 1 is at position N82. In some embodiments of an IL-10 conjugate described herein, the position of the structure of Formula (XIV) or Formula (XV), or a mixture of Formula (XIV) and Formula (XV), in the amino acid sequence of the IL-10 conjugate of SEQ ID NO: 1 is at position K88. In some embodiments of an IL-10 conjugate described herein, the position of the structure of Formula (XIV) or Formula (XV), or a mixture of Formula (XIV) and Formula (XV), in the amino acid sequence of the IL-10 conjugate of SEQ ID NO: 1 is at position A89. In some embodiments of an IL-10 conjugate described herein, the position of the structure of Formula (XIV) or Formula (XV), or a mixture of Formula (XIV) and Formula (XV), in the amino acid sequence of the IL-10 conjugate of SEQ ID NO: 1 is at position K99. In some embodiments of an IL-10 conjugate described herein, the position of the structure of Formula (XIV) or Formula (XV), or a mixture of Formula (XIV) and Formula (XV), in the amino acid sequence of the IL-10 conjugate of SEQ ID NO: 1 is at position K125. In some embodiments of an IL-10 conjugate described herein, the position of the structure of Formula (XIV) or Formula (XV), or a mixture of Formula (XIV) and Formula (XV), in the amino acid sequence of the IL-10 conjugate of SEQ ID NO: 1 is at position N126. In some embodiments of an IL-10 conjugate described herein, the position of the structure of Formula (XIV) or Formula (XV), or a mixture of Formula (XIV) and Formula (XV), in the amino acid sequence of the IL-10 conjugate of SEQ ID NO: 1 is at position N129. In some embodiments of an IL-10 conjugate described herein, the position of the structure of Formula (XIV) or Formula (XV), or a mixture of Formula (XIV) and Formula (XV), in the amino acid sequence of the IL-10 conjugate of SEQ ID NO: 1 is at position K130.

In some embodiments of an IL-10 conjugate described herein, the ratio of the amount of the structure of Formula (XIV) to the amount of the structure of Formula (XV) comprising the total amount of the IL-10 conjugate is about 1:1. In some embodiments of an IL-10 conjugate described herein, the ratio of the amount of the structure of Formula (XIV) to the amount of the structure of Formula (XV) comprising the total amount of the IL-10 conjugate is greater than 1:1. In some embodiments of an IL-10 conjugate described herein, the ratio of the amount of the structure of Formula (XIV) to the amount of the structure of Formula (XV) comprising the total amount of the IL-10 conjugate is less than 1:1.

In some embodiments described herein are IL-10 conjugates comprising the amino acid sequence of SEQ ID NO: 1 in which at least one amino acid residue in the IL-10 conjugate is replaced by the structure of Formula (XIV) or Formula (XV), or a mixture of Formula (XIV) and Formula (XV), wherein the amino acid residue in SEQ ID NO: 1 that is replaced is selected from N82, K88, A89, K99, K125, N126, N129, and K130, and wherein n is an integer from 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments of an IL-10 conjugate described herein, n in the compounds of Formula (XIV) or Formula (XV), or a mixture of Formula (XIV) and Formula (XV), is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments described herein are IL-10 conjugates comprising the amino acid sequence of SEQ ID NO: 1 in which at least one amino acid residue in the IL-10 conjugate is replaced by the structure of Formula (XIV) or Formula (XV), or a mixture of Formula (XIV) and Formula (XV), wherein the amino acid residue in SEQ ID NO: 1 that is replaced is selected from N82, K88, A89, K99, K125, N126, N129, and K130, and wherein n is an integer from about 450 to about 800, or from about 454 to about 796, or from about 454 to about 682, or from about 568 to about 909. In some embodiments of an IL-10 conjugate described herein, n in the compounds of Formula (XIV) or Formula (XV), or a mixture of Formula (XIV) and Formula (XV), is an integer selected from 454, 455, 568, 569, 680, 681, 682, 794, 795, 796, 908, 909, 910, 1021, 1022, 1023, 1135, 1136, 1137, and 1249.

In some embodiments described herein are IL-10 conjugates comprising the amino acid sequence of SEQ ID NO: 1 in which at least one amino acid residue in the IL-10 conjugate is replaced by the structure of Formula (XIV) or Formula (XV), or a mixture of Formula (XIV) and Formula (XV), wherein the amino acid residue in SEQ ID NO: 1 that is replaced is selected from N82, K88, A89, K99, K125, N126, N129, and K130, and wherein n is an integer from about 450 to about 800, or from about 454 to about 796, or from about 454 to about 682, or from about 568 to about 909. In some embodiments of an IL-10 conjugate described herein, n in the compounds of Formula (XIV) or Formula (XV), or a mixture of Formula (XIV) and Formula (XV), is an integer selected from 454, 455, 568, 569, 680, 681, 682, 794, 795, 796, 908, 909, and 910.

In some embodiments described herein are IL-10 conjugates comprising the amino acid sequence of SEQ ID NO: 1 in which at least one amino acid residue in the IL-10 conjugate is replaced by the structure of Formula (XIV) or Formula (XV), or a mixture of Formula (XIV) and Formula (XV), wherein the amino acid residue in SEQ ID NO: 1 that is replaced is selected from N82, K88, A89, K99, K125, N126, N129, and K130, and wherein n is an integer from about 450 to about 800, or from about 454 to about 796, or from about 454 to about 682, or from about 568 to about 909. In some embodiments of an IL-10 conjugate described herein, n in the compounds of Formula (XIV) or Formula (XV), or a mixture of Formula (XIV) and Formula (XV), is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

Described herein are IL-10 conjugates comprising the amino acid sequence of SEQ ID NO: 1 in which at least one amino acid residue in the IL-10 conjugate is replaced by the structure of Formula (XIV) or Formula (XV), or a mixture of Formula (XIV) and Formula (XV), wherein n is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons.

Described herein are IL-10 conjugates comprising the amino acid sequence of SEQ ID NO: 1 in which at least one amino acid residue in the IL-10 conjugate is replaced by the structure of Formula (XIV) or Formula (XV), or a mixture of Formula (XIV) and Formula (XV), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons. Described herein are IL-10 conjugates comprising the amino acid sequence of SEQ ID NO: 1 in which at least one amino acid residue in the IL-10 conjugate is replaced by the structure of Formula (XIV) or Formula (XV), or a mixture of Formula (XIV) and Formula (XV), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

In some embodiments described herein are IL-10 conjugates comprising the amino acid sequence of SEQ ID NO: 1 in which at least one amino acid residue in the IL-10 conjugate is replaced by the structure of Formula (XIV) or Formula (XV), or a mixture of Formula (XIV) and Formula (XV), wherein the amino acid residue in SEQ ID NO: 1 that is replaced is selected from N82, K88, A89, K99, K125, N126, N129, and K130, m is an integer from 1 to 6, p is an integer from 1 to 6, and n is an integer from about 450 to about 800, or from about 454 to about 796, or from about 454 to about 682, or from about 568 to about 909. In some embodiments of an IL-10 conjugate described herein in the compounds of Formula (XIV) or Formula (XV), or a mixture of Formula (XIV) and Formula (XV), m is 2, p is 2, and n is an integer selected from 454, 455, 568, 569, 680, 681, 682, 794, 795, 796, 908, 909, 910, 1021, 1022, 1023, 1135, 1136, 1137, and 1249.

In some embodiments described herein are IL-10 conjugates comprising the amino acid sequence of SEQ ID NO: 1 in which at least one amino acid residue in the IL-10 conjugate is replaced by the structure of Formula (XIV) or Formula (XV), or a mixture of Formula (XIV) and Formula (XV), wherein the amino acid residue in SEQ ID NO: 1 that is replaced is selected from N82, K88, A89, K99, K125, N126, N129, and K130, and wherein m is an integer from 1 to 6, p is an integer from 1 to 6, and n is an integer from about 450 to about 800, or from about 454 to about 796, or from about 454 to about 682, or from about 568 to about 909. In some embodiments of an IL-10 conjugate described herein in the compounds of Formula (XIV) or Formula (XV), or a mixture of Formula (XIV) and Formula (XV), m is 2, p is 2, and n is an integer selected from 454, 455, 568, 569, 680, 681, 682, 794, 795, 796, 908, 909, and 910.

In some embodiments described herein are IL-10 conjugates comprising the amino acid sequence of SEQ ID NO: 1 in which at least one amino acid residue in the IL-10 conjugate is replaced by the structure of Formula (XIV) or Formula (XV), or a mixture of Formula (XIV) and Formula (XV), wherein the amino acid residue in SEQ ID NO: 1 that is replaced is selected from N82, K88, A89, K99, K125, N126, N129, and K130, and wherein m is an integer from 1 to 6, p is an integer from 1 to 6, and n is an integer from about 450 to about 800, or from about 454 to about 796, or from about 454 to about 682, or from about 568 to about 909. In some embodiments of an IL-10 conjugate described herein in the compounds of Formula (XIV) or Formula (XV), or a mixture of Formula (XIV) and Formula (XV), m is 2, p is 2, and n is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

Described herein are IL-10 conjugates comprising the amino acid sequence of SEQ ID NO: 1 in which at least one amino acid residue in the IL-10 conjugate is replaced by the structure of Formula (XVI) or Formula (XVII), or a mixture of Formula (XVI) and Formula (XVII):

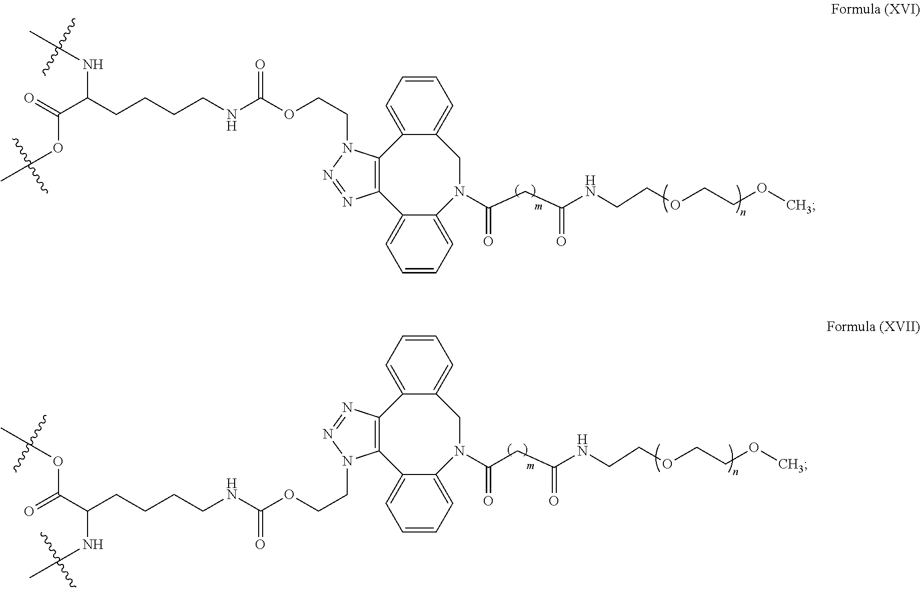

wherein:
m is an integer from 0 to 20;
n is an integer in the range from about 2 to about 5000; and
the wavy lines indicate covalent bonds to amino acid residues within SEQ ID NO: 1 that are not replaced.

Here and throughout, the structure of Formula (XVI) encompasses pharmaceutically acceptable salts, solvates, or hydrates thereof. Here and throughout, the structure of Formula (XVII) encompasses pharmaceutically acceptable salts, solvates, or hydrates thereof. In some embodiments, the IL-10 conjugate is a pharmaceutically acceptable salt, solvate, or hydrate.

In some embodiments, the stereochemistry of the chiral center within Formula (XVI) and Formula (XVII) is racemic, is enriched in (R), is enriched in (S), is substantially (R), is substantially (S), is (R) or is (S). In some embodiments, the stereochemistry of the chiral center within Formula (XVI) and Formula (XVII) is racemic. In some embodiments, the stereochemistry of the chiral center within Formula (XVI) and Formula (XVII) is enriched in (R). In some embodiments, the stereochemistry of the chiral center within Formula (XVI) and Formula (XVII) is enriched in (S). In some embodiments, the stereochemistry of the chiral center within Formula (XVI) and Formula (XVII) is substantially (R). In some embodiments, the stereochemistry of the chiral center within Formula (XVI) and Formula (XVII) is substantially (S). In some embodiments, the stereochemistry of the chiral center within Formula (XVI) and Formula (XVII) is (R). In some embodiments, the stereochemistry of the chiral center within Formula (XVI) and Formula (XVII) is (S).

In some embodiments of an IL-10 conjugate described herein, m in the compounds of Formula (XVI) or Formula (XVII), or a mixture of Formula (XVI) and Formula (XVII), is from 1 to 20, or from 1 to 18, or from 1 to 16, or from 1 to 14, or from 1 to 12, or from 1 to 10, or from 1 to 9, or from 1 to 8, or from 1 to 7, or from 1 to 6, or from 1 to 5, or from 1 to 4, or from 1 to 3, or from 1 to 2. In some embodiments of an IL-10 conjugate described herein, m in the compounds of Formula (XVI) or Formula (XVII), or a mixture of Formula (XVI) and Formula (XVII), is 1. In some embodiments of an IL-10 conjugate described herein, m in the compounds of Formula (XVI) or Formula (XVII), or a mixture of Formula (XVI) and Formula (XVII), is 2. In some embodiments of an IL-10 conjugate described herein, m in the compounds of Formula (XVI) or Formula (XVII), or a mixture of Formula (XVI) and Formula (XVII), is 3. In some embodiments of an IL-10 conjugate described herein, m in the compounds of Formula (XVI) or Formula (XVII), or a mixture of Formula (XVI) and Formula (XVII), is 4. In some embodiments of an IL-10 conjugate described herein, m in the compounds of Formula (XVI) or Formula (XVII), or a mixture of Formula (XVI) and Formula (XVII), is 5. In some embodiments of an IL-10 conjugate described herein, m in the compounds of Formula (XVI) or Formula (XVII), or a mixture of Formula (XVI) and Formula (XVII), is 6. In some embodiments of an IL-10 conjugate described herein, m in the compounds of Formula (XVI) or Formula (XVII), or a mixture of Formula (XVI) and Formula (XVII), is 7. In some embodiments of an IL-10 conjugate described herein, m in the compounds of Formula (XVI) or Formula (XVII), or a mixture of Formula (XVI) and Formula (XVII), is 8. In some embodiments of an IL-10 conjugate described herein, m in the compounds of Formula (XVI) or Formula (XVII), or a mixture of Formula (XVI) and Formula (XVII), is 9. In some embodiments of an IL-10 conjugate described herein, m in the compounds of Formula (XVI) or Formula (XVII), or a mixture of Formula (XVI) and Formula (XVII), is 10. In some embodiments of an IL-10 conjugate described herein, m in the compounds of Formula (XVI) or Formula (XVII), or a mixture of Formula (XVI) and Formula (XVII), is 11. In some embodiments of an IL-10 conjugate described herein, m in the compounds of Formula (XVI) or Formula (XVII), or a mixture of Formula (XVI) and Formula (XVII), is 12. In some embodiments of an IL-10 conjugate described herein, m in the compounds of Formula (XVI) or Formula (XVII), or a mixture of Formula (XVI) and Formula (XVII), is 13. In some embodiments of an IL-10 conjugate described herein, m in the compounds of Formula (XVI) or Formula (XVII), or a mixture of Formula (XVI) and Formula (XVII), is 14. In some embodiments of an IL-10 conjugate described herein, m in the compounds of Formula (XVI) or Formula (XVII), or a mixture of Formula (XVI) and Formula (XVII), is 15. In some embodiments of an IL-10 conjugate described herein, m in the compounds of Formula (XVI) or Formula (XVII), or a mixture of Formula (XVI) and Formula (XVII), is 16. In some embodiments of an IL-10 conjugate described herein, m in the compounds of Formula (XVI) or Formula (XVII), or a mixture of Formula (XVI) and Formula (XVII), is 17. In some embodiments of an IL-10 conjugate described herein, m in the compounds of Formula (XVI) or Formula (XVII), or a mixture of Formula (XVI) and Formula (XVII), is 18. In some embodiments of an IL-10 conjugate described herein, m in the compounds of Formula (XVI) or Formula (XVII), or a mixture of Formula (XVI) and Formula (XVII), is 19. In some embodiments of an IL-10 conjugate described herein, m in the compounds of Formula (XVI) or Formula (XVII), or a mixture of Formula (XVI) and Formula (XVII), is 20.

In some embodiments of an IL-10 conjugate described herein, n in the compounds of Formula (XVI) or Formula (XVII), or a mixture of Formula (XVI) and Formula (XVII), is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575.

In some embodiments of an IL-10 conjugate described herein in the compounds of Formula (XVI) or Formula (XVII), or a mixture of Formula (XVI) and Formula (XVII), m is an integer from 1 to 6, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XVI) or Formula (XVII), or a mixture of Formula (XVI) and Formula (XVII), m is an integer from 2 to 6, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XVI) or Formula (XVII), or a mixture of Formula (XVI) and Formula (XVII), m is an integer from 2 to 4, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XVI) or Formula (XVII), or a mixture of Formula (XVI) and Formula (XVII), m is 1, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XVI) or Formula (XVII), or a mixture of Formula (XVI) and Formula (XVII), m is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XVI) or Formula (XVII), or a mixture of Formula (XVI) and Formula (XVII), m is 3, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XVI) or Formula (XVII), or a mixture of Formula (XVI) and Formula (XVII), m is 4, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XVI) or Formula (XVII), or a mixture of Formula (XVI) and Formula (XVII), m is 5, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XVI) or Formula (XVII), or a mixture of Formula (XVI) and Formula (XVII), m is 6, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XVI) or Formula (XVII), or a mixture of Formula (XVI) and Formula (XVII), m is 7, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XVI) or Formula (XVII), or a mixture of Formula (XVI) and Formula (XVII), m is 8, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XVI) or Formula (XVII), or a mixture of Formula (XVI) and Formula (XVII), m is 9, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XVI) or Formula (XVII), or a mixture of Formula (XVI) and Formula (XVII), m is 10, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XVI) or Formula (XVII), or a mixture of Formula (XVI) and Formula (XVII), m is 11, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XVI) or Formula (XVII), or a mixture of Formula (XVI) and Formula (XVII), m is 12, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-10 conjugate described herein in the compounds of Formula (XVI) or Formula (XVII), or a mixture of Formula (XVI) and Formula (XVII), m is 2, and n is an integer selected from 680, 681, 682, 794, 795, 796, 908, 909, 910, 1021, 1022, 1023, 1135, 1136, and 1137.

In some embodiments of an IL-10 conjugate described herein, n in the compounds of Formula (XVI) or Formula (XVII), or a mixture of Formula (XVI) and Formula (XVII), is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments of an IL-10 conjugate described herein, the position of the structure of Formula (XVI) or Formula (XVII), or a mixture of Formula (XVI) and Formula (XVII), in the amino acid sequence of the IL-10 conjugate is selected from N82, K88, A89, K99, K125, N126, N129, and K130. In some embodiments of an IL-10 conjugate described herein, the position of the structure of Formula (XVI) or Formula (XVII), or a mixture of Formula (XVI) and Formula (XVII), in the amino acid sequence of the IL-10 conjugate of SEQ ID NO: 1 is at position N82. In some embodiments of an IL-10 conjugate described herein, the position of the structure of Formula (XVI) or Formula (XVII), or a mixture of Formula (XVI) and Formula (XVII), in the amino acid sequence of the IL-10 conjugate of SEQ ID NO: 1 is at position K88. In some embodiments of an IL-10 conjugate described herein, the position of the structure of Formula (XVI) or Formula (XVII), or a mixture of Formula (XVI) and Formula (XVII), in the amino acid sequence of the IL-10 conjugate of SEQ ID NO: 1 is at position A89. In some embodiments of an IL-10 conjugate described herein, the position of the structure of Formula (XVI) or Formula (XVII), or a mixture of Formula (XVI) and Formula (XVII), in the amino acid sequence of the IL-10 conjugate of SEQ ID NO: 1 is at position K99. In some embodiments of an IL-10 conjugate described herein, the position of the structure of Formula (XVI) or Formula (XVII), or a mixture of Formula (XVI) and Formula (XVII), in the amino acid sequence of the IL-10 conjugate of SEQ ID NO: 1 is at position K125. In some embodiments of an IL-10 conjugate described herein, the position of the structure of Formula (XVI) or Formula (XVII), or a mixture of Formula (XVI) and Formula (XVII), in the amino acid sequence of the IL-10 conjugate of SEQ ID NO: 1 is at position N126. In some embodiments of an IL-10 conjugate described herein, the position of the structure of Formula (XVI) or Formula (XVII), or a mixture of Formula (XVI) and Formula (XVII), in the amino acid sequence of the IL-10 conjugate of SEQ ID NO: 1 is at position N129. In some embodiments of an IL-10 conjugate described herein, the position of the structure of Formula (XVI) or Formula (XVII), or a mixture of Formula (XVI) and Formula (XVII), in the amino acid sequence of the IL-10 conjugate of SEQ ID NO: 1 is at position K130.

In some embodiments of an IL-10 conjugate described herein, the ratio of the amount of the structure of Formula (XVI) to the amount of the structure of Formula (XVII) comprising the total amount of the IL-10 conjugate is about 1:1. In some embodiments of an IL-10 conjugate described herein, the ratio of the amount of the structure of Formula (XVI) to the amount of the structure of Formula (XVII) comprising the total amount of the IL-10 conjugate is greater than 1:1. In some embodiments of an IL-10 conjugate described herein, the ratio of the amount of the structure of Formula (XVI) to the amount of the structure of Formula (XVII) comprising the total amount of the IL-10 conjugate is less than 1:1.

In some embodiments described herein are IL-10 conjugates comprising the amino acid sequence of SEQ ID NO: 1 in which at least one amino acid residue in the IL-10 conjugate is replaced by the structure of Formula (XVI) or Formula (XVII), or a mixture of Formula (XVI) and Formula (XVII), wherein the amino acid residue in SEQ ID NO: 1 that is replaced is selected from N82, K88, A89, K99, K125, N126, N129, and K130, and wherein n is an integer from 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments of an IL-10 conjugate described herein, n in the compounds of Formula (XVI) or Formula (XVII), or a mixture of Formula (XVI) and Formula (XVII), is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments described herein are IL-10 conjugates comprising the amino acid sequence of SEQ ID NO: 1 in which at least one amino acid residue in the IL-10 conjugate is replaced by the structure of Formula (XVI) or Formula (XVII), or a mixture of Formula (XVI) and Formula (XVII), wherein the amino acid residue in SEQ ID NO: 1 that is replaced is selected from N82, K88, A89, K99, K125, N126, N129, and K130, and wherein n is an integer from about 450 to about 800, or from about 454 to about 796, or from about 454 to about 682, or from about 568 to about 909. In some embodiments of an IL-10 conjugate described herein, n in the compounds of Formula (XVI) or Formula (XVII), or a mixture of Formula (XVI) and Formula (XVII), is an integer selected from 454, 455, 568, 569, 680, 681, 682, 794, 795, 796, 908, 909, 910, 1021, 1022, 1023, 1135, 1136, 1137, and 1249.

In some embodiments described herein are IL-10 conjugates comprising the amino acid sequence of SEQ ID NO: 1 in which at least one amino acid residue in the IL-10 conjugate is replaced by the structure of Formula (XVI) or Formula (XVII), or a mixture of Formula (XVI) and Formula (XVII), wherein the amino acid residue in SEQ ID NO: 1 that is replaced is selected from N82, K88, A89, K99, K125, N126, N129, and K130, and wherein n is an integer from about 450 to about 800, or from about 454 to about 796, or from about 454 to about 682, or from about 568 to about 909. In some embodiments of an IL-10 conjugate described herein, n in the compounds of Formula (XVI) or Formula (XVII), or a mixture of Formula (XVI) and Formula (XVII), is an integer selected from 454, 455, 568, 569, 680, 681, 682, 794, 795, 796, 908, 909, and 910.

In some embodiments described herein are IL-10 conjugates comprising the amino acid sequence of SEQ ID NO: 1 in which at least one amino acid residue in the IL-10 conjugate is replaced by the structure of Formula (XVI) or Formula (XVII), or a mixture of Formula (XVI) and Formula (XVII), wherein the amino acid residue in SEQ ID NO: 1 that is replaced is selected from N82, K88, A89, K99, K125, N126, N129, and K130, and wherein n is an integer from about 450 to about 800, or from about 454 to about 796, or from about 454 to about 682, or from about 568 to about 909. In some embodiments of an IL-10 conjugate described herein, n in the compounds of Formula (XVI) or Formula (XVII), or a mixture of Formula (XVI) and Formula (XVII), is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

Described herein are IL-10 conjugates comprising the amino acid sequence of SEQ ID NO: 1 in which at least one amino acid residue in the IL-10 conjugate is replaced by the structure of Formula (XVI) or Formula (XVII), or a mixture of Formula (XVI) and Formula (XVII), wherein n is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons. Described herein are IL-10 conjugates comprising the amino acid sequence of SEQ ID NO: 1 in which at least one amino acid residue in the IL-10 conjugate is replaced by the structure of Formula (XVI) or Formula (XVII), or a mixture of Formula (XVI) and Formula (XVII), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons. Described herein are IL-10 conjugates comprising the amino acid sequence of SEQ ID NO: 1 in which at least one amino acid residue in the IL-10 conjugate is replaced by the structure of Formula (XVI) or Formula (XVII), or a mixture of Formula (XVI) and Formula (XVII), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

In some embodiments described herein are IL-10 conjugates comprising the amino acid sequence of SEQ ID NO: 1 in which at least one amino acid residue in the IL-10 conjugate is replaced by the structure of Formula (XVI) or Formula (XVII), or a mixture of Formula (XVI) and Formula (XVII), wherein the amino acid residue in SEQ ID NO: 1 that is replaced is selected from N82, K88, A89, K99, K125, N126, N129, and K130, and wherein m is an integer from 1 to 6, and n is an integer from about 450 to about 800, or from about 454 to about 796, or from about 454 to about 682, or from about 568 to about 909. In some embodiments of an IL-10 conjugate described herein in the compounds of Formula (XVI) or Formula (XVII), or a mixture of Formula (XVI) and Formula (XVII), m is 2, and n is an integer selected from 454, 455, 568, 569, 680, 681, 682, 794, 795, 796, 908, 909, and 910.

In some embodiments, described herein are IL-10 conjugates modified at an amino acid position. In some instances, the modification is to a natural amino acid. In some instances, the modification is to an unnatural amino acid. In some cases, the modification is to an unnatural amino acid that is also conjugated. In some cases, the modification is to an unnatural amino acid and conjugation to amino acid residues that are not the unnatural amino acid. In some embodiments, the modification of IL-10 conjugate comprises modifying and conjugating a parental IL-10 comprising the sequences of SEQ ID NO: 1 or SEQ ID NO: 2. In some cases, the parental IL-10 is a wild-type IL-10. In some embodiments, the IL-10 conjugates comprise an optional methionine at the N-terminus as depicted by (M) of SEQ ID NOS: 1 and 3-73. In some embodiments, the IL-10 conjugates comprise a methionine at the N-terminus of the wild-type or parental IL-10 sequence the followed by a serine. In some instances, the IL-10 conjugates comprise the serine at the N-terminus of the wild-type or parental IL-10 sequence. In some embodiments, the IL-10 conjugates comprise a methionine substituting and replacing the serine at the N-terminus of the wild-type or parental IL-10 sequence. In some embodiments, the IL-10 conjugates comprise a methionine at the N-terminus followed by the serine as depicted by (M) of SEQ ID NO: 1. In some instances, the IL-10 conjugates comprise the serine at the N-terminus of SEQ ID NO: 1. In some embodiments, the IL-10 conjugates comprise a methionine substituting and replacing the serine at the N-terminus as depicted by (M) of SEQ ID NO: 1.

In some instances, described herein is an isolated and IL-10 conjugate that comprises at least one unnatural amino acid. In some instances, the IL-10 conjugate is an isolated and purified mammalian IL-10, for example, a rodent IL-10 protein or a human IL-10 protein. In some cases, the IL-10 conjugate is a human IL-10 protein. In some cases, the IL-10 conjugate comprises about 80%, 85%, 90%, 95%, 96%, 97%, 98%, or 99% sequence identity to SEQ ID NO: 19. In some cases, the IL-10 conjugate comprises the sequence of SEQ ID NO: 19. In some cases, the IL-10 conjugate consists of the sequence of SEQ ID NO: 19. In some cases, the IL-10 conjugate comprises about 80%, 85%, 90%, 95%, 96%, 97%, 98%, or 99% sequence identity to SEQ ID NO: 20. In some cases, the IL-10 conjugate comprises the sequence of SEQ ID NO: 20. In some cases, the IL-10 conjugate consists of the sequence of SEQ ID NO: 20. In some cases, the IL-10 conjugate comprises about 80%, 85%, 90%, 95%, 96%, 97%, 98%, or 99% sequence identity to SEQ ID NO: 21. In some cases, the IL-10 conjugate comprises the sequence of SEQ ID NO: 21. In some cases, the IL-10 conjugate consists of the sequence of SEQ ID NO: 21. In some cases, the IL-10 conjugate comprises about 80%, 85%, 90%, 95%, 96%, 97%, 98%, or 99% sequence identity to SEQ ID NO: 22. In some cases, the IL-10 conjugate comprises the sequence of SEQ ID NO: 22. In some cases, the IL-10 conjugate consists of the sequence of SEQ ID NO: 22. In some cases, the IL-10 conjugate comprises about 80%, 85%, 90%, 95%, 96%, 97%, 98%, or 99% sequence identity to SEQ ID NO: 23. In some cases, the IL-10 conjugate comprises the sequence of SEQ ID NO: 23. In some cases, the IL-10 conjugate consists of the sequence of SEQ ID NO: 23. In some cases, the IL-10 conjugate comprises about 80%, 85%, 90%, 95%, 96%, 97%, 98%, or 99% sequence identity to SEQ ID NO: 24. In some cases, the IL-10 conjugate comprises the sequence of SEQ ID NO: 24. In some cases, the IL-10 conjugate consists of the sequence of SEQ ID NO: 24. In additional cases, the IL-10 conjugate comprises about 80%, 85%, 90%, 95%, 96%, 97%, 98%, or 99% sequence identity to SEQ ID NO: 25. In additional cases, the IL-10 conjugate comprises the sequence of SEQ ID NO: 25. In additional cases, the IL-10 conjugate consists of the sequence of SEQ ID NO: 25. In some cases, the IL-10 conjugate comprises about 80%, 85%, 90%, 95%, 96%, 97%, 98%, or 99% sequence identity to SEQ ID NO: 26. In some cases, the IL-10 conjugate comprises the sequence of SEQ ID NO: 26. In some cases, the IL-10 conjugate consists of the sequence of SEQ ID NO: 26. In additional cases, the IL-10 conjugate comprises about 80%, 85%, 90%, 95%, 96%, 97%, 98%, or 99% sequence identity to SEQ ID NO: 27. In additional cases, the IL-10 conjugate comprises the sequence of SEQ ID NO: 27. In additional cases, the IL-10 conjugate consists of the sequence of SEQ ID NO: 27. In some cases, the IL-10 conjugate comprises about 80%, 85%, 90%, 95%, 96%, 97%, 98%, or 99% sequence identity to SEQ ID NO: 28. In some cases, the IL-10 conjugate comprises the sequence of SEQ ID NO: 28. In some cases, the IL-10 conjugate consists of the sequence of SEQ ID NO: 28. In additional cases, the IL-10 conjugate comprises about 80%, 85%, 90%, 95%, 96%, 97%, 98%, or 99% sequence identity to SEQ ID NO: 29. In additional cases, the IL-10 conjugate comprises the sequence of SEQ ID NO: 29. In additional cases, the IL-10 conjugate consists of the sequence of SEQ ID NO: 29. In some cases, the IL-10 conjugate comprises about 80%, 85%, 90%, 95%, 96%, 97%, 98%, or 99% sequence identity to SEQ ID NO: 30. In some cases, the IL-10 conjugate comprises the sequence of SEQ ID NO: 30. In some cases, the IL-10 conjugate consists of the sequence of SEQ ID NO: 30. In some cases, the IL-10 conjugate comprises about 80%, 85%, 90%, 95%, 96%, 97%, 98%, or 99% sequence identity to SEQ ID NO: 31. In some cases, the IL-10 conjugate comprises the sequence of SEQ ID NO: 31. In some cases, the IL-10 conjugate consists of the sequence of SEQ ID NO: 31. In additional cases, the IL-10 conjugate comprises about 80%, 85%, 90%, 95%, 96%, 97%, 98%, or 99% sequence identity to SEQ ID NO: 32. In additional cases, the IL-10 conjugate comprises the sequence of SEQ ID NO: 32. In additional cases, the IL-10 conjugate consists of the sequence of SEQ ID NO: 32. In some cases, the IL-10 conjugate comprises about 80%, 85%, 90%, 95%, 96%, 97%, 98%, or 99% sequence identity to SEQ ID NO: 33. In some cases, the IL-10 conjugate comprises the sequence of SEQ ID NO: 33. In some cases, the IL-10 conjugate consists of the sequence of SEQ ID NO: 33. In additional cases, the IL-10 conjugate comprises about 80%, 85%, 90%, 95%, 96%, 97%, 98%, or 99% sequence identity to SEQ ID NO: 34. In additional cases, the IL-10 conjugate comprises the sequence of SEQ ID NO: 34. In additional cases, the IL-10 conjugate consists of the sequence of SEQ ID NO: 34. In some cases, the IL-10 conjugate comprises about 80%, 85%, 90%, 95%, 96%, 97%, 98%, or 99% sequence identity to SEQ ID NO: 35. In some cases, the IL-10 conjugate comprises the sequence of SEQ ID NO: 35. In some cases, the IL-10 conjugate consists of the sequence of SEQ ID NO: 35. In additional cases, the IL-10 conjugate comprises about 80%, 85%, 90%, 95%, 96%, 97%, 98%, or 99% sequence identity to SEQ ID NO: 36. In additional cases, the IL-10 conjugate comprises the sequence of SEQ ID NO: 36. In additional cases, the IL-10 conjugate consists of the sequence of SEQ ID NO: 36. In some cases, the IL-10 conjugate comprises about 80%, 85%, 90%, 95%, 96%, 97%, 98%, or 99% sequence identity to SEQ ID NO: 37. In some cases, the IL-10 conjugate comprises the sequence of SEQ ID NO: 37. In some cases, the IL-10 conjugate consists of the sequence of SEQ ID NO: 37. In additional cases, the IL-10 conjugate comprises about 80%, 85%, 90%, 95%, 96%, 97%, 98%, or 99% sequence identity to SEQ ID NO: 38. In additional cases, the IL-10 conjugate comprises the sequence of SEQ ID NO: 38. In additional cases, the IL-10 conjugate consists of the sequence of SEQ ID NO: 38. In some cases, the IL-10 conjugate comprises about 80%, 85%, 90%, 95%, 96%, 97%, 98%, or 99% sequence identity to SEQ ID NO: 39. In some cases, the IL-10 conjugate comprises the sequence of SEQ ID NO: 39. In some cases, the IL-10 conjugate consists of the sequence of SEQ ID NO: 39. In some cases, the IL-10 conjugate comprises about 80%, 85%, 90%, 95%, 96%, 97%, 98%, or 99% sequence identity to SEQ ID NO: 40. In some cases, the IL-10 conjugate comprises the sequence of SEQ ID NO: 40. In some cases, the IL-10 conjugate consists of the sequence of SEQ ID NO: 40. In some cases, the IL-10 conjugate comprises about 80%, 85%, 90%, 95%, 96%, 97%, 98%, or 99% sequence identity to SEQ ID NO: 41. In some cases, the IL-10 conjugate comprises the sequence of SEQ ID NO: 41. In some cases, the IL-10 conjugate consists of the sequence of SEQ ID NO: 41. In some cases, the IL-10 conjugate comprises about 80%, 85%, 90%, 95%, 96%, 97%, 98%, or 99% sequence identity to SEQ ID NO: 42. In some cases, the IL-10 conjugate comprises the sequence of SEQ ID NO: 42. In some cases, the IL-10 conjugate consists of the sequence of SEQ ID NO: 42. In some cases, the IL-10 conjugate comprises about 80%, 85%, 90%, 95%, 96%, 97%, 98%, or 99% sequence identity to SEQ ID NO: 43. In some cases, the IL-10 conjugate comprises the sequence of SEQ ID NO: 43. In some cases, the IL-10 conjugate consists of the sequence of SEQ ID NO: 43. In some cases, the IL-10 conjugate comprises about 80%, 85%, 90%, 95%, 96%, 97%, 98%, or 99% sequence identity to SEQ ID NO: 44. In some cases, the IL-10 conjugate comprises the sequence of SEQ ID NO: 44. In some cases, the IL-10 conjugate consists of the sequence of SEQ ID NO: 44. In some cases, the IL-10 conjugate comprises about 80%, 85%, 90%, 95%, 96%, 97%, 98%, or 99% sequence identity to SEQ ID NO: 45. In some cases, the IL-10 conjugate comprises the sequence of SEQ ID NO: 45. In some cases, the IL-10 conjugate consists of the sequence of SEQ ID NO: 45. In some cases, the IL-10 conjugate comprises about 80%, 85%, 90%, 95%, 96%, 97%, 98%, or 99% sequence identity to SEQ ID NO: 46. In some cases, the IL-10 conjugate comprises the sequence of SEQ ID NO: 46. In some cases, the IL-10 conjugate consists of the sequence of SEQ ID NO: 46. In some cases, the IL-10 conjugate comprises about 80%, 85%, 90%, 95%, 96%, 97%, 98%, or 99% sequence identity to SEQ ID NO: 47. In some cases, the IL-10 conjugate comprises the sequence of SEQ ID NO: 47. In some cases, the IL-10 conjugate consists of the sequence of SEQ ID NO: 47. In some cases, the IL-10 conjugate comprises about 80%, 85%, 90%, 95%, 96%, 97%, 98%, or 99% sequence identity to SEQ ID NO: 48. In some cases, the IL-10 conjugate comprises the sequence of SEQ ID NO: 48. In some cases, the IL-10 conjugate consists of the sequence of SEQ ID NO: 48. In some cases, the IL-10 conjugate comprises about 80%, 85%, 90%, 95%, 96%, 97%, 98%, or 99% sequence identity to SEQ ID NO: 49. In some cases, the IL-10 conjugate comprises the sequence of SEQ ID NO: 49. In some cases, the IL-10 conjugate consists of the sequence of SEQ ID NO: 49. In some cases, the IL-10 conjugate comprises about 80%, 85%, 90%, 95%, 96%, 97%, 98%, or 99% sequence identity to SEQ ID NO: 50. In some cases, the IL-10 conjugate comprises the sequence of SEQ ID NO: 50. In some cases, the IL-10 conjugate consists of the sequence of SEQ ID NO: 50. In some cases, the IL-10 conjugate comprises about 80%, 85%, 90%, 95%, 96%, 97%, 98%, or 99% sequence identity to SEQ ID NO: 51. In some cases, the IL-10 conjugate comprises the sequence of SEQ ID NO: 51. In some cases, the IL-10 conjugate consists of the sequence of SEQ ID NO: 51. In some cases, the IL-10 conjugate comprises about 80%, 85%, 90%, 95%, 96%, 97%, 98%, or 99% sequence identity to SEQ ID NO: 52. In some cases, the IL-10 conjugate comprises the sequence of SEQ ID NO: 52. In some cases, the IL-10 conjugate consists of the sequence of SEQ ID NO: 52. In some cases, the IL-10 conjugate comprises about 80%, 85%, 90%, 95%, 96%, 97%, 98%, or 99% sequence identity to SEQ ID NO: 53. In some cases, the IL-10 conjugate comprises the sequence of SEQ ID NO: 53. In some cases, the IL-10 conjugate consists of the sequence of SEQ ID NO: 53. In some cases, the IL-10 conjugate comprises about 80%, 85%, 90%, 95%, 96%, 97%, 98%, or 99% sequence identity to SEQ ID NO: 54. In some cases, the IL-10 conjugate comprises the sequence of SEQ ID NO: 54. In some cases, the IL-10 conjugate consists of the sequence of SEQ ID NO: 54. In additional cases, the IL-10 conjugate comprises about 80%, 85%, 90%, 95%, 96%, 97%, 98%, or 99% sequence identity to SEQ ID NO: 55. In additional cases, the IL-10 conjugate comprises the sequence of SEQ ID NO: 55. In additional cases, the IL-10 conjugate consists of the sequence of SEQ ID NO: 55. In some cases, the IL-10 conjugate comprises about 80%, 85%, 90%, 95%, 96%, 97%, 98%, or 99% sequence identity to SEQ ID NO: 56. In some cases, the IL-10 conjugate comprises the sequence of SEQ ID NO: 56. In some cases, the IL-10 conjugate consists of the sequence of SEQ ID NO: 56. In additional cases, the IL-10 conjugate comprises about 80%, 85%, 90%, 95%, 96%, 97%, 98%, or 99% sequence identity to SEQ ID NO: 57. In additional cases, the IL-10 conjugate comprises the sequence of SEQ ID NO: 57. In additional cases, the IL-10 conjugate consists of the sequence of SEQ ID NO: 57. In some cases, the IL-10 conjugate comprises about 80%, 85%, 90%, 95%, 96%, 97%, 98%, or 99% sequence identity to SEQ ID NO: 58. In some cases, the IL-10 conjugate comprises the sequence of SEQ ID NO: 58. In some cases, the IL-10 conjugate consists of the sequence of SEQ ID NO: 58. In additional cases, the IL-10 conjugate comprises about 80%, 85%, 90%, 95%, 96%, 97%, 98%, or 99% sequence identity to SEQ ID NO: 59. In additional cases, the IL-10 conjugate comprises the sequence of SEQ ID NO: 59. In additional cases, the IL-10 conjugate consists of the sequence of SEQ ID NO: 59. In some cases, the IL-10 conjugate comprises about 80%, 85%, 90%, 95%, 96%, 97%, 98%, or 99% sequence identity to SEQ ID NO: 60. In some cases, the IL-10 conjugate comprises the sequence of SEQ ID NO: 60. In some cases, the IL-10 conjugate consists of the sequence of SEQ ID NO: 60. In some cases, the IL-10 conjugate comprises about 80%, 85%, 90%, 95%, 96%, 97%, 98%, or 99% sequence identity to SEQ ID NO: 61. In some cases, the IL-10 conjugate comprises the sequence of SEQ ID NO: 61. In some cases, the IL-10 conjugate consists of the sequence of SEQ ID NO: 61. In additional cases, the IL-10 conjugate comprises about 80%, 85%, 90%, 95%, 96%, 97%, 98%, or 99% sequence identity to SEQ ID NO: 62. In additional cases, the IL-10 conjugate comprises the sequence of SEQ ID NO: 62. In additional cases, the IL-10 conjugate consists of the sequence of SEQ ID NO: 62. In some cases, the IL-10 conjugate comprises about 80%, 85%, 90%, 95%, 96%, 97%, 98%, or 99% sequence identity to SEQ ID NO: 63. In some cases, the IL-10 conjugate comprises the sequence of SEQ ID NO: 63. In some cases, the IL-10 conjugate consists of the sequence of SEQ ID NO: 63. In additional cases, the IL-10 conjugate comprises about 80%, 85%, 90%, 95%, 96%, 97%, 98%, or 99% sequence identity to SEQ ID NO: 64. In additional cases, the IL-10 conjugate comprises the sequence of SEQ ID NO: 64. In additional cases, the IL-10 conjugate consists of the sequence of SEQ ID NO: 64. In some cases, the IL-10 conjugate comprises about 80%, 85%, 90%, 95%, 96%, 97%, 98%, or 99% sequence identity to SEQ ID NO: 65. In some cases, the IL-10 conjugate comprises the sequence of SEQ ID NO: 65. In some cases, the IL-10 conjugate consists of the sequence of SEQ ID NO: 65. In additional cases, the IL-10 conjugate comprises about 80%, 85%, 90%, 95%, 96%, 97%, 98%, or 99% sequence identity to SEQ ID NO: 66. In additional cases, the IL-10 conjugate comprises the sequence of SEQ ID NO: 66. In additional cases, the IL-10 conjugate consists of the sequence of SEQ ID NO: 66. In some cases, the IL-10 conjugate comprises about 80%, 85%, 90%, 95%, 96%, 97%, 98%, or 99% sequence identity to any one of SEQ ID NOS: 67-73. In additional cases, the IL-10 conjugate comprises the sequence of any one of SEQ ID NOS: 67-73. In additional cases, the IL-10 conjugate consists of the sequence of any one of SEQ ID NOS: 67-73.

In some embodiments, the at least one unnatural amino acid is located proximal to the N-terminus (e.g., proximal to the N-terminal residue). For example, the at least one unnatural amino acid is located optionally within the first 10, 20, 30, 40, or 50 residues from the N-terminus. In some cases, the at least one unnatural amino acid is located at the N-terminus (i.e., the at least one unnatural amino acid is the N-terminal residue of the IL-10 polypeptide).

In other embodiments, the at least one unnatural amino acid is located proximal to the C-terminus (e.g., proximal to the C-terminal residue). For example, the at least one unnatural amino acid is located optionally within the first 10, 20, 30, 40, or 50 residues from the C-terminus. In some cases, the at least one unnatural amino acid is located at the C-terminus (i.e., the at least one unnatural amino acid is the C-terminal residue of the IL-10 polypeptide).

In some instances, the IL-10 conjugate comprises one conjugating moiety bound to an unnatural amino acid.

In some instances, the IL-10 conjugate comprises an IL-10 monomer that is capable of activating the IL-10R signaling pathway. In other instances, the IL-10 conjugate comprises an IL-10 dimer that is functionally active.

In some instances, the IL-10 conjugate comprises two or more conjugating moieties, in which each of the two or more conjugating moieties is bound to a different unnatural amino acid. In some cases, the two or more conjugating moieties are conjugated to the same IL-10 polypeptide (e.g., either in a functionally active IL-10 monomer or in a functionally active IL-10 dimer). In other cases, the two or more conjugating moieties are each conjugated to a different IL-10 polypeptide within the IL-10 dimer. In additional cases, the IL-10 conjugate comprises three, four, five, six, or more conjugating moieties, in which each of the conjugating moieties is bound to a different unnatural amino acid. In such instances, the two IL-10 polypeptides within the dimer has an unequal distribution of the conjugating moieties, e.g., one IL-10 polypeptide has one conjugating moiety while the other IL-10 polypeptide has two or more conjugating moieties.

In some instances, the IL-10 conjugate comprises two or more conjugating moieties. In some cases, each of the two or more conjugating moieties is bound to an unnatural amino acid at the same residue position within the respective IL-10 monomer. In other cases, each of the two or more conjugating moieties is bound to an unnatural amino acid located at a different residue position within the IL-10 dimer.

In some instances, the location of the conjugating moiety does not substantially interfere with dimerization of the IL-10 polypeptide.

In some cases, the location of the conjugating moiety further does not significantly interfere with binding of the IL-10 dimer to IL-10R.

In some embodiments, the location of the conjugating moiety impairs signaling of the IL-10R by less than 90%, 80%, 70%, 60%, 50%, 40%, 30%, 20%, 10%, 5%, 4%, 3%, 2%, 1%, or less. In some instances, the location of the conjugating moiety impairs signaling of the IL-10R by less than 90%. In some instances, the location of the conjugating moiety impairs signaling of the IL-10R by less than 80%. In some instances, the location of the conjugating moiety impairs signaling of the IL-10R by less than 70%. In some instances, the location of the conjugating moiety impairs signaling of the IL-10R by less than 60%. In some instances, the location of the conjugating moiety impairs signaling of the IL-10R by less than 50%. In some instances, the location of the conjugating moiety impairs signaling of the IL-10R by less than 40%. In some instances, the location of the conjugating moiety impairs signaling of the IL-10R by less than 30%. In some instances, the location of the conjugating moiety impairs signaling of the IL-10R by less than 20%. In some instances, the location of the conjugating moiety impairs signaling of the IL-10R by less than 10%. In some instances, the location of the conjugating moiety impairs signaling of the IL-10R by less than 5%. In some instances, the location of the conjugating moiety impairs signaling of the IL-10R by less than 2%. In some instances, the location of the conjugating moiety impairs signaling of the IL-10R by less than 1%. In some cases, the location of the conjugating moiety does not significantly impair signaling of the IL-10R.

In additional cases, the location of the conjugating moiety does not impair signaling of the IL-10R.

In some instances, the IL-10 conjugate has an enhanced plasma half-life. In some cases, the enhanced plasma half-life is compared to a plasma half-life of a wild-type IL-10 conjugate or wild-type IL-10 protein. In some cases, the enhanced plasma half-life of the IL-10 conjugate is at least 90 minutes, 2 hours, 3 hours, 4 hours, 5 hours, 6 hours, 7 hours, 8 hours, 9 hours, 10 hours, 11 hours, 12 hours, 18 hours, 24 hours, 36 hours, 48 hours, 3 days, 4 days, 5 days, 6 days, 7 days, 10 days, 12 days, 14 days, 21 days, 28 days, 30 days, or longer than the plasma half-life of the wild-type IL-10 conjugate or wild-type IL-10 protein. In some cases, the enhanced plasma half-life of the IL-10 conjugate is at least 90 minutes or longer than the plasma half-life of the wild-type IL-10 conjugate or wild-type IL-10 protein. In some cases, the enhanced plasma half-life of the IL-10 conjugate is at least 2 hours or longer than the plasma half-life of the wild-type IL-10 conjugate or wild-type IL-10 protein. In some cases, the enhanced plasma half-life of the IL-10 conjugate is at least 3 hours or longer than the plasma half-life of the wild-type IL-10 conjugate or wild-type IL-10 protein. In some cases, the enhanced plasma half-life of the IL-10 conjugate is at least 4 hours or longer than the plasma half-life of the wild-type IL-10 conjugate or wild-type IL-10 protein. In some cases, the enhanced plasma half-life of the IL-10 conjugate is at least 5 hours or longer than the plasma half-life of the wild-type IL-10 conjugate or wild-type IL-10 protein. In some cases, the enhanced plasma half-life of the IL-10 conjugate is at least 6 hours or longer than the plasma half-life of the wild-type IL-10 conjugate or wild-type IL-10 protein. In some cases, the enhanced plasma half-life of the IL-10 conjugate is at least 10 hours or longer than the plasma half-life of the wild-type IL-10 conjugate or wild-type IL-10 protein. In some cases, the enhanced plasma half-life of the IL-10 conjugate is at least 12 hours or longer than the plasma half-life of the wild-type IL-10 conjugate or wild-type IL-10 protein. In some cases, the enhanced plasma half-life of the IL-10 conjugate is at least 18 hours or longer than the plasma half-life of the wild-type IL-10 conjugate or wild-type IL-10 protein. In some cases, the enhanced plasma half-life of the IL-10 conjugate is at least 24 hours or longer than the plasma half-life of the wild-type IL-10 conjugate or wild-type IL-10 protein. In some cases, the enhanced plasma half-life of the IL-10 conjugate is at least 36 hours or longer than the plasma half-life of the wild-type IL-10 conjugate or wild-type IL-10 protein. In some cases, the enhanced plasma half-life of the IL-10 conjugate is at least 48 hours or longer than the plasma half-life of the wild-type IL-10 conjugate or wild-type IL-10 protein. In some cases, the enhanced plasma half-life of the IL-10 conjugate is at least 3 days or longer than the plasma half-life of the wild-type IL-10 conjugate or wild-type IL-10 protein. In some cases, the enhanced plasma half-life of the IL-10 conjugate is at least 4 days or longer than the plasma half-life of the wild-type IL-10 conjugate or wild-type IL-10 protein. In some cases, the enhanced plasma half-life of the IL-10 conjugate is at least 5 days or longer than the plasma half-life of the wild-type IL-10 conjugate or wild-type IL-10 protein. In some cases, the enhanced plasma half-life of the IL-10 conjugate is at least 6 days or longer than the plasma half-life of the wild-type IL-10 conjugate or wild-type IL-10 protein. In some cases, the enhanced plasma half-life of the IL-10 conjugate is at least 7 days or longer than the plasma half-life of the wild-type IL-10 conjugate or wild-type IL-10 protein. In some cases, the enhanced plasma half-life of the IL-10 conjugate is at least 10 days or longer than the plasma half-life of the wild-type IL-10 conjugate or wild-type IL-10 protein. In some cases, the enhanced plasma half-life of the IL-10 conjugate is at least 12 days or longer than the plasma half-life of the wild-type IL-10 conjugate or wild-type IL-10 protein. In some cases, the enhanced plasma half-life of the IL-10 conjugate is at least 14 days or longer than the plasma half-life of the wild-type IL-10 conjugate or wild-type IL-10 protein. In some cases, the enhanced plasma half-life of the IL-10 conjugate is at least 21 days or longer than the plasma half-life of the wild-type IL-10 conjugate or wild-type IL-10 protein. In some cases, the enhanced plasma half-life of the IL-10 conjugate is at least 28 days or longer than the plasma half-life of the wild-type IL-10 conjugate or wild-type IL-10 protein. In some cases, the enhanced plasma half-life of the IL-10 conjugate is at least 30 days or longer than the plasma half-life of the wild-type IL-10 conjugate or wild-type IL-10 protein.

In some embodiments, also described herein is an IL-10/IL-10R complex comprising a modified IL-10 dimer comprising at least one unnatural amino acid and an IL-10R, wherein the modified IL-10 dimer has an enhanced plasma half-life compared to a plasma half-life of a wild-type IL-10 protein. In some instances, the modified IL-10 dimer further comprises a conjugating moiety covalently attached to the at least one unnatural amino acid.

In some embodiments, the IL-10 conjugate has a plasma half-life that is capable of proliferating and/or expanding tumor infiltration lymphocytes (TILs), T cells, B cells, natural killer cells, macrophages, neutrophils, dendritic cells, mast cells, eosinophils basophils, or CD4+ or CD8+ T cells.

In some embodiments, the IL-10 conjugate is administered to a subject. In some embodiments, the IL-10 conjugate administered to the subject comprises a reduced toxicity compared to a toxicity of the wild-type IL-10 protein administered to a second subject. In some embodiments, the IL-10 conjugate comprises the reduced toxicity that is at least 1-fold, 2-fold, 3-fold, 4-fold, 5-fold, 6-fold, 7-fold, 8-fold, 9-fold, 10-fold, 20-fold, 30-fold, 50-fold, 100-fold, or more reduced relative to the wild type IL-10 dimer. In some cases, the reduced toxicity is at least 10%, 20%, 30%, 40%, 50%, 60%, 70%, 80%, 90%, 100%, 200%, 300%, 400%, 500%, or more reduced relative to the wild-type IL-10 protein.

In some embodiments, the IL-10 conjugate is administered to a subject. In some embodiments, the IL-10 conjugate administered to the subject does not cause grade 3 or grade 4 adverse events. In some embodiments, the IL-10 conjugate administered to the subject comprises a reduced occurrence or severity of grade 3 or grade 4 adverse events compared to an occurrence or severity of grade 3 or grade 4 adverse events caused by the wild-type IL-10 protein administered to a second subject. Exemplary grade 3 and grade 4 adverse events include anemia, leukopenia, thrombocytopenia, increased ALT, anorexia, arthralgia, back pain, chills, diarrhea, dyslipidemia, fatigue, fever, flu-like symptoms, hypoalbuminemia, increased lipase, injection site reaction, myalgia, nausea, night sweats, pruritis, rash, erythematous rash, maculopapular rash, transaminitis, vomiting, and weakness.

In some embodiments, the IL-10 conjugate decreases the occurrence of the grade 3 or grade 4 adverse events in the subject by about 10%, 20%, 30%, 40%, 50%, 60%, 70%, 80%, 90%, 95%, 99%, or about 100%, relative to a second subject administered with a wild-type IL-10 protein. In some instances, the IL-10 conjugate decreases the severity of grade 3 or grade 4 adverse events in the subject by about 10%, 20%, 30%, 40%, 50%, 60%, 70%, 80%, 90%, 95%, 99%, or about 100%, relative to a second subject administered with the wild-type IL-10 protein.

In some embodiments, the IL-10 conjugate as described herein comprises a decreased affinity to the IL-10R compared to an affinity of wild-type IL-10 conjugate or wild-type IL-10 protein to the IL-10R. In some embodiments, the affinity of the IL-10 conjugate to IL-10R compared to the affinity of the wild-type IL-10 conjugate or wild-type IL-10 protein to IL-10R is decreased about 10%, 20%, 30%, 40%, 50%, 60%, 70%, 80%, 90%, 95%, or 99%, or greater than 99%. In some cases, the decreased affinity is about 10%. In some cases, the decreased affinity is about 20%. In some cases, the decreased affinity is about 30%. In some cases, the decreased affinity is about 40%. In some cases, the decreased affinity is about 50%. In some cases, the decreased affinity is about 60%. In some cases, the decreased affinity is about 70%. In some cases, the decreased affinity is about 80%. In some cases, the decreased affinity is about 90%. In some cases, the decreased affinity is about 95%. In some cases, the decreased affinity is about 99%. In some cases, the decreased affinity is about 100%.

In some embodiments, the decreased affinity of the IL-10 conjugate compared to the wild-type IL-10 conjugate or wild-type IL-10 protein is about 1-fold, 2-fold, 3-fold, 4-fold, 5-fold, 6-fold, 7-fold, 8-fold, 9-fold, 10-fold, 30-fold, 50-fold, 100-fold, 200-fold, 300-fold, 400-fold, 500-fold, 1,000-fold, or more. In some cases, the decreased affinity is about 1-fold. In some cases, the decreased affinity is about 2-fold. In some cases, the decreased affinity is about 3-fold. In some cases, the decreased affinity is about 4-fold. In some cases, the decreased affinity is about 5-fold. In some cases, the decreased affinity is about 6-fold. In some cases, the decreased affinity is about 7-fold. In some cases, the decreased affinity is about 8-fold. In some cases, the decreased affinity is about 9-fold. In some cases, the decreased affinity is about 10-fold. In some cases, the decreased affinity is about 30-fold. In some cases, the decreased affinity is about 50-fold. In some cases, the decreased affinity is about 100-fold. In some cases, the decreased affinity is about 200-fold. In some cases, the decreased affinity is about 300-fold. In some cases, the decreased affinity is about 400-fold. In some cases, the decreased affinity is about 500-fold. In some cases, the decreased affinity is about 1000-fold. In some cases, the decreased affinity is more than 1,000-fold.

In some cases, the IL-10 conjugate does not interact with IL-10R. In some cases, the IL-10 conjugate has about the same affinity to IL-10R as the affinity of the wild-type IL-10 to IL-10R.

In some embodiments, the IL-10 conjugate as described herein comprises an increased affinity to the IL-10R compared to an affinity of wild-type IL-10 conjugate or wild-type IL-10 protein to the IL-10R. In some embodiments, the affinity of the IL-10 conjugate to the IL-10R compared to the affinity of the wild-type IL-10 conjugate or wild-type IL-10 protein to IL-10R is increased about 10%, 20%, 30%, 40%, 50%, 60%, 70%, 80%, 90%, 95%, or 99%, or greater than 99%. In some cases, the increased affinity is about 10%. In some cases, the increased affinity is about 20%. In some cases, the increased affinity is about 30%. In some cases, the increased affinity is about 40%. In some cases, the increased affinity is about 50%. In some cases, the increased affinity is about 60%. In some cases, the increased affinity is about 70%. In some cases, the increased affinity is about 80%. In some cases, the increased affinity is about 90%. In some cases, the increased affinity is about 95%. In some cases, the increased affinity is about 99%. In some cases, the increased affinity is about 100%. In some embodiments, the increased affinity of the IL-10 conjugate compared to the wild-type IL-10 conjugate or wild-type IL-10 protein is about 1-fold, 2-fold, 3-fold, 4-fold, 5-fold, 6-fold, 7-fold, 8-fold, 9-fold, 10-fold, 30-fold, 50-fold, 100-fold, 200-fold, 300-fold, 400-fold, 500-fold, 1,000-fold, or more. In some cases, the increased affinity is about 1-fold. In some cases, the increased affinity is about 2-fold. In some cases, the increased affinity is about 3-fold. In some cases, the increased affinity is about 4-fold. In some cases, the increased affinity is about 5-fold. In some cases, the increased affinity is about 6-fold. In some cases, the increased affinity is about 7-fold. In some cases, the increased affinity is about 8-fold. In some cases, the increased affinity is about 9-fold. In some cases, the increased affinity is about 10-fold. In some cases, the increased affinity is about 30-fold. In some cases, the increased affinity is about 50-fold. In some cases, the increased affinity is about 100-fold. In some cases, the increased affinity is about 200-fold. In some cases, the increased affinity is about 300-fold. In some cases, the increased affinity is about 400-fold. In some cases, the increased affinity is about 500-fold. In some cases, the increased affinity is about 1000-fold. In some cases, the increased affinity is more than 1,000-fold.

In some instances, IL-10R signaling potency as mediated by IL-10 is measured by a EC50. In some embodiments, the EC50 of the IL-10 conjugate is decreased compared to EC50 of the wild-type IL-10 conjugate or wild-type IL-10 protein. In some embodiments, the decreased EC50 of the IL-10 conjugate is about 10%, 20%, 30%, 40%, 50%, 60%, 70%, 80%, 90%, 95%, or 99%, or greater than 99%. In some cases, the EC50 of the IL-10 conjugate is decreased about 10%. In some cases, the EC50 of the IL-10 conjugate is decreased about 20%. In some cases, the EC50 of the IL-10 conjugate is decreased about 30%. In some cases, the EC50 of the IL-10 conjugate is decreased about 40%. In some cases, the EC50 of the IL-10 conjugate is decreased about 50%. In some cases, the EC50 of the IL-10 conjugate is decreased about 60%. In some cases, the EC50 of the IL-10 conjugate is decreased about 70%. In some cases, the EC50 of the IL-10 conjugate is decreased about 80%. In some cases, the EC50 of the IL-10 conjugate is decreased about 90%. In some cases, the EC50 of the IL-10 conjugate is decreased about 95%. In some cases, the EC50 of the IL-10 conjugate is decreased about 99%. In some cases, the EC50 of the IL-10 conjugate is decreased about 100%.

In some embodiments, the decreased EC50 of the IL-10 conjugate compared to the wild-type IL-10 conjugate or wild-type IL-10 protein is about 1-fold, 2-fold, 3-fold, 4-fold, 5-fold, 6-fold, 7-fold, 8-fold, 9-fold, 10-fold, 30-fold, 50-fold, 100-fold, 200-fold, 300-fold, 400-fold, 500-fold, 1,000-fold, or more. In some cases, the EC50 of the IL-10 conjugate is decreased about 1-fold. In some cases, the EC50 of the IL-10 conjugate is decreased about 2-fold. In some cases, the EC50 of the IL-10 conjugate is decreased about 3-fold. In some cases, the EC50 of the IL-10 conjugate is decreased about 4-fold. In some cases, the EC50 of the IL-10 conjugate is decreased about 5-fold. In some cases, the EC50 of the IL-10 conjugate is decreased about 6-fold. In some cases, the EC50 of the IL-10 conjugate is decreased about 7-fold. In some cases, the EC50 of the IL-10 conjugate is decreased about 8-fold. In some cases, the EC50 of the IL-10 conjugate is decreased about 9-fold. In some cases, the EC50 of the IL-10 conjugate is decreased about 10-fold. In some cases, the EC50 of the IL-10 conjugate is decreased about 30-fold. In some cases, the EC50 of the IL-10 conjugate is decreased about 50-fold. In some cases, the EC50 of the IL-10 conjugate is decreased about 100-fold. In some cases, the EC50 of the IL-10 conjugate is decreased about 200-fold. In some cases, the EC50 of the IL-10 conjugate is decreased about 300-fold. In some cases, the EC50 of the IL-10 conjugate is decreased about 400-fold. In some cases, the EC50 of the IL-10 conjugate is decreased about 500-fold. In some cases, the EC50 of the IL-10 conjugate is decreased about 1000-fold. In some cases, the EC50 of the IL-10 conjugate is decreased more than 1,000-fold.

In some cases, the EC50 of the IL-10 conjugate is about the same as the EC50 of the wild-type IL-10 protein.

In some instances, the IL-10 conjugate as described herein has an increased EC50 compared to EC50 of the wild-type IL-10 conjugate or wild-type IL-10 protein in activating IL-10R signaling. In some embodiments, the increased EC50 of the IL-10 conjugate is about 10%, 20%, 30%, 40%, 50%, 60%, 70%, 80%, 90%, 95%, or 99%, or greater than 99%. In some cases, the EC50 of the IL-10 conjugate is increased about 10%. In some cases, the EC50 of the IL-10 conjugate is increased about 20%. In some cases, the EC50 of the IL-10 conjugate is increased about 30%. In some cases, the EC50 of the IL-10 conjugate is increased about 40%. In some cases, the EC50 of the IL-10 conjugate is increased about 50%. In some cases, the EC50 of the IL-10 conjugate is increased about 60%. In some cases, the EC50 of the IL-10 conjugate is increased about 70%. In some cases, the EC50 of the IL-10 conjugate is increased about 80%. In some cases, the EC50 of the IL-10 conjugate is increased about 90%. In some cases, the EC50 of the IL-10 conjugate is increased about 95%. In some cases, the EC50 of the IL-10 conjugate is increased about 99%. In some cases, the EC50 of the IL-10 conjugate is increased about 100%.

In some embodiments, the increased EC50 of the IL-10 conjugate compared to the EC50 of the wild-type IL-10 conjugate or wild-type IL-10 protein is about 1-fold, 2-fold, 3-fold, 4-fold, 5-fold, 6-fold, 7-fold, 8-fold, 9-fold, 10-fold, 30-fold, 50-fold, 100-fold, 200-fold, 300-fold, 400-fold, 500-fold, 1,000-fold, or more. In some cases, the EC50 of the IL-10 conjugate is increased about 1-fold. In some cases, the EC50 of the IL-10 conjugate is increased about 2-fold. In some cases, the EC50 of the IL-10 conjugate is increased about 3-fold. In some cases, the EC50 of the IL-10 conjugate is increased about 4-fold. In some cases, the EC50 of the IL-10 conjugate is increased about 5-fold. In some cases, the EC50 of the IL-10 conjugate is increased about 6-fold. In some cases, the EC50 of the IL-10 conjugate is increased about 7-fold. In some cases, the EC50 of the IL-10 conjugate is increased about 8-fold. In some cases, the EC50 of the IL-10 conjugate is increased about 9-fold. In some cases, the EC50 of the IL-10 conjugate is increased about 10-fold. In some cases, the EC50 of the IL-10 conjugate is increased about 30-fold. In some cases, the EC50 of the IL-10 conjugate is increased about 50-fold. In some cases, the EC50 of the IL-10 conjugate is increased about 100-fold. In some cases, the EC50 of the IL-10 conjugate is increased about 200-fold. In some cases, the EC50 of the IL-10 conjugate is increased about 300-fold. In some cases, the EC50 of the IL-10 conjugate is increased about 400-fold. In some cases, the EC50 of the IL-10 conjugate is increased about 500-fold. In some cases, the EC50 of the IL-10 conjugate is increased about 1000-fold. In some cases, the EC50 of the IL-10 conjugate is increased more than 1,000-fold.

In some instances, IL-10R signaling potency as mediated by IL-10 is measured by a ED50. In some embodiments, the IL-10 conjugate as described herein has a decreased ED50 compared to an ED50 of the wild-type IL-10 conjugate or wild-type IL-10 protein. In some embodiments, the decreased ED50 of the IL-10 conjugate is about 10%, 20%, 30%, 40%, 50%, 60%, 70%, 80%, 90%, 95%, or 99%, or greater than 99%. In some cases, the ED50 of the IL-10 conjugate is decreased about 10%. In some cases, the ED50 of the IL-10 conjugate is decreased about 20%. In some cases, the ED50 of the IL-10 conjugate is decreased about 30%. In some cases, the ED50 of the IL-10 conjugate is decreased about 40%. In some cases, the ED50 of the IL-10 conjugate is decreased about 50%. In some cases, the ED50 of the IL-10 conjugate is decreased about 60%. In some cases, the ED50 of the IL-10 conjugate is decreased about 70%. In some cases, the ED50 of the IL-10 conjugate is decreased about 80%. In some cases, the ED50 of the IL-10 conjugate is decreased about 90%. In some cases, the ED50 of the IL-10 conjugate is decreased about 95%. In some cases, the ED50 of the IL-10 conjugate is decreased about 99%. In some cases, the ED50 of the IL-10 conjugate is decreased about 100%.

In some embodiments, the decreased ED50 of the IL-10 conjugate compared to the ED50 of the wild-type IL-10 conjugate or wild-type IL-10 protein is about 1-fold, 2-fold, 3-fold, 4-fold, 5-fold, 6-fold, 7-fold, 8-fold, 9-fold, 10-fold, 30-fold, 50-fold, 100-fold, 200-fold, 300-fold, 400-fold, 500-fold, 1,000-fold, or more. In some cases, the ED50 of the IL-10 conjugate is decreased about 1-fold. In some cases, the ED50 of the IL-10 conjugate is decreased about 2-fold. In some cases, the ED50 of the IL-10 conjugate is decreased about 3-fold. In some cases, the ED50 of the IL-10 conjugate is decreased about 4-fold. In some cases, the ED50 of the IL-10 conjugate is decreased about 5-fold. In some cases, the ED50 of the IL-10 conjugate is decreased about 6-fold. In some cases, the ED50 of the IL-10 conjugate is decreased about 7-fold. In some cases, the ED50 of the IL-10 conjugate is decreased about 8-fold. In some cases, the ED50 of the IL-10 conjugate is decreased about 9-fold. In some cases, the ED50 of the IL-10 conjugate is decreased about 10-fold. In some cases, the ED50 of the IL-10 conjugate is decreased about 30-fold. In some cases, the ED50 of the IL-10 conjugate is decreased about 50-fold. In some cases, the ED50 of the IL-10 conjugate is decreased about 100-fold. In some cases, the ED50 of the IL-10 conjugate is decreased about 200-fold. In some cases, the ED50 of the IL-10 conjugate is decreased about 300-fold. In some cases, the ED50 of the IL-10 conjugate is decreased about 400-fold. In some cases, the ED50 of the IL-10 conjugate is decreased about 500-fold. In some cases, the ED50 of the IL-10 conjugate is decreased about 1000-fold. In some cases, the ED50 of the IL-10 conjugate is decreased more than 1,000-fold.

In some cases, the ED50 of the IL-10 conjugate is about the same as the ED50 of the wild-type IL-10 protein.

In some instances, the IL-10 conjugate as described herein has an increased ED50 compared to ED50 of wild-type IL-10 conjugate or wild-type IL-10 protein. In some embodiments, the increased ED50 of the IL-10 conjugate is about 10%, 20%, 30%, 40%, 50%, 60%, 70%, 80%, 90%, 95%, or 99%, or greater than 99%. In some cases, the ED50 of the IL-10 conjugate is increased about 10%. In some cases, the ED50 of the IL-10 conjugate is increased about 20%. In some cases, the ED50 of the IL-10 conjugate is increased about 30%. In some cases, the ED50 of the IL-10 conjugate is increased about 40%. In some cases, the ED50 of the IL-10 conjugate is increased about 50%. In some cases, the ED50 of the IL-10 conjugate is increased about 60%. In some cases, the ED50 of the IL-10 conjugate is increased about 70%. In some cases, the ED50 of the IL-10 conjugate is increased about 80%. In some cases, the ED50 of the IL-10 conjugate is increased about 90%. In some cases, the ED50 of the IL-10 conjugate is increased about 95%. In some cases, the ED50 of the IL-10 conjugate is increased about 99%. In some cases, the ED50 of the IL-10 conjugate is increased about 100%.

In some embodiments, the increased ED50 of the IL-10 conjugate compared to the ED50 of the wild-type IL-10 conjugate or wild-type IL-10 protein is about 1-fold, 2-fold, 3-fold, 4-fold, 5-fold, 6-fold, 7-fold, 8-fold, 9-fold, 10-fold, 30-fold, 50-fold, 100-fold, 200-fold, 300-fold, 400-fold, 500-fold, 1,000-fold, or more. In some cases, the ED50 of the IL-10 conjugate is increased about 1-fold. In some cases, the ED50 of the IL-10 conjugate is increased about 2-fold. In some cases, the ED50 of the IL-10 conjugate is increased about 3-fold. In some cases, the ED50 of the IL-10 conjugate is increased about 4-fold. In some cases, the ED50 of the IL-10 conjugate is increased about 5-fold. In some cases, the ED50 of the IL-10 conjugate is increased about 6-fold. In some cases, the ED50 of the IL-10 conjugate is increased about 7-fold. In some cases, the ED50 of the IL-10 conjugate is increased about 8-fold. In some cases, the ED50 of the IL-10 conjugate is increased about 9-fold. In some cases, the ED50 of the IL-10 conjugate is increased about 10-fold. In some cases, the ED50 of the IL-10 conjugate is increased about 30-fold. In some cases, the ED50 of the IL-10 conjugate is increased about 50-fold. In some cases, the ED50 of the IL-10 conjugate is increased about 100-fold. In some cases, the ED50 of the IL-10 conjugate is increased about 200-fold. In some cases, the ED50 of the IL-10 conjugate is increased about 300-fold. In some cases, the ED50 of the IL-10 conjugate is increased about 400-fold. In some cases, the ED50 of the IL-10 conjugate is increased about 500-fold. In some cases, the ED50 of the IL-10 conjugate is increased about 1000-fold. In some cases, the ED50 of the IL-10 conjugate is increased more than 1,000-fold.

### Natural and Unnatural Amino Acids

Described herein, in some embodiments, is an amino acid residue within a modified IL-10 polypeptide or IL-10 conjugate mutated to lysine, cysteine, histidine, arginine, aspartic acid, glutamic acid, serine, threonine, or tyrosine prior to binding to (or reacting with) a conjugating moiety. For example, the side chain of lysine, cysteine, histidine, arginine, aspartic acid, glutamic acid, serine, threonine, or tyrosine may bind to a conjugating moiety described herein. In some instances, the amino acid residue is mutated to cysteine, lysine, or histidine. In some cases, the amino acid residue is mutated to cysteine. In some cases, the amino acid residue is mutated to lysine. In some cases, the amino acid residue is mutated to histidine. In some cases, the amino acid residue is mutated to tyrosine. In some cases, the amino acid residue is mutated to tryptophan. In some instances, the amino acid residue is located proximal to the N- or C-terminus, at the N- or C-terminus, or at an internal residue position. In some instances, the amino acid residue is the N- or C-terminal residue and the mutation is to cysteine or lysine. In some instances, the amino acid residue is located proximal to the N- or C-terminal residue (e.g., within 50, 40, 30, 20, or 10 residues from the N- or C-terminal residue) and the mutation is to cysteine or lysine.

In some instances, an amino acid residue is added to the N- or C-terminal residue, i.e., the IL-10 polypeptide comprises an additional amino acid residue at either the N- or C-terminus and the additional amino acid residue is cysteine or lysine. In some cases, the additional amino acid residue is cysteine. In some cases, the additional amino acid is conjugated to a conjugating moiety.

In some embodiments, an amino acid residue described herein (e.g., within an IL-10 polypeptide) is mutated to an unnatural amino acid. In some embodiments, the unnatural amino acid is not conjugated with a conjugating moiety. In some embodiments, an IL-10 polypeptide described herein comprises an unnatural amino acid, wherein the IL-10 is conjugated to the protein, wherein the point of attachment is not the unnatural amino acid.

In some embodiments, an amino acid residue described herein (e.g., within an IL-10 polypeptide) is mutated to an unnatural amino acid prior to binding to a conjugating moiety. In some cases, the mutation to an unnatural amino acid prevents or minimizes a self-antigen response of the immune system. As used herein, the term “unnatural amino acid” refers to an amino acid other than the 20 amino acids that occur naturally in protein. Non-limiting examples of unnatural amino acids include: p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl)alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, p-Boronophenylalanine, O-propargyltyrosine, L-phosphoserine, phosphonoserine, phosphonotyrosine, p-bromophenylalanine, selenocysteine, p-amino-L-phenylalanine, isopropyl-L-phenylalanine, N6-((2-azidoethoxy)-carbonyl)-L-lysine (AzK), N6-(((2-azidobenzyl)oxy)carbonyl)-L-lysine, N6-(((3-azidobenzyl)oxy)carbonyl)-L-lysine, N6-(((4-azidobenzyl)oxy)carbonyl)-L-lysine; an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynyl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or a combination thereof; an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an a-hydroxy containing acid; an amino thio acid; an a, a disubstituted amino acid; a β-amino acid; a cyclic amino acid other than proline or histidine, and an aromatic amino acid other than phenylalanine, tyrosine or tryptophan.

Other examples of unnatural amino acids include N6-((2-azidoethoxy)-carbonyl)-L-lysine (AzK), N6-(propargylethoxy)-L-lysine (PraK), N6-(((2-azidobenzyl)oxy)carbonyl)-L-lysine, N6-(((3-azidobenzyl)oxy)carbonyl)-L-lysine, N6-(((4-azidobenzyl)oxy)carbonyl)-L-lysine, N6-(((2-azidobenzyl)oxy)carbonyl)-L-lysine, N6-(((3-azidobenzyl)oxy)carbonyl)-L-lysine, and N6-(((4-azidobenzyl)oxy)carbonyl)-L-lysine.

In some embodiments, the unnatural amino acid comprises a selective reactive group, or a reactive group for site-selective labeling of a target polypeptide. In some instances, the chemistry is a biorthogonal reaction (e.g., biocompatible and selective reactions). In some cases, the chemistry is a Cu(I)-catalyzed or “copper-free” alkyne-azide triazole-forming reaction, the Staudinger ligation, inverse-electron-demand Diels-Alder (IEDDA) reaction, “photo-click” chemistry, or a metal-mediated process such as olefin metathesis and Suzuki-Miyaura or Sonogashira cross-coupling.

In some embodiments, the unnatural amino acid comprises a photoreactive group, which crosslinks, upon irradiation with, e.g., UV.

In some embodiments, the unnatural amino acid comprises a photo-caged amino acid.

In some instances, the unnatural amino acid is a para-substituted, meta-substituted, or an ortho-substituted amino acid derivative.

In some instances, the unnatural amino acid comprises p-acetyl-L-phenylalanine, p-azidomethyl-L-phenylalanine (pAMF), p-iodo-L-phenylalanine, O-methyl-L-tyrosine, p-methoxyphenylalanine, p-propargyloxyphenylalanine, p-propargyl-phenylalanine, L-3-(2-naphthyl)alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, L-phosphoserine, phosphonoserine, phosphonotyrosine, p-bromophenylalanine, p-amino-L-phenylalanine, or isopropyl-L-phenylalanine.

In some cases, the unnatural amino acid is 3-aminotyrosine, 3-nitrotyrosine, 3,4-dihydroxyphenylalanine, or 3-iodotyrosine.

In some cases, the unnatural amino acid is phenylselenocysteine.

In some instances, the unnatural amino acid is a benzophenone, ketone, iodide, methoxy, acetyl, benzoyl, or azide containing phenylalanine derivative.

In some instances, the unnatural amino acid is a benzophenone, ketone, iodide, methoxy, acetyl, benzoyl, or azide containing lysine derivative.

In some instances, the unnatural amino acid comprises an aromatic side chain.

In some instances, the unnatural amino acid does not comprise an aromatic side chain.

In some instances, the unnatural amino acid comprises an azido group.

In some embodiments, the at least one unnatural amino acid comprises N6-((2-azidoethoxy)-carbonyl)-L-lysine (AzK), N6-(propargylethoxy)-L-lysine (PraK), N6-(((2-azidobenzyl)oxy)carbonyl)-L-lysine, N6-(((3-azidobenzyl)oxy)carbonyl)-L-lysine, N6-(((4-azidobenzyl)oxy)carbonyl)-L-lysine, N6-(((2-azidobenzyl)oxy)carbonyl)-L-lysine, N6-(((3-azidobenzyl)oxy)carbonyl)-L-lysine, or N6-(((4-azidobenzyl)oxy)carbonyl)-L-lysine. In some embodiments, the at least one unnatural amino acid comprises N6-((2-azidoethoxy)-carbonyl)-L-lysine (AzK). In some embodiments, the at least one unnatural amino acid comprises N6-(propargylethoxy)-L-lysine (PraK). In some embodiments, the at least one unnatural amino acid comprises N6-(((2-azidobenzyl)oxy)carbonyl)-L-lysine. In some embodiments, the at least one unnatural amino acid comprises N6-(((3-azidobenzyl)oxy)carbonyl)-L-lysine. In some embodiments, the at least one unnatural amino acid comprises N6-(((4-azidobenzyl)oxy)carbonyl)-L-lysine. In some embodiments, the at least one unnatural amino acid comprises N6-(((2-azidobenzyl)oxy)carbonyl)-L-lysine. In some embodiments, the at least one unnatural amino acid comprises N6-(((3-azidobenzyl)oxy)carbonyl)-L-lysine. In some embodiments, the at least one unnatural amino acid comprises N6-(((4-azidobenzyl)oxy)carbonyl)-L-lysine.

In some instances, the unnatural amino acid comprises a Michael-acceptor group. In some instances, Michael-acceptor groups comprise an unsaturated moiety capable of forming a covalent bond through a 1,2-addition reaction. In some instances, Michael-acceptor groups comprise electron-deficient alkenes or alkynes. In some instances, Michael-acceptor groups include but are not limited to alpha,beta unsaturated: ketones, aldehydes, sulfoxides, sulfones, nitriles, imines, or aromatics.

In some instances, the unnatural amino acid is dehydroalanine.

In some instances, the unnatural amino acid comprises an aldehyde or ketone group.

In some instances, the unnatural amino acid is a lysine derivative comprising an aldehyde or ketone group.

In some instances, the unnatural amino acid is a lysine derivative comprising one or more O, N, Se, or S atoms at the beta, gamma, or delta position. In some instances, the unnatural amino acid is a lysine derivative comprising O, N, Se, or S atoms at the gamma position.

In some instances, the unnatural amino acid is a lysine derivative wherein the epsilon N atom is replaced with an oxygen atom.

In some instances, the unnatural amino acid is a lysine derivative that is not naturally-occurring post-translationally modified lysine.

In some instances, the unnatural amino acid is an amino acid comprising a side chain, wherein the sixth atom from the alpha position comprises a carbonyl group. In some instances, the unnatural amino acid is an amino acid comprising a side chain, wherein the sixth atom from the alpha position comprises a carbonyl group, and the fifth atom from the alpha position is a nitrogen. In some instances, the unnatural amino acid is an amino acid comprising a side chain, wherein the seventh atom from the alpha position is an oxygen atom.

In some instances, the unnatural amino acid is a serine derivative comprising selenium. In some instances, the unnatural amino acid is selenoserine (2-amino-3-hydroselenopropanoic acid). In some instances, the unnatural amino acid is 2-amino-3-((2-((3-(benzyloxy)-3-oxopropyl)amino)ethyl)selanyl)propanoic acid. In some instances, the unnatural amino acid is 2-amino-3-(phenylselanyl)propanoic acid. In some instances, the unnatural amino acid comprises selenium, wherein oxidation of the selenium results in the formation of an unnatural amino acid comprising an alkene.

In some instances, the unnatural amino acid comprises a cyclooctynyl group.

In some instances, the unnatural amino acid comprises a transcycloctenyl group.

In some instances, the unnatural amino acid comprises a norbornenyl group.

In some instances, the unnatural amino acid comprises a cyclopropenyl group.

In some instances, the unnatural amino acid comprises a diazirine group.

In some instances, the unnatural amino acid comprises a tetrazine group.

In some instances, the unnatural amino acid is a lysine derivative, wherein the side-chain nitrogen is carbamylated. In some instances, the unnatural amino acid is a lysine derivative, wherein the side-chain nitrogen is acylated. In some instances, the unnatural amino acid is 2-amino-6-{[(tert-butoxy)carbonyl]amino}hexanoic acid. In some instances, the unnatural amino acid is 2-amino-6-{[(tert-butoxy)carbonyl]amino}hexanoic acid. In some instances, the unnatural amino acid is N6-Boc-N6-methyllysine. In some instances, the unnatural amino acid is N6-acetyllysine. In some instances, the unnatural amino acid is pyrrolysine. In some instances, the unnatural amino acid is N6-trifluoroacetyllysine. In some instances, the unnatural amino acid is 2-amino-6-{[(benzyloxy)carbonyl]amino}hexanoic acid. In some instances, the unnatural amino acid is 2-amino-6-{[(p-iodobenzyloxy)carbonyl]amino}hexanoic acid. In some instances, the unnatural amino acid is 2-amino-6-{[(p-nitrobenzyloxy)carbonyl]amino}hexanoic acid. In some instances, the unnatural amino acid is N6-prolyllysine. In some instances, the unnatural amino acid is 2-amino-6-{[(cyclopentyloxy)carbonyl]amino}hexanoic acid. In some instances, the unnatural amino acid is N6-(cyclopentanecarbonyl)lysine. In some instances, the unnatural amino acid is N6-(tetrahydrofuran-2-carbonyl)lysine. In some instances, the unnatural amino acid is N6-(3-ethynyltetrahydrofuran-2-carbonyl)lysine. In some instances, the unnatural amino acid is N6-((prop-2-yn-1-yloxy)carbonyl)lysine. In some instances, the unnatural amino acid is 2-amino-6-{[(2-azidocyclopentyloxy)carbonyl]amino}hexanoic acid. In some instances, the unnatural amino acid is N6-((2-azidoethoxy)-carbonyl)-L-lysine. In some instances, the unnatural amino acid is 2-amino-6-{[(2-nitrobenzyloxy)carbonyl]amino}hexanoic acid. In some instances, the unnatural amino acid is 2-amino-6-{[(2-cyclooctynyloxy)carbonyl]amino}hexanoic acid. In some instances, the unnatural amino acid is N6-(2-aminobut-3-ynoyl)lysine. In some instances, the unnatural amino acid is 2-amino-6-((2-aminobut-3-ynoyl)oxy)hexanoic acid. In some instances, the unnatural amino acid is N6-(allyloxycarbonyl)lysine. In some instances, the unnatural amino acid is N6-(butenyl-4-oxycarbonyl)lysine. In some instances, the unnatural amino acid is N6-(pentenyl-5-oxycarbonyl)lysine. In some instances, the unnatural amino acid is N6-((but-3-yn-1-yloxy)carbonyl)-lysine. In some instances, the unnatural amino acid is N6-((pent-4-yn-1-yloxy)carbonyl)-lysine. In some instances, the unnatural amino acid is N6-(thiazolidine-4-carbonyl)lysine. In some instances, the unnatural amino acid is 2-amino-8-oxononanoic acid. In some instances, the unnatural amino acid is 2-amino-8-oxooctanoic acid. In some instances, the unnatural amino acid is N6-(2-oxoacetyl)lysine. In some instances, the unnatural amino acid is N6-(((2-azidobenzyl)oxy)carbonyl)-L-lysine. In some instances, the unnatural amino acid is N6-(((3-azidobenzyl)oxy)carbonyl)-L-lysine. In some instances, the unnatural amino acid is N6-(((4-azidobenzyl)oxy)carbonyl)-L-lysine.

In some instances, the unnatural amino acid is N6-propionyllysine. In some instances, the unnatural amino acid is N6-butyryllysine. In some instances, the unnatural amino acid is N6-(but-2-enoyl)lysine. In some instances, the unnatural amino acid is N6-((bicyclo[2.2.1]hept-5-en-2-yloxy)carbonyl)lysine. In some instances, the unnatural amino acid is N6-((spiro[2.3]hex-1-en-5-ylmethoxy)carbonyl)lysine. In some instances, the unnatural amino acid is N6-(((4-(1-(trifluoromethyl)cycloprop-2-en-1-yl)benzyl)oxy)carbonyl)lysine. In some instances, the unnatural amino acid is N6-((bicyclo[2.2.1]hept-5-en-2-ylmethoxy)carbonyl)lysine. In some instances, the unnatural amino acid is cysteinyllysine. In some instances, the unnatural amino acid is N6-((1-(6-nitrobenzo[d][1,3]dioxol-5-yl)ethoxy)carbonyl)lysine. In some instances, the unnatural amino acid is N6-((2-(3-methyl-3H-diazirin-3-yl)ethoxy)carbonyl)lysine. In some instances, the unnatural amino acid is N6-((3-(3-methyl-3H-diazirin-3-yl)propoxy)carbonyl)lysine. In some instances, the unnatural amino acid is N6-((meta nitrobenyloxy)N6-methylcarbonyl)lysine. In some instances, the unnatural amino acid is N6-((bicyclo[6.1.0]non-4-yn-9-ylmethoxy)carbonyl)-lysine. In some instances, the unnatural amino acid is N6-((cyclohept-3-en-1-yloxy)carbonyl)-L-lysine.

In some instances, the unnatural amino acid is 2-amino-3-(((((benzyloxy)carbonyl)amino)methyl)selanyl)propanoic acid.

In some embodiments, the unnatural amino acid is incorporated into the IL-10 polypeptide by a repurposed amber, opal, or ochre stop codon.

In some embodiments, the unnatural amino acid is incorporated into the IL-10 polypeptide by a 4-base codon.

In some embodiments, the unnatural amino acid is incorporated into the IL-10 polypeptide by a repurposed rare sense codon.

In some embodiments, the unnatural amino acid is incorporated into the IL-10 polypeptide by a synthetic codon comprising an unnatural nucleic acid.

Orthogonal Synthetase and tRNA Pair

In some instances, an unnatural amino acid is incorporated into an IL-10 polypeptide by a naturally occurring synthetase. In some embodiments, an unnatural amino acid is incorporated into a cytokine by an organism that is auxotrophic for one or more amino acids. In some embodiments, synthetases corresponding to the auxotrophic amino acid are capable of charging the corresponding tRNA with an unnatural amino acid. In some embodiments, the unnatural amino acid is selenocysteine, or a derivative thereof. In some embodiments, the unnatural amino acid is selenomethionine, or a derivative thereof. In some embodiments, the unnatural amino acid is an aromatic amino acid, wherein the aromatic amino acid comprises an aryl halide, such as an iodide. In embodiments, the unnatural amino acid is structurally similar to the auxotrophic amino acid.

### Conjugating Moieties

In certain embodiments, disclosed herein are conjugating moieties that are bound to an IL-10 polypeptide described herein. In some instances, the conjugating moiety is a molecule that perturbs the interaction of the IL-10 with its receptor. In some instances, the conjugating moiety is any molecule that when bound to the IL-10, enables the IL-10 conjugate to modulate an immune response. In some instances, the conjugating moiety is bound to the IL-10 through a covalent bond. In some instances, an IL-10 described herein is attached to a conjugating moiety with a triazole group. In some instances, an IL-10 described herein is attached to a conjugating moiety with a dihydropyridazine or pyridazine group. In some instances, the conjugating moiety comprises a water-soluble polymer. In other instances, the conjugating moiety comprises a protein or a binding fragment thereof. In additional instances, the conjugating moiety comprises a peptide. In additional instances, the conjugating moiety comprises a nucleic acid. In additional instances, the conjugating moiety comprises a small molecule. In additional instances, the conjugating moiety comprises a bioconjugate (e.g., a TLR agonist such as a TLR1, TLR2, TLR3, TLR4, TLR5, TLR6, TLR7, TLR8, or TLR9 agonist; or a synthetic ligand such as Pam3Cys, CFA, MALP2, Pam2Cys, FSL-1, Hib-OMPC, Poly I:C, poly A:U, AGP, MPL A, RC-529, MDF2β, CFA, or Flagellin). In some cases, the conjugating moiety increases serum half-life, and/or improves stability. In some cases, the conjugating moiety reduces cytokine interaction with one or more cytokine receptor domains or subunits. In additional cases, the conjugating moiety blocks IL-10 interaction with one or more IL-10 domains or subunits with its cognate receptor(s). In some embodiments, IL-10 conjugates described herein comprise multiple conjugating moieties. In some embodiments, a conjugating moiety is attached to an unnatural or natural amino acid in the IL-10 polypeptide. In some embodiments, an IL-10 conjugate comprises a conjugating moiety attached to a natural amino acid. In some embodiments, an IL-10 conjugate is attached to an unnatural amino acid in the cytokine peptide. In some embodiments, a conjugating moiety is attached to the N or C terminal amino acid of the IL-10 polypeptide. Various combinations sites are disclosed herein, for example, a first conjugating moiety is attached to an unnatural or natural amino acid in the IL-10 polypeptide, and a second conjugating moiety is attached to the N or C terminal amino acid of the IL-10 polypeptide. In some embodiments, a single conjugating moiety is attached to multiple residues of the IL-10 polypeptide (e.g. a staple). In some embodiments, a conjugating moiety is attached to both the N and C terminal amino acids of the IL-10 polypeptide.

### Water-Soluble Polymers

In some embodiments, a conjugating moiety descried herein is a water-soluble polymer. In some instances, the water-soluble polymer is a nonpeptidic, nontoxic, and biocompatible. As used herein, a substance is considered biocompatible if the beneficial effects associated with use of the substance alone or with another substance (e.g., an active agent such as an IL-10 moiety) in connection with living tissues (e.g., administration to a patient) outweighs any deleterious effects as evaluated by a clinician, e.g., a physician, a toxicologist, or a clinical development specialist. In some instances, a water-soluble polymer is further non-immunogenic. In some instances, a substance is considered non-immunogenic if the intended use of the substance in vivo does not produce an undesired immune response (e.g., the formation of antibodies) or, if an immune response is produced, that such a response is not deemed clinically significant or important as evaluated by a clinician, e.g., a physician, a toxicologist, or a clinical development specialist.

In some instances, the water-soluble polymer is characterized as having from about 2 to about 300 termini. Exemplary water soluble polymers include, but are not limited to, poly(alkylene glycols) such as polyethylene glycol (“PEG”), poly(propylene glycol) (“PPG”), copolymers of ethylene glycol and propylene glycol and the like, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol) (PVA), polyacrylamide (PAAm), poly(N-(2-hydroxypropyl) methacrylamide) (PHPMA), polydimethylacrylamide (PDAAm), polyphosphazene, polyoxazolines (“POZ”) (which are described in WO 2008/106186), poly(N-acryloylmorpholine), and combinations of any of the foregoing.

In some cases, the water-soluble polymer is not limited to a particular structure. In some cases, the water-soluble polymer is linear (e.g., an end capped, e.g., alkoxy PEG or a bifunctional PEG), branched or multi-armed (e.g., forked PEG or PEG attached to a polyol core), a dendritic (or star) architecture, each with or without one or more degradable linkages. Moreover, the internal structure of the water-soluble polymer can be organized in any number of different repeat patterns and can be selected from the group consisting of homopolymer, alternating copolymer, random copolymer, block copolymer, alternating tripolymer, random tripolymer, and block tripolymer.

In some embodiments, W of any of IL-10 conjugates described herein, such as any IL-10 conjugates comprising Formula (II), Formula (III), Formula (IV), or Formula (V), is a linear or branched PEG group. In some embodiments, W is a linear PEG group. In some embodiments, W is a branched PEG group. In some embodiments, W is a methoxy PEG group. In some embodiments, the methoxy PEG group is linear or branched. In some embodiments, the methoxy PEG group is linear. In some embodiments, the methoxy PEG group is branched.

In some embodiments, the weight-average molecular weight of the water-soluble polymer in the IL-10 conjugate is from about 100 Daltons to about 150,000 Daltons. Exemplary ranges include, for example, weight-average molecular weights in the range of greater than 5,000 Daltons to about 100,000 Daltons, in the range of from about 6,000 Daltons to about 90,000 Daltons, in the range of from about 10,000 Daltons to about 85,000 Daltons, in the range of greater than 10,000 Daltons to about 85,000 Daltons, in the range of from about 20,000 Daltons to about 85,000 Daltons, in the range of from about 53,000 Daltons to about 85,000 Daltons, in the range of from about 25,000 Daltons to about 120,000 Daltons, in the range of from about 29,000 Daltons to about 120,000 Daltons, in the range of from about 35,000 Daltons to about 120,000 Daltons, and in the range of from about 40,000 Daltons to about 120,000 Daltons.

Exemplary weight-average molecular weights for the water-soluble polymer include about 100 Daltons, about 200 Daltons, about 300 Daltons, about 400 Daltons, about 500 Daltons, about 600 Daltons, about 700 Daltons, about 750 Daltons, about 800 Daltons, about 900 Daltons, about 1,000 Daltons, about 1,500 Daltons, about 2,000 Daltons, about 2,200 Daltons, about 2,500 Daltons, about 3,000 Daltons, about 4,000 Daltons, about 4,400 Daltons, about 4,500 Daltons, about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, and about 75,000 Daltons. Branched versions of the water-soluble polymer (e.g., a branched 40,000 Dalton water-soluble polymer comprised of two 20,000 Dalton polymers) having a total molecular weight of any of the foregoing can also be used. In one or more embodiments, the conjugate will not have any PEG moieties attached, either directly or indirectly, with a PEG having a weight average molecular weight of less than about 6,000 Daltons.

PEGs will typically comprise a number of (OCH₂CH₂) monomers [or (CH₂CH₂O) monomers, depending on how the PEG is defined]. As used herein, the number of repeating units is identified by the subscript “n” in “(OCH₂CH₂)ₙ.” Thus, the value of (n) typically falls within one or more of the following ranges: from 2 to about 3400, from about 100 to about 2300, from about 100 to about 2270, from about 136 to about 2050, from about 225 to about 1930, from about 450 to about 1930, from about 1200 to about 1930, from about 568 to about 2727, from about 660 to about 2730, from about 795 to about 2730, from about 795 to about 2730, from about 909 to about 2730, and from about 1,200 to about 1,900. For any given polymer in which the molecular weight is known, it is possible to determine the number of repeating units (i.e., “n”) by dividing the total weight-average molecular weight of the polymer by the molecular weight of the repeating monomer.

In some instances, the water-soluble polymer is an end-capped polymer, that is, a polymer having at least one terminus capped with a relatively inert group, such as a lower C₁₋₆ alkoxy group, or a hydroxyl group. When the polymer is PEG, for example, a methoxy-PEG (commonly referred to as mPEG) may be used, which is a linear form of PEG wherein one terminus of the polymer is a methoxy (—OCH₃) group, while the other terminus is a hydroxyl or other functional group that can be optionally chemically modified.

In some embodiments, exemplary water-soluble polymers include, but are not limited to, linear or branched discrete PEG (dPEG) from Quanta Biodesign, Ltd; linear, branched, or forked PEGs from Nektar Therapeutics; linear, branched, or Y-shaped PEG derivatives from JenKem Technology.

In some embodiments, an IL-10 polypeptide described herein is conjugated to a water-soluble polymer selected from poly(alkylene glycols) such as polyethylene glycol (“PEG”), poly(propylene glycol) (“PPG”), copolymers of ethylene glycol and propylene glycol and the like, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol) (PVA), polyacrylamide (PAAm), polydimethylacrylamide (PDAAm), poly(N-(2-hydroxypropyl) methacrylamide) (PHPMA), polyphosphazene, polyoxazolines (“POZ”), poly(N-acryloylmorpholine), and a combination thereof. In some instances, the IL-10 polypeptide is conjugated to PEG (e.g., PEGylated). In some instances, the IL-10 polypeptide is conjugated to PPG. In some instances, the IL-10 polypeptide is conjugated to POZ. In some instances, the IL-10 polypeptide is conjugated to PVP.

In some instances, a water-soluble polymer comprises a polyglycerol (PG). In some cases, the polyglycerol is a hyperbranched PG (HPG) (e.g., as described by Imran, et al. “Influence of architecture of high molecular weight linear and branched polyglycerols on their biocompatibility and biodistribution,” Biomaterials 33:9135-9147 (2012), the disclosure of which is incorporated herein by reference). In other cases, the polyglycerol is a linear PG (LPG). In additional cases, the polyglycerol is a midfunctional PG, a linear-block-hyperbranched PG (e.g., as described by Wurm et. Al., “Squaric acid mediated synthesis and biological activity of a library of linear and hyperbranched poly(glycerol)-protein conjugates,” Biomacromolecules 13:1161-1171 (2012), the disclosure of which is incorporated herein by reference), or a side-chain functional PG (e.g., as described by Li, et. al., “Synthesis of linear polyether polyol derivatives as new materials for bioconjugation,” Bioconjugate Chem. 20:780-789 (2009), the disclosure of which is incorporated herein by reference).

In some instances, an IL-10 polypeptide described herein is conjugated to a PG, e.g., a HPG, a LPG, a midfunctional PG, a linear-block-hyperbranched PG, or a side-chain functional PG.

In some embodiments, a water-soluble polymer is a degradable synthetic PEG alternative. Exemplary degradable synthetic PEG alternatives include, but are not limited to, poly[oligo(ethylene glycol)methyl methacrylate] (POEGMA); backbone modified PEG derivatives generated by polymerization of telechelic, or di-end-functionalized PEG-based macromonomers; PEG derivatives comprising comonomers comprising degradable linkage such as poly[(ethylene oxide)-co-(methylene ethylene oxide)][P(EO-co-MEO)], cyclic ketene acetals such as 5,6-benzo-2-methylene-1,3-dioxepane (BMDO), 2-methylene-1,3-dioxepane (MDO), and 2-methylene-4-phenyl-1,3-dioxolane (MPDL) copolymerized with OEGMA; or poly-(ε-caprolactone)-graft-poly(ethylene oxide) (PCL-g-PEO).

In some instances, an IL-10 polypeptide described herein is conjugated to a degradable synthetic PEG alternative, such as for example, POEGM; backbone modified PEG derivatives generated by polymerization of telechelic, or di-end-functionalized PEG-based macromonomers; P(EO-co-MEO); cyclic ketene acetals such as BMDO, MDO, and MPDL copolymerized with OEGMA; or PCL-g-PEO.

In some embodiments, a water-soluble polymer comprises a poly(zwitterions). Exemplary poly(zwitterions) include, but are not limited to, poly(sulfobetaine methacrylate) (PSBMA), poly(carboxybetaine methacrylate) (PCBMA), and poly(2-methyacryloyloxyethyl phosphorylcholine) (PMPC). In some instances, an IL-10 polypeptide is conjugated to a poly(zwitterion) such as PSBMA, PCBMA, or PMPC.

In some embodiments, a water-soluble polymer comprises a polycarbonate. Exemplary polycarbonates include, but are not limited to, pentafluorophenyl 5-methyl-2-oxo-1,3-dioxane-5-carboxylate (MTC-OC₆F₅). In some instances, an IL-10 polypeptide described herein is conjugated to a polycarbonate such as MTC-OC₆F₅.

In some embodiments, a water-soluble polymer comprises a polymer hybrid, such as for example, a polycarbonate/PEG polymer hybrid, a peptide/protein-polymer conjugate, or a hydroxyl containing and/or zwitterionic derivatized polymer (e.g., a hydroxyl containing and/or zwitterionic derivatized PEG polymer). In some instances, an IL-10 polypeptide described herein is conjugated to a polymer hybrid such as a polycarbonate/PEG polymer hybrid, a peptide/protein-polymer conjugate, or a hydroxyl containing and/or zwitterionic derivatized polymer (e.g., a hydroxyl containing and/or zwitterionic derivatized PEG polymer).

In some instances, a water-soluble polymer comprises a polysaccharide. Exemplary polysaccharides include, but are not limited to, dextran, polysialic acid (PSA), hyaluronic acid (HA), amylose, heparin, heparan sulfate (HS), dextrin, or hydroxyethyl-starch (HES). In some cases, an IL-10 polypeptide is conjugated to a polysaccharide. In some cases, an IL-10 polypeptide is conjugated to dextran. In some cases, an IL-10 polypeptide is conjugated to PSA. In some cases, an IL-10 polypeptide is conjugated to HA. In some cases, an IL-10 polypeptide is conjugated to amylose. In some cases, an IL-10 polypeptide is conjugated to heparin. In some cases, an IL-10 polypeptide is conjugated to HS. In some cases, an IL-10 polypeptide is conjugated to dextrin. In some cases, an IL-10 polypeptide is conjugated to HES.

In some cases, a water-soluble polymer comprises a glycan. Exemplary classes of glycans include N-linked glycans, O-linked glycans, glycolipids, O-GlcNAc, and glycosaminoglycans. In some cases, an IL-10 polypeptide is conjugated to a glycan. In some cases, an IL-10 polypeptide is conjugated to N-linked glycans. In some cases, an IL-10 polypeptide is conjugated to O-linked glycans. In some cases, an IL-10 polypeptide is conjugated to glycolipids. In some cases, an IL-10 polypeptide is conjugated to O-GlcNAc. In some cases, an IL-10 polypeptide is conjugated to glycosaminoglycans.

In some embodiments, a water-soluble polymer comprises a polyoxazoline polymer. A polyoxazoline polymer is a linear synthetic polymer, and similar to PEG, comprises a low polydispersity. In some instances, a polyoxazoline polymer is a polydispersed polyoxazoline polymer, characterized with an average molecule weight. In some cases, the average molecule weight of a polyoxazoline polymer includes, for example, 1000, 1500, 2000, 2500, 3000, 3500, 4000, 4500, 5000, 5500, 6000, 6500, 7000, 7500, 8000, 10,000, 12,000, 20,000, 35,000, 40,000, 50,000, 60,000, 100,000, 200,000, 300,000, 400,000, or 500,000 Da. In some instances, a polyoxazoline polymer comprises poly(2-methyl 2-oxazoline) (PMOZ), poly(2-ethyl 2-oxazoline) (PEOZ), or poly(2-propyl 2-oxazoline) (PPOZ). In some cases, an IL-10 polypeptide is conjugated to a polyoxazoline polymer. In some cases, an IL-10 polypeptide is conjugated to PMOZ. In some cases, an IL-10 polypeptide is conjugated to PEOZ. In some cases, an IL-10 polypeptide is conjugated to PPOZ.

In some instances, a water-soluble polymer comprises a polyacrylic acid polymer. In some cases, an IL-10 polypeptide is conjugated to a polyacrylic acid polymer.

In some instances, a water-soluble polymer comprises polyamine. Polyamine is an organic polymer comprising two or more primary amino groups. In some embodiments, a polyamine includes a branched polyamine, a linear polyamine, or cyclic polyamine. In some cases, a polyamine is a low-molecular-weight linear polyamine. Exemplary polyamines include putrescine, cadaverine, spermidine, spermine, ethylene diamine, 1,3-diaminopropane, hexamethylenediamine, tetraethylmethylenediamine, and piperazine. In some cases, an IL-10 polypeptide is conjugated to a polyamine. In some cases, an IL-10 polypeptide is conjugated to putrescine, cadaverine, spermidine, spermine, ethylene diamine, 1,3-diaminopropane, hexamethylenediamine, tetraethylmethylenediamine, or piperazine.

In some instances, a water-soluble polymer is described in U.S. Pat. Nos. 7,744,861, 8,273,833, and 7,803,777. In some instances, an IL-10 polypeptide is conjugated to a linker described in U.S. Pat. Nos. 7,744,861, 8,273,833, or 7,803,777.

### Lipids

In some embodiments, a conjugating moiety descried herein is a lipid. In some instances, the lipid is a fatty acid. In some cases, the fatty acid is a saturated fatty acid. In other cases, the fatty acid is an unsaturated fatty acid. Exemplary fatty acids include, but are not limited to, fatty acids comprising from about 6 to about 26 carbon atoms, from about 6 to about 24 carbon atoms, from about 6 to about 22 carbon atoms, from about 6 to about 20 carbon atoms, from about 6 to about 18 carbon atoms, from about 20 to about 26 carbon atoms, from about 12 to about 26 carbon atoms, from about 12 to about 24 carbon atoms, from about 12 to about 22 carbon atoms, from about 12 to about 20 carbon atoms, or from about 12 to about 18 carbon atoms. In some cases, the lipid binds to one or more serum proteins, thereby increasing serum stability and/or serum half-life.

In some embodiments, the lipid is conjugated to an IL-10 polypeptide described herein. In some instances, the lipid is a fatty acid, e.g., a saturated fatty acid or an unsaturated fatty acid. In some cases, the fatty acid is from about 6 to about 26 carbon atoms, from about 6 to about 24 carbon atoms, from about 6 to about 22 carbon atoms, from about 6 to about 20 carbon atoms, from about 6 to about 18 carbon atoms, from about 20 to about 26 carbon atoms, from about 12 to about 26 carbon atoms, from about 12 to about 24 carbon atoms, from about 12 to about 22 carbon atoms, from about 12 to about 20 carbon atoms, or from about 12 to about 18 carbon atoms. In some cases, the fatty acid comprises about 6, 7, 8, 9, 10, 11, 12, 13, 14, 15, 16, 17, 18, 19, 20, 21, 22, 23, 24, 25, or 26 carbon atoms in length. In some cases, the fatty acid comprises caproic acid (hexanoic acid), enanthic acid (heptanoic acid), caprylic acid (octanoic acid), pelargonic acid (nonanoic acid), capric acid (decanoic acid), undecylic acid (undecanoic acid), lauric acid (dodecanoic acid), tridecylic acid (tridecanoic acid), myristic acid (tetradecanoic acid), pentadecylic acid (pentadecanoic acid), palmitic acid (hexadecanoic acid), margaric acid (heptadecanoic acid), stearic acid (octadecanoic acid), nonadecylic acid (nonadecanoic acid), arachidic acid (eicosanoic acid), heneicosylic acid (heneicosanoic acid), behenic acid (docosanoic acid), tricosylic acid (tricosanoic acid), lignoceric acid (tetracosanoic acid), pentacosylic acid (pentacosanoic acid), or cerotic acid (hexacosanoic acid).

In some embodiments, the IL-10 lipid conjugate enhances serum stability and/or serum half-life.

### Proteins

In some embodiments, a conjugating moiety descried herein is a protein or a binding fragment thereof. Exemplary proteins include albumin, transferrin, or transthyretin. In some instances, the protein or a binding fragment thereof comprises an antibody, or its binding fragments thereof. In some cases, an IL-10 conjugate comprises a protein or a binding fragment thereof. In some cases, an IL-10 conjugate comprising a protein or a binding fragment thereof has an increased serum half-life, and/or stability. In some cases, an IL-10 conjugate comprising a protein or a binding fragment thereof has a reduced IL-10 interaction with one or more IL-10R subunits. In additional cases, the protein or a binding fragment thereof blocks IL-10 interaction with one or more IL-10R subunits.

In some embodiments, the conjugating moiety is albumin. Albumin is a family of water-soluble globular proteins. It is commonly found in blood plasma, comprising about 55-60% of all plasma proteins. Human serum albumin (HSA) is a 585 amino acid polypeptide in which the tertiary structure is divided into three domains, domain I (amino acid residues 1-195), domain II (amino acid residues 196-383), and domain III (amino acid residues 384-585). Each domain further comprises a binding site, which can interact either reversibly or irreversibly with endogenous ligands such as long- and medium-chain fatty acids, bilirubin, or hemin, or exogenous compounds such as heterocyclic or aromatic compounds.

In some cases, an IL-10 polypeptide is conjugated to albumin. In some cases, the IL-10 polypeptide is conjugated to human serum albumin (HSA). In additional cases, the IL-10 polypeptide is conjugated to a functional fragment of albumin.

In some embodiments, the conjugating moiety is transferrin. Transferrin is a 679 amino acid polypeptide that is about 80 kDa in size and comprises two Fe³⁺ binding sites with one at the N-terminal domain and the other at the C-terminal domain. In some instances, human transferrin has a half-life of about 7-12 days.

In some instances, an IL-10 polypeptide is conjugated to transferrin. In some cases, the IL-10 polypeptide is conjugated to human transferrin. In additional cases, the IL-10 polypeptide is conjugated to a functional fragment of transferrin.

In some embodiments, the conjugating moiety is transthyretin (TTR). Transthyretin is a transport protein located in the serum and cerebrospinal fluid which transports the thyroid hormone thyroxine (T₄) and retinol-binding protein bound to retinol.

In some instances, an IL-10 polypeptide is conjugated to transthyretin (via one of its termini or via an internal hinge region). In some cases, the IL-10 polypeptide is conjugated to a functional fragment of transthyretin.

In some embodiments, the conjugating moiety is an antibody, or its binding fragments thereof. In some instances, an antibody or its binding fragments thereof comprise a humanized antibody or binding fragment thereof, murine antibody or binding fragment thereof, chimeric antibody or binding fragment thereof, monoclonal antibody or binding fragment thereof, monovalent Fab′, divalent Fab₂, F(ab)′₃ fragments, single-chain variable fragment (scFv), bis-scFv, (scFv)₂, diabody, minibody, nanobody, triabody, tetrabody, humabody, disulfide stabilized Fv protein (dsFv), single-domain antibody (sdAb), Ig NAR, camelid antibody or binding fragment thereof, bispecific antibody or biding fragment thereof, or a chemically modified derivative thereof.

In some instances, the conjugating moiety comprises a scFv, bis-scFv, (scFv)₂, dsFv, or sdAb. In some cases, the conjugating moiety comprises a scFv. In some cases, the conjugating moiety comprises a bis-scFv. In some cases, the conjugating moiety comprises a (scFv)₂. In some cases, the conjugating moiety comprises a dsFv. In some cases, the conjugating moiety comprises a sdAb.

In some instances, the conjugating moiety comprises an Fc portion of an antibody, e.g., of IgG, IgA, IgM, IgE, or IgD. In some instances, the moiety comprises an Fc portion of IgG (e.g., IgG₁, IgG₃, or IgG₄).

In some cases, an IL-10 polypeptide is conjugated to an antibody, or its binding fragments thereof. In some cases, the IL-10 polypeptide is conjugated to a humanized antibody or binding fragment thereof, murine antibody or binding fragment thereof, chimeric antibody or binding fragment thereof, monoclonal antibody or binding fragment thereof, monovalent Fab′, divalent Fab₂, F(ab)′₃ fragments, single-chain variable fragment (scFv), bis-scFv, (scFv)₂, diabody, minibody, nanobody, triabody, tetrabody, humabody, disulfide stabilized Fv protein (dsFv), single-domain antibody (sdAb), Ig NAR, camelid antibody or binding fragment thereof, bispecific antibody or biding fragment thereof, or a chemically modified derivative thereof. In additional cases, the IL-10 polypeptide is conjugated to an Fc portion of an antibody. In additional cases, the IL-10 polypeptide is conjugated to an Fc portion of IgG (e.g., IgG₁, IgG₃, or IgG₄).

In some embodiments, an IL-10 polypeptide is conjugated to a water-soluble polymer (e.g., PEG) and an antibody or binding fragment thereof. In some cases, the antibody or binding fragments thereof comprises a humanized antibody or binding fragment thereof, murine antibody or binding fragment thereof, chimeric antibody or binding fragment thereof, monoclonal antibody or binding fragment thereof, monovalent Fab′, divalent Fab₂, F(ab)′₃ fragments, single-chain variable fragment (scFv), bis-scFv, (scFv)₂, diabody, minibody, nanobody, triabody, tetrabody, humabody, disulfide stabilized Fv protein (dsFv), single-domain antibody (sdAb), Ig NAR, camelid antibody or binding fragment thereof, bispecific antibody or biding fragment thereof, or a chemically modified derivative thereof. In some cases, the antibody or binding fragments thereof comprises a scFv, bis-scFv, (scFv)₂, dsFv, or sdAb. In some cases, the antibody or binding fragments thereof comprises a scFv. In some cases, the antibody or binding fragment thereof guides the IL-10 conjugate to a target cell of interest and the water-soluble polymer enhances stability and/or serum half-life.

In some instances, one or more IL-10 polypeptide—water-soluble polymer (e.g., PEG) conjugates are further bound to an antibody or binding fragments thereof. In some instances, the ratio of the IL-10 conjugate to the antibody is about 1:1, 2:1, 3:1, 4:1, 5:1, 6:1, 7:1, 8:1, 9:1, 10:1, 11:1, or 12:1. In some cases, the ratio of the IL-10 conjugate to the antibody is about 1:1. In other cases, the ratio of the IL-10 conjugate to the antibody is about 2:1, 3:1, or 4:1. In additional cases, the ratio of the IL-10 conjugate to the antibody is about 6:1 or higher.

In some embodiments, the one or more IL-10 polypeptide—water-soluble polymer (e.g., PEG) conjugates are directly bound to the antibody or binding fragments thereof. In other instances, the IL-10 conjugate is indirectly bound to the antibody or binding fragments thereof with a linker. Exemplary linkers include homobifunctional linkers, heterobifunctional linkers, maleimide-based linkers, zero-trace linkers, self-immolative linkers, spacers, and the like.

In some embodiments, the antibody or binding fragments thereof is bound either directly or indirectly to the IL-10 polypeptide portion of the IL-10 polypeptide—water-soluble polymer (e.g., PEG) conjugate. In such cases, the conjugation site of the antibody to the IL-10 polypeptide is at a site that will not impede binding of the IL-10 polypeptide with the IL-10R. In additional cases, the conjugation site of the antibody to the IL-10 polypeptide is at a site that partially blocks binding of the IL-10 polypeptide with the IL-10R. In other embodiments, the antibody or binding fragments thereof is bound either directly or indirectly to the water-soluble polymer portion of the IL-10 polypeptide—water-soluble polymer (e.g., PEG) conjugate.

### Peptides

In some embodiments, a conjugating moiety descried herein is a peptide. In some instances, the peptide is a non-structured peptide. In some cases, an IL-10 polypeptide is conjugated to a peptide. In some cases, the IL-10 conjugate comprising a peptide has an increased serum half-life, and/or stability. In some cases, the IL-10 conjugate comprising a peptide has a reduced IL-10 interaction with one or more IL-10R subunits. In additional cases, the peptide blocks IL-10 interaction with one or more IL-10R subunits.

In some instances, the conjugating moiety is a XTEN™ peptide (Amunix Operating Inc.) and the modification is referred to as XTENylation. XTENylation is the genetic fusion of a nucleic acid encoding a polypeptide of interest with a nucleic acid encoding a XTEN™ peptide (Amunix Operating Inc.), a long unstructured hydrophilic peptide comprising different percentage of six amino acids: Ala, Glu, Gly, Ser, and Thr. In some instances, a XTEN™ peptide is selected based on properties such as expression, genetic stability, solubility, aggregation resistance, enhanced half-life, increased potency, and/or increased in vitro activity in combination with a polypeptide of interest. In some cases, an IL-10 polypeptide is conjugated to a XTEN peptide.

In some instances, the conjugating moiety is a glycine-rich homoamino acid polymer (HAP) and the modification is referred to as HAPylation. HAPylation is the genetic fusion of a nucleic acid encoding a polypeptide of interest with a nucleic acid encoding a glycine-rich homoamino acid polymer (HAP). In some instances, the HAP polymer comprises a (Gly₄Ser)ₙ repeat motif (SEQ ID NO: 67) and sometimes are about 50, 100, 150, 200, 250, 300, or more residues in length. In some cases, an IL-10 polypeptide is conjugated to HAP.

In some embodiments, the conjugating moiety is a PAS polypeptide and the modification is referred to as PASylation. PASylation is the genetic fusion of a nucleic acid encoding a polypeptide of interest with a nucleic acid encoding a PAS polypeptide. A PAS polypeptide is a hydrophilic uncharged polypeptide consisting of Pro, Ala and Ser residues. In some instances, the length of a PAS polypeptide is at least about 100, 200, 300, 400, 500, or 600 amino acids. In some cases, an IL-10 polypeptide is conjugated to a PAS polypeptide.

In some embodiments, the conjugating moiety is an elastin-like polypeptide (ELP) and the modification is referred to as ELPylation. ELPylation is the genetic fusion of a nucleic acid encoding a polypeptide of interest with a nucleic acid encoding an elastin-like polypeptide (ELPs). An ELP comprises a VPGxG repeat motif (SEQ ID NO: 77) in which x is any amino acid except proline. In some cases, an IL-10 polypeptide is conjugated to ELP.

In some embodiments, the conjugating moiety is a CTP peptide. A CTP peptide comprises a 30 or 31 amino acid residue peptide (FQSSSS*KAPPPS*LPSPS*RLPGPS*DTPILPQ (SEQ ID NO: 78) or FQDSSSS*KAPPPS*LPSPS*RLPGPS*DTPILPQ (SEQ ID NO: 79)) in which the S* denotes O-glycosylation sites (OPKO). In some instances, a CTP peptide is genetically fused to an IL-10 polypeptide). In some cases, an IL-10 polypeptide is conjugated to a CTP peptide.

In some embodiments, an IL-10 polypeptide is modified by glutamylation. Glutamylation (or polyglutamylation) is a reversible posttranslational modification of glutamate, in which the γ-carboxy group of glutamate forms a peptide-like bond with the amino group of a free glutamate in which the α-carboxy group extends into a polyglutamate chain.

In some embodiments, an IL-10 polypeptide is modified by a gelatin-like protein (GLK) polymer. In some instances, the GLK polymer comprises multiple repeats of Gly-Xaa-Yaa wherein Xaa and Yaa primarily comprise proline and 4-hydroxyproline, respectively. In some cases, the GLK polymer further comprises amino acid residues Pro, Gly, Glu, Gln, Asn, Ser, and Lys. In some cases, the length of the GLK polymer is about 20, 30, 40, 50, 60, 70, 80, 90, 100, 110, 120, 150 residues or longer.

### Additional Conjugating Moieties

In some instances, the conjugating moiety comprises an extracellular biomarker. In some instances, the extracellular biomarker is a tumor antigen. In some instances, exemplary extracellular biomarker comprises CD19, PSMA, B7-H3, B7-H6, CD70, CEA, CSPG4, EGFRvIII, EphA3, EpCAM, EGFR, ErbB2 (HER2), FAP, FRα, GD2, GD3, Lewis-Y, mesothelin, Muc1, Muc 16, ROR1, TAG72, VEGFR2, CD11, Gr-1, CD204, CD16, CD49b, CD3, CD4, CD8, and B220. In some instances, the conjugating moiety is bond or conjugated to the IL-10. In some cases, the conjugating moiety is genetically fused, for example, at the N-terminus or the C-terminus, of the IL-10.

In some instances, the conjugating moiety comprises a molecule from a post-translational modification. In some instances, examples of post-translational modification include myristoylation, palmitoylation, isoprenylation (or prenylation) (e.g., farnesylation or geranylgeranylation), glypiation, acylation (e.g., O-acylation, N-acylation, S-acylation), alkylation (e.g., additional of alkyl groups such as methyl or ethyl groups), amidation, glycosylation, hydroxylation, iodination, nucleotide addition, oxidation, phosphorylation, succinylation, sulfation, glycation, carbamylation, glutamylation, or deamidation. In some instances, the IL-10 is modified by a post-translational modification such as myristoylation, palmitoylation, isoprenylation (or prenylation) (e.g., farnesylation or geranylgeranylation), glypiation, acylation (e.g., O-acylation, N-acylation, S-acylation), alkylation (e.g., additional of alkyl groups such as methyl or ethyl groups), amidation, glycosylation, hydroxylation, iodination, nucleotide addition, oxidation, phosphorylation, succinylation, sulfation, glycation, carbamylation, glutamylation, or deamidation.

### Conjugation

### Linkers

In some embodiments, useful functional reactive groups for conjugating or binding a conjugating moiety to an IL-10 polypeptide described herein include, for example, zero or higher-order linkers. In some instances, an unnatural amino acid incorporated into an interleukin described herein comprises a functional reactive group. In some instances, a linker comprises a functional reactive group that reacts with an unnatural amino acid incorporated into an interleukin described herein. In some instances, a conjugating moiety comprises a functional reactive group that reacts with an unnatural amino acid incorporated into an interleukin described herein. In some instances, a conjugating moiety comprises a functional reactive group that reacts with a linker (optionally pre-attached to a cytokine peptide) described herein. In some embodiments, a linker comprises a reactive group that reacts with a natural amino acid in an IL-10 polypeptide described herein. In some cases, higher-order linkers comprise bifunctional linkers, such as homobifunctional linkers or heterobifunctional linkers. Exemplary homobifuctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[P-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the bifunctional linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-a-methyl-a-(2-pyridyldithio)toluene (sMPT), sulfosuccinimidyl-6-[a-methyl-a-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C₂H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(ρ-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), ρ-nitrophenyl diazopyruvate (pNPDP), ρ-nitrophenyl-2-diazo-3,3,3-trifluoropropionate (PNP-DTP), sulfhydryl-reactive and photoreactive cross-linkers such as1-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some instances, the reactive functional group comprises a nucleophilic group that is reactive to an electrophilic group present on a binding moiety (e.g., on a conjugating moiety or on IL-10). Exemplary electrophilic groups include carbonyl groups—such as aldehyde, ketone, carboxylic acid, ester, amide, enone, acyl halide or acid anhydride. In some embodiments, the reactive functional group is aldehyde. Exemplary nucleophilic groups include hydrazide, oxime, amino, hydrazine, thiosemicarbazone, hydrazine carboxylate, and arylhydrazide. In some embodiments, an unnatural amino acid incorporated into an interleukin described herein comprises an electrophilic group.

In some embodiments, the linker is a cleavable linker. In some embodiments, the cleavable linker is a dipeptide linker. In some embodiments, the dipeptide linker is valine-citrulline (Val-Cit), phenylalanine-lysine (Phe-Lys), valine-alanine (Val-Ala) and valine-lysine (Val-Lys). In some embodiments, the dipeptide linker is valine-citrulline.

In some embodiments, the linker is a peptide linker comprising, e.g., at least 2, 3, 4, 5, 6, 7, 8, 9, 10, 11, 12, 15, 20, 25, 30, 35, 40, 45, 50, or more amino acids. In some instances, the peptide linker comprises at most 2, 3, 4, 5, 6, 7, 8, 9, 10, 11, 12, 15, 20, 25, 30, 35, 40, 45, 50, or less amino acids. In additional cases, the peptide linker comprises about 2, 3, 4, 5, 6, 7, 8, 9, 10, 11, 12, 15, 20, 25, 30, 35, 40, 45, or 50 amino acids.

In some embodiments, the linker comprises a self-immolative linker moiety. In some embodiments, the self-immolative linker moiety comprises p-aminobenzyl alcohol (PAB), p-aminobenzyoxycarbonyl (PABC), or derivatives or analogs thereof. In some embodiments, the linker comprises a dipeptide linker moiety and a self-immolative linker moiety. In some embodiments, the self-immolative linker moiety is such as described in U.S. Pat. No. 9,089,614 and WIPO Application No. WO2015038426, the disclosure of each of which is incorporated herein by reference.

In some embodiments, the cleavable linker is glucuronide. In some embodiments, the cleavable linker is an acid-cleavable linker. In some embodiments, the acid-cleavable linker is hydrazine. In some embodiments, the cleavable linker is a reducible linker.

In some embodiments, the linker comprises a maleimide group. In some instances, the maleimide group is also referred to as a maleimide spacer. In some instances, the maleimide group further comprises a caproic acid, forming maleimidocaproyl (mc). In some cases, the linker comprises maleimidocaproyl (mc). In some cases, linker is maleimidocaproyl (mc). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some embodiments, the maleimide group is a self-stabilizing maleimide. In some instances, the self-stabilizing maleimide utilizes diaminopropionic acid (DPR) to incorporate a basic amino group adjacent to the maleimide to provide intramolecular catalysis of thiosuccinimide ring hydrolysis, thereby eliminating maleimide from undergoing an elimination reaction through a retro-Michael reaction. In some instances, the self-stabilizing maleimide is a maleimide group described in Lyon, et al., “Self-hydrolyzing maleimides improve the stability and pharmacological properties of antibody-drug conjugates,” Nat. Biotechnol. 32(10):1059-1062 (2014), the disclosure of which is incorporated herein by reference. In some instances, the linker comprises a self-stabilizing maleimide. In some instances, the linker is a self-stabilizing maleimide.

### Conjugation Chemistry

Various conjugation reactions are used to conjugate linkers, conjugation moieties, and unnatural amino acids incorporated into cytokine peptides described herein. Such conjugation reactions are often compatible with aqueous conditions, such as “bioorthogonal” reactions. In some embodiments, conjugation reactions are mediated by chemical reagents such as catalysts, light, or reactive chemical groups found on linkers, conjugation moieties, or unnatural amino acids. In some embodiments, conjugation reactions are mediated by enzymes. In some embodiments, a conjugation reaction used herein is described in Gong, Y., Pan, L. Tett. Lett. 2015, 56, 2123, the disclosure of which is incorporated herein by reference. In some embodiments, a conjugation reaction used herein is described in Chen, X.; Wu. Y-W. Org. Biomol. Chem. 2016, 14, 5417, the disclosure of which is incorporated herein by reference.

In some embodiments described herein, a conjugation reaction described herein comprises a 1,3-dipolar cycloaddition reaction. In some embodiments, the 1,3-dipolar cycloaddition reaction comprises reaction of an azide and a phosphine (“Click” reaction). In some embodiments, the conjugation reaction is catalyzed by copper. In some embodiments, a conjugation reaction described herein results in cytokine peptide comprising a linker or conjugation moiety attached via a triazole. In some embodiments, a conjugation reaction described herein comprises reaction of an azide with a strained olefin. In some embodiments, a conjugation reaction described herein comprises reaction of an azide with a strained alkyne. In some embodiments, a conjugation reaction described herein comprises reaction of an azide with a cycloalkyne, for example DBCO.

In some embodiments described herein, a conjugation reaction described herein comprises the reaction outlined in Scheme 1:

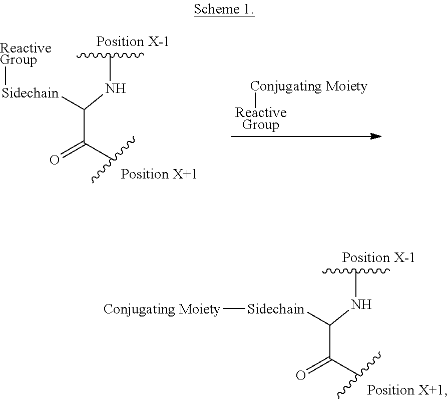

wherein X is the position in the IL-10 conjugate comprising an unnatural amino acid, such as in any one of SEQ ID NOS: 3 to 10. In some embodiments, the conjugating moiety comprises a water soluble polymer. In some embodiments, a reactive group comprises an alkyne or azide.

In some embodiments described herein, a conjugation reaction described herein comprises the reaction outlined in Scheme 2:

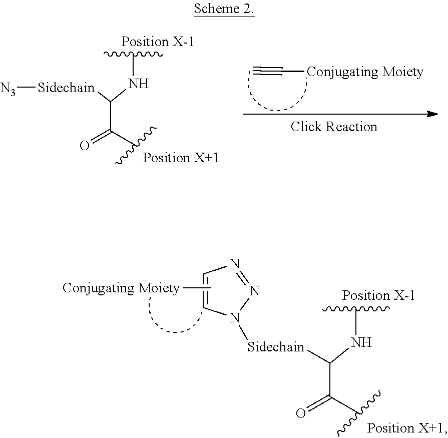

wherein X is the position in the IL-10 conjugate comprising an unnatural amino acid, such as in any one of SEQ ID NOS: 3 to 10.

In some embodiments described herein, a conjugation reaction described herein comprises the reaction outlined in Scheme 3:

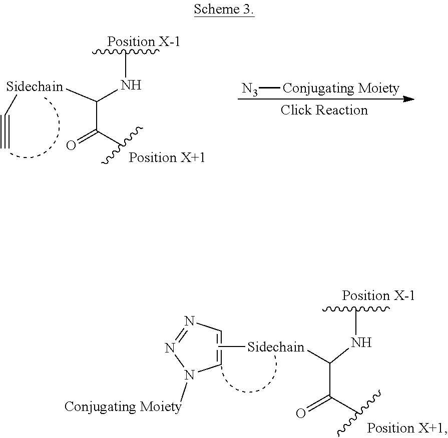

wherein X is the position in the IL-10 conjugate comprising an unnatural amino acid, such as in any one of SEQ ID NOS: 3 to 10.

In some embodiments described herein, a conjugation reaction described herein comprises the reaction outlined in Scheme 4:

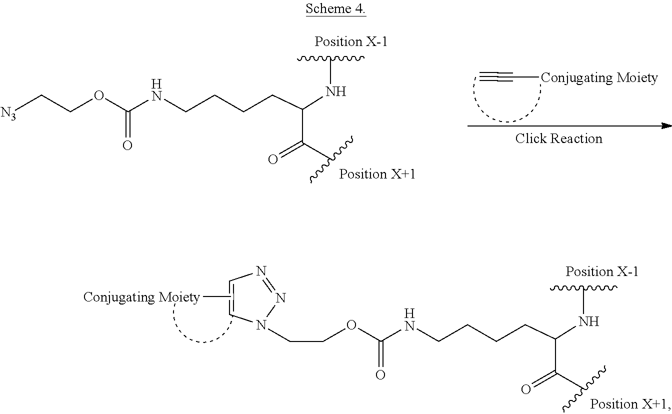

wherein X is the position in the IL-10 conjugate comprising an unnatural amino acid, such as in any one of SEQ ID NOS: 3 to 10.

In some embodiments described herein, a conjugation reaction described herein comprises a cycloaddition reaction between an azide moiety, such as that contained in a protein containing an amino acid residue derived from N6-((2-azidoethoxy)-carbonyl)-L-lysine (AzK), and a strained cycloalkyne, such as that derived from DBCO, which is a chemical moiety comprising a dibenzocyclooctyne group. PEG groups comprising a DBCO moiety are commercially available or may be prepared by methods know to those of ordinary skill in the art. Exemplary reactions are shown in Schemes 5a-b and 6a-b.

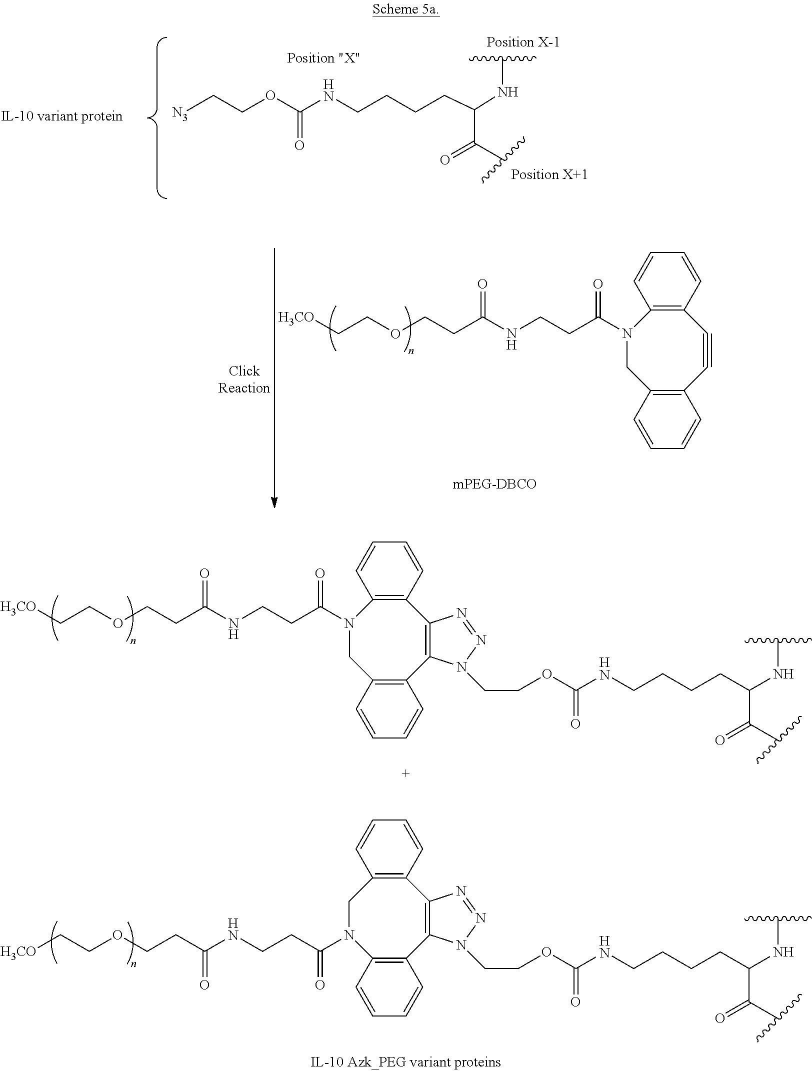

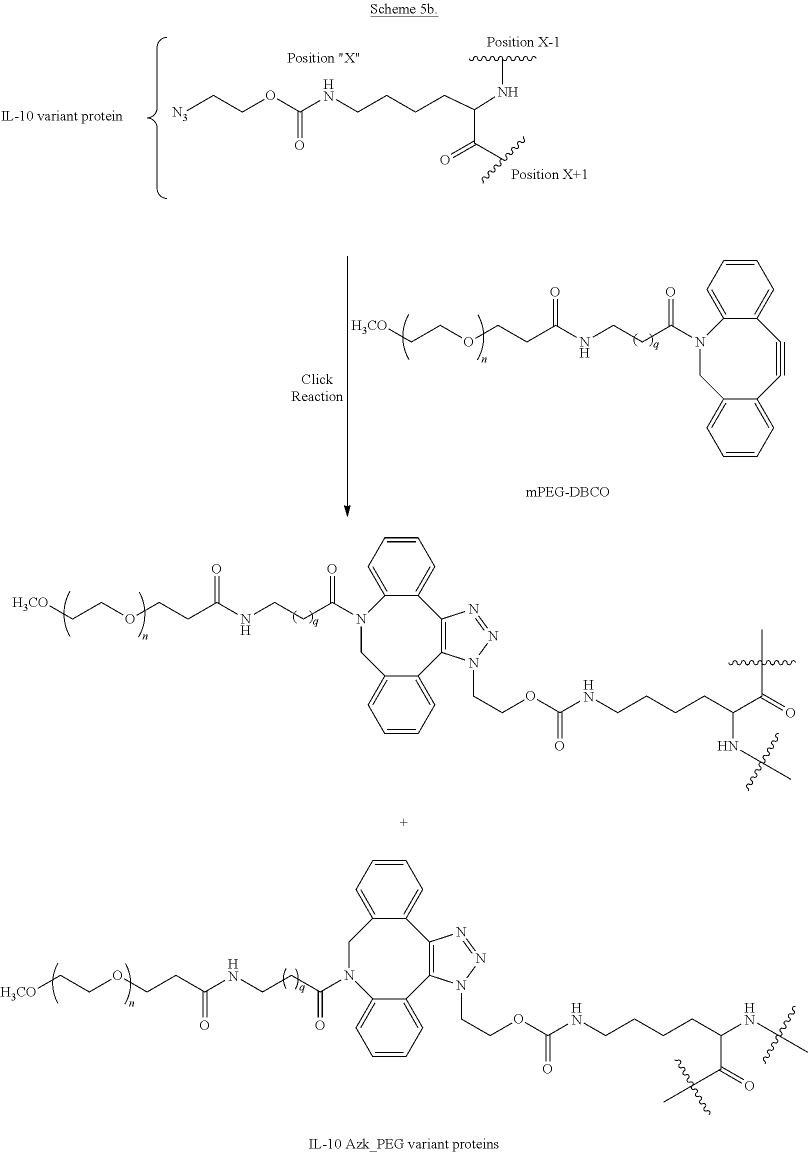

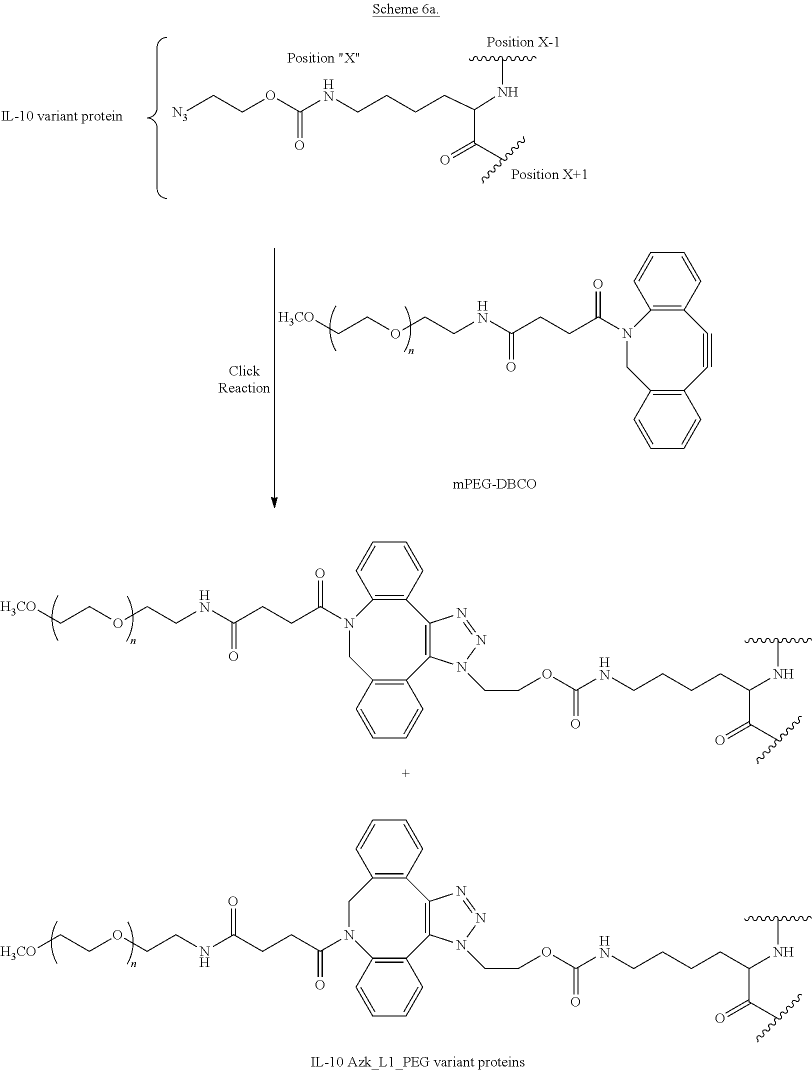

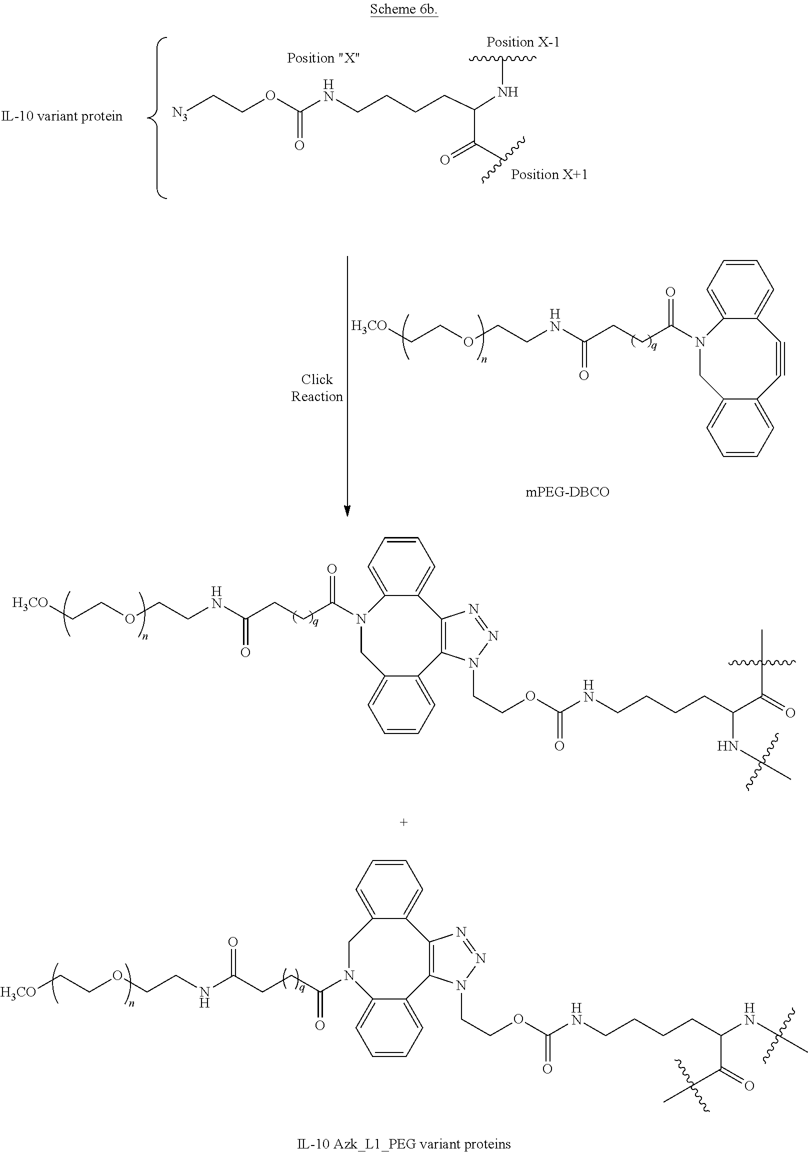

Conjugation reactions such as a click reaction described herein may generate a single regioisomer or a mixture of regioisomers. In some instances, the ratio of regioisomers is about 1:1. In some instances, the ratio of regioisomers is about 2:1. In some instances, the ratio of regioisomers is about 1.5:1. In some instances, the ratio of regioisomers is about 1.2:1. In some instances, the ratio of regioisomers is about 1.1:1. In some instances, the ratio of regioisomers is greater than 1:1.

In one aspect, provided herein is a method of making an IL-10 conjugate as described herein, comprising:

reacting an IL-10 polypeptide comprising an unnatural amino acid of formula

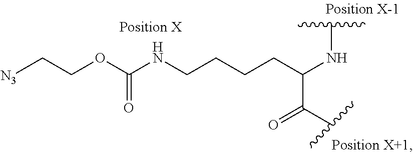 [SMILES: *NC(CCCCNC(=O)OCCN=[N+]=[N-])C(*)=O]

wherein the IL-10 polypeptide comprises the amino acid sequence of SEQ ID NO: 1 in which at least one amino acid residue in the IL-10 polypeptide is replaced by the unnatural amino acid, Position X−1 indicates the point of attachment to the preceding amino acid residue, Position X+1 indicates the point of attachment to the following amino acid residue, and Position X indicates the position of the amino acid for which the unnatural amino acid substitutes, with an mPEG-DBCO of formula

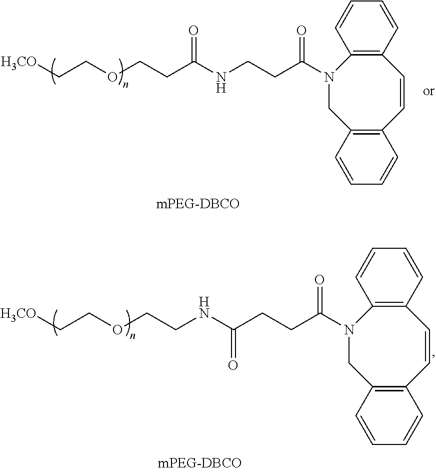

wherein n is such that the mPEG-DBCO comprises a PEG having a molecular weight of about 5 kDa, 10 kDa, 15 kDa, 20 kDa, 25 kDa, 30 kDa, 35 kDa, 40 kDa, 45 kDa, 50 kDa, or 60 kDa, thereby producing the IL-10 conjugate.

In a further aspect, provided herein is a method of making an IL-10 conjugate as described herein, comprising:

reacting an IL-10 polypeptide comprising an unnatural amino acid of formula

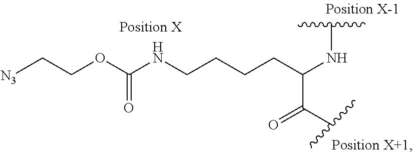 [SMILES: *NC(CCCCNC(=O)OCCN=[N+]=[N-])C(*)=O]

wherein the IL-10 polypeptide comprises the amino acid sequence of SEQ ID NO: 1 in which at least one amino acid residue in the IL-10 polypeptide is replaced by the unnatural amino acid, Position X−1 indicates the point of attachment to the preceding amino acid residue, Position X+1 indicates the point of attachment to the following amino acid residue, and Position X indicates the position of the amino acid for which the unnatural amino acid substitutes, with an mPEG-DBCO of formula

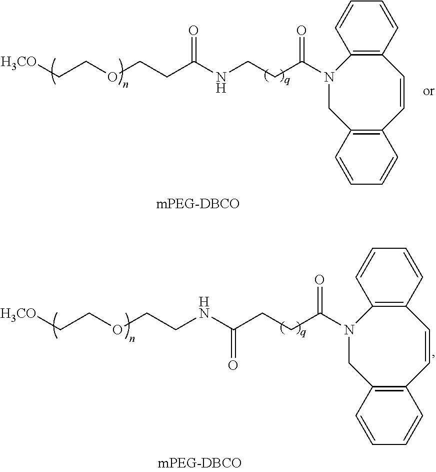

wherein n is such that the mPEG-DBCO comprises a PEG having a molecular weight of about 5 kDa, 10 kDa, 15 kDa, 20 kDa, 25 kDa, 30 kDa, 35 kDa, 40 kDa, 45 kDa, 50 kDa, or 60 kDa, thereby producing the IL-10 conjugate.

### IL-10 Polypeptide Production

In some instances, the IL-10 conjugates described herein, either containing a natural amino acid mutation or an unnatural amino acid mutation, are generated recombinantly or are synthesized chemically. In some instances, IL-10 conjugates described herein are generated recombinantly, for example, either by a host cell system, or in a cell-free system.

In some instances, IL-10 conjugates are generated recombinantly through a host cell system. In some cases, the host cell is a eukaryotic cell (e.g., mammalian cell, insect cells, yeast cells or plant cell) or a prokaryotic cell (e.g., gram-positive bacterium or a gram-negative bacterium). In some cases, a eukaryotic host cell is a mammalian host cell. In some cases, a mammalian host cell is a stable cell line, or a cell line that has incorporated a genetic material of interest into its own genome and has the capability to express the product of the genetic material after many generations of cell division. In other cases, a mammalian host cell is a transient cell line, or a cell line that has not incorporated a genetic material of interest into its own genome and does not have the capability to express the product of the genetic material after many generations of cell division.

Exemplary mammalian host cells include 293T cell line, 293A cell line, 293FT cell line, 293F cells, 293 H cells, A549 cells, MDCK cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some embodiments, a eukaryotic host cell is an insect host cell. Exemplary insect host cells include Drosophila S2 cells, Sf9 cells, Sf21 cells, High Five™ cells, and expresSF+® cells.

In some embodiments, a eukaryotic host cell is a yeast host cell. Exemplary yeast host cells include Pichia pastoris (K. phaffii) yeast strains such as GS 115, KM71H, SMD 1168, SMD 1168H, and X−33, and Saccharomyces cerevisiae yeast strain such as INVSc1.

In some embodiments, a eukaryotic host cell is a plant host cell. In some instances, the plant cells comprise a cell from algae. Exemplary plant cell lines include strains from Chlamydomonas reinhardtii 137c, or Synechococcus elongatus PPC 7942.

In some embodiments, a host cell is a prokaryotic host cell. Exemplary prokaryotic host cells include BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, suitable polynucleic acid molecules or vectors for the production of an IL-10 polypeptide described herein include any suitable vectors derived from either a eukaryotic or prokaryotic source. Exemplary polynucleic acid molecules or vectors include vectors from bacteria (e.g., E. coli), insects, yeast (e.g., Pichia pastoris, K. phaffii), algae, or mammalian source. Bacterial vectors include, for example, pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Insect vectors include, for example, pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

Yeast vectors include, for example, Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris (K. phaffii) vector, pGAPZA, B, & C Pichia pastoris (K. phaffii) vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Algae vectors include, for example, pChlamy-4 vector or MCS vector.

Mammalian vectors include, for example, transient expression vectors or stable expression vectors. Exemplary mammalian transient expression vectors include p3×FLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3×FLAG-CMV 7.1, pFLAG-CMV 20, p3×FLAG-Myc-CMV 24, pCMV-FLAG-MAT1, pCMV-FLAG-MAT2, pBICEP-CMV 3, or pBICEP-CMV 4. Exemplary mammalian stable expression vectors include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In some instances, a cell-free system is used for the production of a cytokine (e.g., IL-10) polypeptide described herein. In some cases, a cell-free system comprises a mixture of cytoplasmic and/or nuclear components from a cell and is suitable for in vitro nucleic acid synthesis. In some instances, a cell-free system utilizes prokaryotic cell components. In other instances, a cell-free system utilizes eukaryotic cell components. Nucleic acid synthesis is obtained in a cell-free system based on, for example, Drosophila cell, Xenopus egg, Archaea, or HeLa cells. Exemplary cell-free systems include E. coli S30 Extract system, E. coli T7 S30 system, or PURExpress®, XpressCF, and XpressCF+.

Cell-free translation systems variously comprise components such as plasmids, mRNA, DNA, tRNAs, synthetases, release factors, ribosomes, chaperone proteins, translation initiation and elongation factors, natural and/or unnatural amino acids, and/or other components used for protein expression. Such components are optionally modified to improve yields, increase synthesis rate, increase protein product fidelity, or incorporate unnatural amino acids. In some embodiments, cytokines described herein are synthesized using cell-free translation systems described in U.S. Pat. No. 8,778,631; US 2017/0283469; US 2018/0051065; US 2014/0315245; or U.S. Pat. No. 8,778,631, the disclosure of each of which is incorporated herein by reference. In some embodiments, cell-free translation systems comprise modified release factors, or even removal of one or more release factors from the system. In some embodiments, cell-free translation systems comprise a reduced protease concentration. In some embodiments, cell-free translation systems comprise modified tRNAs with re-assigned codons used to code for unnatural amino acids. In some embodiments, the synthetases described herein for the incorporation of unnatural amino acids are used in cell-free translation systems. In some embodiments, tRNAs are pre-loaded with unnatural amino acids using enzymatic or chemical methods before being added to a cell-free translation system. In some embodiments, components for a cell-free translation system are obtained from modified organisms, such as modified bacteria, yeast, or other organism.

In some embodiments, a cytokine (e.g., IL-10) polypeptide is generated as a circularly permuted form, either via an expression host system or through a cell-free system.

### Production of IL-10 Polypeptide Comprising an Unnatural Amino Acid

An orthogonal or expanded genetic code can be used in the present disclosure, in which one or more specific codons present in the nucleic acid sequence of a cytokine (e.g., IL-10) polypeptide are allocated to encode the unnatural amino acid so that it can be genetically incorporated into the cytokine (e.g., IL-10) by using an orthogonal tRNA synthetase/tRNA pair. The orthogonal tRNA synthetase/tRNA pair is capable of charging a tRNA with an unnatural amino acid and is capable of incorporating that unnatural amino acid into the polypeptide chain in response to the codon.

In some embodiments, a polynucleotide is provided comprising the sequence of SEQ ID NO: 76 in which a codon is substituted with a codon that encodes an unnatural amino acid. In some embodiments, a polynucleotide is provided comprising a sequence having at least 85% identity to SEQ ID NO: 76, wherein the polynucleotide comprises a codon that encodes an unnatural amino acid, optionally wherein T residues are replaced with U residues. The polynucleotide may encode any of the IL-10 sequences comprising an unnatural amino acid described herein. In some embodiments, the sequence has at least 90%, 95%, 96%, 97%, 98%, or 99% identity to SEQ ID NO: 76. The polynucleotide may be a DNA, such as a plasmid, expression vector, or integrated expression construct. The polynucleotide may be an RNA, such as an mRNA.

In some instances, the codon is the codon amber, ochre, opal or a quadruplet codon. In some cases, the codon corresponds to the orthogonal tRNA which will be used to carry the unnatural amino acid. In some cases, the codon is amber. In other cases, the codon is an orthogonal codon.

In some instances, the codon is a quadruplet codon, which can be decoded by an orthogonal ribosome ribo-Q1. In some cases, the quadruplet codon is as illustrated in Neumann, et al., “Encoding multiple unnatural amino acids via evolution of a quadruplet-decoding ribosome,” Nature, 464(7287): 441-444 (2010), the disclosure of which is incorporated herein by reference.

In some instances, a codon used in the present disclosure is a recoded codon, e.g., a synonymous codon or a rare codon that is replaced with alternative codon. In some cases, the recoded codon is as described in Napolitano, et al., “Emergent rules for codon choice elucidated by editing rare arginine codons in Escherichia coli,” PNAS, 113(38): E5588-5597 (2016), the disclosure of which is incorporated herein by reference. In some cases, the recoded codon is as described in Ostrov et al., “Design, synthesis, and testing toward a 57-codon genome,” Science 353(6301): 819-822 (2016), the disclosure of which is incorporated herein by reference.

In some instances, unnatural nucleic acids are utilized leading to incorporation of one or more unnatural amino acids into the cytokine (e.g., IL-10). Exemplary unnatural nucleic acids include, but are not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifiuoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, 0-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and 0-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, 2-amino-2′-deoxyadenosine, and those described in U.S. Pat. Nos. 3,687,808; 4,845,205; 4,910,300; 4,948,882; 5,093,232; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096; WO 99/62923; Kandimalla et al., (2001) Bioorg. Med. Chem. 9:807-813; The Concise Encyclopedia of Polymer Science and Engineering, Kroschwitz, J. I., Ed., John Wiley & Sons, 1990, 858-859; Englisch et al., Angewandte Chemie, International Edition, 1991, 30, 613; and Sanghvi, Chapter 15, Antisense Research and Applications, Crooke and Lebleu Eds., CRC Press, 1993, 273-288, the disclosure of each of which is incorporated herein by reference. Additional base modifications can be found, for example, in U.S. Pat. No. 3,687,808; Englisch et al., Angewandte Chemie, International Edition, 1991, 30, 613; and Sanghvi, Chapter 15, Antisense Research and Applications, pages 289-302, Crooke and Lebleu ed., CRC Press, 1993, the disclosure of each of which is incorporated herein by reference.

Unnatural nucleic acids comprising various heterocyclic bases and various sugar moieties (and sugar analogs) are available in the art, and the nucleic acids in some cases include one or several heterocyclic bases other than the principal five base components of naturally-occurring nucleic acids. For example, the heterocyclic base includes, in some cases, uracil-5-yl, cytosin-5-yl, adenin-7-yl, adenin-8-yl, guanin-7-yl, guanin-8-yl, 4-aminopyrrolo [2.3-d] pyrimidin-5-yl, 2-amino-4-oxopyrolo [2, 3-d] pyrimidin-5-yl, 2-amino-4-oxopyrrolo [2.3-d] pyrimidin-3-yl groups, where the purines are attached to the sugar moiety of the nucleic acid via the 9-position, the pyrimidines via the 1-position, the pyrrolopyrimidines via the 7-position and the pyrazolopyrimidines via the 1-position.

In some embodiments, nucleotide analogs are also modified at the phosphate moiety. Modified phosphate moieties include, but are not limited to, those with modification at the linkage between two nucleotides and contains, for example, a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides are through a 3′-5′ linkage or a 2′-5′ linkage, and the linkage contains inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, the disclosure of each of which is incorporated herein by reference.

In some embodiments, unnatural nucleic acids include 2′,3′-dideoxy-2′,3′-didehydro-nucleosides (PCT/US2002/006460), 5′-substituted DNA and RNA derivatives (PCT/US2011/033961; Saha et al., J. Org Chem., 1995, 60, 788-789; Wang et al., Bioorganic & Medicinal Chemistry Letters, 1999, 9, 885-890; and Mikhailov et al., Nucleosides & Nucleotides, 1991, 10(1-3), 339-343; Leonid et al., 1995, 14(3-5), 901-905; and Eppacher et al., Helvetica Chimica Acta, 2004, 87, 3004-3020; PCT/JP2000/004720; PCT/JP2003/002342; PCT/JP2004/013216; PCT/JP2005/020435; PCT/JP2006/315479; PCT/JP2006/324484; PCT/JP2009/056718; PCT/JP2010/067560), or 5′-substituted monomers made as the monophosphate with modified bases (Wang et al., Nucleosides Nucleotides & Nucleic Acids, 2004, 23 (1 & 2), 317-337), the disclosure of each of which is incorporated herein by reference.

In some embodiments, unnatural nucleic acids include modifications at the 5′-position and the 2′-position of the sugar ring (PCT/US94/02993), such as 5′-CH₂-substituted 2′-O-protected nucleosides (Wu et al., Helvetica Chimica Acta, 2000, 83, 1127-1143 and Wu et al., Bioconjugate Chem. 1999, 10, 921-924). In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃ (Mesmaeker et al., Synlett, 1997, 1287-1290). Unnatural nucleic acids can include 2′-substituted 5′-CH₂ (or O) modified nucleosides (PCT/US92/01020). Unnatural nucleic acids can include 5′-methylenephosphonate DNA and RNA monomers, and dimers (Bohringer et al., Tet. Lett., 1993, 34, 2723-2726; Collingwood et al., Synlett, 1995, 7, 703-705; and Hutter et al., Helvetica Chimica Acta, 2002, 85, 2777-2806). Unnatural nucleic acids can include 5′-phosphonate monomers having a 2′-substitution (US2006/0074035) and other modified 5′-phosphonate monomers (WO1997/35869). Unnatural nucleic acids can include 5′-modified methylenephosphonate monomers (EP614907 and EP629633). Unnatural nucleic acids can include analogs of 5′ or 6′-phosphonate ribonucleosides comprising a hydroxyl group at the 5′ and/or 6′-position (Chen et al., Phosphorus, Sulfur and Silicon, 2002, 777, 1783-1786; Jung et al., Bioorg. Med. Chem., 2000, 8, 2501-2509; Gallier et al., Eur. J. Org. Chem., 2007, 925-933; and Hampton et al., J. Med. Chem., 1976, 19(8), 1029-1033). Unnatural nucleic acids can include 5′-phosphonate deoxyribonucleoside monomers and dimers having a 5′-phosphate group (Nawrot et al., Oligonucleotides, 2006, 16(1), 68-82). Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH2) (and analogs thereof) or a cyano group (CN) (and analogs thereof) (Fairhurst et al., Synlett, 2001, 4, 467-472; Kappler et al., J. Med. Chem., 1986, 29, 1030-1038; Kappler et al., J. Med. Chem., 1982, 25, 1179-1184; Vrudhula et al., J. Med. Chem., 1987, 30, 888-894; Hampton et al., J. Med. Chem., 1976, 19, 1371-1377; Geze et al., J. Am. Chem. Soc, 1983, 105(26), 7638-7640; and Hampton et al., J. Am. Chem. Soc, 1973, 95(13), 4404-4414). The disclosure of each of these references is incorporated herein by reference.

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids (BNA); replacement of the ribosyl ring oxygen atom with S, N(R), or C(R₁)(R₂) (R=H, C₁-C₁₂ alkyl or a protecting group); and combinations thereof. Examples of chemically modified sugars can be found in WO2008/101157, US2005/0130923, and WO2007/134181, the disclosure of each of which is incorporated herein by reference.

In some instances, a modified nucleic acid comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety may be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol like ribonucleosides. The preparation of these sugars or sugar analogs and the respective “nucleosides” wherein such sugars or analogs are attached to a heterocyclic base (nucleic acid base) is known. Sugar modifications may also be made and combined with other modifications.

Modifications to the sugar moiety include natural modifications of the ribose and deoxy ribose as well as unnatural modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]m CH₃, —O(CH₂)ₙOCH₃, —O(CH₂)ₙNH2, —O(CH₂)ₙCH₃, —O(CH₂)ₙONH₂, and —O(CH₂)ₙON[(CH₂)ₙ CH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Modified sugars also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures and which detail and describe a range of base modifications, such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,700,920, the disclosure of each of which is herein incorporated by reference in its entirety.

Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, 0-allyl, O—(C₁-C_{1O} alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′, and analogs thereof (see, U.S. Pat. No. 7,399,845); 4′-C(CH₃)(CH₃)—O-2′ and analogs thereof, (see WO2009/006478, WO2008/150729, US2004/0171570, U.S. Pat. No. 7,427,672, Chattopadhyaya et al., J. Org. Chem., 209, 74, 118-134, and WO2008/154401). Also see, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invens. Drugs, 2001, 2, 558-561; Braasch et al., Chem. Biol, 2001, 8, 1-7; Oram et al., Curr. Opinion Mol. Ther., 2001, 3, 239-243; U.S. Pat. Nos. 4,849,513; 5,015,733; 5,118,800; 5,118,802; 7,053,207; 6,268,490; 6,770,748; 6,794,499; 7,034,133; 6,525,191; 6,670,461; and 7,399,845; International Publication Nos. WO2004/106356, WO1994/14226, WO2005/021570, WO2007/090071, and WO2007/134181; U.S. Patent Publication Nos. US2004/0171570, US2007/0287831, and US2008/0039618; U.S. Provisional Application Nos. 60/989,574, 61/026,995, 61/026,998, 61/056,564, 61/086,231, 61/097,787, and 61/099,844; and International Applications Nos. PCT/US2008/064591, PCT US2008/066154, PCT US2008/068922, and PCT/DK98/00393; the disclosure of each of which is incorporated herein by reference.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and may be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like. Exemplary polynucleotides containing modified phosphate linkages or non-phosphate linkages can be found in Peyrottes et al., 1996, Nucleic Acids Res. 24: 1841-1848; Chaturvedi et al., 1996, Nucleic Acids Res. 24:2318-2323; and Schultz et al., (1996) Nucleic Acids Res. 24:2966-2973; Matteucci, 1997, “Oligonucleotide Analogs: an Overview” in Oligonucleotides as Therapeutic Agents, (Chadwick and Cardew, ed.) John Wiley and Sons, New York, N.Y.; Zon, 1993, “Oligonucleoside Phosphorothioates” in Protocols for Oligonucleotides and Analogs, Synthesis and Properties, Humana Press, pp. 165-190; Miller et al., 1971, JACS 93:6657-6665; Jager et al., 1988, Biochem. 27:7247-7246; Nelson et al., 1997, JOC 62:7278-7287; U.S. Pat. No. 5,453,496; and Micklefield, 2001, Curr. Med. Chem. 8: 1157-1179; the disclosure of each of which is incorporated herein by reference.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos (Micklefield, 2001, Current Medicinal Chemistry 8: 1157-1179, the disclosure of which is incorporated herein by reference). A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439. It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. See also Nielsen et al., Science, 1991, 254, 1497-1500. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. KY. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EM5OJ, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1-di-O-hexadecyl-rac-glycero-S—H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochem. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941. The disclosure of each of these references is incorporated herein by reference

In some cases, the unnatural nucleic acids further form unnatural base pairs. Exemplary unnatural nucleotides capable of forming an unnatural DNA or RNA base pair (UBP) under conditions in vivo includes, but is not limited to, TAT1, dTAT1, 5FM, d5FM, TPT3, dTPT3, 55ICS, d5SICS, NaM, dNaM, CNMO, dCNMO, and combinations thereof. In some embodiments, unnatural nucleotides include:

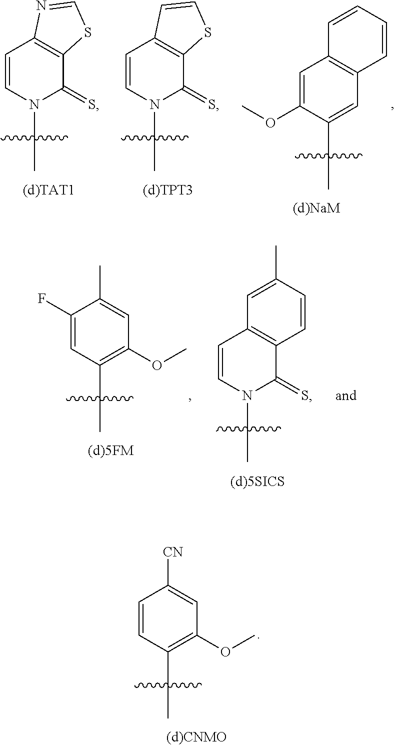

Exemplary unnatural base pairs include: (d)TPT3-(d)NaM; (d)5SICS-(d)NaM; (d)CNMO-(d)TAT1; (d)NaM-(d)TAT1; (d)CNMO-(d)TPT3; and (d)5FM-(d)TAT1.

Other examples of unnatural nucleotides capable of forming unnatural UBPs that may be used to prepare the IL-10 conjugates disclosed herein may be found in Dien et al., J Am Chem Soc., 2018, 140:16115-16123; Feldman et al., J Am Chem Soc, 2017, 139:11427-11433; Ledbetter et al., J Am Chem Soc., 2018, 140:758-765; Dhami et al., Nucleic Acids Res. 2014, 42:10235-10244; Malyshev et al., Nature, 2014, 509:385-388; Betz et al., J Am Chem Soc., 2013, 135:18637-18643; Lavergne et al., J Am Chem Soc. 2013, 135:5408-5419; and Malyshev et al. Proc Natl Acad Sci USA, 2012, 109:12005-12010; the disclosure of each of which is incorporated herein by reference. In some embodiments, unnatural nucleotides include:

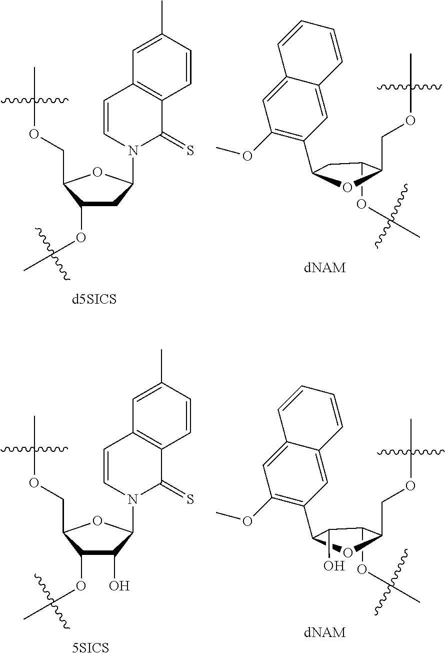

In some embodiments, the unnatural nucleotides that may be used to prepare the IL-10 conjugates disclosed herein may be derived from a compound of the formula

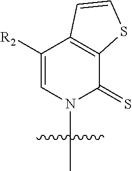

wherein R₂ is selected from the group consisting of hydrogen, alkyl, alkenyl, alkynyl, methoxy, methanethiol, methaneseleno, halogen, cyano, and azido; and the wavy line indicates a bond to a ribosyl or 2′-deoxyribosyl, wherein the 5′-hydroxy group of the ribosyl or 2′-deoxyribosyl moiety is in free form, or is optionally bound to a monophosphate, a diphosphate, or a triphosphate group, or is included in an RNA or a DNA or in an RNA analog or a DNA analog.

In some embodiments, each X is carbon. In some embodiments, at least one X is carbon. In some embodiments, one X is carbon. In some embodiments, at least two X are carbon. In some embodiments, two X are carbon. In some embodiments, at least one X is nitrogen. In some embodiments, one X is nitrogen. In some embodiments, at least two X are nitrogen. In some embodiments, two X are nitrogen.

In some embodiments, Y is sulfur. In some embodiments, Y is oxygen. In some embodiments, Y is selenium. In some embodiments, Y is a secondary amine.

In some embodiments, E is sulfur. In some embodiments, E is oxygen. In some embodiments, E is selenium.

In some embodiments, R₂ is present when X is carbon. In some embodiments, R² is absent when X is nitrogen. In some embodiments, each R₂, where present, is hydrogen. In some embodiments, R₂ is alkyl, such as methyl, ethyl, or propyl. In some embodiments, R₂ is alkenyl, such as —CH₂═CH₂. In some embodiments, R₂ is alkynyl, such as ethynyl. In some embodiments, R₂ is methoxy. In some embodiments, R₂ is methanethiol. In some embodiments, R₂ is methaneseleno. In some embodiments, R₂ is halogen, such as chloro, bromo, or fluoro. In some embodiments, R₂ is cyano. In some embodiments, R₂ is azide.

In some embodiments, E is sulfur, Y is sulfur, and each X is independently carbon or nitrogen. In some embodiments, E is sulfur, Y is sulfur, and each X is carbon.

In some embodiments, the unnatural nucleotides that may be used to prepare the IL-10 conjugates disclosed herein may be derived from

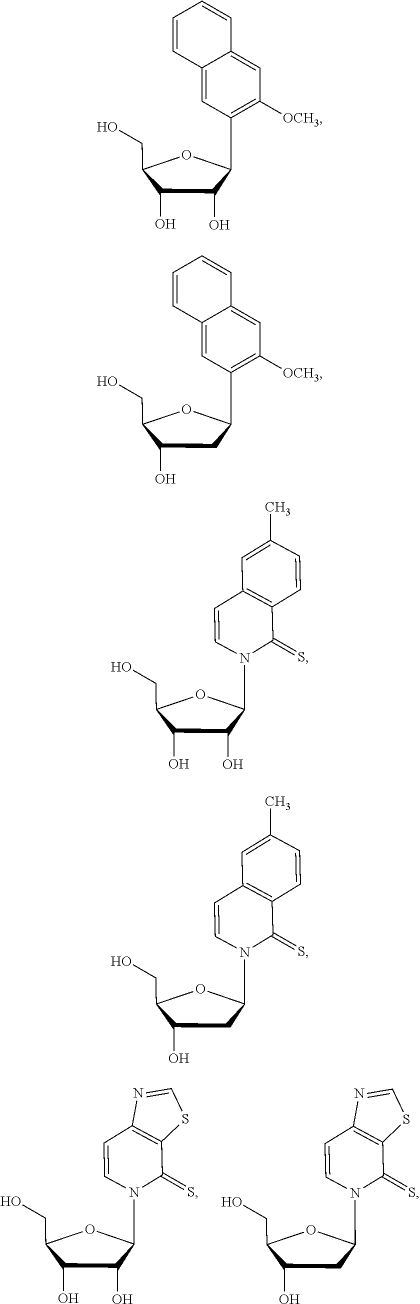

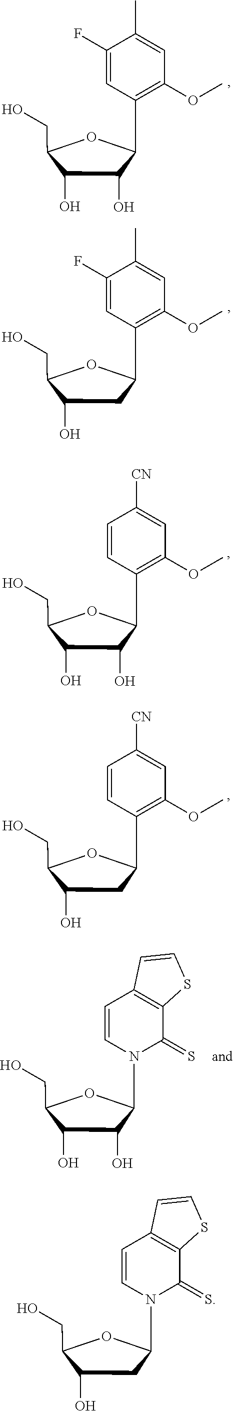

In some embodiments, the unnatural nucleotides that may be used to prepare the IL-10 conjugates disclosed herein include

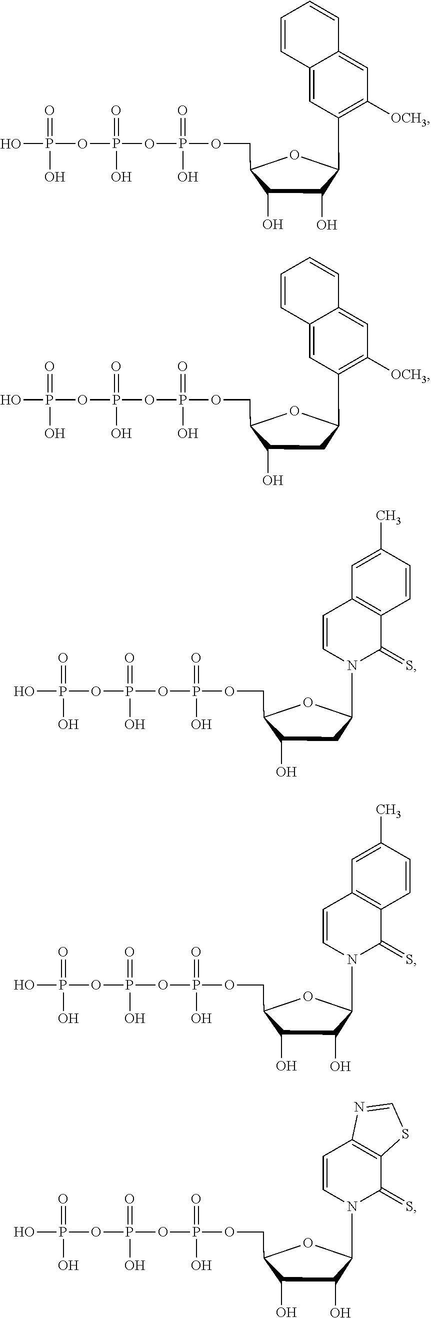

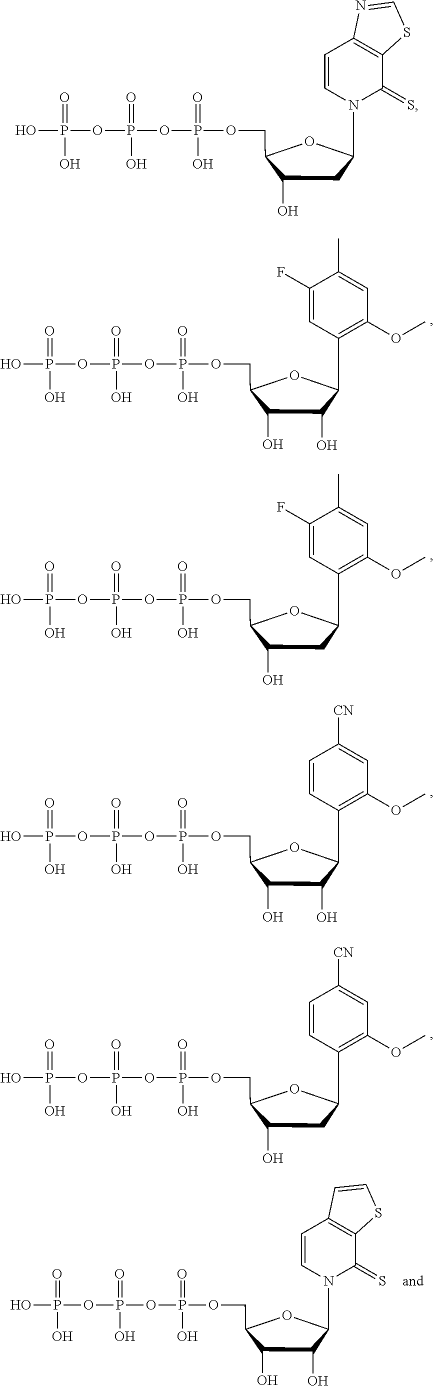

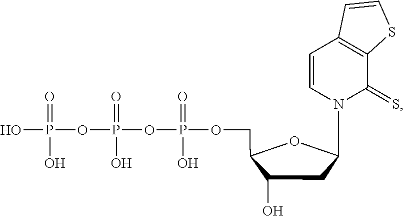 [SMILES: O=P(O)(O)OP(=O)(O)OP(=O)(O)OC[C@H]1O[C@@H](n2ccc3ccsc3c2=S)C[C@H]1O]

or salts thereof.

In some embodiments, an unnatural base pair generate an unnatural amino acid described in Dumas et al., “Designing logical codon reassignment—Expanding the chemistry in biology,” Chemical Science, 6: 50-69 (2015), the disclosure of which is incorporated herein by reference.

In some embodiments, the unnatural amino acid is incorporated into the cytokine (e.g., the IL polypeptide) by a synthetic codon comprising an unnatural nucleic acid. In some instances, the unnatural amino acid is incorporated into the cytokine by an orthogonal, modified synthetase/tRNA pair. Such orthogonal pairs comprise an unnatural synthetase that is capable of charging the unnatural tRNA with the unnatural amino acid, while minimizing charging of a) other endogenous amino acids onto the unnatural tRNA and b) unnatural amino acids onto other endogenous tRNAs. Such orthogonal pairs comprise tRNAs that are capable of being charged by the unnatural synthetase, while avoiding being charged with a) other endogenous amino acids by endogenous synthetases. In some embodiments, such pairs are identified from various organisms, such as bacteria, yeast, Archaea, or human sources. In some embodiments, an orthogonal synthetase/tRNA pair comprises components from a single organism. In some embodiments, an orthogonal synthetase/tRNA pair comprises components from two different organisms. In some embodiments, an orthogonal synthetase/tRNA pair comprising components that prior to modification, promote translation of two different amino acids. In some embodiments, an orthogonal synthetase is a modified alanine synthetase. In some embodiments, an orthogonal synthetase is a modified arginine synthetase. In some embodiments, an orthogonal synthetase is a modified asparagine synthetase. In some embodiments, an orthogonal synthetase is a modified aspartic acid synthetase. In some embodiments, an orthogonal synthetase is a modified cysteine synthetase. In some embodiments, an orthogonal synthetase is a modified glutamine synthetase. In some embodiments, an orthogonal synthetase is a modified glutamic acid synthetase. In some embodiments, an orthogonal synthetase is a modified alanine glycine. In some embodiments, an orthogonal synthetase is a modified histidine synthetase. In some embodiments, an orthogonal synthetase is a modified leucine synthetase. In some embodiments, an orthogonal synthetase is a modified isoleucine synthetase. In some embodiments, an orthogonal synthetase is a modified lysine synthetase. In some embodiments, an orthogonal synthetase is a modified methionine synthetase. In some embodiments, an orthogonal synthetase is a modified phenylalanine synthetase. In some embodiments, an orthogonal synthetase is a modified proline synthetase. In some embodiments, an orthogonal synthetase is a modified serine synthetase. In some embodiments, an orthogonal synthetase is a modified threonine synthetase. In some embodiments, an orthogonal synthetase is a modified tryptophan synthetase. In some embodiments, an orthogonal synthetase is a modified tyrosine synthetase. In some embodiments, an orthogonal synthetase is a modified valine synthetase. In some embodiments, an orthogonal synthetase is a modified phosphoserine synthetase. In some embodiments, an orthogonal tRNA is a modified alanine tRNA. In some embodiments, an orthogonal tRNA is a modified arginine tRNA. In some embodiments, an orthogonal tRNA is a modified asparagine tRNA. In some embodiments, an orthogonal tRNA is a modified aspartic acid tRNA. In some embodiments, an orthogonal tRNA is a modified cysteine tRNA. In some embodiments, an orthogonal tRNA is a modified glutamine tRNA. In some embodiments, an orthogonal tRNA is a modified glutamic acid tRNA. In some embodiments, an orthogonal tRNA is a modified alanine glycine. In some embodiments, an orthogonal tRNA is a modified histidine tRNA. In some embodiments, an orthogonal tRNA is a modified leucine tRNA. In some embodiments, an orthogonal tRNA is a modified isoleucine tRNA. In some embodiments, an orthogonal tRNA is a modified lysine tRNA. In some embodiments, an orthogonal tRNA is a modified methionine tRNA. In some embodiments, an orthogonal tRNA is a modified phenylalanine tRNA. In some embodiments, an orthogonal tRNA is a modified proline tRNA. In some embodiments, an orthogonal tRNA is a modified serine tRNA. In some embodiments, an orthogonal tRNA is a modified threonine tRNA. In some embodiments, an orthogonal tRNA is a modified tryptophan tRNA. In some embodiments, an orthogonal tRNA is a modified tyrosine tRNA. In some embodiments, an orthogonal tRNA is a modified valine tRNA. In some embodiments, an orthogonal tRNA is a modified phosphoserine tRNA.

In some embodiments, the unnatural amino acid is incorporated into the cytokine (e.g., the IL polypeptide) by an aminoacyl (aaRS or RS)-tRNA synthetase-tRNA pair. Exemplary aaRS-tRNA pairs include, but are not limited to, Methanococcus jannaschii (Mj-Tyr) aaRS/tRNA pairs, E. coli TyrRS (Ec-Tyr)/B. stearothermophilus tRNA_{CUA} pairs, E. coli LeuRS (Ec-Leu)/B. stearothermophilus tRNA_{CUA} pairs, and pyrrolysyl-tRNA pairs. In some instances, the unnatural amino acid is incorporated into the cytokine (e.g., the IL polypeptide) by a Mj-TyrRS/tRNA pair. Exemplary UAAs that can be incorporated by a Mj-TyrRS/tRNA pair include, but are not limited to, para-substituted phenylalanine derivatives such asp-aminophenylalanine and p-methoxyphenylalanine; meta-substituted tyrosine derivatives such as 3-aminotyrosine, 3-nitrotyrosine, 3,4-dihydroxyphenylalanine, and 3-iodotyrosine; phenylselenocysteine; p-boronophenylalanine; and o-nitrobenzyltyrosine.

In some instances, the unnatural amino acid is incorporated into the cytokine (e.g., the TL polypeptide) by a Ec-Tyr/tRNA_{CUA} or a Ec-Leu/tRNA_{CUA} pair. Exemplary UAAs that can be incorporated by a Ec-Tyr/tRNA_{CUA} or a Ec-Leu/tRNA_{CUA} pair include, but are not limited to, phenylalanine derivatives containing benzophenone, ketone, iodide, or azide substituents; O-propargyltyrosine; α-aminocaprylic acid, O-methyl tyrosine, O-nitrobenzyl cysteine; and 3-(naphthalene-2-ylamino)-2-amino-propanoic acid.

In some instances, the unnatural amino acid is incorporated into the cytokine (e.g., the IL polypeptide) by a pyrrolysyl-tRNA pair. In some cases, the PylRS is obtained from an archaebacterial, e.g., from a methanogenic archaebacterial. In some cases, the PylRS is obtained from Methanosarcina barkeri, Methanosarcina mazei, or Methanosarcina acetivorans. Exemplary UAAs that can be incorporated by a pyrrolysyl-tRNA pair include, but are not limited to, amide and carbamate substituted lysines such as 2-amino-6-((R)-tetrahydrofuran-2-carboxamido)hexanoic acid, N-ε-D-prolyl-L-lysine, and N-ε-cyclopentyloxycarbonyl-L-lysine; N-ε-Acryloyl-L-lysine; N-ε-[(1-(6-nitrobenzo[d][1,3]dioxol-5-yl)ethoxy)carbonyl]-L-lysine; and N-ε-(1-methylcyclopro-2-enecarboxamido)lysine. In some embodiments, the IL-10 conjugates disclosed herein may be prepared by use of M. mazei tRNA which is selectively charged with a non-natural amino acid such as N6-((2-azidoethoxy)-carbonyl)-L-lysine (AzK) by the M. barkeri pyrrolysyl-tRNA synthetase (Mb PylRS). Other methods are known to those of ordinary skill in the art, such as those disclosed in Zhang et al., Nature 2017, 551(7682): 644-647, the disclosure of which is incorporated herein by reference.

In some instances, an unnatural amino acid is incorporated into a cytokine described herein (e.g., the IL polypeptide) by a synthetase disclosed in U.S. Pat. Nos. 9,988,619 and 9,938,516, the disclosure of each of which is incorporated herein by reference.

The host cell into which the constructs or vectors disclosed herein are introduced is cultured or maintained in a suitable medium such that the tRNA, the tRNA synthetase and the protein of interest are produced. The medium also comprises the unnatural amino acid(s) such that the protein of interest incorporates the unnatural amino acid(s). In some embodiments, a nucleoside triphosphate transporter (NTT) from bacteria, plant, or algae is also present in the host cell. In some embodiments, the IL-10 conjugates disclosed herein are prepared by use of a host cell that expresses a NTT. In some embodiments, the nucleotide nucleoside triphosphate transporter used in the host cell may be selected from TpNTT1, TpNTT2, TpNTT3, TpNTT4, TpNTT5, TpNTT6, TpNTT7, TpNTT8 (T. pseudonana), PtNTT1, PtNTT2, PtNTT3, PtNTT4, PtNTT5, PtNTT6 (P. tricornutum), GsNTT (Galdieria sulphuraria), AtNTT1, AtNTT2 (Arabidopsis thaliana), CtNTT1, CtNTT2 (Chlamydia trachomatis), PamNTT1, PamNTT2 (Protochlamydia amoebophila), CcNTT (Caedibacter caryophilus), RpNTT1 (Rickettsia prowazekii). In some embodiments, the NTT is selected from PtNTT1, PtNTT2, PtNTT3, PtNTT4, PtNTT5, and PtNTT6. In some embodiments, the NTT is PtNTT1. In some embodiments, the NTT is PtNTT2. In some embodiments, the NTT is PtNTT3. In some embodiments, the NTT is PtNTT4. In some embodiments, the NTT is PtNTT5. In some embodiments, the NTT is PtNTT6. Other NTTs that may be used are disclosed in Zhang et al., Nature 2017, 551(7682): 644-647; Malyshev et al. Nature 2014 (509(7500), 385-388; and Zhang et al. Proc Natl Acad Sci USA, 2017, 114:1317-1322; the disclosure of each of which is incorporated herein by reference.

The orthogonal tRNA synthetase/tRNA pair charges a tRNA with an unnatural amino acid and incorporates the unnatural amino acid into the polypeptide chain in response to the codon. Exemplary aaRS-tRNA pairs include, but are not limited to, Methanococcus jannaschii (Mj-Tyr) aaRS/tRNA pairs, E. coli TyrRS (Ec-Tyr)/B. stearothermophilus tRNA_{CUA} pairs, E. coli LeuRS (Ec-Leu)/B. stearothermophilus tRNA_{CUA} pairs, and pyrrolysyl-tRNA pairs. Other aaRS-tRNA pairs that may be used according to the present disclosure include those derived from M. mazei those described in Feldman et al., J Am Chem Soc., 2018 140:1447-1454; and Zhang et al. Proc Natl Acad Sci USA, 2017, 114:1317-1322; the disclosure of each of which is incorporated herein by reference.

In some embodiments are provided methods of preparing the IL-10 conjugates disclosed herein in a cellular system that expresses a NTT and a tRNA synthetase. In some embodiments described herein, the NTT is selected from PtNTT1, PtNTT2, PtNTT3, PtNTT4, PtNTT5, and PtNTT6, and the tRNA synthetase is selected from Methanococcus jannaschii, E. coli TyrRS (Ec-Tyr)/B. stearothermophilus, and M. mazei. In some embodiments, the NTT is PtNTT1 and the tRNA synthetase is derived from Methanococcus jannaschii, E. coli TyrRS (Ec-Tyr)/B. stearothermophilus, or M. mazei. In some embodiments, the NTT is PtNTT2 and the tRNA synthetase is derived from Methanococcus jannaschii, E. coli TyrRS (Ec-Tyr)/B. stearothermophilus, or M. mazei. In some embodiments, the NTT is PtNTT3 and the tRNA synthetase is derived from Methanococcus jannaschii, E. coli TyrRS (Ec-Tyr)/B. stearothermophilus, or M. mazei. In some embodiments, the NTT is PtNTT3 and the tRNA synthetase is derived from Methanococcus jannaschii, E. coli TyrRS (Ec-Tyr)/B. stearothermophilus, or M. mazei. In some embodiments, the NTT is PtNTT4 and the tRNA synthetase is derived from Methanococcus jannaschii, E. coli TyrRS (Ec-Tyr)/B. stearothermophilus, or M. mazei. In some embodiments, the NTT is PtNTT5 and the tRNA synthetase is derived from Methanococcus jannaschii, E. coli TyrRS (Ec-Tyr)/B. stearothermophilus, or M. mazei. In some embodiments, the NTT is PtNTT6 and the tRNA synthetase is derived from Methanococcus jannaschii, E. coli TyrRS (Ec-Tyr)/B. stearothermophilus, or M. mazei.

In some embodiments, the NTTs as used herein is an NTT that is truncated at N-terminus, at C-terminus, or at both N and C-terminus. In some embodiments, the truncated NTT is at least 60%, at least 65%, at least 70%, at least 75%, at least 80%, at least 85%, or at least 90% identical the untruncated NTT. In some instances, the NTTs as used herein is PtNTT1, PtNTT2, PtNTT3, PtNTT4, PtNTT5, or PtNTT6. In some cases, the PtNTTs as used herein is truncated at N-terminus, at C-terminus, or at both N and C-terminus. In some embodiments, the truncated PtNTTs is at least 60%, at least 65%, at least 70%, at least 75%, at least 80%, at least 85%, or at least 90% identical the untruncated PtNTTs. In some cases, the NTT as used herein is a truncated PtNTT2, where the truncated PtNTT2 has an amino acid sequence that is at least 60%, at least 65%, at least 70%, at least 75%, at least 80%, at least 85%, or at least 90% identical to the amino acid sequence of untruncated PtNTT2. An example of untruncated PtNTT2 (NCBI accession number EEC49227.1, GI:217409295) has the amino acid sequence of SEQ ID NO: 74:

| | |
|---|---|
| 1 | MRPYPTIALI SVFLSAATRI SATSSHQASA LPVKKGTHVP |
| 41 | DSPKLSKLYI MAKTKSVSSS FDPPRGGSTV APTTPLATGG |
| 81 | ALRKVRQAVF PIYGNQEVTK FLLIGSIKFF IILALTLTRD |
| 121 | TKDTLIVTQC GAEAIAFLKI YGVLPAATAF IALYSKMSNA |
| 161 | MGKKMLFYST CIPFFTFFGL FDVFIYPNAE RLHPSLEAVQ |
| 201 | AILPGGAASG GMAVLAKIAT HWTSALFYVM AEIYSSVSVG |
| 241 | LLFWQFANDV VNVDQAKRFY PLFAQMSGLA PVLAGQYVVR |
| 281 | FASKAVNFEA SMHRLTAAVT FAGIMICIFY QLSSSYVERT |
| 321 | ESAKPAADNE QSIKPKKKKP KMSMVESGKF LASSQYLRLI |
| 361 | AMLVLGYGLS INFTEIMWKS LVKKQYPDPL DYQRFMGNFS |
| 401 | SAVGLSTCIV IFFGVHVIRL LGWKVGALAT PGIMAILALP |
| 441 | FFACILLGLD SPARLEIAVI FGTIQSLLSK TSKYALFDPT |
| 481 | TQMAYIPLDD ESKVKGKAAI DVLGSRIGKS GGSLIQQGLV |
| 521 | FVFGNIINAA PVVGVVYYSV LVAWMSAAGR LSGLFQAQTE |
| 561 | MDKADKMEAK TNKEK |

In some embodiments, the IL-10 conjugates disclosed herein may be prepared in a cell, such as E. coli, comprising (a) nucleotide triphosphate transporter PtNTT2 (including a truncated variant in which the first 65 amino acid residues of the full-length protein are deleted), (b) a plasmid comprising a double-stranded oligonucleotide that encodes an IL-10 variant having a desired amino acid sequence and that contains a unnatural base pair comprising a first unnatural nucleotide and a second unnatural nucleotide to provide a codon at the desired position at which an unnatural amino acid, such as N6-((2-azidoethoxy)-carbonyl)-L-lysine (AzK), N6-(propargylethoxy)-L-lysine (PraK), N6-(((2-azidobenzyl)oxy)carbonyl)-L-lysine, N6-(((3-azidobenzyl)oxy)carbonyl)-L-lysine, N6-(((4-azidobenzyl)oxy)carbonyl)-L-lysine, or N6-(((2-azidobenzyl)oxy)carbonyl)-L-lysine, N6-(((3-azidobenzyl)oxy)carbonyl)-L-lysine, or N6-(((4-azidobenzyl)oxy)carbonyl)-L-lysine, will be incorporated, (c) a plasmid encoding a tRNA derived from M. mazei and which comprises an unnatural nucleotide to provide a recognized anticodon (to the codon of the IL-10 variant) in place of its native sequence, and (d) a plasmid encoding a M. barkeri derived pyrrolysyl-tRNA synthetase (Mb PylRS), which may be the same plasmid that encodes the tRNA or a different plasmid. In some embodiments, the cell is further supplemented with deoxyribo triphosphates comprising one or more unnatural bases. In some embodiments, the cell is further supplemented with ribo triphosphates comprising one or more unnatural bases. In some embodiments, the cells is further supplemented with one or more unnatural amino acids, such as N6-((2-azidoethoxy)-carbonyl)-L-lysine (AzK) N6-(propargylethoxy)-L-lysine (PraK), N6-(((2-azidobenzyl)oxy)carbonyl)-L-lysine, N6-(((3-azidobenzyl)oxy)carbonyl)-L-lysine, N6-(((4-azidobenzyl)oxy)carbonyl)-L-lysine, or N6-(((2-azidobenzyl)oxy)carbonyl)-L-lysine, N6-(((3-azidobenzyl)oxy)carbonyl)-L-lysine, or N6-(((4-azidobenzyl)oxy)carbonyl)-L-lysine. In some embodiments, the double-stranded oligonucleotide that encodes the amino acid sequence of the desired IL-10 variant contains a codon AXC at, for example, position 67, 70, 74, 75, 79, 82, 88, 89, 99, 125, 126, 129, 130, or 132 of the sequence that encodes the protein having SEQ ID NO: 1, wherein X is an unnatural nucleotide such those disclosed herein, such NaM. In some embodiments, the cell further comprises a plasmid, which may be the protein expression plasmid or another plasmid, that encodes an orthogonal tRNA gene from M. mazei that comprises an AXC-matching anticodon GYT in place of its native sequence, wherein Y is an unnatural nucleotide as disclosed herein, such as TPT3, that is complementary and may be the same or different as the unnatural nucleotide in the codon. In some embodiments, the unnatural nucleotide in the codon is different than and complimentary to the unnatural nucleotide in the anti-codon. In some embodiments, the unnatural nucleotide in the codon is the same as the unnatural nucleotide in the anti-codon. In some embodiments, the first and second unnatural nucleotides comprising the unnatural base pair in the double-stranded oligonucleotide may be derived from

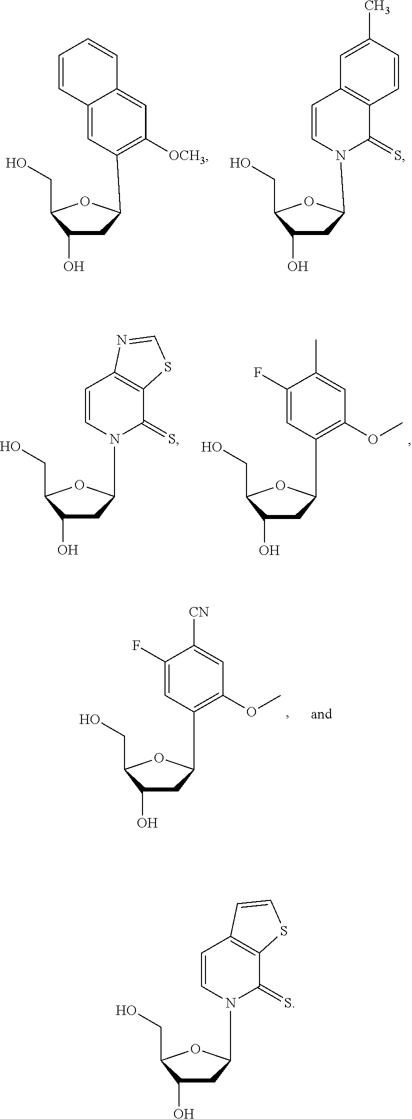

In some embodiments, the first and second unnatural nucleotides comprising the unnatural base pair in the double-stranded oligonucleotide may be derived from

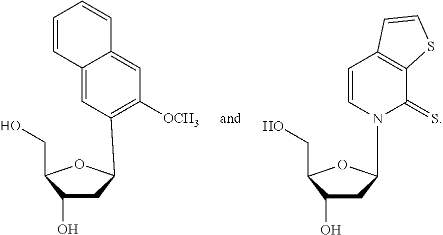 [SMILES: COc1cc2ccccc2cc1[C@H]1C[C@@H](O)[C@@H](CO)O1 ; OC[C@H]1O[C@@H](n2ccc3ccsc3c2=S)C[C@H]1O]

In some embodiments, the triphosphates of the first and second unnatural nucleotides include,

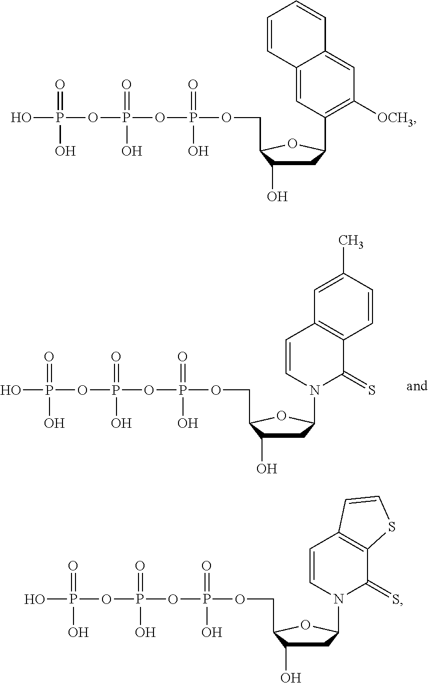

or salts thereof. In some embodiments, the triphosphates of the first and second unnatural nucleotides include,

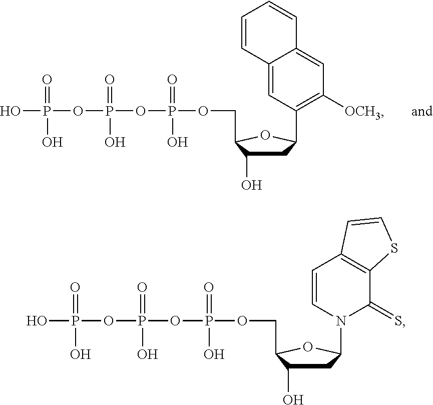

or salts thereof. In some embodiments, the mRNA derived the double-stranded oligonucleotide comprising a first unnatural nucleotide and a second unnatural nucleotide may comprise a codon comprising an unnatural nucleotide derived from

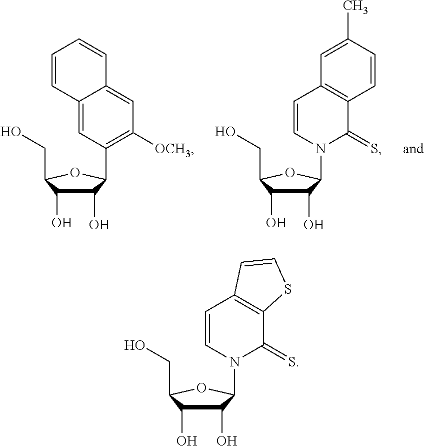

In some embodiments, the M. mazei tRNA may comprise an anti-codon comprising an unnatural nucleotide that recognizes the codon comprising the unnatural nucleotide of the mRNA. The anti-codon in the M. mazei tRNA may comprise an unnatural nucleotide derived from

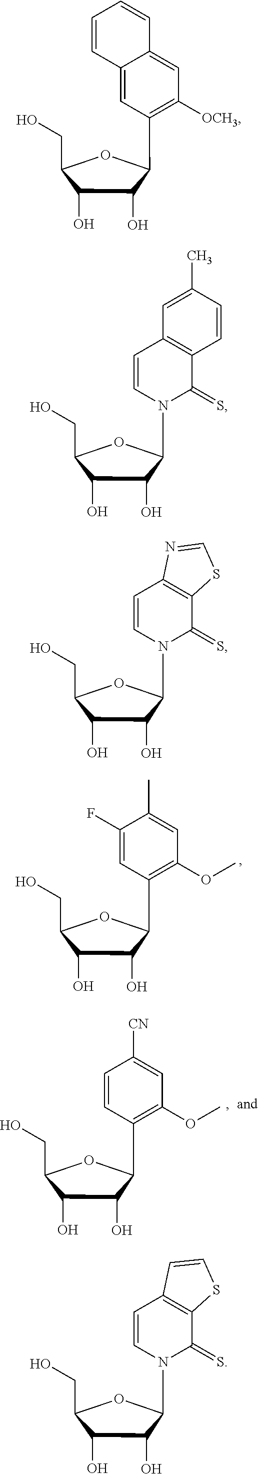

In some embodiments, the mRNA comprises an unnatural nucleotide derived from

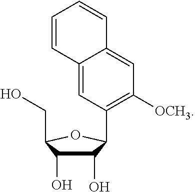

In some embodiments, the mRNA comprises an unnatural nucleotide derived from

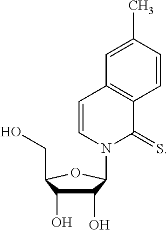

In some embodiments, the mRNA comprises an unnatural nucleotide derived from

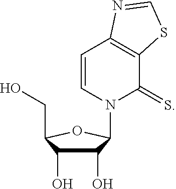

In some embodiments, the mRNA comprises an unnatural nucleotide derived from

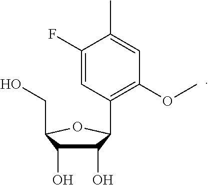

In some embodiments, the mRNA comprises an unnatural nucleotide derived from

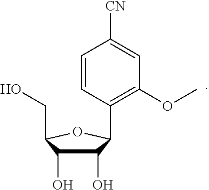

In some embodiments, the mRNA comprises an unnatural nucleotide derived from

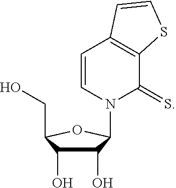

In some embodiments, the tRNA comprises an unnatural nucleotide derived from

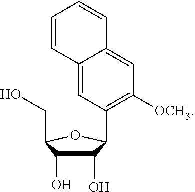

In some embodiments, the tRNA comprises an unnatural nucleotide derived from

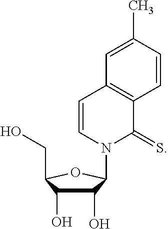

In some embodiments, the tRNA comprises an unnatural nucleotide derived from

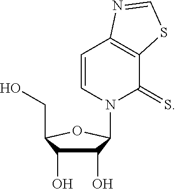

In some embodiments, the tRNA comprises an unnatural nucleotide derived from

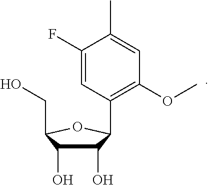

In some embodiments, the tRNA comprises an unnatural nucleotide derived from

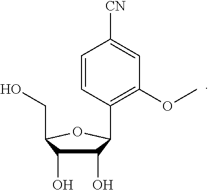

In some embodiments, the tRNA comprises an unnatural nucleotide derived from

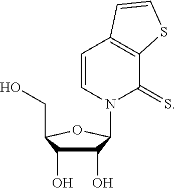

In some embodiments, the mRNA comprises an unnatural nucleotide derived from

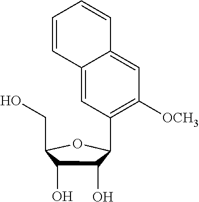

and the tRNA comprises an unnatural nucleotide derived from

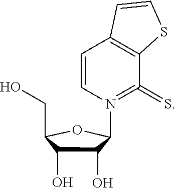

In some embodiments, the mRNA comprises an unnatural nucleotide derived from

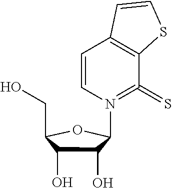

and the tRNA comprises an unnatural nucleotide derived from

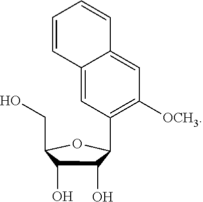

In some embodiments, the mRNA comprises an unnatural nucleotide derived from

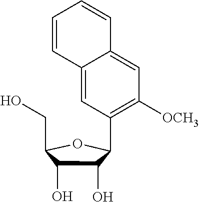

and the tRNA comprises an unnatural nucleotide derived from

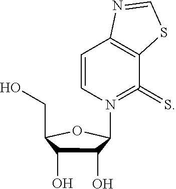

In some embodiments, the mRNA comprises an unnatural nucleotide derived from

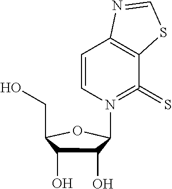

and the tRNA comprises an unnatural nucleotide derived from

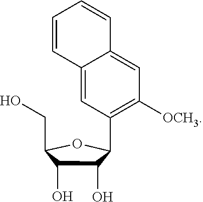

The host cell is cultured in a medium containing appropriate nutrients, and is supplemented with (a) the triphosphates of the deoxyribo nucleosides comprising one or more unnatural bases that are necessary for replication of the plasmid(s) encoding the cytokine gene harboring the codon, (b) the triphosphates of the ribo nucleosides comprising one or more unnatural bases necessary for transcription of (i) the mRNA corresponding to the coding sequence of the cytokine and containing the codon comprising one or more unnatural bases, and (ii) the tRNA containing the anticodon comprising one or more unnatural bases, and (c) the unnatural amino acid(s) to be incorporated in to the polypeptide sequence of the cytokine of interest. The host cells are then maintained under conditions which permit expression of the protein of interest.

The resulting protein comprising the one or more unnatural amino acids, AzK, N6-(propargylethoxy)-L-lysine (PraK), N6-(((2-azidobenzyl)oxy)carbonyl)-L-lysine, N6-(((3-azidobenzyl)oxy)carbonyl)-L-lysine, or N6-(((4-azidobenzyl)oxy)carbonyl)-L-lysine for example, that is expressed may be purified by methods known to those of ordinary skill in the art and may then be allowed to react with an alkyne, such as DBCO comprising a PEG chain having a desired average molecular weight as disclosed herein, under conditions known to those of ordinary skill in the art, to afford the IL-10 conjugates disclosed herein. Other methods are known to those of ordinary skill in the art, such as those disclosed in Zhang et al., Nature 2017, 551(7682): 644-647; WO 2015157555; WO 2015021432; WO 2016115168; WO 2017106767; WO 2017223528; WO 2019014262; WO 2019014267; WO 2019028419; and WO2019/028425; the disclosure of each of which is incorporated herein by reference.

Alternatively, a cytokine (e.g., IL-10) polypeptide comprising an unnatural amino acid(s) are prepared by introducing the nucleic acid constructs described herein comprising the tRNA and aminoacyl tRNA synthetase and comprising a nucleic acid sequence of interest with one or more in-frame orthogonal (stop) codons into a host cell. The host cell is cultured in a medium containing appropriate nutrients, is supplemented with (a) the triphosphates of the deoxyribo nucleosides comprising one or more unnatural bases required for replication of the plasmid(s) encoding the cytokine gene harboring the new codon and anticodon, (b) the triphosphates of the ribo nucleosides required for transcription of the mRNA corresponding to (i) the cytokine sequence containing the codon, and (ii) the orthogonal tRNA containing the anticodon, and (c) the unnatural amino acid(s). The host cells are then maintained under conditions which permit expression of the protein of interest. The unnatural amino acid(s) is incorporated into the polypeptide chain in response to the unnatural codon. For example, one or more unnatural amino acids are incorporated into the cytokine (e.g., IL-10) polypeptide. Alternatively, two or more unnatural amino acids may be incorporated into the cytokine (e.g., IL-10) polypeptide at two or more sites in the protein.

Once the cytokine (e.g., IL-10) polypeptide incorporating the unnatural amino acid(s) has been produced in the host cell it can be extracted therefrom by a variety of techniques known in the art, including enzymatic, chemical and/or osmotic lysis and physical disruption. The cytokine (e.g., IL-10) polypeptide can be purified by standard techniques known in the art such as preparative ion exchange chromatography, hydrophobic chromatography, affinity chromatography, or any other suitable technique known to those of ordinary skill in the art.

In some embodiments, the IL-10 conjugates disclosed herein may be prepared in a cell, such as E. coli, comprising (a) nucleotide triphosphate transporter PtNTT2 (including a truncated variant in which the first 65 amino acid residues of the full-length protein are deleted), (b) a plasmid comprising a double-stranded oligonucleotide that encodes an IL-10 variant having a desired amino acid sequence and that contains a unnatural base pair comprising a first unnatural nucleotide and a second unnatural nucleotide to provide a codon at the desired position at which an unnatural amino acid, such as N6-((2-azidoethoxy)-carbonyl)-L-lysine (AzK), will be incorporated, (c) a plasmid encoding a tRNA derived from M. mazei and which comprises an unnatural nucleotide to provide a recognized anticodon (to the codon of the IL-10 variant) in place of its native sequence, and (d) a plasmid encoding a M. barkeri derived pyrrolysyl-tRNA synthetase (Mb PylRS), which may be the same plasmid that encodes the tRNA or a different plasmid. In some embodiments, the cell is further supplemented with deoxyribo triphosphates comprising one or more unnatural bases. In some embodiments, the cell is further supplemented with ribo triphosphates comprising one or more unnatural bases. In some embodiments, the cells is further supplemented with one or more unnatural amino acids, such as N6-((2-azidoethoxy)-carbonyl)-L-lysine (AzK). In some embodiments, the double-stranded oligonucleotide that encodes the amino acid sequence of the desired IL-10 variant contains a codon AXC at, for example, position N82, K88, A89, K99, K125, N126, N129, or K130 of the sequence that encodes the protein having SEQ ID NO: 1, wherein X is an unnatural nucleotide.

In some embodiments, the cell further comprises a plasmid, which may be the protein expression plasmid or another plasmid, that encodes an orthogonal tRNA gene from M. mazei that comprises an AXC-matching anticodon GYT in place of its native sequence, wherein Y is an unnatural nucleotide that is complementary and may be the same or different as the unnatural nucleotide in the codon. In some embodiments, the unnatural nucleotide in the codon is different than and complimentary to the unnatural nucleotide in the anti-codon. In some embodiments, the unnatural nucleotide in the codon is the same as the unnatural nucleotide in the anti-codon. In some embodiments, the unnatural nucleotides comprising the unnatural base pair in the double-stranded oligonucleotide may be derived from

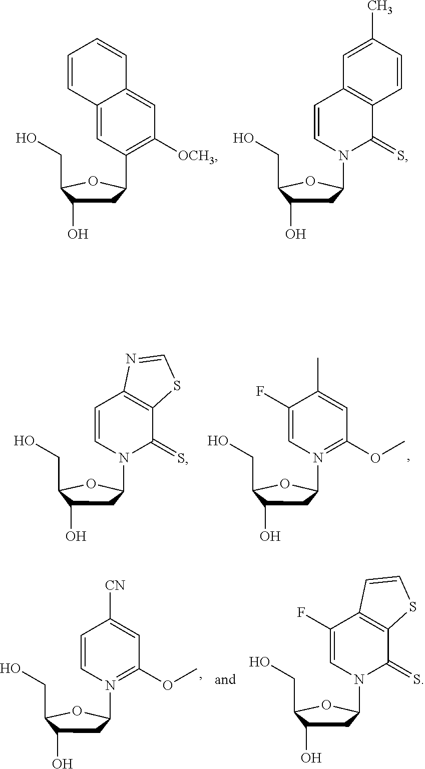

In some embodiments, the first and second unnatural nucleotides comprising the unnatural base pair in the double-stranded oligonucleotide may be derived from

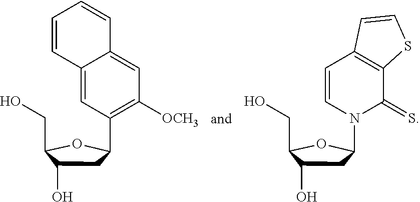

In some embodiments, the first and second unnatural nucleotides comprising the unnatural base pair in the double-stranded oligonucleotide may be derived from

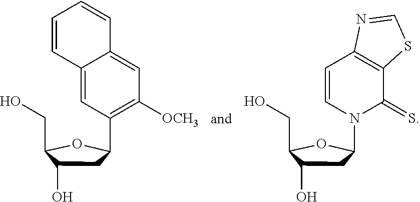

In some embodiments, the triphosphates of the first and second unnatural nucleotides include,

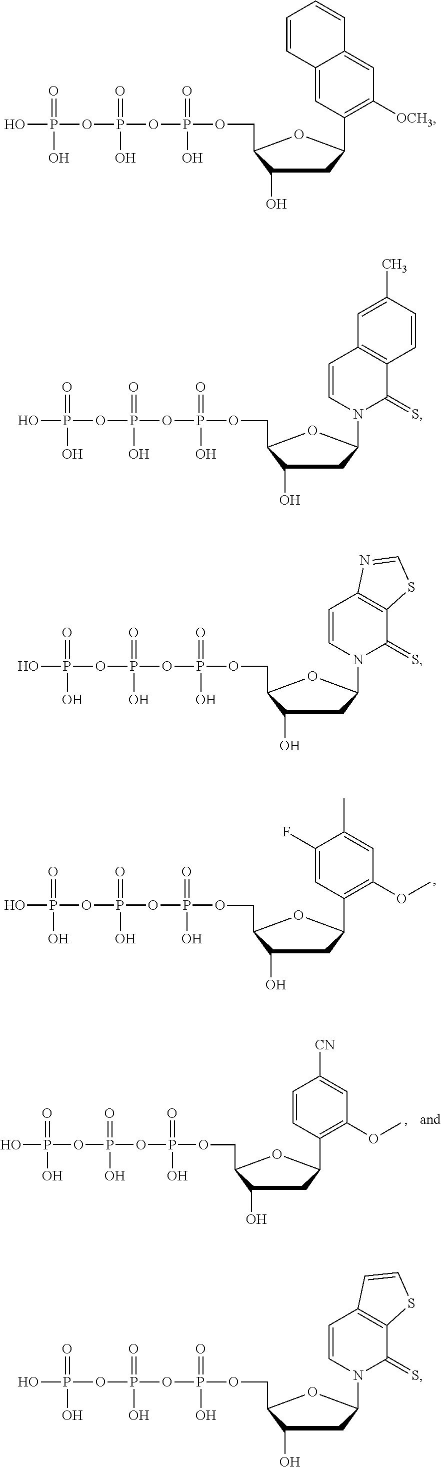 [SMILES: COc1cc2ccccc2cc1[C@H]1C[C@@H](O)[C@@H](COP(=O)(O)OP(=O)(O)OP(=O)(O)O)O1 ; Cc1ccc2c(=S)n([C@H]3C[C@@H](O)[C@@H](COP(=O)(O)OP(=O)(O)OP(=O)(O)O)O3)ccc2c1 ; O=P(O)(O)OP(=O)(O)OP(=O)(O)OC[C@H]1O[C@@H](n2ccc3ncsc3c2=S)C[C@H]1O ; COc1cc(C)c(F)cc1[C@H]1C[C@@H](O)[C@@H](COP(=O)(O)OP(=O)(O)OP(=O)(O)O)O1 ; COc1cc(C#N)ccc1[C@H]1C[C@@H](O)[C@@H](COP(=O)(O)OP(=O)(O)OP(=O)(O)O)O1 ; O=P(O)(O)OP(=O)(O)OP(=O)(O)OC[C@H]1O[C@@H](n2ccc3ccsc3c2=S)C[C@H]1O]

or salts thereof. In some embodiments, the triphosphates of the first and second unnatural nucleotides include,

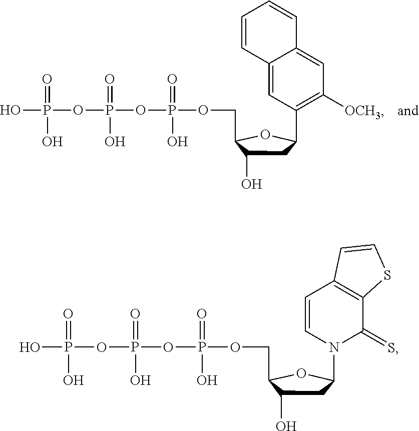

or salts thereof. In some embodiments, the triphosphates of the first and second unnatural nucleotides include,

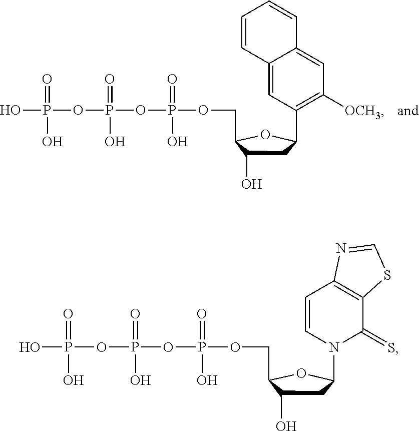

or salts thereof. In some embodiments, the mRNA derived the double-stranded oligonucleotide comprising a first unnatural nucleotide and a second unnatural nucleotide may comprise a codon comprising an unnatural nucleotide derived from

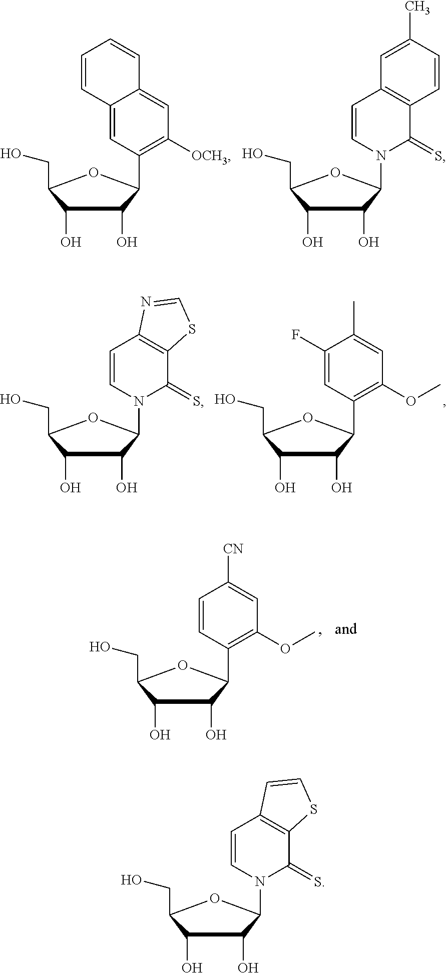

In some embodiments, the M. mazei tRNA may comprise an anti-codon comprising an unnatural nucleotide that recognizes the codon comprising the unnatural nucleotide of the mRNA. The anti-codon in the M. mazei tRNA may comprise an unnatural nucleotide derived from

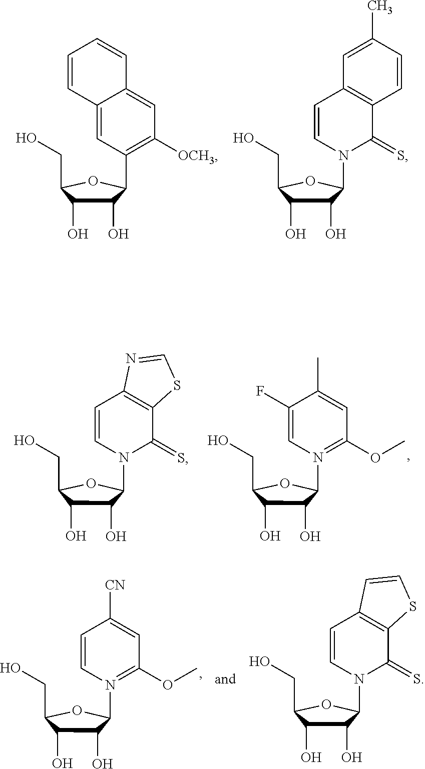

In some embodiments, the mRNA comprises an unnatural nucleotide derived from

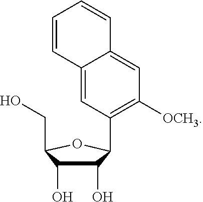

In some embodiments, the mRNA comprises an unnatural nucleotide derived from

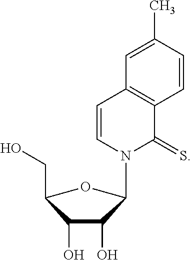

In some embodiments, the mRNA comprises an unnatural nucleotide derived from

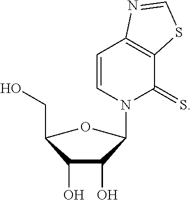

In some embodiments, the mRNA comprises an unnatural nucleotide derived from

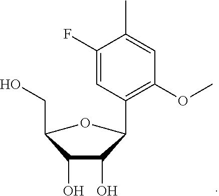

In some embodiments, the mRNA comprises an unnatural nucleotide derived from

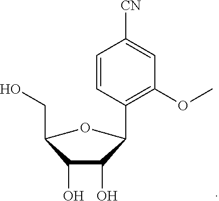

In some embodiments, the mRNA comprises an unnatural nucleotide derived from

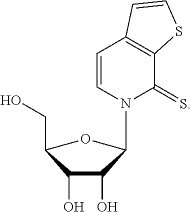

In some embodiments, the tRNA comprises an unnatural nucleotide derived from

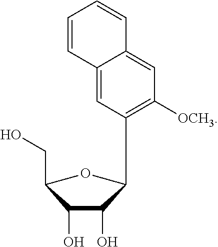

In some embodiments, the tRNA comprises an unnatural nucleotide derived from

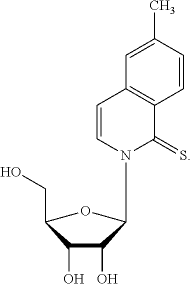

In some embodiments, the tRNA comprises an unnatural nucleotide derived from

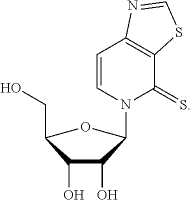

In some embodiments, the tRNA comprises an unnatural nucleotide derived from

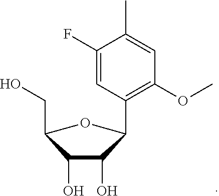

In some embodiments, the tRNA comprises an unnatural nucleotide derived from

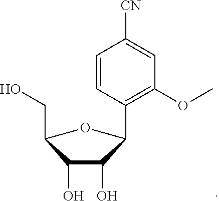

In some embodiments, the tRNA comprises an unnatural nucleotide derived from

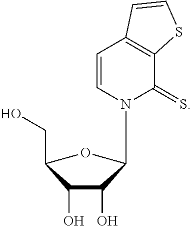

In some embodiments, the mRNA comprises an unnatural nucleotide derived from

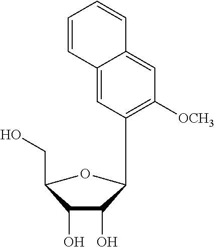

and the tRNA comprises an unnatural nucleotide derived from

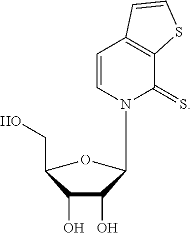

In some embodiments, the mRNA comprises an unnatural nucleotide derived from

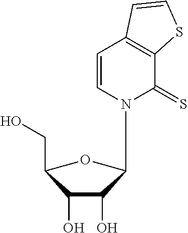

and the tRNA comprises an unnatural nucleotide derived from

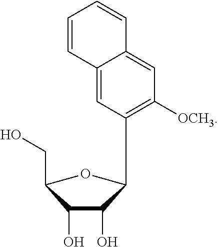

In some embodiments, the mRNA comprises an unnatural nucleotide derived from

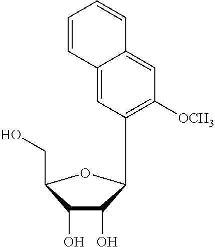

and the tRNA comprises an unnatural nucleotide derived from

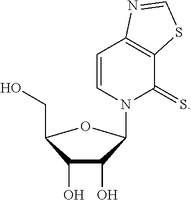

In some embodiments, the mRNA comprises an unnatural nucleotide derived from

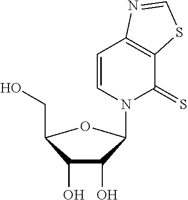

and the tRNA comprises an unnatural nucleotide derived from

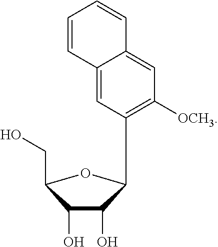

The host cell is cultured in a medium containing appropriate nutrients, and is supplemented with (a) the triphosphates of the deoxyribo nucleosides comprising one or more unnatural bases that are necessary for replication of the plasmid(s) encoding the cytokine gene harboring the codon, (b) the triphosphates of the ribo nucleosides comprising one or more unnatural bases necessary for transcription of (i) the mRNA corresponding to the coding sequence of the cytokine and containing the codon comprising one or more unnatural bases, and (ii) the tRNA containing the anticodon comprising one or more unnatural bases, and (c) the unnatural amino acid(s) to be incorporated in to the polypeptide sequence of the cytokine of interest. The host cells are then maintained under conditions which permit expression of the protein of interest.

In some cases, the codon comprising an unnatural base and the anticodon comprising an unnatural base may be selected from the following pairs, wherein X and Y each comprise a base independently selected from the group consisting of:

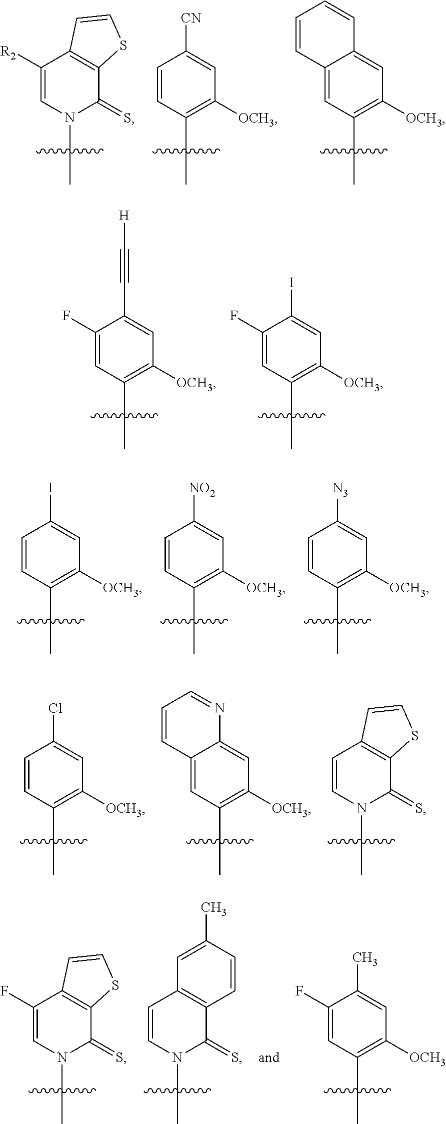

wherein R₂ is selected from the group consisting of hydrogen, alkyl, alkenyl, alkynyl, methoxy, methanethiol, methaneseleno, halogen, cyano, and azido; and in each case the wavy line indicates a bond to a ribosyl when X and Y comprise mRNA or tRNA, or 2′-deoxyribosyl when X and Y comprise DNA (Table 2).

TABLE 2
Listing of Non-Limiting Examples of Codons
and Anticodons Comprising X and Y.

| Codon (mRNA) | Anticodon (tRNA) |
|---|---|
| UUX | YAA or XAA |
| UGX | YCA or XCA |
| CGX | YCG or XCG |
| AGX | YCU or XCU |
| GAX | YUC or XUC |
| CAX | YUG or XUG |
| GXU | AYC |
| CXU | AYG |
| GXG | CYC |
| AXG | CYU |
| GXC | GYC |
| AXC | GYU |
| GXA | UYC |
| CXC | GYG |
| UXC | GYA |
| AUX | YAU or XAU |
| CUX | XAG or YAG |
| GUX | XAC or YAC |
| UAX | XUA or YUA |
| GGX | XCC or YCC |

The resulting protein comprising the one or more unnatural amino acids, Azk for example, that is expressed may be purified by methods known to those of ordinary skill in the art and may then be allowed to react with an alkyne, such as DBCO comprising a PEG chain having a desired average molecular weight as disclosed herein, under conditions known to those of ordinary skill in the art, to afford the IL-10 conjugates disclosed herein. Other methods are known to those of ordinary skill in the art, such as those disclosed in Zhang et al., Nature 2017, 551(7682): 644-647; WO 2015157555; WO 2015021432; WO 2016115168; WO 2017106767; WO 2017223528; WO 2019014262; WO 2019014267; WO 2019028419; and WO2019/028425; the disclosure of each of which is incorporated herein by reference.

Alternatively, a cytokine (e.g., IL-10) polypeptide comprising an unnatural amino acid(s) are prepared by introducing the nucleic acid constructs described herein comprising the tRNA and aminoacyl tRNA synthetase and comprising a nucleic acid sequence of interest with one or more in-frame orthogonal (stop) codons into a host cell. The host cell is cultured in a medium containing appropriate nutrients, is supplemented with (a) the triphosphates of the deoxyribo nucleosides comprising one or more unnatural bases required for replication of the plasmid(s) encoding the cytokine gene harboring the new codon and anticodon, (b) the triphosphates of the ribo nucleosides required for transcription of the mRNA corresponding to (i) the cytokine sequence containing the codon, and (ii) the orthogonal tRNA containing the anticodon, and (c) the unnatural amino acid(s). The host cells are then maintained under conditions which permit expression of the protein of interest. The unnatural amino acid(s) is incorporated into the polypeptide chain in response to the unnatural codon. For example, one or more unnatural amino acids are incorporated into the cytokine (e.g., IL-10) polypeptide. Alternatively, two or more unnatural amino acids may be incorporated into the cytokine (e.g., IL-10) polypeptide at two or more sites in the protein.

Once the cytokine (e.g., IL-10) polypeptide incorporating the unnatural amino acid(s) has been produced in the host cell it can be extracted therefrom by a variety of techniques known in the art, including enzymatic, chemical and/or osmotic lysis and physical disruption. The cytokine (e.g., IL-10) polypeptide can be purified by standard techniques known in the art such as preparative ion exchange chromatography, hydrophobic chromatography, affinity chromatography, or any other suitable technique known to those of ordinary skill in the art.

Suitable host cells may include bacterial cells (e.g., E. coli, BL21(DE3)), but most suitably host cells are eukaryotic cells, for example insect cells (e.g. Drosophila such as Drosophila melanogaster), yeast cells, nematodes (e.g. C. elegans), mice (e.g. Mus musculus), or mammalian cells (such as Chinese hamster ovary cells (CHO) or COS cells, human 293T cells, HeLa cells, NIH 3T3 cells, and mouse erythroleukemia (MEL) cells) or human cells or other eukaryotic cells. Other suitable host cells are known to those skilled in the art. Suitably, the host cell is a mammalian cell—such as a human cell or an insect cell. In some embodiments, the suitable host cells comprise E co/i.

Other suitable host cells which may be used generally in the embodiments of the invention are those mentioned in the examples section. Vector DNA can be introduced into host cells via conventional transformation or transfection techniques. As used herein, the terms “transformation” and “transfection” are intended to refer to a variety of well-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells are well known in the art.

When creating cell lines, it is generally preferred that stable cell lines are prepared. For stable transfection of mammalian cells for example, it is known that, depending upon the expression vector and transfection technique used, only a small fraction of cells may integrate the foreign DNA into their genome. In order to identify and select these integrants, a gene that encodes a selectable marker (for example, for resistance to antibiotics) is generally introduced into the host cells along with the gene of interest. Preferred selectable markers include those that confer resistance to drugs, such as G418, hygromycin, or methotrexate. Nucleic acid molecules encoding a selectable marker can be introduced into a host cell on the same vector or can be introduced on a separate vector. Cells stably transfected with the introduced nucleic acid molecule can be identified by drug selection (for example, cells that have incorporated the selectable marker gene will survive, while the other cells die).

In one embodiment, the constructs described herein are integrated into the genome of the host cell. An advantage of stable integration is that the uniformity between individual cells or clones is achieved. Another advantage is that selection of the best producers may be carried out. Accordingly, it is desirable to create stable cell lines. In another embodiment, the constructs described herein are transfected into a host cell. An advantage of transfecting the constructs into the host cell is that protein yields may be maximized. In one aspect, there is described a cell comprising the nucleic acid construct or the vector described herein.

### Methods of Use

Described herein, in some embodiments, is a method of treating cancer in a subject, comprising administering to a subject in need thereof an effective amount of any one of modified IL-10 polypeptide or IL-10 conjugates as described herein. In some cases, the cancer is a solid tumor or a liquid tumor. In some cases, the solid tumor is a metastatic cancer. In some cases, the cancer is a relapsed or refractory cancer from a prior treatment. In some embodiments, the cancer being treated as described herein is selected from renal cell carcinoma, bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, squamous cell carcinoma, pancreatic cancer, and prostate cancer. In some embodiments, the cancer being treated as described herein is selected from renal cell carcinoma (RCC), non-small cell lung cancer (NSCLC), head and neck squamous cell cancer (HNSCC), classical Hodgkin lymphoma (cHL), primary mediastinal large B-cell lymphoma (PMBCL), urothelial carcinoma, microsatellite unstable cancer, microsatellite stable cancer, microsatellite-stable colorectal cancer, gastric cancer, cervical cancer, hepatocellular carcinoma (HCC), Merkel cell carcinoma (MCC), melanoma, small cell lung cancer (SCLC), esophageal, glioblastoma, mesothelioma, breast cancer, triple-negative breast cancer, prostate cancer, bladder cancer, ovarian cancer, tumors of moderate to low mutational burden, cutaneous squamous cell carcinoma (CSCC), squamous cell skin cancer (SCSC), tumors of low- to non-expressing PD-L1, tumors disseminated systemically to the liver and CNS beyond their primary anatomic originating site, and diffuse large B-cell lymphoma.

In some embodiments, the cancer being treated as described herein is a hematologic malignancy. In some cases, the hematologic malignancy comprises a leukemia, a lymphoma, or a myeloma. In some embodiments, the hematologic malignancy is a T-cell malignancy. In some embodiments, the hematological malignancy is a B-cell malignancy. In some embodiments, the hematologic malignancy is a metastatic hematologic malignancy. In some embodiments, the hematologic malignancy is a relapsed hematologic malignancy. In some embodiments, the hematologic malignancy is a refractory hematologic malignancy. In some cases, the cancer being treated by any one of modified IL-10 polypeptide or IL-10 conjugate is a cancer selected from chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

### Proliferative Diseases or Conditions

In some embodiments, described herein is a method of treating a proliferative disease or condition in a subject in need thereof, which comprises administering to the subject a therapeutically effective amount of any one of modified IL-10 polypeptides or IL-10 conjugates described herein. In some embodiments, the proliferative disease or condition is a cancer. In some cases, the cancer is a solid tumor. Exemplary solid tumors include, but are not limited to, bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, or prostate cancer. In some cases, the solid tumor is a metastatic cancer. In some cases, the solid tumor is a relapsed or refractory cancer from a prior treatment.

In some instances, any one of modified IL-10 polypeptide or IL-10 conjugates described herein is administered to a subject in need thereof, for treating a solid tumor. In such cases, the subject has a bladder cancer, a bone cancer, a brain cancer, a breast cancer, a colorectal cancer, an esophageal cancer, an eye cancer, a head and neck cancer, a kidney cancer (or renal cell carcinoma), a lung cancer, a melanoma, an ovarian cancer, a pancreatic cancer, or a prostate cancer. In some cases, the IL-10 conjugate is administered to a subject for the treatment of a bladder cancer. In some cases, the IL-10 conjugate is administered to a subject for the treatment of a breast cancer. In some cases, the IL-10 conjugate is administered to a subject for the treatment of a colorectal cancer. In some cases, the IL-10 conjugate is administered to a subject for the treatment of an esophageal cancer. In some cases, the IL-10 conjugate is administered to a subject for the treatment of a head and neck cancer. In some cases, the IL-10 conjugate is administered to a subject for the treatment of a kidney cancer (or renal cell carcinoma or RCC). In some cases, the IL-10 conjugate is administered to a subject for the treatment of a lung cancer. In some cases, the IL-10 conjugate is administered to a subject for the treatment of a melanoma. In some cases, the IL-10 conjugate is administered to a subject for the treatment of an ovarian cancer. In some cases, the IL-10 conjugate is administered to a subject for the treatment of a pancreatic cancer. In some cases, the IL-10 conjugate is administered to a subject for the treatment of a prostate cancer. In some instances, the cancer is a metastatic cancer. In other instances, the cancer is a relapsed cancer. In additional cases, the cancer is a refractory cancer.

In some embodiments, the cancer is a treatment-naïve cancer. In such cases, the treatment-naive cancer is a cancer that has not been treated by a therapy. In some cases, the treatment-naive cancer is a solid tumor, such as bladder cancer, a bone cancer, a brain cancer, a breast cancer, a colorectal cancer, an esophageal cancer, an eye cancer, a head and neck cancer, a kidney cancer (or RCC), a lung cancer, a melanoma, an ovarian cancer, a pancreatic cancer, or a prostate cancer. In some embodiments, described herein is a method of treating a treatment-naive solid tumor in a subject in need thereof which comprises administering to the subject an IL-10 conjugate described herein.

In some embodiments, the cancer is a hematologic malignancy. In some instances, an IL-10 conjugate described herein is administered to a subject in need thereof, for treating a hematologic malignancy. In some instances, the hematologic malignancy comprises a leukemia, a lymphoma, or a myeloma. In some cases, the hematologic malignancy is a T-cell malignancy. In other cases, the hematological malignancy is a B-cell malignancy. In some instances, the hematologic malignancy is a metastatic hematologic malignancy. In other instances, the hematologic malignancy is a relapsed hematologic malignancy. In additional cases, the hematologic malignancy is a refractory hematologic malignancy. In some cases, the subject has a T-cell malignancy. In some cases, the subject has a B-cell malignancy. In some cases, the subject has chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some cases, the IL-10 conjugate is administered to a subject for the treatment of CLL. In some cases, the IL-10 conjugate is administered to a subject for the treatment of SLL. In some cases, the IL-10 conjugate is administered to a subject for the treatment of FL. In some cases, the IL-10 conjugate is administered to a subject for the treatment of DLBCL. In some cases, the IL-10 conjugate is administered to a subject for the treatment of MCL. In some cases, the IL-10 conjugate is administered to a subject for the treatment of Waldenstrom's macroglobulinemia. In some cases, the IL-10 conjugate is administered to a subject for the treatment of multiple myeloma. In some cases, the IL-10 conjugate is administered to a subject for the treatment of Burkitt's lymphoma.

### Additional Therapeutic Agents

In some embodiments, an additional therapeutic agent is further administered to the subject. In some cases, the additional therapeutic agent is administered simultaneously with an IL-10 conjugate and/or is co-formulated. In other cases, the additional therapeutic agent and the IL-10 conjugate are administered sequentially, e.g., the IL-10 conjugate is administered prior to the additional therapeutic agent or that the IL-10 conjugate is administered after administration of the additional therapeutic agent. In some cases, the one or more additional agents is one or more immune checkpoint inhibitors selected from the group consisting of PD-1 inhibitors, PD-L1 inhibitors, PD-L2 inhibitors, CTLA-4 inhibitors, OX40 agonists, and 4-1BB agonists. In some cases, the one or more immune checkpoint inhibitors is selected from PD-1 inhibitors. Exemplary PD-1 inhibitors include pembrolizumab, nivolumab, cemiplimab, lambrolizumab, AMP-224, sintilimab, toripalimab, camrelizumab, tislelizumab, dostarlimab (GSK), PDR001 (Novartis), MGA012 (Macrogenics/Incyte), GLS-010 (Arcus/Wuxi), AGEN2024 (Agenus), cetrelimab (Janssen), ABBV-181 (Abbvie), AMG-404 (Amgen). BI-754091 (Boehringer Ingelheim), CC-90006 (Celgene), JTX-4014 (Jounce), PF-06801591 (Pfizer), and genolimzumab (Apollomics/Genor BioPharma). In some cases, the one or more immune checkpoint inhibitors is selected from PD-L1 inhibitors. Exemplary PD-L1 inhibitors include atezolizumab, avelumab, durvalumab, ASC22 (Alphamab/Ascletis), CX-072 (Cytomx), CS1001 (Cstone), cosibelimab (Checkpoint Therapeutics), INCB86550 (Incyte), and TG-1501 (TG Therapeutics). In some cases, the one or more immune checkpoint inhibitors is selected from CTLA-4 inhibitors. In some embodiments, CTLA-4 inhibitors is selected from tremelimumab, ipilimumab, and AGEN-1884 (Agenus). In some cases, the one or more additional agents comprises folinic acid, 5-fluorouracil, and oxaliplatin for treating pancreatic cancer and pancreatic ductal adenocarcinoma (PDAC).

In some cases, the additional therapeutic agent comprises a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

In some cases, the additional therapeutic agent comprises a first-line therapy. As used herein, “first-line therapy” comprises a primary treatment for a subject with a cancer. In some instances, the cancer is a primary or local cancer. In other instances, the cancer is a metastatic or recurrent cancer. In some cases, the first-line therapy comprises chemotherapy. In other cases, the first-line treatment comprises immunotherapy, targeted therapy, or radiation therapy. A skilled artisan would readily understand that different first-line treatments may be applicable to different type of cancers.

In some cases, an IL-10 conjugate is administered with an additional therapeutic agent selected from an alkylating agent such as altretamine, busulfan, carboplatin, carmustine, chlorambucil, cisplatin, cyclophosphamide, dacarbazine, lomustine, melphalan, oxalaplatin, temozolomide, or thiotepa; an antimetabolite such as 5-fluorouracil (5-FU), 6-mercaptopurine (6-MP), capecitabine, cytarabine, floxuridine, fludarabine, gemcitabine, hydroxyurea, methotrexate, or pemetrexed; an anthracycline such as daunorubicin, doxorubicin, epirubicin, or idarubicin; a topoisomerase I inhibitor such as topotecan or irinotecan (CPT-11); a topoisomerase II inhibitor such as etoposide (VP-16), teniposide, or mitoxantrone; a mitotic inhibitor such as docetaxel, estramustine, ixabepilone, paclitaxel, vinblastine, vincristine, or vinorelbine; or a corticosteroid such as prednisone, methylprednisolone, or dexamethasone.

In some instances, an IL-10 conjugate described herein is administered with an inhibitor of the enzyme poly ADP ribose polymerase (PARP). Exemplary PARP inhibitors include, but are not limited to, olaparib (AZD-2281, Lynparza®, from Astra Zeneca), rucaparib (PF-01367338, Rubraca®, from Clovis Oncology), niraparib (MK-4827, Zejula®, from Tesaro), talazoparib (BMN-673, from BioMarin Pharmaceutical Inc.), veliparib (ABT-888, from AbbVie), CK-102 (formerly CEP 9722, from Teva Pharmaceutical Industries Ltd.), E7016 (from Eisai), iniparib (BSI 201, from Sanofi), and pamiparib (BGB-290, from BeiGene). In some cases, the IL-10 conjugate is administered in combination with a PARP inhibitor such as olaparib, rucaparib, niraparib, talazoparib, veliparib, CK-102, E7016, iniparib, or pamiparib.

In some embodiments, an IL-10 conjugate described herein is administered with a tyrosine kinase inhibitor (TKI). Exemplary TKIs include, but are not limited to, afatinib, alectinib, axitinib, bosutinib, cabozantinib, ceritinib, cobimetinib, crizotinib, dabrafenib, dasatinib, erlotinib, gefitinib, ibrutinib, imatinib, lapatinib, lenvatinib, nilotinib, nintedanib, osimertinib, pazopanib, ponatinib, regorafenib, ruxolitinib, sorafenib, sunitinib, tofacitinib, and vandetanib.

In some instances, an IL-10 conjugate described herein is administered with an immune checkpoint inhibitor. Exemplary checkpoint inhibitors include: PD-L1 inhibitors such as durvalumab (Imfinzi) from AstraZeneca, atezolizumab (MPDL3280A) from Genentech, avelumab from EMD Serono/Pfizer, CX-072 from CytomX Therapeutics, FAZ053 from Novartis Pharmaceuticals, KN035 from 3D Medicine/Alphamab, LY3300054 from Eli Lilly, or M7824 (anti-PD-L1/TGFbeta trap) from EMD Serono; PD-L2 inhibitors such as GlaxoSmithKline's AMP-224 (Amplimmune), and rHIgM12B7; PD-1 inhibitors such as nivolumab (Opdivo) from Bristol-Myers Squibb, pembrolizumab (Keytruda) from Merck, AGEN 2034 from Agenus, BGB-A317 from BeiGene, Bl-754091 from Boehringer-Ingelheim Pharmaceuticals, CBT-501 (genolimzumab) from CBT Pharmaceuticals, INCSHR1210 from Incyte, JNJ-63723283 from Janssen Research & Development, MEDI0680 from MedImmune, MGA 012 from MacroGenics, PDR001 from Novartis Pharmaceuticals, PF-06801591 from Pfizer, REGN2810 (SAR439684) from Regeneron Pharmaceuticals/Sanofi, or TSR-042 from TESARO; CTLA-4 inhibitors such as ipilimumab (also known as Yervoy®, MDX-010, BMS-734016 and MDX-101) from Bristol Meyers Squibb, tremelimumab (CP-675,206, ticilimumab) from Pfizer, or AGEN 1884 from Agenus; LAG3 inhibitors such as BMS-986016 from Bristol-Myers Squibb, IMP701 from Novartis Pharmaceuticals, LAG525 from Novartis Pharmaceuticals, or REGN3767 from Regeneron Pharmaceuticals; B7-H3 inhibitors such as enoblituzumab (MGA271) from MacroGenics; KIR inhibitors such as Lirilumab (IPH2101; BMS-986015) from Innate Pharma; CD137 inhibitors such as urelumab (BMS-663513, Bristol-Myers Squibb), PF-05082566 (anti-4-1BB, PF-2566, Pfizer), or XmAb-5592 (Xencor); PS inhibitors such as Bavituximab; and inhibitors such as an antibody or fragments (e.g., a monoclonal antibody, a human, humanized, or chimeric antibody) thereof, RNAi molecules, or small molecules to TIM3, CD52, CD30, CD20, CD33, CD27, OX40, GITR, ICOS, BTLA (CD272), CD160, 2B4, LAIR1, TIGHT, LIGHT, DR3, CD226, CD2, or SLAM.

In some embodiments, the PD-1 inhibitor is pembrolizumab, nivolumab, or cemiplimab. In some embodiments, the PD-1 inhibitor is pembrolizumab. In some embodiments, the PD-1 inhibitor is nivolumab. In some embodiments, the PD-1 inhibitor is cemiplimab.

In some embodiments, the PD-L1 inhibitor is atezolizumab. In some embodiments, the PD-L1 inhibitor is avelumab. In some embodiments, the PD-L1 inhibitor is durvalumab.

In some embodiments, the CTLA-4 inhibitors are selected from tremelimumab, ipilimumab, and AGEN-1884 (Agenus). In some embodiments, the CTLA-4 inhibitor is tremelimumab. In some embodiments, the CTLA-4 inhibitor is ipilimumab. In some instances, the IL-10 conjugate is administered in combination with pembrolizumab, nivolumab, tremelimumab, or ipilimumab.

In some instances, an IL-10 conjugate described herein is administered with an antibody such as alemtuzumab, trastuzumab, ibritumomab tiuxetan, brentuximab vedotin, ado-trastuzumab emtansine, or blinatumomab.

In some instances, an IL-10 conjugate is administered with an additional therapeutic agent selected from an additional cytokine. In some instances, the additional cytokine enhances and/or synergizes T effector cell expansion and/or proliferation. In some cases, the additional cytokine comprises IL-1β, IL-2, IL-6, IL-7, IL-12, IL-15, IL-18, IL-21, or TNFα. In some cases, the additional cytokine is IL-7. In some cases, the additional cytokine is IL-15. In some cases, the additional cytokine is IL-21. In some cases, the additional cytokine is TNFα.

In some instances, an IL-10 conjugate is administered with an additional therapeutic agent selected from a receptor agonist. In some instances, the receptor agonist comprises a Toll-like receptor (TLR) ligand. In some cases, the TLR ligand comprises TLR1, TLR2, TLR3, TLR4, TLR5, TLR6, TLR7, TLR8, or TLR9. In some cases, the TLR ligand comprises a synthetic ligand such as, for example, Pam3Cys, CFA, MALP2, Pam2Cys, FSL-1, Hib-OMPC, Poly I:C, poly A:U, AGP, MPL A, RC-529, MDF2β, CFA, or Flagellin. In some cases, the IL-10 conjugate is administered with one or more TLR agonists selected from TLR1, TLR2, TLR3, TLR4, TLR5, TLR6, TLR7, TLR8, and TLR9. In some cases, the IL-10 conjugate is administered with one or more TLR agonists selected from Pam3Cys, CFA, MALP2, Pam2Cys, FSL-1, Hib-OMPC, Poly I:C, poly A:U, AGP, MPL A, RC-529, MDF2β, CFA, and Flagellin.

In some embodiments, an IL-10 conjugate is used in conjunction with an adoptive T cell transfer (ACT) therapy. In one embodiment, ACT involves identification of autologous T lymphocytes in a subject with, e.g., anti-tumor activity, expansion of the autologous T lymphocytes in vitro, and subsequent reinfusion of the expanded T lymphocytes into the subject. In another embodiment, ACT comprises use of allogeneic T lymphocytes with, e.g., anti-tumor activity, expansion of the T lymphocytes in vitro, and subsequent infusion of the expanded allogeneic T lymphocytes into a subject in need thereof. In some instances, an IL-10 conjugate described herein is used in conjunction with autologous T lymphocytes as part of an ACT therapy. In other instances, an IL-10 conjugate described herein is used in conjunction with allogeneic T lymphocytes as part of an ACT therapy. In some cases, the IL-10 conjugate is administered simultaneously with the ACT therapy to a subject in need thereof. In other cases, the IL-10 conjugate is administered sequentially with the ACT therapy to a subject in need thereof.

In some embodiments, an IL-10 conjugate is used for an ex vivo activation and/or expansion of an autologous and/or allogenic T cell transfer. In such cases, the IL-10 conjugate is used to activate and/or expand a sample comprising autologous and/or allogenic T cells and the IL-10 conjugate is optionally removed from the sample prior to administering the sample to a subject in need thereof.

In some embodiments, an IL-10 conjugate is administered with a vaccine. In some instances, an IL-10 conjugate is utilized in combination with an oncolytic virus. In such cases, the IL-10 conjugate acts as a stimulatory agent to modulate the immune response. In some instances, the IL-10 conjugate is used with an oncolytic virus as part of an adjuvant therapy. Exemplary oncolytic viruses include T-Vec (Amgen), G47A (Todo et al.), JX-594 (Sillajen), CG0070 (Cold Genesys), and Reolysin (Oncolytics Biotech). In some cases, the IL-10 conjugate is used in combination with an oncolytic virus such as T-Vec, G47A, JX-594, CG0070, or Reolysin.

In some embodiments, an IL-10 conjugate is administered in combination with a radiation therapy.

### Methods of Treating Other Diseases

Described herein, in some embodiments, is a method of treating a fibrotic disorder in a subject by administering any one of the modified IL-10 polypeptides or IL-10 conjugates described herein. In some cases, the fibrotic disorder can include liver fibrosis, idiopathic pulmonary fibrosis, and periportal fibrosis. Described herein, in some embodiments, is a method of treating non-alcoholic steatohepatitis (NASH) in a subject by administering any one of the modified IL-10 polypeptides or IL-10 conjugates described herein. Described herein, in some embodiments, is a method of treating nonalcoholic fatty liver disease (NAFLD) in a subject by administering any one of the modified IL-10 polypeptides or IL-10 conjugates described herein.

### Pharmaceutical Compositions and Formulations

In some embodiments, the pharmaceutical composition and formulations described herein are administered to a subject by multiple administration routes, including but not limited to, parenteral, oral, or transdermal administration routes. In some cases, parenteral administration comprises intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration.

In some embodiments, the pharmaceutical formulations include, but are not limited to, aqueous liquid dispersions, self-emulsifying dispersions, liposomal dispersions, aerosols, immediate release formulations, controlled release formulations, delayed release formulations, extended release formulations, pulsatile release formulations, and mixed immediate and controlled release formulations.

In some embodiments, the pharmaceutical formulations include a carrier or carrier materials selected on the basis of compatibility with the composition disclosed herein, and the release profile properties of the desired dosage form. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pa. 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999); the disclosure of each of which is incorporated herein by reference.

In some cases, the pharmaceutical composition is formulated as an immunoliposome, which comprises a plurality of IL-10 conjugates bound either directly or indirectly to lipid bilayer of liposomes. Exemplary lipids include, but are not limited to, fatty acids; phospholipids; sterols such as cholesterols; sphingolipids such as sphingomyelin; glycosphingolipids such as gangliosides, globosides, and cerebrosides; surfactant amines such as stearyl, oleyl, and linoleyl amines. In some instances, the lipid comprises a cationic lipid. In some instances, the lipid comprises a phospholipid. Exemplary phospholipids include, but are not limited to, phosphatidic acid (“PA”), phosphatidylcholine (“PC”), phosphatidylglycerol (“PG”), phophatidylethanolamine (“PE”), phophatidylinositol (“PI”), and phosphatidylserine (“PS”), sphingomyelin (including brain sphingomyelin), lecithin, lysolecithin, lysophosphatidylethanolamine, cerebrosides, diarachidoylphosphatidylcholine (“DAPC”), didecanoyl-L-alpha-phosphatidylcholine (“DDPC”), dielaidoylphosphatidylcholine (“DEPC”), dilauroylphosphatidylcholine (“DLPC”), dilinoleoylphosphatidylcholine, dimyristoylphosphatidylcholine (“DMPC”), dioleoylphosphatidylcholine (“DOPC”), dipalmitoylphosphatidylcholine (“DPPC”), distearoylphosphatidylcholine (“DSPC”), 1-palmitoyl-2-oleoyl-phosphatidylcholine (“POPC”), diarachidoylphosphatidylglycerol (“DAPG”), didecanoyl-L-alpha-phosphatidylglycerol (“DDPG”), dielaidoylphosphatidylglycerol (“DEPG”), dilauroylphosphatidylglycerol (“DLPG”), dilinoleoylphosphatidylglycerol, dimyristoylphosphatidylglycerol (“DMPG”), dioleoylphosphatidylglycerol (“DOPG”), dipalmitoylphosphatidylglycerol (“DPPG”), distearoylphosphatidylglycerol (“DSPG”), 1-palmitoyl-2-oleoyl-phosphatidylglycerol (“POPG”), diarachidoylphosphatidylethanolamine (“DAPE”), didecanoyl-L-alpha-phosphatidylethanolamine (“DDPE”), dielaidoylphosphatidylethanolamine (“DEPE”), dilauroylphosphatidylethanolamine (“DLPE”), dilinoleoylphosphatidylethanolamine, dimyristoylphosphatidylethanolamine (“DMPE”), dioleoylphosphatidylethanolamine (“DOPE”), dipalmitoylphosphatidylethanolamine (“DPPE”), distearoylphosphatidylethanolamine (“DSPE”), 1-palmitoyl-2-oleoyl-phosphatidylethanolamine (“POPE”), diarachidoylphosphatidylinositol (“DAPI”), didecanoyl-L-alpha-phosphatidylinositol (“DDPI”), dielaidoylphosphatidylinositol (“DEPI”), dilauroylphosphatidylinositol (“DLPI”), dilinoleoylphosphatidylinositol, dimyristoylphosphatidylinositol (“DMPI”), dioleoylphosphatidylinositol (“DOPI”), dipalmitoylphosphatidylinositol (“DPPI”), distearoylphosphatidylinositol (“DSPI”), 1-palmitoyl-2-oleoyl-phosphatidylinositol (“POPI”), diarachidoylphosphatidylserine (“DAPS”), didecanoyl-L-alpha-phosphatidylserine (“DDPS”), dielaidoylphosphatidylserine (“DEPS”), dilauroylphosphatidylserine (“DLPS”), dilinoleoylphosphatidylserine, dimyristoylphosphatidylserine (“DMPS”), dioleoylphosphatidylserine (“DOPS”), dipalmitoylphosphatidylserine (“DPPS”), distearoylphosphatidylserine (“DSPS”), 1-palmitoyl-2-oleoyl-phosphatidylserine (“POPS”), diarachidoyl sphingomyelin, didecanoyl sphingomyelin, dielaidoyl sphingomyelin, dilauroyl sphingomyelin, dilinoleoyl sphingomyelin, dimyristoyl sphingomyelin, sphingomyelin, dioleoyl sphingomyelin, dipalmitoyl sphingomyelin, distearoyl sphingomyelin, and 1-palmitoyl-2-oleoyl-sphingomyelin.

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, sorbitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20 or Tween® 20, or trometamol.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil, and polyoxyethylene alkylethers and alkylphenyl ethers, e.g., octoxynol 10, octoxynol 40. Sometimes, surfactants are included to enhance physical stability or for other purposes.

### Therapeutic Regimens

In some embodiments, the pharmaceutical compositions described herein are administered for therapeutic applications. In some embodiments, the pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously, alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

In some embodiments, an effective amount of the IL-10 conjugate is administered to a subject in need thereof once per week, once every two weeks, once every three weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, once every 20 weeks, once every 21 weeks, once every 22 weeks, once every 23 weeks, once every 24 weeks, once every 25 weeks, once every 26 weeks, once every 27 weeks, or once every 28 weeks. In some embodiments, an effective amount of the IL-10 conjugate is administered to a subject in need thereof once per week. In some embodiments, an effective amount of the IL-10 conjugate is administered to a subject in need thereof once every two weeks. In some embodiments, an effective amount of the IL-10 conjugate is administered to a subject in need thereof once every three weeks. In some embodiments, an effective amount of the IL-10 conjugate is administered to a subject in need thereof once every 4 weeks. In some embodiments, an effective amount of the IL-10 conjugate is administered to a subject in need thereof once every 5 weeks. In some embodiments, an effective amount of the IL-10 conjugate is administered to a subject in need thereof once every 6 weeks. In some embodiments, an effective amount of the IL-10 conjugate is administered to a subject in need thereof once every 7 weeks. In some embodiments, an effective amount of the IL-10 conjugate is administered to a subject in need thereof once every 8 weeks. In some embodiments, an effective amount of the IL-10 conjugate is administered to a subject in need thereof once every 9 weeks. In some embodiments, an effective amount of the IL-10 conjugate is administered to a subject in need thereof once every 10 weeks. In some embodiments, an effective amount of the IL-10 conjugate is administered to a subject in need thereof once every 11 weeks. In some embodiments, an effective amount of the IL-10 conjugate is administered to a subject in need thereof once every 12 weeks. In some embodiments, an effective amount of the IL-10 conjugate is administered to a subject in need thereof once every 13 weeks. In some embodiments, an effective amount of the IL-10 conjugate is administered to a subject in need thereof once every 14 weeks. In some embodiments, an effective amount of the IL-10 conjugate is administered to a subject in need thereof once every 15 weeks. In some embodiments, an effective amount of the IL-10 conjugate is administered to a subject in need thereof once every 16 weeks. In some embodiments, an effective amount of the IL-10 conjugate is administered to a subject in need thereof once every 17 weeks. In some embodiments, an effective amount of the IL-10 conjugate is administered to a subject in need thereof once every 18 weeks. In some embodiments, an effective amount of the IL-10 conjugate is administered to a subject in need thereof once every 19 weeks. In some embodiments, an effective amount of the IL-10 conjugate is administered to a subject in need thereof once every 20 weeks. In some embodiments, an effective amount of the IL-10 conjugate is administered to a subject in need thereof once every 21 weeks. In some embodiments, an effective amount of the IL-10 conjugate is administered to a subject in need thereof once every 22 weeks. In some embodiments, an effective amount of the IL-10 conjugate is administered to a subject in need thereof once every 23 weeks. In some embodiments, an effective amount of the IL-10 conjugate is administered to a subject in need thereof once every 24 weeks.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

In some embodiments, the methods include the dosing of an IL-10 conjugate to a subject in need thereof at a dose in the range from 1 μg of the IL-10 conjugate per kg of the subject's body weight to about 200 μg of the IL-10 conjugate per kg of the subject's body weight, or from about 2 μg of the IL-10 conjugate per kg of the subject's body weight to about 200 μg of the IL-10 conjugate per kg of the subject's body weight, or from about 4 μg of the IL-10 conjugate per kg of the subject's body weight to about 200 μg of the IL-10 conjugate per kg of the subject's body weight, or from about 6 μg of the IL-10 conjugate per kg of the subject's body weight to about 200 μg of the IL-10 conjugate per kg of the subject's body weight, or from about 8 μg of the IL-10 conjugate per kg of the subject's body weight to about 200 μg of the IL-10 conjugate per kg of the subject's body weight, or from about 10 μg of the IL-10 conjugate per kg of the subject's body weight to about 200 μg of the IL-10 conjugate per kg of the subject's body weight, or from about 12 μg of the IL-10 conjugate per kg of the subject's body weight to about 200 μg of the IL-10 conjugate per kg of the subject's body weight, or from about 14 μg of the IL-10 conjugate per kg of the subject's body weight to about 200 μg of the IL-10 conjugate per kg of the subject's body weight, or from about 16 μg of the IL-10 conjugate per kg of the subject's body weight to about 200 μg of the IL-10 conjugate per kg of the subject's body weight, or from about 18 μg of the IL-10 conjugate per kg of the subject's body weight to about 200 μg of the IL-10 conjugate per kg of the subject's body weight, or from about 20 μg of the IL-10 conjugate per kg of the subject's body weight to about 200 μg of the IL-10 conjugate per kg of the subject's body weight, or from about 22 μg of the IL-10 conjugate per kg of the subject's body weight to about 200 μg of the IL-10 conjugate per kg of the subject's body weight, or from about 24 μg of the IL-10 conjugate per kg of the subject's body weight to about 200 μg of the IL-10 conjugate per kg of the subject's body weight, or from about 26 μg of the IL-10 conjugate per kg of the subject's body weight to about 200 μg of the IL-10 conjugate per kg of the subject's body weight, or from about 28 μg of the IL-10 conjugate per kg of the subject's body weight to about 200 μg of the IL-10 conjugate per kg of the subject's body weight, or from about 32 μg of the IL-10 conjugate per kg of the subject's body weight to about 200 μg of the IL-10 conjugate per kg of the subject's body weight, or from about 34 μg of the IL-10 conjugate per kg of the subject's body weight to about 200 μg of the IL-10 conjugate per kg of the subject's body weight, or from about 36 μg of the IL-10 conjugate per kg of the subject's body weight to about 200 μg of the IL-10 conjugate per kg of the subject's body weight, or from about 40 μg of the IL-10 conjugate per kg of the subject's body weight to about 200 μg of the IL-10 conjugate per kg of the subject's body weight, or from about 45 μg of the IL-10 conjugate per kg of the subject's body weight to about 200 μg of the IL-10 conjugate per kg of the subject's body weight, or from about 50 μg of the IL-10 conjugate per kg of the subject's body weight to about 200 μg of the IL-10 conjugate per kg of the subject's body weight, or from about 55 μg of the IL-10 conjugate per kg of the subject's body weight to about 200 μg of the IL-10 conjugate per kg of the subject's body weight, or from about 60 μg of the IL-10 conjugate per kg of the subject's body weight to about 200 μg of the IL-10 conjugate per kg of the subject's body weight, or from about 65 μg of the IL-10 conjugate per kg of the subject's body weight to about 200 μg of the IL-10 conjugate per kg of the subject's body weight, or from about 70 μg of the IL-10 conjugate per kg of the subject's body weight to about 200 μg of the IL-10 conjugate per kg of the subject's body weight, or from about 75 μg of the IL-10 conjugate per kg of the subject's body weight to about 200 μg of the IL-10 conjugate per kg of the subject's body weight, or from about 80 μg of the IL-10 conjugate per kg of the subject's body weight to about 200 μg of the IL-10 conjugate per kg of the subject's body weight, or from about 85 μg of the IL-10 conjugate per kg of the subject's body weight to about 200 μg of the IL-10 conjugate per kg of the subject's body weight, or from about 90 μg of the IL-10 conjugate per kg of the subject's body weight to about 200 μg of the IL-10 conjugate per kg of the subject's body weight, or from about 95 μg of the IL-10 conjugate per kg of the subject's body weight to about 200 μg of the IL-10 conjugate per kg of the subject's body weight, or from about 100 μg of the IL-10 conjugate per kg of the subject's body weight to about 200 μg of the IL-10 conjugate per kg of the subject's body weight, or from about 110 μg of the IL-10 conjugate per kg of the subject's body weight to about 200 μg of the IL-10 conjugate per kg of the subject's body weight, or from about 120 μg of the IL-10 conjugate per kg of the subject's body weight to about 200 μg of the IL-10 conjugate per kg of the subject's body weight, or from about 130 μg of the IL-10 conjugate per kg of the subject's body weight to about 200 μg of the IL-10 conjugate per kg of the subject's body weight, or from about 140 μg of the IL-10 conjugate per kg of the subject's body weight to about 200 μg of the IL-10 conjugate per kg of the subject's body weight, or from about 150 μg of the IL-10 conjugate per kg of the subject's body weight to about 200 μg of the IL-10 conjugate per kg of the subject's body weight, or from about 160 μg of the IL-10 conjugate per kg of the subject's body weight to about 200 μg of the IL-10 conjugate per kg of the subject's body weight, or from about 170 μg of the IL-10 conjugate per kg of the subject's body weight to about 200 μg of the IL-10 conjugate per kg of the subject's body weight, or from about 180 μg of the IL-10 conjugate per kg of the subject's body weight to about 200 μg of the IL-10 conjugate per kg of the subject's body weight, or from about 190 μg of the IL-10 conjugate per kg of the subject's body weight to about 200 μg of the IL-10 conjugate per kg of the subject's body weight. The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, the methods include the dosing of an IL-10 conjugate to a subject in need thereof at a dose of about 1 μg of the IL-10 conjugate per kg of the subject's body weight, or about 2 μg of the IL-10 conjugate per kg of the subject's body weight, about 4 μg of the IL-10 conjugate per kg of the subject's body weight, about 6 μg of the IL-10 conjugate per kg of the subject's body weight, about 8 μg of the IL-10 conjugate per kg of the subject's body weight, about 10 μg of the IL-10 conjugate per kg of the subject's body weight, about 12 μg of the IL-10 conjugate per kg of the subject's body weight, about 14 μg of the IL-10 conjugate per kg of the subject's body weight, about 16 μg of the IL-10 conjugate per kg of the subject's body weight, about 18 μg of the IL-10 conjugate per kg of the subject's body weight, about 20 μg of the IL-10 conjugate per kg of the subject's body weight, about 22 μg of the IL-10 conjugate per kg of the subject's body weight, about 24 μg of the IL-10 conjugate per kg of the subject's body weight, about 26 μg of the IL-10 conjugate per kg of the subject's body weight, about 28 μg of the IL-10 conjugate per kg of the subject's body weight, about 30 μg of the IL-10 conjugate per kg of the subject's body weight, about 32 μg of the IL-10 conjugate per kg of the subject's body weight, about 34 μg of the IL-10 conjugate per kg of the subject's body weight, about 36 μg of the IL-10 conjugate per kg of the subject's body weight, about 38 μg of the IL-10 conjugate per kg of the subject's body weight, about 40 μg of the IL-10 conjugate per kg of the subject's body weight, about 42 μg of the IL-10 conjugate per kg of the subject's body weight, about 44 μg of the IL-10 conjugate per kg of the subject's body weight, about 46 μg of the IL-10 conjugate per kg of the subject's body weight, about 48 μg of the IL-10 conjugate per kg of the subject's body weight, about 50 μg of the IL-10 conjugate per kg of the subject's body weight, about 55 μg of the IL-10 conjugate per kg of the subject's body weight, about 60 μg of the IL-10 conjugate per kg of the subject's body weight, about 65 μg of the IL-10 conjugate per kg of the subject's body weight, about 70 μg of the IL-10 conjugate per kg of the subject's body weight, about 75 μg of the IL-10 conjugate per kg of the subject's body weight, about 80 μg of the IL-10 conjugate per kg of the subject's body weight, about 85 μg of the IL-10 conjugate per kg of the subject's body weight, about 90 μg of the IL-10 conjugate per kg of the subject's body weight, about 95 μg of the IL-10 conjugate per kg of the subject's body weight, about 100 μg of the IL-10 conjugate per kg of the subject's body weight, about 110 μg of the IL-10 conjugate per kg of the subject's body weight, about 120 μg of the IL-10 conjugate per kg of the subject's body weight, about 130 μg of the IL-10 conjugate per kg of the subject's body weight, about 140 μg of the IL-10 conjugate per kg of the subject's body weight, about 150 μg of the IL-10 conjugate per kg of the subject's body weight, about 160 μg of the IL-10 conjugate per kg of the subject's body weight, about 170 μg of the IL-10 conjugate per kg of the subject's body weight, about 180 μg of the IL-10 conjugate per kg of the subject's body weight, about 190 μg of the IL-10 conjugate per kg of the subject's body weight, or about 200 μg of the IL-10 conjugate per kg of the subject's body weight. The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner. In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

In some embodiments, the dosage can be at least partially determined by occurrence or severity of grade 3 or grade 4 adverse events in the subject. Non-limiting examples of adverse events include hypothermia; shock; bradycardia; ventricular extrasystoles; myocardial ischemia; syncope; hemorrhage; atrial arrhythmia; phlebitis; atrioventricular (AV) block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; acute tubular necrosis; duodenal ulceration; bowel necrosis; myocarditis; supraventricular tachycardia; permanent or transient blindness secondary to optic neuritis; transient ischemic attacks; meningitis; cerebral edema; pericarditis; allergic interstitial nephritis; tracheo-esophageal fistula; malignant hyperthermia; cardiac arrest; myocardial infarction; pulmonary emboli; stroke; liver or renal failure; severe depression leading to suicide; pulmonary edema; respiratory arrest; respiratory failure; leukopenia, thrombocytopenia, increased alanine aminotransferase (ALT), anorexia, arthralgia, back pain, chills, diarrhea, dyslipidemia, fatigue, fever, flu-like symptoms, hypoalbuminemia, increased lipase, injection site reaction, myalgia, nausea, night sweats, pruritis, rash, erythematous rash, maculopapular rash, transaminitis, vomiting, and weakness.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain embodiments, are kits and articles of manufacture for use with one or more methods and compositions described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include one or more IL-10 polypeptides or conjugates disclosed herein, and optionally one or more pharmaceutical excipients described herein to facilitate the delivery of the IL-10 polypeptides or conjugates. Such kits further optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, compositions containing a compound provided herein formulated in a compatible pharmaceutical carrier are also prepared, placed in an appropriate container, and labeled for treatment of an indicated condition.

In some embodiments, the kits comprise articles of manufacture that are useful for developing adoptive cell therapies. In some embodiments, kits comprise one or more of the cytokine (e.g., IL-10) polypeptides or cytokine (e.g., IL-10) conjugates disclosed herein, and optionally one or more pharmaceutical excipients described herein to facilitate the delivery of cytokine (e.g., IL-10) polypeptides or cytokine (e.g., IL-10) conjugates. Such kits might optionally include one or more accessory components comprising inducers of tumor infiltration lymphocytes (TILs), T cells, B cells, natural killer cells, macrophages, neutrophils, dendritic cells, mast cells, eosinophils basophils, or CD4+ or CD8+ T cells. Such kits further optionally include an identifying description or label or instructions relating to its use in the methods described herein. In some embodiments, kits comprise one or more polynucleic acid sequences encoding the IL-10 conjugates disclosed herein, an activator of tumor infiltration lymphocytes (TILs), T cells, B cells, natural killer cells, macrophages, neutrophils, dendritic cells, mast cells, eosinophils basophils, or CD4+ or CD8+ T cells and/or a pharmaceutical composition thereof.

In some embodiments, the kits and articles described herein comprise a modified IL-10 polypeptide comprising at least one unnatural amino acid. In some embodiments, the at least one unnatural amino acid: is a lysine analogue; comprises an aromatic side chain; comprises an azido group; comprises an alkyne group; or comprises an aldehyde or ketone group. In some embodiments, the at least one unnatural amino acid does not comprise an aromatic side chain. In some embodiments, the at least one unnatural amino acid comprises N6-((2-azidoethoxy)-carbonyl)-L-lysine (AzK), N6-((propargyloxy)-carbonyl)-L-lysine (PraK), BCN-L-lysine, norbornene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, 0-allyltyrosine, O-methyl-L-tyrosine, 0-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, or N6-(((4-azidobenzyl)oxy)carbonyl)-L-lysine. In some embodiments, the at least one unnatural amino acid comprises N6-((2-azidoethoxy)-carbonyl)-L-lysine (AzK) or N6-((propargyloxy)-carbonyl)-L-lysine (PraK). In some embodiments, the at least one unnatural amino acid comprises N6-((2-azidoethoxy)-carbonyl)-L-lysine (AzK). In some embodiments, the at least one unnatural amino acid comprises N6-((propargyloxy)-carbonyl)-L-lysine (PraK).

In some embodiments, the at least one unnatural amino acid comprises an alkyne that is allowed to react with a conjugating moiety that comprises a water-soluble polymer comprises polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), or a combination thereof. In some embodiments, the water-soluble polymer comprises a PEG molecule

In some embodiments, the modified IL-10 polypeptide comprises a conjugating moiety. In some embodiments, the conjugating moiety comprises a water-soluble polymer, a lipid, a protein, and/or a peptide. In some embodiments, the water-soluble polymer comprises polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), or a combination thereof. In some embodiments, the water-soluble polymer comprises a PEG molecule.

In some embodiments, the molecular weight of the PEG determines, at least in part, the in vivo plasma half-life of the modified IL-0 polypeptide. In some instances, the conjugating moiety comprises a PEG molecule that corresponds with a longer in vivo plasma half-life of the modified IL-10 polypeptide, as compared to the in vivo plasma half-life of a PEG that is smaller than the conjugating moiety. In some instances, the conjugating moiety comprises a PEG molecule that corresponds with a shorter in vivo plasma half-life of the modified IL-10 polypeptide, as compared to the in vivo plasma half-life of a PEG that is larger than the conjugating moiety.

In some embodiments, the molecular weight of the PEG does not affect, or has minimal effect, on the receptor signaling potency to the IL-10R signaling. In some embodiments, the molecular weight of the PEG does not affect, or has minimal effect, on the desired reduced binding to IL-10R or the maintained binding with IL-10R, wherein the reduced binding to IL-10R is compared to binding between a wild-type IL-10 protein and IL-10R.

In some embodiments, the PEG molecule is a linear PEG. In some embodiments, wherein the PEG molecule is a branched PEG. In some embodiments, the PEG comprises between about 2,000-50,000 Daltons (Da). In some embodiments, the PEG has a molecular weight comprising about 5,000 Da, 10,000 Da, 15,000 Da, 20,000 Da, 25,000 Da, 30,000 Da, 35,000 Da, 40,000 Da, 45,000 Da, or 50,000 Da. In some instances, the PEG is 5,000 Da. In some instances, the PEG is 10,000 Da. In some instances, the PEG is 15,000 Da. In some instances, the PEG is 20,000 Da. In some instances, the PEG is 25,000 Da. In some instances, the PEG is 30,000 Da. In some instances, the PEG is 35,000 Da. In some instances, the PEG is 40,000 Da. In some instances, the PEG is 45,000 Da. In some instances, the PEG is 50,000 Da.

For example, the container(s) include one or more modified IL-10 polypeptides comprising a mutated amino acid residue E67, Q70, E74, E75, Q79, N82, K88, A89, K99, K125, N126, N129, K130, or Q132 with residue positions corresponding with 67, 70, 74, 75, 79, 82, 88, 89, 99, 125, 126, 129, 130, and 132 as set forth in SEQ ID NO: 1. In some embodiments, the modified IL-10 polypeptide comprises a conjugating moiety comprising a PEG having a molecular weight of about 2,000-50,000 Da. In some embodiments, the molecular weight comprises 5,000 Da. In some embodiments, the molecular weight comprises 10,000 Da. In some embodiments, the molecular weight comprises 15,000 Da. In some embodiments, the molecular weight comprises 20,000 Da. In some embodiments, the molecular weight comprises 25,000 Da. In some embodiments, the molecular weight comprises 30,000 Da. In some embodiments, the molecular weight comprises 35,000 Da. In some embodiments, the molecular weight comprises 40,000 Da. In some embodiments, the molecular weight comprises 45,000 Da. In some embodiments, the molecular weight comprises 50,000 Da. In some embodiments, the molecular weight of the PEG determines, at least in part, the in vivo plasma half-life of the modified IL-10 polypeptide. In some instances, the PEG corresponds with a longer in vivo plasma half-life of the modified IL-10 polypeptide, as compared to the in vivo plasma half-life of a smaller PEG. In some instances, the PEG corresponds with a shorter in vivo plasma half-life of the modified IL-10 polypeptide, as compared to the in vivo plasma half-life of a larger PEG. In some embodiments, the molecular weight of the PEG does not affect, or has minimal effect, on the receptor signaling potency of the modified IL-10 polypeptide to the IL-10R signaling. In some embodiments, the molecular weight of the PEG does not affect, or has minimal effect, on the desired reduced binding of the modified IL-10 polypeptide to IL-10R or the maintained binding with IL-10R, wherein the reduced binding to IL-10R is compared to binding between a wild-type IL-10 protein and IL-10R.

### EXEMPLARY EMBODIMENTS

The present disclosure is further described by the following embodiments. The features of each of the embodiments are combinable with any of the other embodiments where appropriate and practical.

Embodiment 1. An IL-10 conjugate comprising the amino acid sequence of SEQ ID NO: 1 in which at least one amino acid residue in the IL-10 conjugate is replaced by the structure of Formula (I):

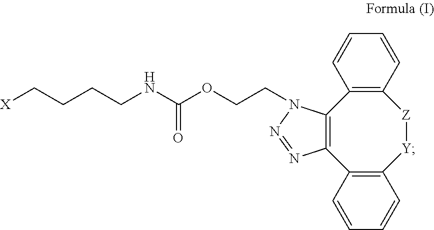

wherein:

### Z is CH₂ and Y is

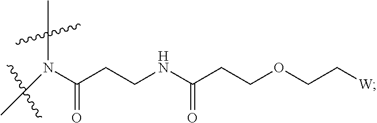

### Y is CH₂ and Z is

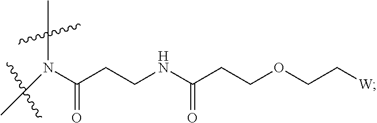

### Z is CH₂ and Y is

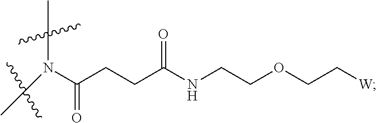

or

### Y is CH₂ and Z is

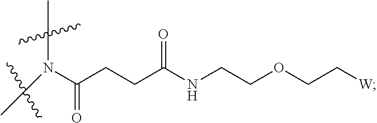

W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

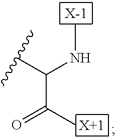

or a pharmaceutically acceptable salt, solvate, or hydrate thereof.

Embodiment 1.1. An IL-10 conjugate comprising the amino acid sequence of SEQ ID NO: 1 in which at least one amino acid residue in the IL-10 conjugate is replaced by the structure of Formula (I):

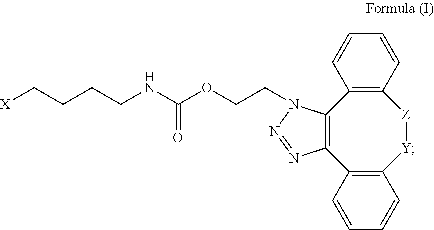

wherein:

### Z is CH₂ and Y is

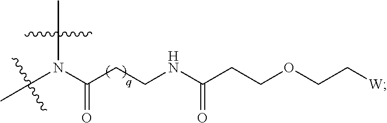

### Y is CH₂ and Z is

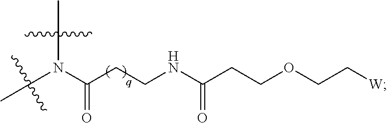

### Z is CH₂ and Y is

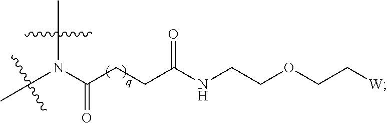

or

### Y is CH₂ and Z is H

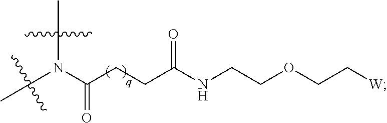

W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa;
q is 1, 2, or 3; and
X has the structure:

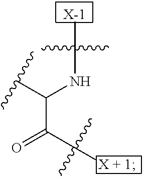

or a pharmaceutically acceptable salt, solvate, or hydrate thereof.

Embodiment 2. The IL-10 conjugate of embodiment 1 or 1.1, wherein Z is CH₂ and Y is

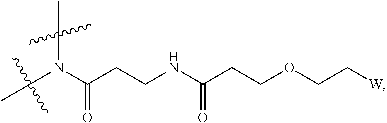

or a pharmaceutically acceptable salt, solvate, or hydrate thereof.

Embodiment 3 The IL-10 conjugate of embodiment 1 or 1.1, wherein Y is CH₂ and Z is

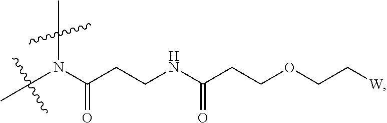

or a pharmaceutically acceptable salt, solvate, or hydrate thereof.

Embodiment 4. The IL-10 conjugate of embodiment 1, wherein Z is CH₂ and Y is

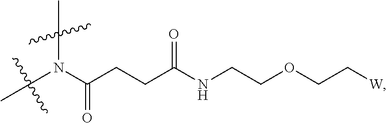

or a pharmaceutically acceptable salt, solvate, or hydrate thereof.

Embodiment 5. The IL-10 conjugate of embodiment 1 or 1.1, wherein Y is CH₂ and Z is

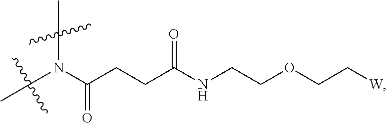

or a pharmaceutically acceptable salt, solvate, or hydrate thereof.

Embodiment 6. The IL-10 conjugate of embodiment 1 or 1.1, wherein the PEG group has an average molecular weight selected from 5 kDa, 10 kDa, 20 kDa and 30 kDa, or a pharmaceutically acceptable salt, solvate, or hydrate thereof.

Embodiment 7. The IL-10 conjugate of embodiment 6, wherein the PEG group has an average molecular weight of 20 kDa, or a pharmaceutically acceptable salt, solvate, or hydrate thereof.

Embodiment 8. The IL-10 conjugate of embodiment 6, wherein the PEG group has an average molecular weight of 30 kDa, or a pharmaceutically acceptable salt, solvate, or hydrate thereof.

Embodiment 9. The IL-10 conjugate of embodiment 1 or 1.1, wherein the position of the structure of Formula (I) in the amino acid sequence of the IL-10 conjugate is selected from N82, K88, A89, K99, K125, N126, N129, and K130, wherein the position of the structure of Formula (I) in the amino acid sequence of the IL-10 conjugate is in reference to the positions in SEQ ID NO: 1, or a pharmaceutically acceptable salt, solvate, or hydrate thereof.

Embodiment 10. The IL-10 conjugate of embodiment 9, wherein the position of the structure of Formula (I) in the amino acid sequence of the IL-10 conjugate is selected from N82, K88, K99, N126, N129, and K130, wherein the position of the structure of Formula (I) in the amino acid sequence of the IL-10 conjugate is in reference to the positions in SEQ ID NO: 1, or a pharmaceutically acceptable salt, solvate, or hydrate thereof.

Embodiment 11. An IL-10 conjugate comprising the amino acid sequence of any one of SEQ ID NOS: 19 to 26, wherein [AzK_PEG] has the structure of Formula (II), Formula (III), or a mixture of Formula (II) and Formula (III):

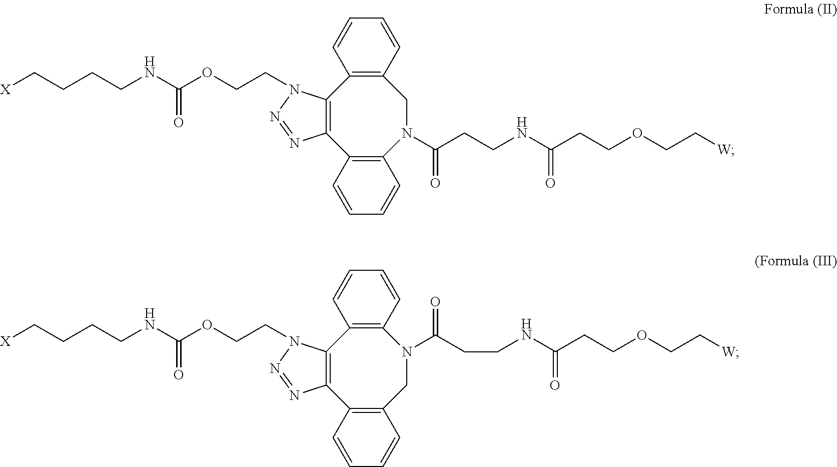

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

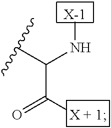

or a pharmaceutically acceptable salt, solvate, or hydrate thereof.

Embodiment 11.1. An IL-10 conjugate comprising the amino acid sequence of any one of SEQ ID NOS: 19 to 26, wherein [AzK_PEG] has the structure of Formula (II), Formula (III), or a mixture of Formula (II) and Formula (III):

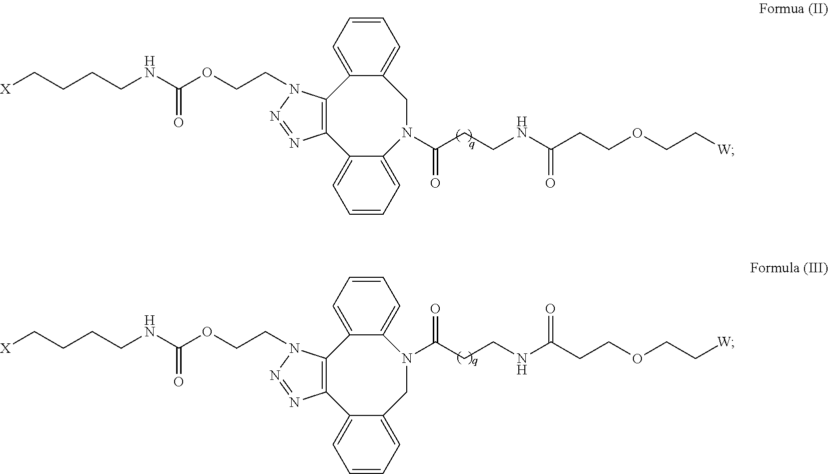

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa;
q is 1, 2, or 3; and
X has the structure:

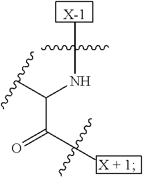

or a pharmaceutically acceptable salt, solvate, or hydrate thereof.

Embodiment 12. The IL-10 conjugate of embodiment 11 or 11.1, wherein the [AzK_PEG] is a mixture of Formula (II) and Formula (III), or a pharmaceutically acceptable salt, solvate, or hydrate thereof.

Embodiment 13. The IL-10 conjugate of embodiment 11 or 11.1, wherein the [AzK_PEG] has the structure of formula (II):

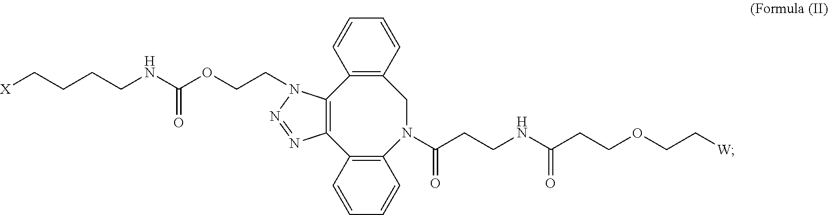

or a pharmaceutically acceptable salt, solvate, or hydrate thereof.

Embodiment 14. The IL-10 conjugate of embodiment 13, wherein the IL-10 conjugate has the amino acid sequence of SEQ ID NO: 19, or a pharmaceutically acceptable salt, solvate, or hydrate thereof.

Embodiment 15. The IL-10 conjugate of embodiment 14, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 16. The IL-10 conjugate of embodiment 15, wherein W is a PEG group having an average molecular weight selected from 20 kDa and 30 kDa, or a pharmaceutically acceptable salt, solvate, or hydrate thereof.

Embodiment 17. The IL-10 conjugate of embodiment 16, wherein W is a PEG group having an average molecular weight of 20 kDa, or a pharmaceutically acceptable salt, solvate, or hydrate thereof.

Embodiment 18. The IL-10 conjugate of embodiment 16, wherein W is a PEG group having an average molecular weight of 30 kDa, or a pharmaceutically acceptable salt, solvate, or hydrate thereof.

Embodiment 19. The IL-10 conjugate of embodiment 13, wherein the IL-10 conjugate has the amino acid sequence of SEQ ID NO: 20, or a pharmaceutically acceptable salt, solvate, or hydrate thereof.

Embodiment 20. The IL-10 conjugate of embodiment 19, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 21. The IL-10 conjugate of embodiment 20, wherein W is a PEG group having an average molecular weight selected from 20 kDa and 30 kDa, or a pharmaceutically acceptable salt, solvate, or hydrate thereof.

Embodiment 22. The IL-10 conjugate of embodiment 21, wherein W is a PEG group having an average molecular weight of 20 kDa, or a pharmaceutically acceptable salt, solvate, or hydrate thereof.

Embodiment 23. The IL-10 conjugate of embodiment 21, wherein W is a PEG group having an average molecular weight of 30 kDa, or a pharmaceutically acceptable salt, solvate, or hydrate thereof.

Embodiment 24. The IL-10 conjugate of embodiment 13, wherein the IL-10 conjugate has the amino acid sequence of SEQ ID NO: 21, or a pharmaceutically acceptable salt, solvate, or hydrate thereof.

Embodiment 25. The IL-10 conjugate of embodiment 24, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 26. The IL-10 conjugate of embodiment 25, wherein W is a PEG group having an average molecular weight selected from 20 kDa and 30 kDa, or a pharmaceutically acceptable salt, solvate, or hydrate thereof.

Embodiment 27. The IL-10 conjugate of embodiment 26, wherein W is a PEG group having an average molecular weight of 20 kDa, or a pharmaceutically acceptable salt, solvate, or hydrate thereof.

Embodiment 28. The IL-10 conjugate of embodiment 26, wherein W is a PEG group having an average molecular weight of 30 kDa, or a pharmaceutically acceptable salt, solvate, or hydrate thereof.

Embodiment 29. The IL-10 conjugate of embodiment 13, wherein the IL-10 conjugate has the amino acid sequence of SEQ ID NO: 22, or a pharmaceutically acceptable salt, solvate, or hydrate thereof.

Embodiment 30. The IL-10 conjugate of embodiment 29, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 31. The IL-10 conjugate of embodiment 30, wherein W is a PEG group having an average molecular weight selected from 20 kDa and 30 kDa, or a pharmaceutically acceptable salt, solvate, or hydrate thereof.

Embodiment 32. The IL-10 conjugate of embodiment 31, wherein W is a PEG group having an average molecular weight of 20 kDa, or a pharmaceutically acceptable salt, solvate, or hydrate thereof.

Embodiment 33. The IL-10 conjugate of embodiment 31, wherein W is a PEG group having an average molecular weight of 30 kDa, or a pharmaceutically acceptable salt, solvate, or hydrate thereof.

Embodiment 34. The IL-10 conjugate of embodiment 13, wherein the IL-10 conjugate has the amino acid sequence of SEQ ID NO: 23, or a pharmaceutically acceptable salt, solvate, or hydrate thereof.

Embodiment 35. The IL-10 conjugate of embodiment 34, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 36. The IL-10 conjugate of embodiment 35, wherein W is a PEG group having an average molecular weight selected from 20 kDa and 30 kDa, or a pharmaceutically acceptable salt, solvate, or hydrate thereof.

Embodiment 37. The IL-10 conjugate of embodiment 36, wherein W is a PEG group having an average molecular weight of 20 kDa, or a pharmaceutically acceptable salt, solvate, or hydrate thereof.

Embodiment 38. The IL-10 conjugate of embodiment 36, wherein W is a PEG group having an average molecular weight of 30 kDa, or a pharmaceutically acceptable salt, solvate, or hydrate thereof.

Embodiment 39. The IL-10 conjugate of embodiment 13, wherein the IL-10 conjugate has the amino acid sequence of SEQ ID NO: 24, or a pharmaceutically acceptable salt, solvate, or hydrate thereof.

Embodiment 40. The IL-10 conjugate of embodiment 39, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 41. The IL-10 conjugate of embodiment 40, wherein W is a PEG group having an average molecular weight selected from 20 kDa and 30 kDa, or a pharmaceutically acceptable salt, solvate, or hydrate thereof.

Embodiment 42. The IL-10 conjugate of embodiment 41, wherein W is a PEG group having an average molecular weight of 20 kDa, or a pharmaceutically acceptable salt, solvate, or hydrate thereof.

Embodiment 43. The IL-10 conjugate of embodiment 41, wherein W is a PEG group having an average molecular weight of 30 kDa, or a pharmaceutically acceptable salt, solvate, or hydrate thereof.

Embodiment 44. The IL-10 conjugate of embodiment 13, wherein the IL-10 conjugate has the amino acid sequence of SEQ ID NO: 25, or a pharmaceutically acceptable salt, solvate, or hydrate thereof.

Embodiment 45. The IL-10 conjugate of embodiment 44, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 46. The IL-10 conjugate of embodiment 45, wherein W is a PEG group having an average molecular weight selected from 20 kDa and 30 kDa, or a pharmaceutically acceptable salt, solvate, or hydrate thereof.

Embodiment 47. The IL-10 conjugate of embodiment 46, wherein W is a PEG group having an average molecular weight of 20 kDa, or a pharmaceutically acceptable salt, solvate, or hydrate thereof.

Embodiment 48. The IL-10 conjugate of embodiment 46, wherein W is a PEG group having an average molecular weight of 30 kDa, or a pharmaceutically acceptable salt, solvate, or hydrate thereof.

Embodiment 49. The IL-10 conjugate of embodiment 13, wherein the IL-10 conjugate has the amino acid sequence of SEQ ID NO: 26, or a pharmaceutically acceptable salt, solvate, or hydrate thereof.

Embodiment 50. The IL-10 conjugate of embodiment 49, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 51. The IL-10 conjugate of embodiment 50, wherein W is a PEG group having an average molecular weight selected from 20 kDa and 30 kDa, or a pharmaceutically acceptable salt, solvate, or hydrate thereof.

Embodiment 52. The IL-10 conjugate of embodiment 51, wherein W is a PEG group having an average molecular weight of 20 kDa, or a pharmaceutically acceptable salt, solvate, or hydrate thereof.

Embodiment 53. The IL-10 conjugate of embodiment 51, wherein W is a PEG group having an average molecular weight of 30 kDa, or a pharmaceutically acceptable salt, solvate, or hydrate thereof.

Embodiment 54. The IL-10 conjugate of embodiment 11 or 11.1, wherein the [AzK_PEG] has the structure of Formula (III):

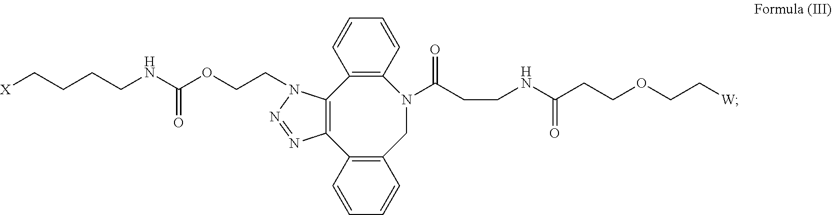

or a pharmaceutically acceptable salt, solvate, or hydrate thereof.

Embodiment 55. The IL-10 conjugate of embodiment 54, wherein the IL-10 conjugate has the amino acid sequence of SEQ ID NO: 19, or a pharmaceutically acceptable salt, solvate, or hydrate thereof.

Embodiment 56. The IL-10 conjugate of embodiment 55, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 57. The IL-10 conjugate of embodiment 56, wherein W is a PEG group having an average molecular weight selected from 20 kDa and 30 kDa, or a pharmaceutically acceptable salt, solvate, or hydrate thereof.

Embodiment 58. The IL-10 conjugate of embodiment 57, wherein W is a PEG group having an average molecular weight of 20 kDa, or a pharmaceutically acceptable salt, solvate, or hydrate thereof.

Embodiment 59. The IL-10 conjugate of embodiment 57, wherein W is a PEG group having an average molecular weight of 30 kDa, or a pharmaceutically acceptable salt, solvate, or hydrate thereof.

Embodiment 60. The IL-10 conjugate of embodiment 54, wherein the IL-10 conjugate has the amino acid sequence of SEQ ID NO: 20, or a pharmaceutically acceptable salt, solvate, or hydrate thereof.

Embodiment 61. The IL-10 conjugate of embodiment 60, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 62. The IL-10 conjugate of embodiment 61, wherein W is a PEG group having an average molecular weight selected from 20 kDa and 30 kDa, or a pharmaceutically acceptable salt, solvate, or hydrate thereof.

Embodiment 63. The IL-10 conjugate of embodiment 62, wherein W is a PEG group having an average molecular weight of 20 kDa, or a pharmaceutically acceptable salt, solvate, or hydrate thereof.

Embodiment 64. The IL-10 conjugate of embodiment 62, wherein W is a PEG group having an average molecular weight of 30 kDa, or a pharmaceutically acceptable salt, solvate, or hydrate thereof.

Embodiment 65. The IL-10 conjugate of embodiment 54, wherein the IL-10 conjugate has the amino acid sequence of SEQ ID NO: 21, or a pharmaceutically acceptable salt, solvate, or hydrate thereof.

Embodiment 66. The IL-10 conjugate of embodiment 65, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 67. The IL-10 conjugate of embodiment 66, wherein W is a PEG group having an average molecular weight selected from 20 kDa and 30 kDa, or a pharmaceutically acceptable salt, solvate, or hydrate thereof.

Embodiment 68. The IL-10 conjugate of embodiment 67, wherein W is a PEG group having an average molecular weight of 20 kDa, or a pharmaceutically acceptable salt, solvate, or hydrate thereof.

Embodiment 69. The IL-10 conjugate of embodiment 67, wherein W is a PEG group having an average molecular weight of 30 kDa, or a pharmaceutically acceptable salt, solvate, or hydrate thereof.

Embodiment 70. The IL-10 conjugate of embodiment 54, wherein the IL-10 conjugate has the amino acid sequence of SEQ ID NO: 22, or a pharmaceutically acceptable salt, solvate, or hydrate thereof.

Embodiment 71. The IL-10 conjugate of embodiment 70, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 72. The IL-10 conjugate of embodiment 71, wherein W is a PEG group having an average molecular weight selected from 20 kDa and 30 kDa, or a pharmaceutically acceptable salt, solvate, or hydrate thereof.

Embodiment 73. The IL-10 conjugate of embodiment 72, wherein W is a PEG group having an average molecular weight of 20 kDa, or a pharmaceutically acceptable salt, solvate, or hydrate thereof.

Embodiment 74. The IL-10 conjugate of embodiment 72, wherein W is a PEG group having an average molecular weight of 30 kDa, or a pharmaceutically acceptable salt, solvate, or hydrate thereof.

Embodiment 75. The IL-10 conjugate of embodiment 54, wherein the IL-10 conjugate has the amino acid sequence of SEQ ID NO: 23, or a pharmaceutically acceptable salt, solvate, or hydrate thereof.

Embodiment 76. The IL-10 conjugate of embodiment 75, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 77. The IL-10 conjugate of embodiment 76, wherein W is a PEG group having an average molecular weight selected from 20 kDa and 30 kDa, or a pharmaceutically acceptable salt, solvate, or hydrate thereof.

Embodiment 78. The IL-10 conjugate of embodiment 77, wherein W is a PEG group having an average molecular weight of 20 kDa, or a pharmaceutically acceptable salt, solvate, or hydrate thereof.

Embodiment 79. The IL-10 conjugate of embodiment 77, wherein W is a PEG group having an average molecular weight of 30 kDa, or a pharmaceutically acceptable salt, solvate, or hydrate thereof.

Embodiment 80. The IL-10 conjugate of embodiment 54, wherein the IL-10 conjugate has the amino acid sequence of SEQ ID NO: 24, or a pharmaceutically acceptable salt, solvate, or hydrate thereof.

Embodiment 81. The IL-10 conjugate of embodiment 80, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 82. The IL-10 conjugate of embodiment 81, wherein W is a PEG group having an average molecular weight selected from 20 kDa and 30 kDa, or a pharmaceutically acceptable salt, solvate, or hydrate thereof.

Embodiment 83. The IL-10 conjugate of embodiment 82, wherein W is a PEG group having an average molecular weight of 20 kDa, or a pharmaceutically acceptable salt, solvate, or hydrate thereof.

Embodiment 84. The IL-10 conjugate of embodiment 82, wherein W is a PEG group having an average molecular weight of 30 kDa, or a pharmaceutically acceptable salt, solvate, or hydrate thereof.

Embodiment 85. The IL-10 conjugate of embodiment 54, wherein the IL-10 conjugate has the amino acid sequence of SEQ ID NO: 25, or a pharmaceutically acceptable salt, solvate, or hydrate thereof.

Embodiment 86. The IL-10 conjugate of embodiment 85, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 87. The IL-10 conjugate of embodiment 86, wherein W is a PEG group having an average molecular weight selected from 20 kDa and 30 kDa, or a pharmaceutically acceptable salt, solvate, or hydrate thereof.

Embodiment 88. The IL-10 conjugate of embodiment 87, wherein W is a PEG group having an average molecular weight of 20 kDa, or a pharmaceutically acceptable salt, solvate, or hydrate thereof.

Embodiment 89. The IL-10 conjugate of embodiment 87, wherein W is a PEG group having an average molecular weight of 30 kDa, or a pharmaceutically acceptable salt, solvate, or hydrate thereof.

Embodiment 90. The IL-10 conjugate of embodiment 54, wherein the IL-10 conjugate has the amino acid sequence of SEQ ID NO: 26, or a pharmaceutically acceptable salt, solvate, or hydrate thereof.

Embodiment 91. The IL-10 conjugate of embodiment 90, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 92. The IL-10 conjugate of embodiment 91, wherein W is a PEG group having an average molecular weight selected from 20 kDa and 30 kDa, or a pharmaceutically acceptable salt, solvate, or hydrate thereof.

Embodiment 93. The IL-10 conjugate of embodiment 92, wherein W is a PEG group having an average molecular weight of 20 kDa, or a pharmaceutically acceptable salt, solvate, or hydrate thereof.

Embodiment 94. The IL-10 conjugate of embodiment 92, wherein W is a PEG group having an average molecular weight of 30 kDa, or a pharmaceutically acceptable salt, solvate, or hydrate thereof.

Embodiment 95. The IL-10 conjugate of any one of embodiments 1 to 94, wherein W is a linear or branched PEG group, or a pharmaceutically acceptable salt, solvate, or hydrate thereof.

Embodiment 96. The IL-10 conjugate of any one of embodiments 1 to 94, wherein W is a linear PEG group, or a pharmaceutically acceptable salt, solvate, or hydrate thereof.

Embodiment 97. The IL-10 conjugate of any one of embodiments 1 to 94, wherein W is a branched PEG group, or a pharmaceutically acceptable salt, solvate, or hydrate thereof.

Embodiment 98. The IL-10 conjugate of any one of embodiments 1 to 94, wherein W is a methoxy PEG group, or a pharmaceutically acceptable salt, solvate, or hydrate thereof.

Embodiment 99. The IL-10 conjugate of embodiment 98, wherein the methoxy PEG group is linear or branched, or a pharmaceutically acceptable salt, solvate, or hydrate thereof.

Embodiment 100. The IL-10 conjugate of embodiment 98, wherein the methoxy PEG group is linear, or a pharmaceutically acceptable salt, solvate, or hydrate thereof.

Embodiment 101. The IL-10 conjugate of embodiment 98, wherein the methoxy PEG group is branched, or a pharmaceutically acceptable salt, solvate, or hydrate thereof.

Embodiment 102. An IL-10 conjugate comprising the amino acid sequence of any one of SEQ ID NOS: 27 to 34, wherein [AzK_PEG20 kDa] has the structure of Formula (II), Formula (III), or a mixture of Formula (II) and Formula (III):

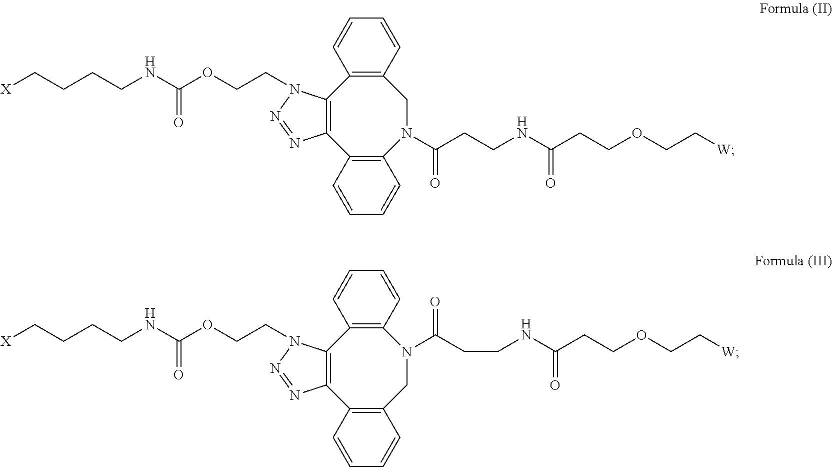

wherein:
W is a PEG group having an average molecular weight of 20 kDa; and
X has the structure:

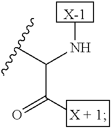

or a pharmaceutically acceptable salt, solvate, or hydrate thereof.

Embodiment 102.1. An IL-10 conjugate comprising the amino acid sequence of any one of SEQ ID NOS: 27 to 34, wherein [AzK_PEG20 kDa] has the structure of Formula (II), Formula (III), or a mixture of Formula (II) and Formula (III):

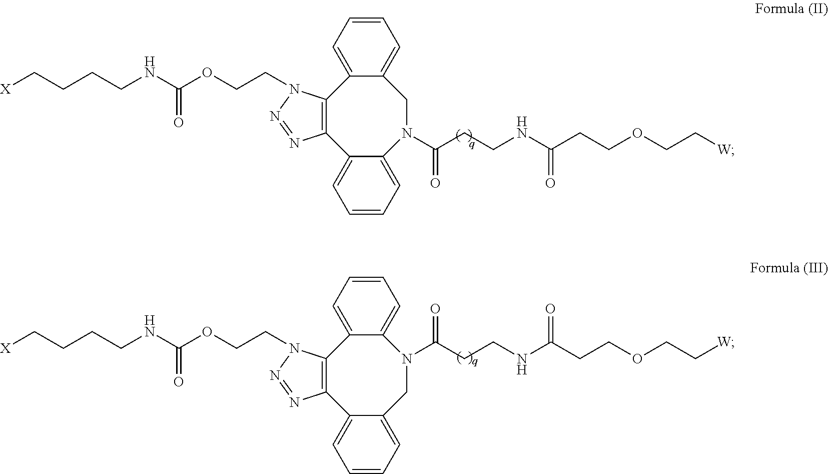

wherein:
W is a PEG group having an average molecular weight of 20 kDa;
q is 1, 2, or 3; and
X has the structure:

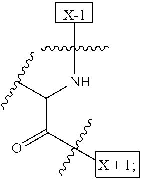

or a pharmaceutically acceptable salt, solvate, or hydrate thereof.

Embodiment 103. The IL-10 conjugate of embodiment 102 or 102.1, wherein the IL-10 conjugate has the amino acid sequence of SEQ ID NO: 27, or a pharmaceutically acceptable salt, solvate, or hydrate thereof.

Embodiment 104. The IL-10 conjugate of embodiment 102 or 102.1, wherein the IL-10 conjugate has the amino acid sequence of SEQ ID NO: 28, or a pharmaceutically acceptable salt, solvate, or hydrate thereof.

Embodiment 105. The IL-10 conjugate of embodiment 102 or 102.1, wherein the IL-10 conjugate has the amino acid sequence of SEQ ID NO: 29, or a pharmaceutically acceptable salt, solvate, or hydrate thereof.

Embodiment 106. The IL-10 conjugate of embodiment 102 or 102.1, wherein the IL-10 conjugate has the amino acid sequence of SEQ ID NO: 30, or a pharmaceutically acceptable salt, solvate, or hydrate thereof.

Embodiment 107. The IL-10 conjugate of embodiment 102 or 102.1, wherein the IL-10 conjugate has the amino acid sequence of SEQ ID NO: 31, or a pharmaceutically acceptable salt, solvate, or hydrate thereof.

Embodiment 108. The IL-10 conjugate of embodiment 102 or 102.1, wherein the IL-10 conjugate has the amino acid sequence of SEQ ID NO: 32, or a pharmaceutically acceptable salt, solvate, or hydrate thereof.

Embodiment 109. The IL-10 conjugate of embodiment 102 or 102.1, wherein the IL-10 conjugate has the amino acid sequence of SEQ ID NO: 33, or a pharmaceutically acceptable salt, solvate, or hydrate thereof.

Embodiment 110. The IL-10 conjugate of embodiment 102 or 102.1, wherein the IL-10 conjugate has the amino acid sequence of SEQ ID NO: 34, or a pharmaceutically acceptable salt, solvate, or hydrate thereof.

Embodiment 111. The IL-10 conjugate of embodiment 102 or 102.1, wherein the [AzK_PEG20 kDa] has the structure of Formula (II):

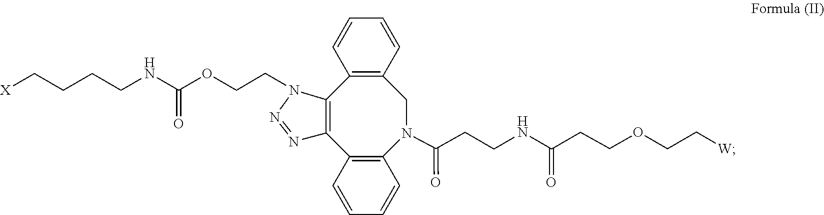

or a pharmaceutically acceptable salt, solvate, or hydrate thereof.

Embodiment 112. The IL-10 conjugate of embodiment 111, wherein the IL-10 conjugate has the amino acid sequence of SEQ ID NO: 27, or a pharmaceutically acceptable salt, solvate, or hydrate thereof.

Embodiment 113. The IL-10 conjugate of embodiment 111, wherein the IL-10 conjugate has the amino acid sequence of SEQ ID NO: 28, or a pharmaceutically acceptable salt, solvate, or hydrate thereof.

Embodiment 114. The IL-10 conjugate of embodiment 111, wherein the IL-10 conjugate has the amino acid sequence of SEQ ID NO: 29, or a pharmaceutically acceptable salt, solvate, or hydrate thereof.

Embodiment 115. The IL-10 conjugate of embodiment 111, wherein the IL-10 conjugate has the amino acid sequence of SEQ ID NO: 30, or a pharmaceutically acceptable salt, solvate, or hydrate thereof.

Embodiment 116. The IL-10 conjugate of embodiment 111, wherein the IL-10 conjugate has the amino acid sequence of SEQ ID NO: 31, or a pharmaceutically acceptable salt, solvate, or hydrate thereof.

Embodiment 117. The IL-10 conjugate of embodiment 111, wherein the IL-10 conjugate has the amino acid sequence of SEQ ID NO: 32, or a pharmaceutically acceptable salt, solvate, or hydrate thereof.

Embodiment 118. The IL-10 conjugate of embodiment 111, wherein the IL-10 conjugate has the amino acid sequence of SEQ ID NO: 33, or a pharmaceutically acceptable salt, solvate, or hydrate thereof.

Embodiment 119. The IL-10 conjugate of embodiment 111, wherein the IL-10 conjugate has the amino acid sequence of SEQ ID NO: 34, or a pharmaceutically acceptable salt, solvate, or hydrate thereof.

Embodiment 120. The IL-10 conjugate of embodiment 102 or 102.1, wherein the [AzK_PEG20 kDa] has the structure of Formula (III):

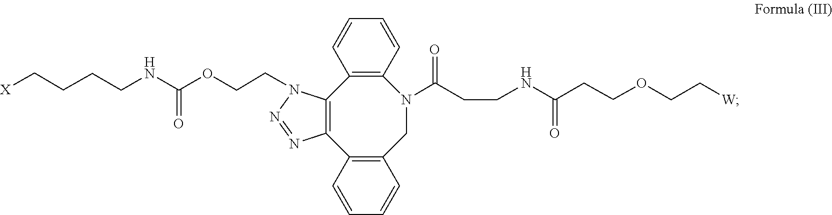

or a pharmaceutically acceptable salt, solvate, or hydrate thereof.

Embodiment 121. The IL-10 conjugate of embodiment 120, wherein the IL-10 conjugate has the amino acid sequence of SEQ ID NO: 27, or a pharmaceutically acceptable salt, solvate, or hydrate thereof.

Embodiment 122. The IL-10 conjugate of embodiment 120, wherein the IL-10 conjugate has the amino acid sequence of SEQ ID NO: 28, or a pharmaceutically acceptable salt, solvate, or hydrate thereof.

Embodiment 123. The IL-10 conjugate of embodiment 120, wherein the IL-10 conjugate has the amino acid sequence of SEQ ID NO: 29, or a pharmaceutically acceptable salt, solvate, or hydrate thereof.

Embodiment 124. The IL-10 conjugate of embodiment 120, wherein the IL-10 conjugate has the amino acid sequence of SEQ ID NO: 30, or a pharmaceutically acceptable salt, solvate, or hydrate thereof.

Embodiment 125. The IL-10 conjugate of embodiment 120, wherein the IL-10 conjugate has the amino acid sequence of SEQ ID NO: 31, or a pharmaceutically acceptable salt, solvate, or hydrate thereof.

Embodiment 126. The IL-10 conjugate of embodiment 120, wherein the IL-10 conjugate has the amino acid sequence of SEQ ID NO: 32, or a pharmaceutically acceptable salt, solvate, or hydrate thereof.

Embodiment 127. The IL-10 conjugate of embodiment 120, wherein the IL-10 conjugate has the amino acid sequence of SEQ ID NO: 33, or a pharmaceutically acceptable salt, solvate, or hydrate thereof.

Embodiment 128. The IL-10 conjugate of embodiment 120, wherein the IL-10 conjugate has the amino acid sequence of SEQ ID NO: 34, or a pharmaceutically acceptable salt, solvate, or hydrate thereof.

Embodiment 129. An IL-10 conjugate comprising the amino acid sequence of any one of SEQ ID NOS: 35 to 42, wherein [AzK_PEG30 kDa] has the structure of Formula (II), Formula (III), or a mixture of the structures of Formula (II) and Formula (III):

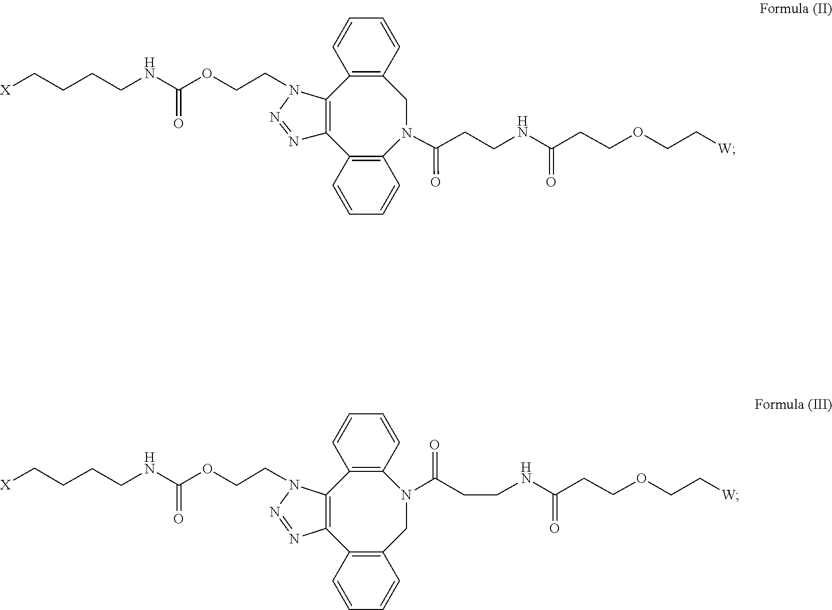

wherein:
W is a PEG group having an average molecular weight of 30 kDa; and
X has the structure:

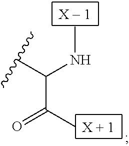 [SMILES: *C(NC)C(C)=O]

or a pharmaceutically acceptable salt, solvate, or hydrate thereof.

Embodiment 129.1. An IL-10 conjugate comprising the amino acid sequence of any one of SEQ ID NOS: 35 to 42, wherein [AzK_PEG30 kDa] has the structure of Formula (II), Formula (III), or a mixture of the structures of Formula (II) and Formula (III):

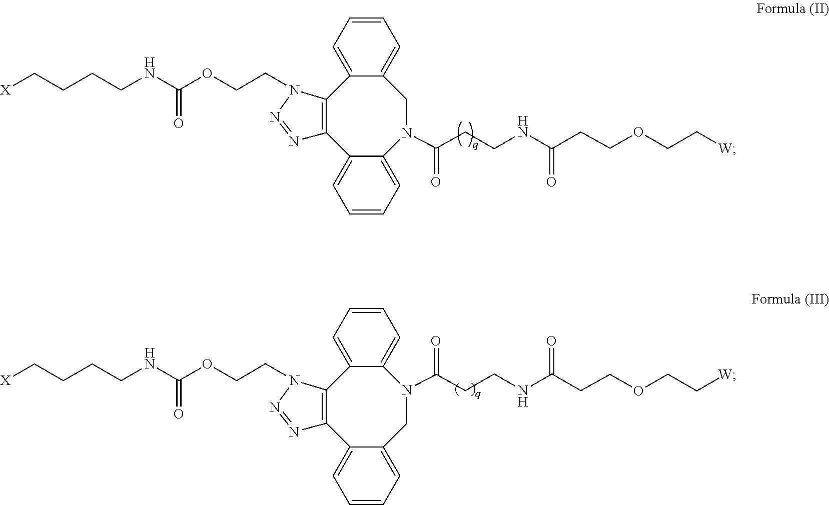

wherein:
W is a PEG group having an average molecular weight of 30 kDa;
q is 1, 2, or 3; and
X has the structure:

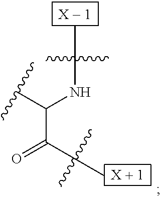 [SMILES: *NC(*)C(*)=O]

or a pharmaceutically acceptable salt, solvate, or hydrate thereof.

Embodiment 130. The IL-10 conjugate of embodiment 129 or 129.1, wherein the IL-10 conjugate has the amino acid sequence of SEQ ID NO: 35, or a pharmaceutically acceptable salt, solvate, or hydrate thereof.

Embodiment 131. The IL-10 conjugate of embodiment 129 or 129.1, wherein the IL-10 conjugate has the amino acid sequence of SEQ ID NO: 36, or a pharmaceutically acceptable salt, solvate, or hydrate thereof.

Embodiment 132. The IL-10 conjugate of embodiment 129 or 129.1, wherein the IL-10 conjugate has the amino acid sequence of SEQ ID NO: 37, or a pharmaceutically acceptable salt, solvate, or hydrate thereof.

Embodiment 133. The IL-10 conjugate of embodiment 129 or 129.1, wherein the IL-10 conjugate has the amino acid sequence of SEQ ID NO: 38, or a pharmaceutically acceptable salt, solvate, or hydrate thereof.

Embodiment 134. The IL-10 conjugate of embodiment 129 or 129.1, wherein the IL-10 conjugate has the amino acid sequence of SEQ ID NO: 39, or a pharmaceutically acceptable salt, solvate, or hydrate thereof.

Embodiment 135. The IL-10 conjugate of embodiment 129 or 129.1, wherein the IL-10 conjugate has the amino acid sequence of SEQ ID NO: 40, or a pharmaceutically acceptable salt, solvate, or hydrate thereof.

Embodiment 136. The IL-10 conjugate of embodiment 129 or 129.1, wherein the IL-10 conjugate has the amino acid sequence of SEQ ID NO: 41, or a pharmaceutically acceptable salt, solvate, or hydrate thereof.

Embodiment 137. The IL-10 conjugate of embodiment 129 or 129.1, wherein the IL-10 conjugate has the amino acid sequence of SEQ ID NO: 42, or a pharmaceutically acceptable salt, solvate, or hydrate thereof.

Embodiment 138. The IL-10 conjugate of embodiment 129 or 129.1, wherein the [AzK_PEG30 kDa] has the structure of Formula (II):

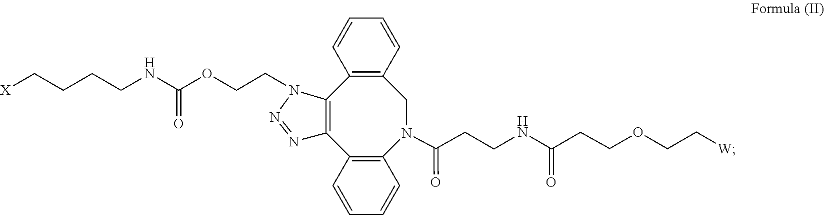

or a pharmaceutically acceptable salt, solvate, or hydrate thereof.

Embodiment 139. The IL-10 conjugate of embodiment 138, wherein the IL-10 conjugate has the amino acid sequence of SEQ ID NO: 35, or a pharmaceutically acceptable salt, solvate, or hydrate thereof.

Embodiment 140. The IL-10 conjugate of embodiment 138, wherein the IL-10 conjugate has the amino acid sequence of SEQ ID NO: 36, or a pharmaceutically acceptable salt, solvate, or hydrate thereof.

Embodiment 141. The IL-10 conjugate of embodiment 138, wherein the IL-10 conjugate has the amino acid sequence of SEQ ID NO: 37, or a pharmaceutically acceptable salt, solvate, or hydrate thereof.

Embodiment 142. The IL-10 conjugate of embodiment 138, wherein the IL-10 conjugate has the amino acid sequence of SEQ ID NO: 38, or a pharmaceutically acceptable salt, solvate, or hydrate thereof.

Embodiment 143. The IL-10 conjugate of embodiment 138, wherein the IL-10 conjugate has the amino acid sequence of SEQ ID NO: 39, or a pharmaceutically acceptable salt, solvate, or hydrate thereof.

Embodiment 144. The IL-10 conjugate of embodiment 138, wherein the IL-10 conjugate has the amino acid sequence of SEQ ID NO: 40, or a pharmaceutically acceptable salt, solvate, or hydrate thereof.

Embodiment 145. The IL-10 conjugate of embodiment 138, wherein the IL-10 conjugate has the amino acid sequence of SEQ ID NO: 41, or a pharmaceutically acceptable salt, solvate, or hydrate thereof.

Embodiment 146. The IL-10 conjugate of embodiment 138, wherein the IL-10 conjugate has the amino acid sequence of SEQ ID NO: 42, or a pharmaceutically acceptable salt, solvate, or hydrate thereof.

Embodiment 147. The IL-10 conjugate of embodiment 129 or 129.1, wherein the [AzK_PEG30 kDa] has the structure of Formula (III):

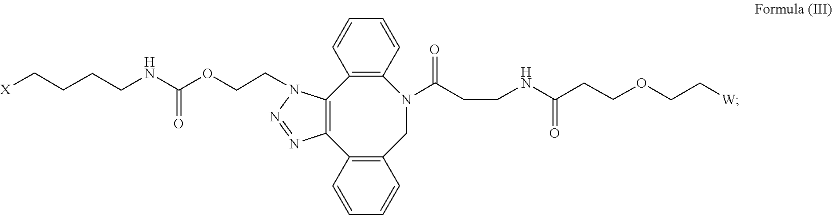

or a pharmaceutically acceptable salt, solvate, or hydrate thereof.

Embodiment 148. The IL-10 conjugate of embodiment 147, wherein the IL-10 conjugate has the amino acid sequence of SEQ TD NO: 35, or a pharmaceutically acceptable salt, solvate, or hydrate thereof.

Embodiment 149. The IL-10 conjugate of embodiment 147, wherein the IL-10 conjugate has the amino acid sequence of SEQ TD NO: 36, or a pharmaceutically acceptable salt, solvate, or hydrate thereof.

Embodiment 150. The IL-10 conjugate of embodiment 147, wherein the IL-10 conjugate has the amino acid sequence of SEQ TD NO: 37, or a pharmaceutically acceptable salt, solvate, or hydrate thereof.

Embodiment 151. The IL-10 conjugate of embodiment 147, wherein the IL-10 conjugate has the amino acid sequence of SEQ ID NO: 38, or a pharmaceutically acceptable salt, solvate, or hydrate thereof.

Embodiment 152. The IL-10 conjugate of embodiment 147, wherein the IL-10 conjugate has the amino acid sequence of SEQ ID NO: 39, or a pharmaceutically acceptable salt, solvate, or hydrate thereof.

Embodiment 153. The IL-10 conjugate of embodiment 147, wherein the IL-10 conjugate has the amino acid sequence of SEQ ID NO: 40, or a pharmaceutically acceptable salt, solvate, or hydrate thereof.

Embodiment 154. The IL-10 conjugate of embodiment 147, wherein the IL-10 conjugate has the amino acid sequence of SEQ ID NO: 41, or a pharmaceutically acceptable salt, solvate, or hydrate thereof.

Embodiment 155. The IL-10 conjugate of embodiment 147, wherein the IL-10 conjugate has the amino acid sequence of SEQ ID NO: 42, or a pharmaceutically acceptable salt, solvate, or hydrate thereof.

Embodiment 156. An TL-10 conjugate comprising the amino acid sequence of any one of SEQ ID NOS: 19 to 26, wherein [AzK_PEG] is a mixture of the structures of Formula (II) and Formula (III):

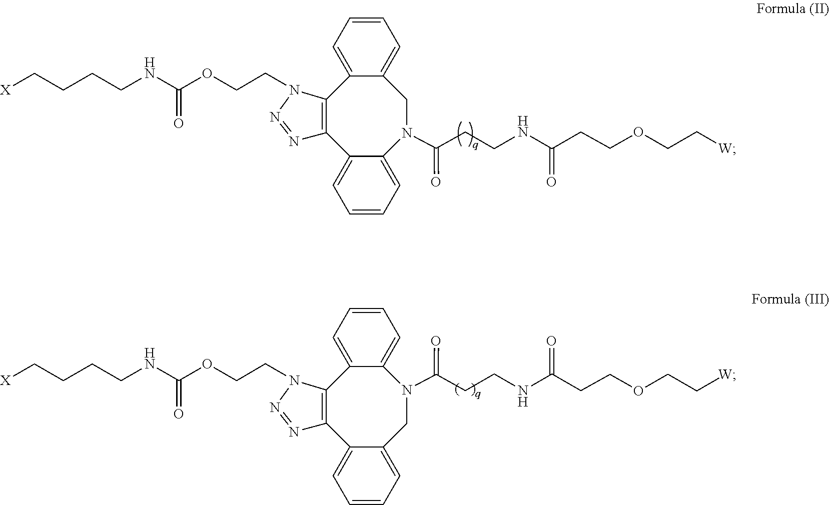

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa;
q is 1, 2, or 3; and
X has the structure:

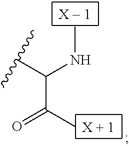 [SMILES: *C(NC)C(C)=O]

or a pharmaceutically acceptable salt, solvate, or hydrate thereof.

Embodiment 156.1. An IL-10 conjugate comprising the amino acid sequence of any one of SEQ ID NOS: 19 to 26, wherein [AzK_PEG] is a mixture of the structures of Formula (II) and Formula (III):

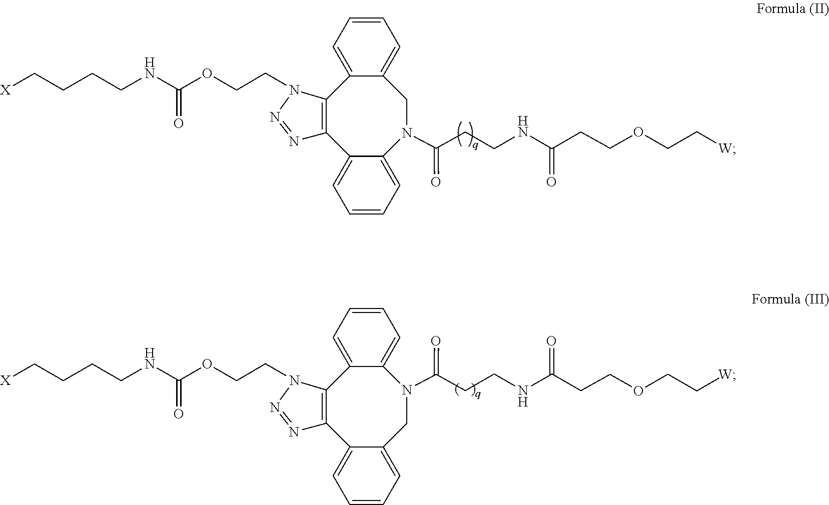

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa;
q is 1, 2, or 3; and
X has the structure:

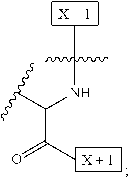 [SMILES: *NC(*)C(C)=O]

or a pharmaceutically acceptable salt, solvate, or hydrate thereof.

Embodiment 157. The IL-10 conjugate of embodiment 156 or 156.1, wherein the ratio of the amount of the structure of Formula (II) to the amount of the structure of Formula (III) comprising the total amount of [AzK_PEG] in the IL-10 conjugate is about 1:1.

Embodiment 158. The IL-10 conjugate of embodiment 156 or 156.1, wherein the ratio of the amount of the structure of Formula (II) to the amount of the structure of Formula (III) comprising the total amount of [AzK_PEG] in the IL-10 conjugate is greater than 1:1.

Embodiment 159. The IL-10 conjugate of embodiment 156 or 156.1, wherein the ratio of the amount of the structure of Formula (II) to the amount of the structure of Formula (III) comprising the total amount of [AzK_PEG] in the IL-10 conjugate is less than 1:1.

Embodiment 160. The IL-10 conjugate of any one of embodiments 156 to 159, wherein W is a linear or branched PEG group, or a pharmaceutically acceptable salt, solvate, or hydrate thereof.

Embodiment 161. The IL-10 conjugate of any one of embodiments 156 to 159, wherein W is a linear PEG group, or a pharmaceutically acceptable salt, solvate, or hydrate thereof.

Embodiment 162. The IL-10 conjugate of any one of embodiments 156 to 159, wherein W is a branched PEG group, or a pharmaceutically acceptable salt, solvate, or hydrate thereof.

Embodiment 163. The IL-10 conjugate of any one of embodiments 156 to 159, wherein W is a methoxy PEG group, or a pharmaceutically acceptable salt, solvate, or hydrate thereof.

Embodiment 164. The IL-10 conjugate of embodiment 163, wherein the methoxy PEG group is linear or branched, or a pharmaceutically acceptable salt, solvate, or hydrate thereof.

Embodiment 165. The IL-10 conjugate of embodiment 164, wherein the methoxy PEG group is linear, or a pharmaceutically acceptable salt, solvate, or hydrate thereof.

Embodiment 166. The IL-10 conjugate of embodiment 164, wherein the methoxy PEG group is branched, or a pharmaceutically acceptable salt, solvate, or hydrate thereof.

Embodiment 167. An IL-10 conjugate comprising the amino acid sequence of any one of SEQ ID NOS: 27 to 34, wherein [AzK_PEG20 kDa] is a mixture of the structures of Formula (II) and Formula (III):

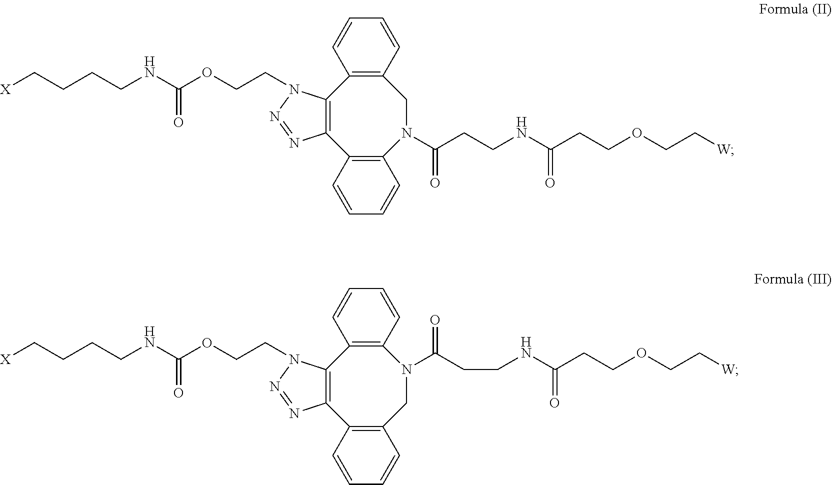

wherein:
W is a PEG group having an average molecular weight of 20 kDa; and
X has the structure:

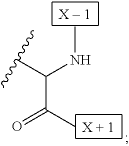 [SMILES: *C(NC)C(C)=O]

or a pharmaceutically acceptable salt, solvate, or hydrate thereof.

Embodiment 167.1. An IL-10 conjugate comprising the amino acid sequence of any one of SEQ ID NOS: 27 to 34, wherein [AzK_PEG20 kDa] is a mixture of the structures of Formula (II) and Formula (III):

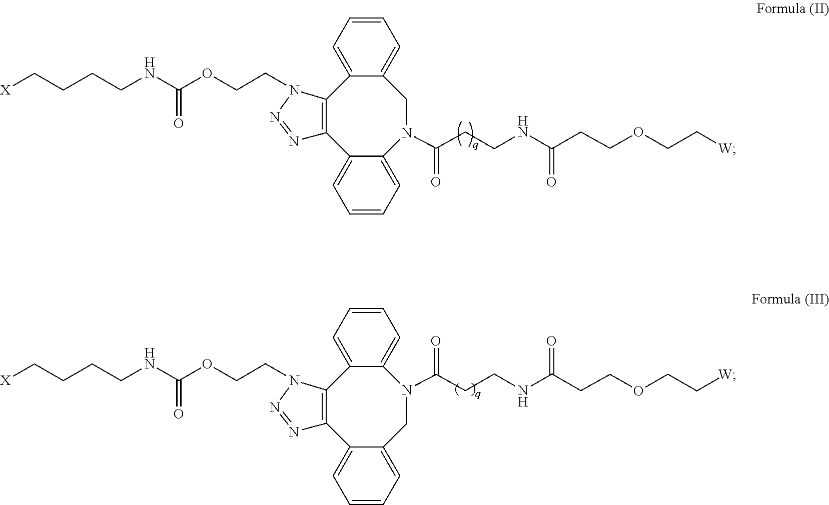

wherein:
W is a PEG group having an average molecular weight of 20 kDa;
q is 1, 2, or 3; and
X has the structure:

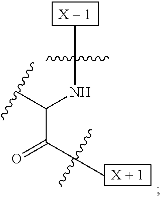 [SMILES: *NC(*)C(*)=O]

or a pharmaceutically acceptable salt, solvate, or hydrate thereof.

Embodiment 168. The IL-10 conjugate of embodiment 167 or 167.1, wherein the ratio of the amount of the structure of Formula (II) to the amount of the structure of Formula (III) comprising the total amount of [AzK_PEG20 kDa] in the IL-10 conjugate is about 1:1.

Embodiment 169. The IL-10 conjugate of embodiment 167 or 167.1, wherein the ratio of the amount of the structure of Formula (II) to the amount of the structure of Formula (III) comprising the total amount of [AzK_PEG20 kDa] in the IL-10 conjugate is greater than 1:1.

Embodiment 170. The IL-10 conjugate of embodiment 167 or 167.1, wherein the ratio of the amount of the structure of Formula (II) to the amount of the structure of Formula (III) comprising the total amount of [AzK_PEG20 kDa] in the IL-10 conjugate is less than 1:1.

Embodiment 171. An IL-10 conjugate comprising the amino acid sequence of any one of SEQ ID NOS: 35 to 42, wherein [AzK_PEG30 kDa] is a mixture of the structures of Formula (II) and Formula (III):

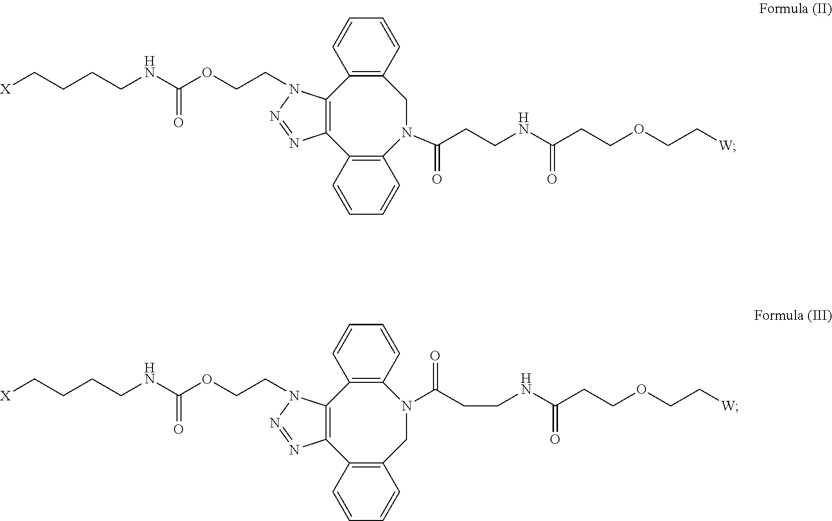

wherein:
W is a PEG group having an average molecular weight of 30 kDa; and
X has the structure:

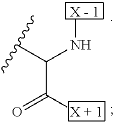

or a pharmaceutically acceptable salt, solvate, or hydrate thereof.

Embodiment 171.1. An IL-10 conjugate comprising the amino acid sequence of any one of SEQ ID NOS: 35 to 42, wherein [AzK_PEG30 kDa] is a mixture of the structures of Formula (II) and Formula (III):

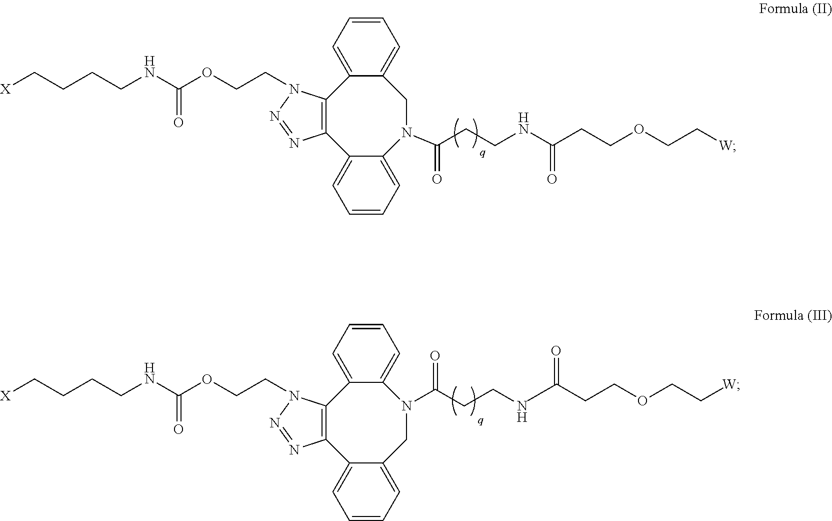

wherein:
W is a PEG group having an average molecular weight of 30 kDa;
q is 1, 2, or 3; and
X has the structure:

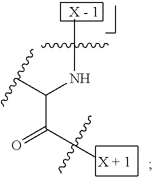

or a pharmaceutically acceptable salt, solvate, or hydrate thereof.

Embodiment 172. The IL-10 conjugate of embodiment 171 or 171.1, wherein the ratio of the amount of the structure of Formula (II) to the amount of the structure of Formula (III) comprising the total amount of [AzK_PEG30 kDa] in the IL-10 conjugate is about 1:1.

Embodiment 173. The IL-10 conjugate of embodiment 171 or 171.1, wherein the ratio of the amount of the structure of Formula (II) to the amount of the structure of Formula (III) comprising the total amount of [AzK_PEG30 kDa] in the IL-10 conjugate is greater than 1:1.

Embodiment 174. The IL-10 conjugate of embodiment 171 or 171.1, wherein the ratio of the amount of the structure of Formula (II) to the amount of the structure of Formula (III) comprising the total amount of [AzK_PEG30 kDa] in the IL-10 conjugate is less than 1:1.

Embodiment 175. An IL-10 conjugate comprising the amino acid sequence of any one of SEQ ID NOS: 59 to 66, wherein [AzK_L1_PEG] has the structure of Formula (IV), Formula (V), or a mixture of Formula (IV) and Formula (V):

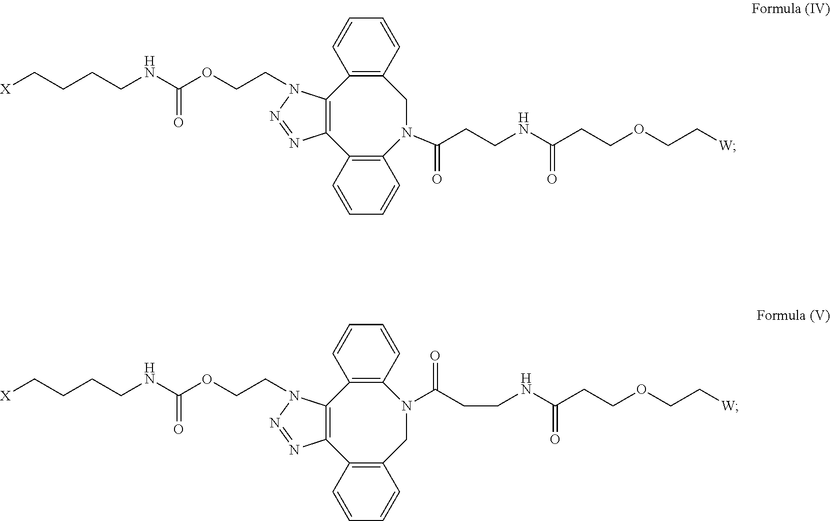

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

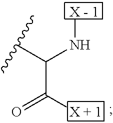

or a pharmaceutically acceptable salt, solvate, or hydrate thereof.

Embodiment 175.1. An IL-10 conjugate comprising the amino acid sequence of any one of SEQ ID NOS: 59 to 66, wherein [AzK_L1_PEG] has the structure of Formula (IV), Formula (V), or a mixture of Formula (IV) and Formula (V):

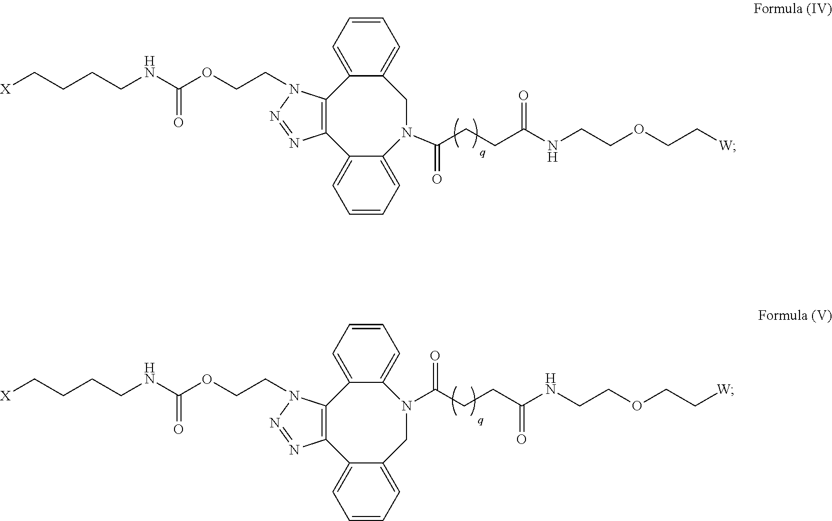

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa;
q is 1, 2, or 3; and
X has the structure:

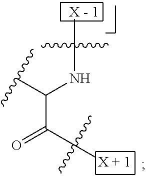

or a pharmaceutically acceptable salt, solvate, or hydrate thereof.

Embodiment 176. The IL-10 conjugate of embodiment 175 or 175.1, wherein the [AzK_L1_PEG] is a mixture of Formula (IV) and Formula (V), or a pharmaceutically acceptable salt, solvate, or hydrate thereof.

Embodiment 177. The IL-10 conjugate of embodiment 175 or 175.1, wherein the [AzK_L1_PEG] has the structure of Formula (IV):

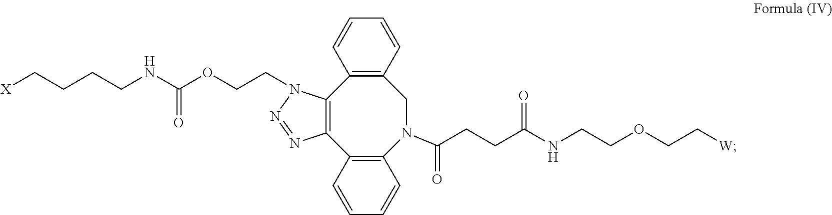

or a pharmaceutically acceptable salt, solvate, or hydrate thereof.

Embodiment 178. The IL-10 conjugate of embodiment 177, wherein the IL-10 conjugate has the amino acid sequence of SEQ ID NO: 59, or a pharmaceutically acceptable salt, solvate, or hydrate thereof.

Embodiment 179. The IL-10 conjugate of embodiment 178, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 180. The IL-10 conjugate of embodiment 179, wherein W is a PEG group having an average molecular weight selected from 20 kDa and 30 kDa, or a pharmaceutically acceptable salt, solvate, or hydrate thereof.

Embodiment 181. The IL-10 conjugate of embodiment 180, wherein W is a PEG group having an average molecular weight of 20 kDa, or a pharmaceutically acceptable salt, solvate, or hydrate thereof.

Embodiment 182. The IL-10 conjugate of embodiment 180, wherein W is a PEG group having an average molecular weight of 30 kDa, or a pharmaceutically acceptable salt, solvate, or hydrate thereof.

Embodiment 183. The IL-10 conjugate of embodiment 177, wherein the IL-10 conjugate has the amino acid sequence of SEQ ID NO: 60, or a pharmaceutically acceptable salt, solvate, or hydrate thereof.

Embodiment 184. The IL-10 conjugate of embodiment 183, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 185. The IL-10 conjugate of embodiment 184, wherein W is a PEG group having an average molecular weight selected from 20 kDa and 30 kDa, or a pharmaceutically acceptable salt, solvate, or hydrate thereof.

Embodiment 186. The IL-10 conjugate of embodiment 185, wherein W is a PEG group having an average molecular weight of 20 kDa, or a pharmaceutically acceptable salt, solvate, or hydrate thereof.

Embodiment 187. The IL-10 conjugate of embodiment 185, wherein W is a PEG group having an average molecular weight of 30 kDa, or a pharmaceutically acceptable salt, solvate, or hydrate thereof.

Embodiment 188. The IL-10 conjugate of embodiment 177, wherein the IL-10 conjugate has the amino acid sequence of SEQ ID NO: 61, or a pharmaceutically acceptable salt, solvate, or hydrate thereof.

Embodiment 189. The IL-10 conjugate of embodiment 188, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 190. The IL-10 conjugate of embodiment 189, wherein W is a PEG group having an average molecular weight selected from 20 kDa and 30 kDa, or a pharmaceutically acceptable salt, solvate, or hydrate thereof.

Embodiment 191. The IL-10 conjugate of embodiment 190, wherein W is a PEG group having an average molecular weight of 20 kDa, or a pharmaceutically acceptable salt, solvate, or hydrate thereof.

Embodiment 192. The IL-10 conjugate of embodiment 190, wherein W is a PEG group having an average molecular weight of 30 kDa, or a pharmaceutically acceptable salt, solvate, or hydrate thereof.

Embodiment 193. The IL-10 conjugate of embodiment 177, wherein the IL-10 conjugate has the amino acid sequence of SEQ ID NO: 62, or a pharmaceutically acceptable salt, solvate, or hydrate thereof.

Embodiment 194. The IL-10 conjugate of embodiment 193, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 195. The IL-10 conjugate of embodiment 194, wherein W is a PEG group having an average molecular weight selected from 20 kDa and 30 kDa, or a pharmaceutically acceptable salt, solvate, or hydrate thereof.

Embodiment 196. The IL-10 conjugate of embodiment 195, wherein W is a PEG group having an average molecular weight of 20 kDa, or a pharmaceutically acceptable salt, solvate, or hydrate thereof.

Embodiment 197. The IL-10 conjugate of embodiment 195, wherein W is a PEG group having an average molecular weight of 30 kDa, or a pharmaceutically acceptable salt, solvate, or hydrate thereof.

Embodiment 198. The IL-10 conjugate of embodiment 177, wherein the IL-10 conjugate has the amino acid sequence of SEQ ID NO: 63, or a pharmaceutically acceptable salt, solvate, or hydrate thereof.

Embodiment 199. The IL-10 conjugate of embodiment 198, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 200. The IL-10 conjugate of embodiment 199, wherein W is a PEG group having an average molecular weight selected from 20 kDa and 30 kDa, or a pharmaceutically acceptable salt, solvate, or hydrate thereof.

Embodiment 201. The IL-10 conjugate of embodiment 200, wherein W is a PEG group having an average molecular weight of 20 kDa, or a pharmaceutically acceptable salt, solvate, or hydrate thereof.

Embodiment 202. The IL-10 conjugate of embodiment 200, wherein W is a PEG group having an average molecular weight of 30 kDa, or a pharmaceutically acceptable salt, solvate, or hydrate thereof.

Embodiment 203. The IL-10 conjugate of embodiment 177, wherein the IL-10 conjugate has the amino acid sequence of SEQ ID NO: 64, or a pharmaceutically acceptable salt, solvate, or hydrate thereof.

Embodiment 204. The IL-10 conjugate of embodiment 203, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 205. The IL-10 conjugate of embodiment 204, wherein W is a PEG group having an average molecular weight selected from 20 kDa and 30 kDa, or a pharmaceutically acceptable salt, solvate, or hydrate thereof.

Embodiment 206. The IL-10 conjugate of embodiment 205, wherein W is a PEG group having an average molecular weight of 20 kDa, or a pharmaceutically acceptable salt, solvate, or hydrate thereof.

Embodiment 207. The IL-10 conjugate of embodiment 205, wherein W is a PEG group having an average molecular weight of 30 kDa, or a pharmaceutically acceptable salt, solvate, or hydrate thereof.

Embodiment 208. The IL-10 conjugate of embodiment 177, wherein the IL-10 conjugate has the amino acid sequence of SEQ ID NO: 65, or a pharmaceutically acceptable salt, solvate, or hydrate thereof.

Embodiment 209. The IL-10 conjugate of embodiment 208, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 210. The IL-10 conjugate of embodiment 209, wherein W is a PEG group having an average molecular weight selected from 20 kDa and 30 kDa, or a pharmaceutically acceptable salt, solvate, or hydrate thereof.

Embodiment 211. The IL-10 conjugate of embodiment 210, wherein W is a PEG group having an average molecular weight of 20 kDa, or a pharmaceutically acceptable salt, solvate, or hydrate thereof.

Embodiment 212. The IL-10 conjugate of embodiment 210, wherein W is a PEG group having an average molecular weight of 30 kDa, or a pharmaceutically acceptable salt, solvate, or hydrate thereof.

Embodiment 213. The IL-10 conjugate of embodiment 177, wherein the IL-10 conjugate has the amino acid sequence of SEQ ID NO: 66, or a pharmaceutically acceptable salt, solvate, or hydrate thereof.

Embodiment 214. The IL-10 conjugate of embodiment 213, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 215. The IL-10 conjugate of embodiment 214, wherein W is a PEG group having an average molecular weight selected from 20 kDa and 30 kDa, or a pharmaceutically acceptable salt, solvate, or hydrate thereof.

Embodiment 216. The IL-10 conjugate of embodiment 215, wherein W is a PEG group having an average molecular weight of 20 kDa, or a pharmaceutically acceptable salt, solvate, or hydrate thereof.

Embodiment 217. The IL-10 conjugate of embodiment 215, wherein W is a PEG group having an average molecular weight of 30 kDa, or a pharmaceutically acceptable salt, solvate, or hydrate thereof.

Embodiment 218. The IL-10 conjugate of embodiment 175 or 175.1, wherein the [AzK_L1_PEG] has the structure of Formula (V):

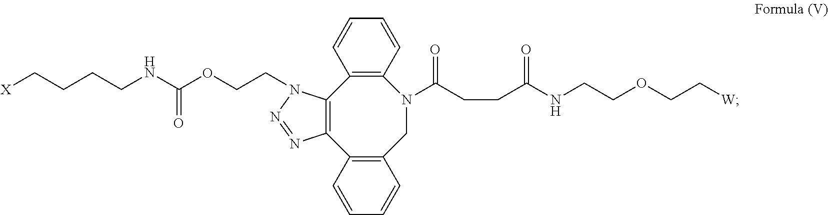

or a pharmaceutically acceptable salt, solvate, or hydrate thereof.

Embodiment 219. The IL-10 conjugate of embodiment 218, wherein the IL-10 conjugate has the amino acid sequence of SEQ ID NO: 59, or a pharmaceutically acceptable salt, solvate, or hydrate thereof.

Embodiment 220. The IL-10 conjugate of embodiment 219, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 221. The IL-10 conjugate of embodiment 220, wherein W is a PEG group having an average molecular weight selected from 20 kDa and 30 kDa, or a pharmaceutically acceptable salt, solvate, or hydrate thereof.

Embodiment 222. The IL-10 conjugate of embodiment 221, wherein W is a PEG group having an average molecular weight of 20 kDa, or a pharmaceutically acceptable salt, solvate, or hydrate thereof.

Embodiment 223. The IL-10 conjugate of embodiment 221, wherein W is a PEG group having an average molecular weight of 30 kDa, or a pharmaceutically acceptable salt, solvate, or hydrate thereof.

Embodiment 224. The IL-10 conjugate of embodiment 218, wherein the IL-10 conjugate has the amino acid sequence of SEQ ID NO: 60, or a pharmaceutically acceptable salt, solvate, or hydrate thereof.

Embodiment 225. The IL-10 conjugate of embodiment 224, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 226. The IL-10 conjugate of embodiment 225, wherein W is a PEG group having an average molecular weight selected from 20 kDa and 30 kDa, or a pharmaceutically acceptable salt, solvate, or hydrate thereof.

Embodiment 227. The IL-10 conjugate of embodiment 226, wherein W is a PEG group having an average molecular weight of 20 kDa, or a pharmaceutically acceptable salt, solvate, or hydrate thereof.

Embodiment 228. The IL-10 conjugate of embodiment 226, wherein W is a PEG group having an average molecular weight of 30 kDa, or a pharmaceutically acceptable salt, solvate, or hydrate thereof.

Embodiment 229. The IL-10 conjugate of embodiment 218, wherein the IL-10 conjugate has the amino acid sequence of SEQ ID NO: 61, or a pharmaceutically acceptable salt, solvate, or hydrate thereof.

Embodiment 230. The IL-10 conjugate of embodiment 229, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 231. The IL-10 conjugate of embodiment 230, wherein W is a PEG group having an average molecular weight selected from 20 kDa and 30 kDa, or a pharmaceutically acceptable salt, solvate, or hydrate thereof.

Embodiment 232. The IL-10 conjugate of embodiment 231, wherein W is a PEG group having an average molecular weight of 20 kDa, or a pharmaceutically acceptable salt, solvate, or hydrate thereof.

Embodiment 233. The IL-10 conjugate of embodiment 231, wherein W is a PEG group having an average molecular weight of 30 kDa, or a pharmaceutically acceptable salt, solvate, or hydrate thereof.

Embodiment 234. The IL-10 conjugate of embodiment 218, wherein the IL-10 conjugate has the amino acid sequence of SEQ ID NO: 62, or a pharmaceutically acceptable salt, solvate, or hydrate thereof.

Embodiment 235. The IL-10 conjugate of embodiment 234, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 236. The IL-10 conjugate of embodiment 235, wherein W is a PEG group having an average molecular weight selected from 20 kDa and 30 kDa, or a pharmaceutically acceptable salt, solvate, or hydrate thereof.

Embodiment 237. The IL-10 conjugate of embodiment 236, wherein W is a PEG group having an average molecular weight of 20 kDa, or a pharmaceutically acceptable salt, solvate, or hydrate thereof.

Embodiment 238. The IL-10 conjugate of embodiment 236, wherein W is a PEG group having an average molecular weight of 30 kDa, or a pharmaceutically acceptable salt, solvate, or hydrate thereof.

Embodiment 239. The IL-10 conjugate of embodiment 218, wherein the IL-10 conjugate has the amino acid sequence of SEQ ID NO: 63, or a pharmaceutically acceptable salt, solvate, or hydrate thereof.

Embodiment 240. The IL-10 conjugate of embodiment 239, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 241. The IL-10 conjugate of embodiment 240, wherein W is a PEG group having an average molecular weight selected from 20 kDa and 30 kDa, or a pharmaceutically acceptable salt, solvate, or hydrate thereof.

Embodiment 242. The IL-10 conjugate of embodiment 241, wherein W is a PEG group having an average molecular weight of 20 kDa, or a pharmaceutically acceptable salt, solvate, or hydrate thereof.

Embodiment 243. The IL-10 conjugate of embodiment 241, wherein W is a PEG group having an average molecular weight of 30 kDa, or a pharmaceutically acceptable salt, solvate, or hydrate thereof.

Embodiment 244. The IL-10 conjugate of embodiment 218, wherein the IL-10 conjugate has the amino acid sequence of SEQ ID NO: 64, or a pharmaceutically acceptable salt, solvate, or hydrate thereof.

Embodiment 245. The IL-10 conjugate of embodiment 244, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 246. The IL-10 conjugate of embodiment 245, wherein W is a PEG group having an average molecular weight selected from 20 kDa and 30 kDa, or a pharmaceutically acceptable salt, solvate, or hydrate thereof.

Embodiment 247. The IL-10 conjugate of embodiment 246, wherein W is a PEG group having an average molecular weight of 20 kDa, or a pharmaceutically acceptable salt, solvate, or hydrate thereof.

Embodiment 248. The IL-10 conjugate of embodiment 246, wherein W is a PEG group having an average molecular weight of 30 kDa, or a pharmaceutically acceptable salt, solvate, or hydrate thereof.

Embodiment 249. The IL-10 conjugate of embodiment 218, wherein the IL-10 conjugate has the amino acid sequence of SEQ ID NO: 65, or a pharmaceutically acceptable salt, solvate, or hydrate thereof.

Embodiment 250. The IL-10 conjugate of embodiment 249, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 251. The IL-10 conjugate of embodiment 250, wherein W is a PEG group having an average molecular weight selected from 20 kDa and 30 kDa, or a pharmaceutically acceptable salt, solvate, or hydrate thereof.

Embodiment 252. The IL-10 conjugate of embodiment 251, wherein W is a PEG group having an average molecular weight of 20 kDa, or a pharmaceutically acceptable salt, solvate, or hydrate thereof.

Embodiment 253. The IL-10 conjugate of embodiment 251, wherein W is a PEG group having an average molecular weight of 30 kDa, or a pharmaceutically acceptable salt, solvate, or hydrate thereof.

Embodiment 254. The IL-10 conjugate of embodiment 218, wherein the IL-10 conjugate has the amino acid sequence of SEQ ID NO: 66, or a pharmaceutically acceptable salt, solvate, or hydrate thereof.

Embodiment 255. The IL-10 conjugate of embodiment 254, wherein W is a PEG group having an average molecular weight selected from 5 kDa, 10 kDa, 15 kDa, 20 kDa, 25 kDa, and 30 kDa, or a pharmaceutically acceptable salt, solvate, or hydrate thereof.

Embodiment 256. The IL-10 conjugate of embodiment 255, wherein W is a PEG group having an average molecular weight selected from 20 kDa and 30 kDa, or a pharmaceutically acceptable salt, solvate, or hydrate thereof.

Embodiment 257. The IL-10 conjugate of embodiment 256, wherein W is a PEG group having an average molecular weight of 20 kDa, or a pharmaceutically acceptable salt, solvate, or hydrate thereof.

Embodiment 258. The IL-10 conjugate of embodiment 256, wherein W is a PEG group having an average molecular weight of 30 kDa, or a pharmaceutically acceptable salt, solvate, or hydrate thereof.

Embodiment 259. The IL-10 conjugate of any one of embodiments 175 to 258, wherein W is a linear or branched PEG group, or a pharmaceutically acceptable salt, solvate, or hydrate thereof.

Embodiment 260. The IL-10 conjugate of any one of embodiments 178 to 258, wherein W is a linear PEG group, or a pharmaceutically acceptable salt, solvate, or hydrate thereof.

Embodiment 261. The IL-10 conjugate of any one of embodiments 175 to 258, wherein W is a branched PEG group, or a pharmaceutically acceptable salt, solvate, or hydrate thereof.

Embodiment 262. The IL-10 conjugate of any one of embodiments 175 to 258, wherein W is a methoxy PEG group, or a pharmaceutically acceptable salt, solvate, or hydrate thereof.

Embodiment 263. The IL-10 conjugate of embodiment 262, wherein the methoxy PEG group is linear or branched, or a pharmaceutically acceptable salt, solvate, or hydrate thereof.

Embodiment 264. The IL-10 conjugate of embodiment 263, wherein the methoxy PEG group is linear, or a pharmaceutically acceptable salt, solvate, or hydrate thereof.

Embodiment 265. The IL-10 conjugate of embodiment 263, wherein the methoxy PEG group is branched, or a pharmaceutically acceptable salt, solvate, or hydrate thereof.

Embodiment 266. An IL-10 conjugate comprising the amino acid sequence of any one of SEQ ID NOS: 43 to 50, wherein [AzK_L1_PEG20 kDa] has the structure of Formula (IV), Formula (V), or a mixture of Formula (IV) and Formula (V):

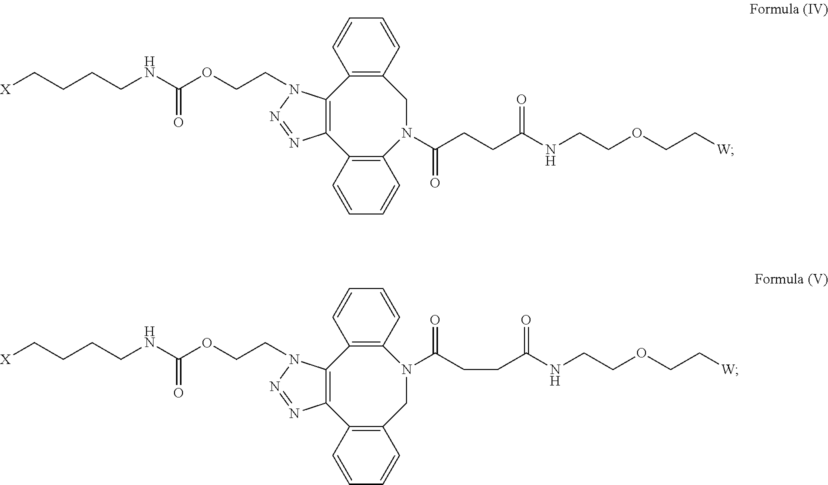

wherein:
W is a PEG group having an average molecular weight of 20 kDa; and
X has the structure:

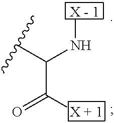

or a pharmaceutically acceptable salt, solvate, or hydrate thereof.

Embodiment 266.1. An IL-10 conjugate comprising the amino acid sequence of any one of SEQ ID NOS: 43 to 50, wherein [AzK_L1_PEG20 kDa] has the structure of Formula (IV), Formula (V), or a mixture of Formula (IV) and Formula (V):

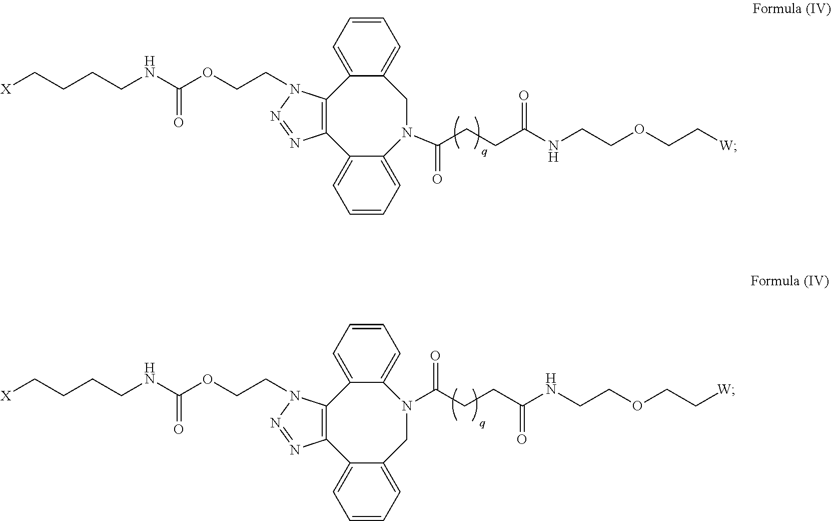

wherein:
W is a PEG group having an average molecular weight of 20 kDa;
q is 1, 2, or 3; and
X has the structure:

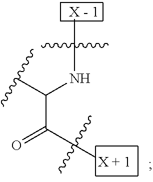

or a pharmaceutically acceptable salt, solvate, or hydrate thereof.

Embodiment 267. The IL-10 conjugate of embodiment 266 or 266.1, wherein the IL-10 conjugate has the amino acid sequence of SEQ ID NO: 43, or a pharmaceutically acceptable salt, solvate, or hydrate thereof.

Embodiment 268. The IL-10 conjugate of embodiment 266 or 266.1, wherein the IL-10 conjugate has the amino acid sequence of SEQ ID NO: 44, or a pharmaceutically acceptable salt, solvate, or hydrate thereof.

Embodiment 269. The IL-10 conjugate of embodiment 266 or 266.1, wherein the IL-10 conjugate has the amino acid sequence of SEQ ID NO: 45, or a pharmaceutically acceptable salt, solvate, or hydrate thereof.

Embodiment 270. The IL-10 conjugate of embodiment 266 or 266.1, wherein the IL-10 conjugate has the amino acid sequence of SEQ ID NO: 46, or a pharmaceutically acceptable salt, solvate, or hydrate thereof.

Embodiment 271. The IL-10 conjugate of embodiment 266 or 266.1, wherein the IL-10 conjugate has the amino acid sequence of SEQ ID NO: 47, or a pharmaceutically acceptable salt, solvate, or hydrate thereof.

Embodiment 272. The IL-10 conjugate of embodiment 266 or 266.1, wherein the IL-10 conjugate has the amino acid sequence of SEQ ID NO: 48, or a pharmaceutically acceptable salt, solvate, or hydrate thereof.

Embodiment 273. The IL-10 conjugate of embodiment 266 or 266.1, wherein the IL-10 conjugate has the amino acid sequence of SEQ ID NO: 49, or a pharmaceutically acceptable salt, solvate, or hydrate thereof.

Embodiment 274. The IL-10 conjugate of embodiment 266 or 266.1, wherein the IL-10 conjugate has the amino acid sequence of SEQ ID NO: 50, or a pharmaceutically acceptable salt, solvate, or hydrate thereof.

Embodiment 275. The IL-10 conjugate of embodiment 266 or 266.1, wherein the [AzK_L1_PEG20 kDa] has the structure of Formula (IV):

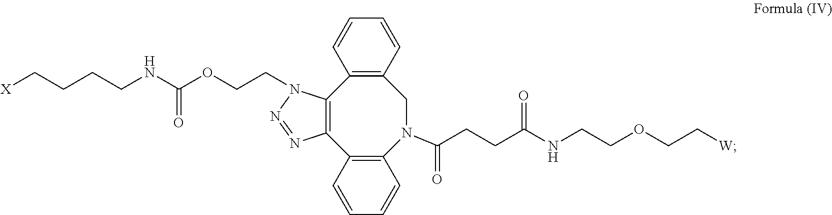

or a pharmaceutically acceptable salt, solvate, or hydrate thereof.

Embodiment 276. The IL-10 conjugate of embodiment 275, wherein the IL-10 conjugate has the amino acid sequence of SEQ ID NO: 43, or a pharmaceutically acceptable salt, solvate, or hydrate thereof.

Embodiment 277. The IL-10 conjugate of embodiment 275, wherein the IL-10 conjugate has the amino acid sequence of SEQ ID NO: 44, or a pharmaceutically acceptable salt, solvate, or hydrate thereof.

Embodiment 278. The IL-10 conjugate of embodiment 275, wherein the IL-10 conjugate has the amino acid sequence of SEQ ID NO: 45, or a pharmaceutically acceptable salt, solvate, or hydrate thereof.

Embodiment 279. The IL-10 conjugate of embodiment 275, wherein the IL-10 conjugate has the amino acid sequence of SEQ ID NO: 46, or a pharmaceutically acceptable salt, solvate, or hydrate thereof.

Embodiment 280. The IL-10 conjugate of embodiment 275, wherein the IL-10 conjugate has the amino acid sequence of SEQ ID NO: 47, or a pharmaceutically acceptable salt, solvate, or hydrate thereof.

Embodiment 281. The IL-10 conjugate of embodiment 275, wherein the IL-10 conjugate has the amino acid sequence of SEQ ID NO: 48, or a pharmaceutically acceptable salt, solvate, or hydrate thereof.

Embodiment 282. The IL-10 conjugate of embodiment 275, wherein the IL-10 conjugate has the amino acid sequence of SEQ ID NO: 49, or a pharmaceutically acceptable salt, solvate, or hydrate thereof.

Embodiment 283. The IL-10 conjugate of embodiment 275, wherein the IL-10 conjugate has the amino acid sequence of SEQ ID NO: 50, or a pharmaceutically acceptable salt, solvate, or hydrate thereof.

Embodiment 284. The IL-10 conjugate of embodiment 266 or 266.1, wherein the [AzK_L1_PEG20 kDa] has the structure of Formula (V):

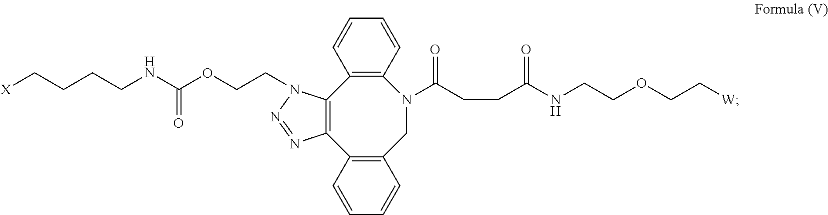

or a pharmaceutically acceptable salt, solvate, or hydrate thereof.

Embodiment 285. The IL-10 conjugate of embodiment 284, wherein the IL-10 conjugate has the amino acid sequence of SEQ ID NO: 43, or a pharmaceutically acceptable salt, solvate, or hydrate thereof.

Embodiment 286. The IL-10 conjugate of embodiment 284, wherein the IL-10 conjugate has the amino acid sequence of SEQ ID NO: 44, or a pharmaceutically acceptable salt, solvate, or hydrate thereof.

Embodiment 287. The IL-10 conjugate of embodiment 284, wherein the IL-10 conjugate has the amino acid sequence of SEQ ID NO: 45, or a pharmaceutically acceptable salt, solvate, or hydrate thereof.

Embodiment 288. The IL-10 conjugate of embodiment 284, wherein the IL-10 conjugate has the amino acid sequence of SEQ ID NO: 46, or a pharmaceutically acceptable salt, solvate, or hydrate thereof.

Embodiment 289. The IL-10 conjugate of embodiment 284, wherein the IL-10 conjugate has the amino acid sequence of SEQ ID NO: 47, or a pharmaceutically acceptable salt, solvate, or hydrate thereof.

Embodiment 290. The IL-10 conjugate of embodiment 284, wherein the IL-10 conjugate has the amino acid sequence of SEQ ID NO: 48, or a pharmaceutically acceptable salt, solvate, or hydrate thereof.

Embodiment 291. The IL-10 conjugate of embodiment 284, wherein the IL-10 conjugate has the amino acid sequence of SEQ ID NO: 49, or a pharmaceutically acceptable salt, solvate, or hydrate thereof.

Embodiment 292. The IL-10 conjugate of embodiment 284, wherein the IL-10 conjugate has the amino acid sequence of SEQ ID NO: 50, or a pharmaceutically acceptable salt, solvate, or hydrate thereof.

Embodiment 293. An IL-10 conjugate comprising the amino acid sequence of any one of SEQ ID NOS: 51 to 58, wherein [AzK_L1_PEG30 kDa] has the structure of Formula (IV), Formula (V), or a mixture of the structures of Formula (IV) and Formula (V):

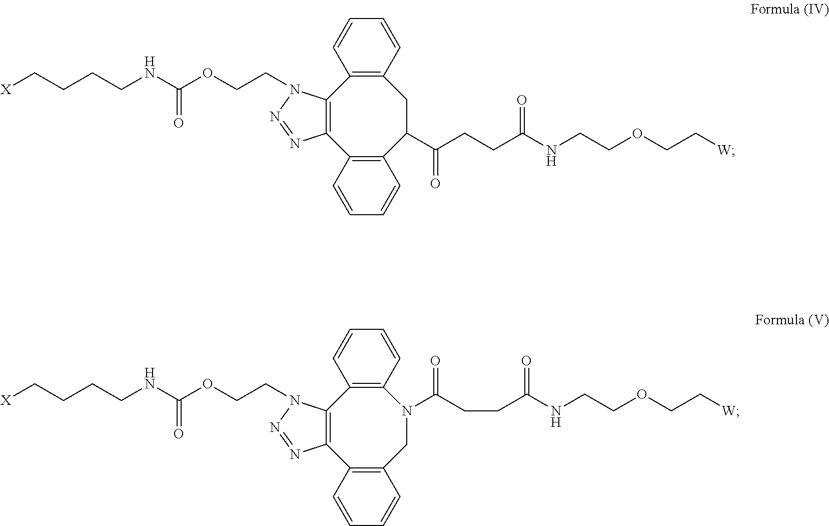

wherein:
W is a PEG group having an average molecular weight of 30 kDa; and
X has the structure:

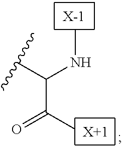

or a pharmaceutically acceptable salt, solvate, or hydrate thereof.

Embodiment 293.1. An IL-10 conjugate comprising the amino acid sequence of any one of SEQ ID NOS: 51 to 58, wherein [AzK_L1_PEG30 kDa] has the structure of Formula (IV), Formula (V), or a mixture of the structures of Formula (IV) and Formula (V):

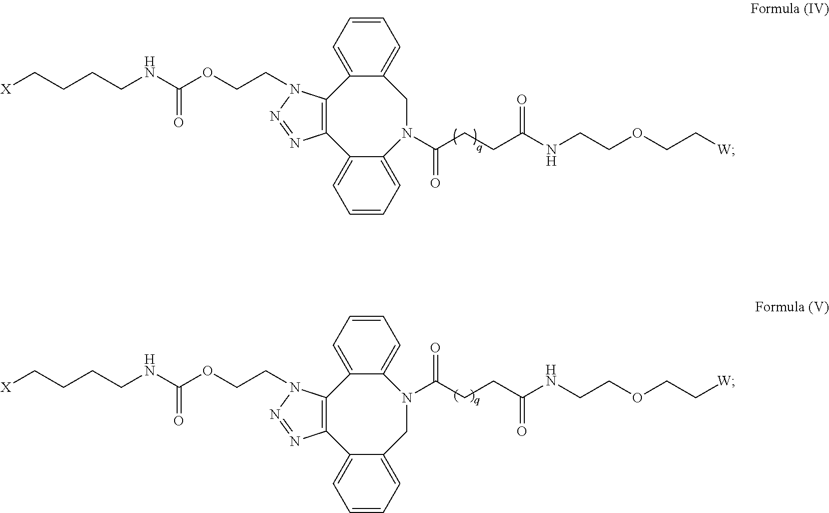

wherein:
W is a PEG group having an average molecular weight of 30 kDa;
q is 1, 2, or 3; and
X has the structure:

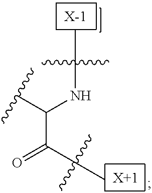

or a pharmaceutically acceptable salt, solvate, or hydrate thereof.

Embodiment 294. The IL-10 conjugate of embodiment 293 or 293.1, wherein the IL-10 conjugate has the amino acid sequence of SEQ ID NO: 51, or a pharmaceutically acceptable salt, solvate, or hydrate thereof.

Embodiment 295. The IL-10 conjugate of embodiment 293 or 293.1, wherein the IL-10 conjugate has the amino acid sequence of SEQ ID NO: 52, or a pharmaceutically acceptable salt, solvate, or hydrate thereof.

Embodiment 296. The IL-10 conjugate of embodiment 293 or 293.1, wherein the IL-10 conjugate has the amino acid sequence of SEQ ID NO: 53, or a pharmaceutically acceptable salt, solvate, or hydrate thereof.

Embodiment 297. The IL-10 conjugate of embodiment 293 or 293.1, wherein the IL-10 conjugate has the amino acid sequence of SEQ ID NO: 54, or a pharmaceutically acceptable salt, solvate, or hydrate thereof.

Embodiment 298. The IL-10 conjugate of embodiment 293 or 293.1, wherein the IL-10 conjugate has the amino acid sequence of SEQ ID NO: 55, or a pharmaceutically acceptable salt, solvate, or hydrate thereof.

Embodiment 299. The IL-10 conjugate of embodiment 293 or 293.1, wherein the IL-10 conjugate has the amino acid sequence of SEQ ID NO: 56, or a pharmaceutically acceptable salt, solvate, or hydrate thereof.

Embodiment 300. The IL-10 conjugate of embodiment 293 or 293.1, wherein the IL-10 conjugate has the amino acid sequence of SEQ ID NO: 57, or a pharmaceutically acceptable salt, solvate, or hydrate thereof.

Embodiment 301. The IL-10 conjugate of embodiment 293 or 293.1, wherein the IL-10 conjugate has the amino acid sequence of SEQ ID NO: 58, or a pharmaceutically acceptable salt, solvate, or hydrate thereof.

Embodiment 302. The IL-10 conjugate of embodiment 293 or 293.1, wherein the [AzK_L1_PEG30 kDa] has the structure of Formula (IV):

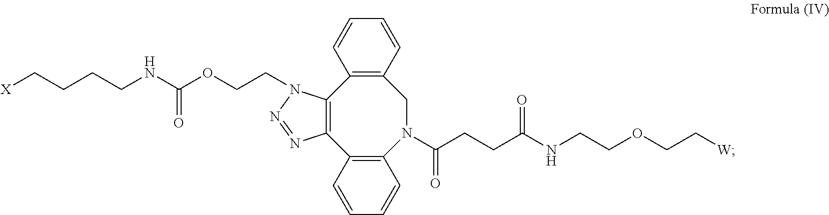

or a pharmaceutically acceptable salt, solvate, or hydrate thereof.

Embodiment 303. The IL-10 conjugate of embodiment 302, wherein the IL-10 conjugate has the amino acid sequence of SEQ ID NO: 51, or a pharmaceutically acceptable salt, solvate, or hydrate thereof.

Embodiment 304. The IL-10 conjugate of embodiment 302, wherein the IL-10 conjugate has the amino acid sequence of SEQ ID NO: 52, or a pharmaceutically acceptable salt, solvate, or hydrate thereof.

Embodiment 305. The IL-10 conjugate of embodiment 302, wherein the IL-10 conjugate has the amino acid sequence of SEQ ID NO: 53, or a pharmaceutically acceptable salt, solvate, or hydrate thereof.

Embodiment 306. The IL-10 conjugate of embodiment 302, wherein the IL-10 conjugate has the amino acid sequence of SEQ ID NO: 54, or a pharmaceutically acceptable salt, solvate, or hydrate thereof.

Embodiment 307. The IL-10 conjugate of embodiment 302, wherein the IL-10 conjugate has the amino acid sequence of SEQ ID NO: 55, or a pharmaceutically acceptable salt, solvate, or hydrate thereof.

Embodiment 308. The IL-10 conjugate of embodiment 302, wherein the IL-10 conjugate has the amino acid sequence of SEQ ID NO: 56, or a pharmaceutically acceptable salt, solvate, or hydrate thereof.

Embodiment 309. The IL-10 conjugate of embodiment 302, wherein the IL-10 conjugate has the amino acid sequence of SEQ ID NO: 57, or a pharmaceutically acceptable salt, solvate, or hydrate thereof.

Embodiment 310. The IL-10 conjugate of embodiment 302, wherein the IL-10 conjugate has the amino acid sequence of SEQ ID NO: 58, or a pharmaceutically acceptable salt, solvate, or hydrate thereof.

Embodiment 311. The IL-10 conjugate of embodiment 293 or 293.1, wherein the [AzK_L1_PEG30 kDa] has the structure of Formula (V):

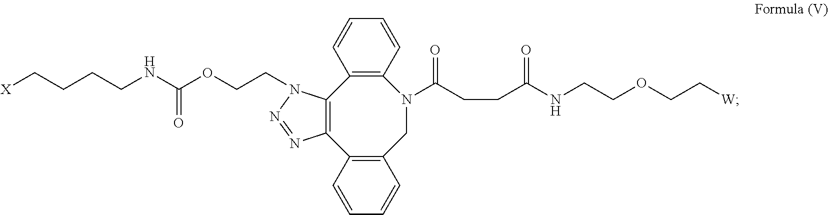

or a pharmaceutically acceptable salt, solvate, or hydrate thereof.

Embodiment 312. The IL-10 conjugate of embodiment 311, wherein the IL-10 conjugate has the amino acid sequence of SEQ ID NO: 51, or a pharmaceutically acceptable salt, solvate, or hydrate thereof.

Embodiment 313. The IL-10 conjugate of embodiment 311, wherein the IL-10 conjugate has the amino acid sequence of SEQ ID NO: 52, or a pharmaceutically acceptable salt, solvate, or hydrate thereof.

Embodiment 314. The IL-10 conjugate of embodiment 311, wherein the IL-10 conjugate has the amino acid sequence of SEQ ID NO: 53, or a pharmaceutically acceptable salt, solvate, or hydrate thereof.

Embodiment 315. The IL-10 conjugate of embodiment 311, wherein the IL-10 conjugate has the amino acid sequence of SEQ ID NO: 54, or a pharmaceutically acceptable salt, solvate, or hydrate thereof.

Embodiment 316. The IL-10 conjugate of embodiment 311, wherein the IL-10 conjugate has the amino acid sequence of SEQ ID NO: 55, or a pharmaceutically acceptable salt, solvate, or hydrate thereof.

Embodiment 317. The IL-10 conjugate of embodiment 311, wherein the IL-10 conjugate has the amino acid sequence of SEQ ID NO: 56, or a pharmaceutically acceptable salt, solvate, or hydrate thereof.

Embodiment 318. The IL-10 conjugate of embodiment 311, wherein the IL-10 conjugate has the amino acid sequence of SEQ ID NO: 57, or a pharmaceutically acceptable salt, solvate, or hydrate thereof.

Embodiment 319. The IL-10 conjugate of embodiment 311, wherein the IL-10 conjugate has the amino acid sequence of SEQ ID NO: 58, or a pharmaceutically acceptable salt, solvate, or hydrate thereof.

Embodiment 320. An IL-10 conjugate comprising the amino acid sequence of any one of SEQ ID NOS: 43 to 50, wherein [AzK_L1_PEG20 kDa] is a mixture of the structures of Formula (IV) and Formula (V):

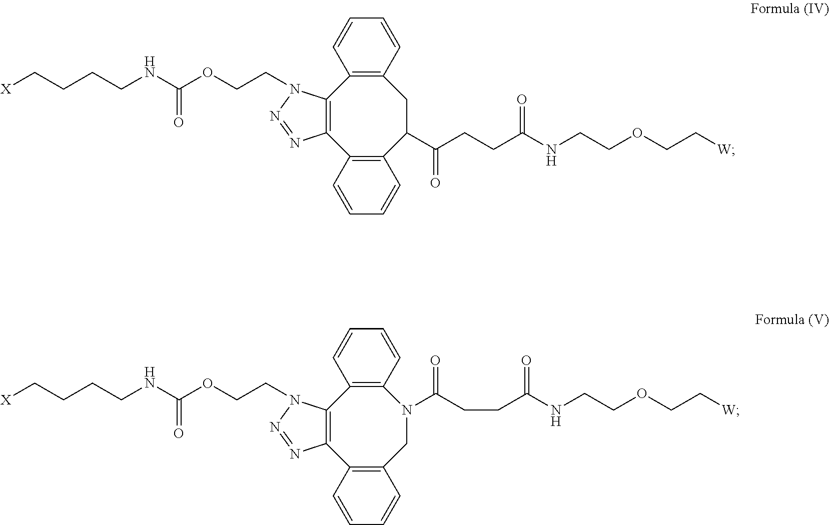

wherein:
W is a PEG group having an average molecular weight of 20 kDa; and
X has the structure.

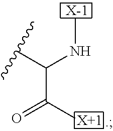

or a pharmaceutically acceptable salt, solvate, or hydrate thereof.

Embodiment 320.1. An IL-10 conjugate comprising the amino acid sequence of any one of SEQ ID NOS: 43 to 50, wherein [AzK_L1_PEG20 kDa] is a mixture of the structures of Formula (IV) and Formula (V):

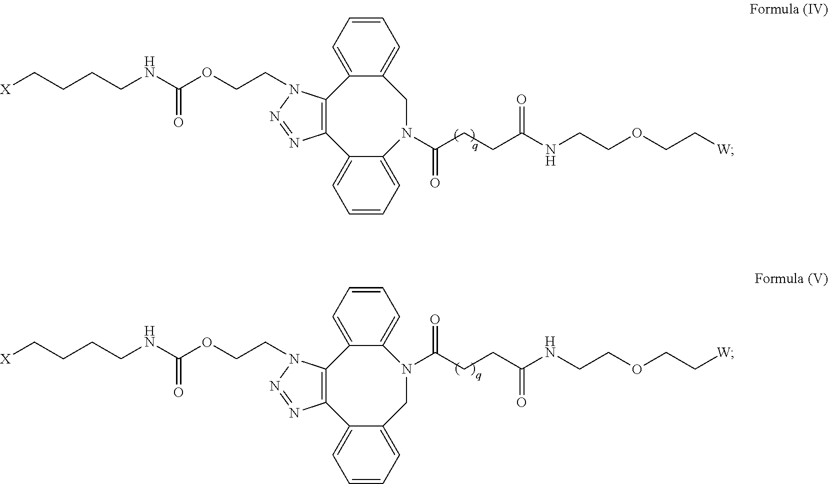

wherein:
W is a PEG group having an average molecular weight of 20 kDa;
q is 1, 2, or 3; and
X has the structure:

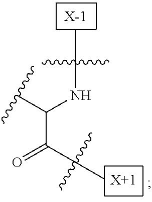

or a pharmaceutically acceptable salt, solvate, or hydrate thereof.

Embodiment 321. The IL-10 conjugate of embodiment 320 or 320.1, wherein the ratio of the amount of the structure of Formula (IV) to the amount of the structure of Formula (V) comprising the total amount of [AzK_L1_PEG20 kDa] in the IL-10 conjugate is about 1:1.

Embodiment 322. The IL-10 conjugate of embodiment 320 or 320.1, wherein the ratio of the amount of the structure of Formula (IV) to the amount of the structure of Formula (V) comprising the total amount of [AzK_L1_PEG20 kDa] in the IL-10 conjugate is greater than 1:1.

Embodiment 323. The IL-10 conjugate of embodiment 320 or 320.1, wherein the ratio of the amount of the structure of Formula (IV) to the amount of the structure of Formula (V) comprising the total amount of [AzK_L1_PEG20 kDa] in the IL-10 conjugate is less than 1:1.

Embodiment 324. An IL-10 conjugate comprising the amino acid sequence of any one of SEQ ID NOS: 51 to 58, wherein [AzK_L1 PEG30 kDa] is a mixture of the structures of Formula (IV) and Formula (V):

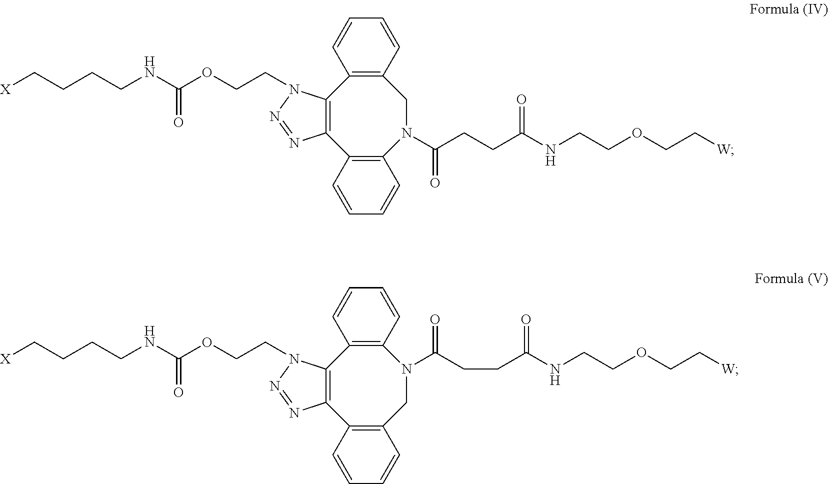

wherein:
W is a PEG group having an average molecular weight of 30 kDa; and
X has the structure:

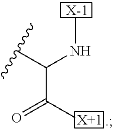

or a pharmaceutically acceptable salt, solvate, or hydrate thereof.

Embodiment 324.1. An IL-10 conjugate comprising the amino acid sequence of any one of SEQ ID NOS: 51 to 58, wherein [AzK_L1 PEG30 kDa] is a mixture of the structures of Formula (IV) and Formula (V):

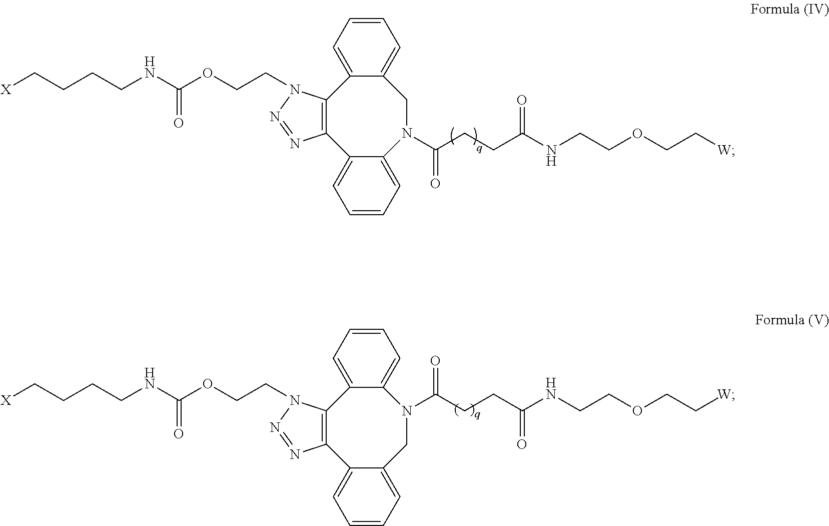

wherein:
W is a PEG group having an average molecular weight of 30 kDa;
q is 1, 2, or 3; and
X has the structure:

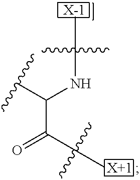

or a pharmaceutically acceptable salt, solvate, or hydrate thereof.

Embodiment 325. The IL-10 conjugate of embodiment 324 or 324.1, wherein the ratio of the amount of the structure of Formula (IV) to the amount of the structure of Formula (V) comprising the total amount of [AzK_L1_PEG30 kDa] in the IL-10 conjugate is about 1:1.

Embodiment 326. The IL-10 conjugate of embodiment 324 or 324.1, wherein the ratio of the amount of the structure of Formula (IV) to the amount of the structure of Formula (V) comprising the total amount of [AzK_L1_PEG30 kDa] in the IL-10 conjugate is greater than 1:1.

Embodiment 327. The IL-10 conjugate of embodiment 324 or 324.1, wherein the ratio of the amount of the structure of Formula (IV) to the amount of the structure of Formula (V) comprising the total amount of [AzK_L1_PEG30 kDa] in the IL-10 conjugate is less than 1:1.

Embodiment 328. An IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII):

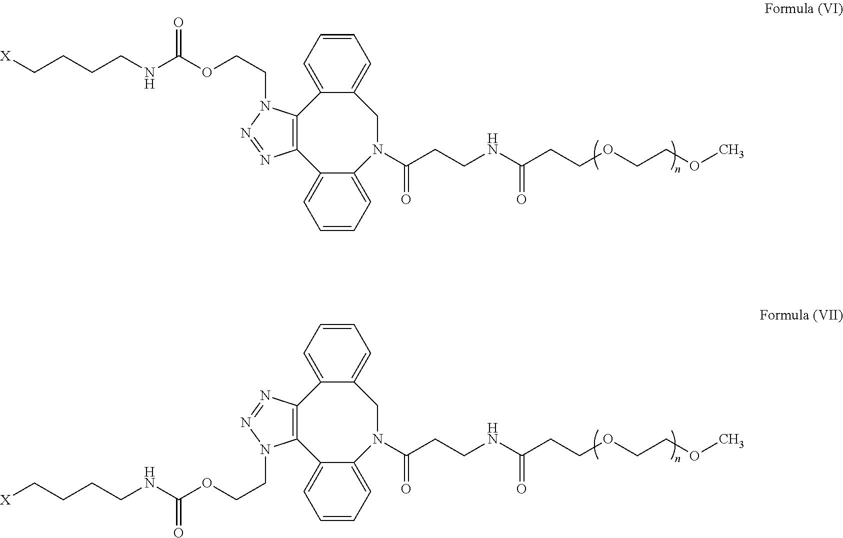

wherein:
n is an integer such that the molecular weight of the PEG group is from about 5,000 Daltons to about 60,000 Daltons; and
X has the structure:

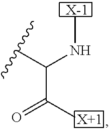

or a pharmaceutically acceptable salt, solvate, or hydrate thereof.

Embodiment 328.1. An IL-10 conjugate comprising the amino acid sequence of SEQ ID NO: 1 in which at least one amino acid residue in the IL-10 conjugate is replaced by the structure of Formula (VI), Formula (VII), or a mixture of Formula (VI) and Formula (VII):

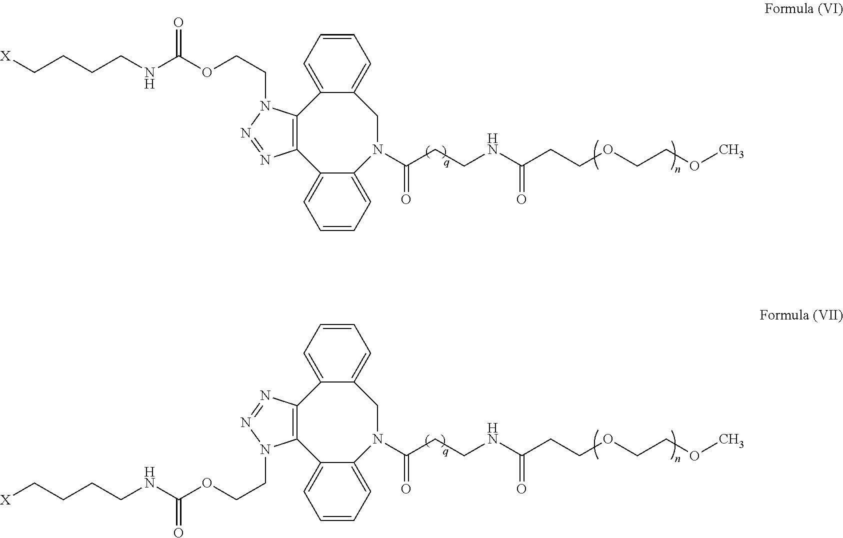

wherein:
q is 1, 2, or 3;
n is an integer such that the molecular weight of the PEG group is from about 5,000 Daltons to about 60,000 Daltons; and
X has the structure:

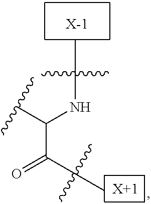

or a pharmaceutically acceptable salt, solvate, or hydrate thereof.

Embodiment 329. The IL-10 conjugate of embodiment 328 or 328.1, wherein the position of the structure Formula (VI), Formula (VII), or a mixture of Formula (VI) and Formula (VII), in the amino acid sequence of the IL-10 conjugate is selected from N82, K88, A89, K99, K125, N126, N129, and K130, or a pharmaceutically acceptable salt, solvate, or hydrate thereof.

Embodiment 330. The IL-10 conjugate of embodiment 329, wherein the position of the structure of Formula (VI), Formula (VII), or a mixture of Formula (VI) and Formula (VII), in the amino acid sequence of the IL-10 conjugate is selected from N82, K88, K99, N126, N129, and K130, or a pharmaceutically acceptable salt, solvate, or hydrate thereof.

Embodiment 331. The IL-10 conjugate of any one of embodiments 328 to 330, wherein the ratio of the amount of the structure of Formula (VI) to the amount of the structure of Formula (VII) comprising the total amount of the IL-10 conjugate is about 1:1.

Embodiment 332. The IL-10 conjugate of any one of embodiments 328 to 330, wherein the ratio of the amount of the structure of Formula (VI) to the amount of the structure of Formula (VII) comprising the total amount of the IL-10 conjugate is greater than 1:1.

Embodiment 333. The IL-10 conjugate of any one of embodiments 328 to 330, wherein the ratio of the amount of the structure of Formula (VI) to the amount of the structure of Formula (VII) comprising the total amount of the IL-10 conjugate is less than 1:1.

Embodiment 334. The IL-10 conjugate of any one of embodiments 328 to 333, wherein n is an integer such that the molecular weight of the PEG group is from about 5,000 Daltons to about 40,000 Daltons.

Embodiment 335. The IL-10 conjugate of embodiment 334, wherein n is an integer such that the molecular weight of the PEG group is from about 5,000 Daltons to about 30,000 Daltons.

Embodiment 336. The IL-10 conjugate of embodiment 334, wherein n is an integer such that the molecular weight of the PEG group is from about 5,000 Daltons to about 25,000 Daltons.

Embodiment 337. The IL-10 conjugate of embodiment 334, wherein n is an integer such that the molecular weight of the PEG group is from about 7,500 Daltons to about 30,000 Daltons.

Embodiment 338. The IL-10 conjugate of embodiment 334, wherein n is an integer such that the molecular weight of the PEG group is from about 10,000 Daltons to about 20,000 Daltons.

Embodiment 339. The IL-10 conjugate of embodiment 328 or 328.1, wherein the position of the structure Formula (VI) or Formula (VII), or a mixture of Formula (VI) and Formula (VII), in the amino acid sequence of the IL-10 conjugate is selected from N82, K88, A89, N129, and K130, and wherein n is an integer such that the molecular weight of the PEG group is from about 7,500 Daltons to about 30,000 Daltons.

Embodiment 340. The IL-10 conjugate of embodiment 339, wherein n is an integer such that the molecular weight of the PEG group is from about 10,000 Daltons to about 20,000 Daltons.

Embodiment 341. The IL-10 conjugate of embodiment 340, wherein the position of the structure Formula (VI) or Formula (VII), or a mixture of Formula (VI) and Formula (VII), in the amino acid sequence of the IL-10 conjugate is N82.

Embodiment 342. The IL-10 conjugate of embodiment 340, wherein the position of the structure Formula (VI) or Formula (VII), or a mixture of Formula (VI) and Formula (VII), in the amino acid sequence of the IL-10 conjugate is K88.

Embodiment 343. The IL-10 conjugate of embodiment 340, wherein the position of the structure Formula (VI) or Formula (VII), or a mixture of Formula (VI) and Formula (VII), in the amino acid sequence of the IL-10 conjugate is A89.

Embodiment 344. The IL-10 conjugate of embodiment 340, wherein the position of the structure Formula (VI) or Formula (VII), or a mixture of Formula (VI) and Formula (VII), in the amino acid sequence of the IL-10 conjugate is N129.

Embodiment 345. The IL-10 conjugate of embodiment 340, wherein the position of the structure Formula (VI) or Formula (VII), or a mixture of Formula (VI) and Formula (VII), in the amino acid sequence of the IL-10 conjugate is K130.

Embodiment 346. An IL-10 conjugate comprising the amino acid sequence of SEQ ID NO: 1 in which at least one amino acid residue in the IL-10 conjugate is replaced by the structure of Formula (VIII) or Formula (IX), or a mixture of Formula (VIII) and Formula (IX):

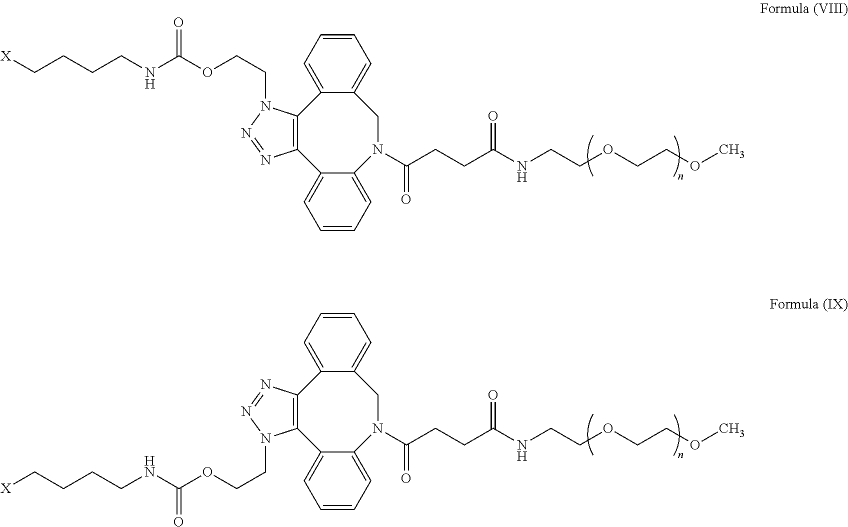

wherein:
n is an integer such that the molecular weight of the PEG group is from about 5,000 Daltons to about 60,000 Daltons; and
X has the structure:

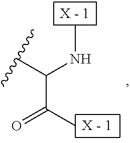 [SMILES: *C(NC)C(C)=O]

or a pharmaceutically acceptable salt, solvate, or hydrate thereof.

Embodiment 346.1. An IL-10 conjugate comprising the amino acid sequence of SEQ ID NO: 1 in which at least one amino acid residue in the IL-10 conjugate is replaced by the structure of Formula (VIII) or Formula (IX), or a mixture of Formula (VIII) and Formula (IX):

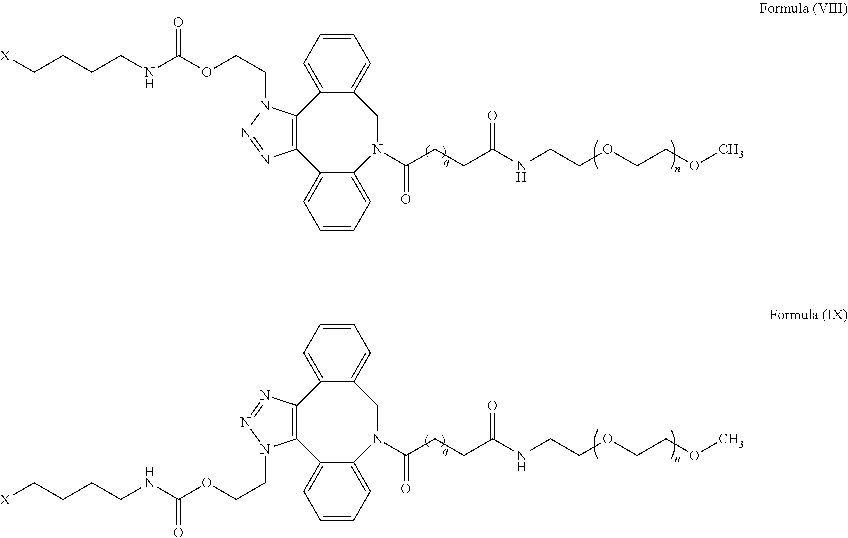

wherein:
q is 1, 2, or 3;
n is an integer such that the molecular weight of the PEG group is from about 5,000 Daltons to about 60,000 Daltons; and
X has the structure:

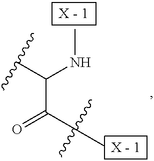 [SMILES: *C(=O)C(*)NC]

or a pharmaceutically acceptable salt, solvate, or hydrate thereof.

Embodiment 347. The IL-10 conjugate of embodiment 346 or 346.1, wherein the position of the structure Formula (VIII) or Formula (IX), or a mixture of Formula (VIII) and Formula (IX), in the amino acid sequence of the IL-10 conjugate is selected from N82, K88, A89, K99, K125, N126, N129, and K130, or a pharmaceutically acceptable salt, solvate, or hydrate thereof.

Embodiment 348. The IL-10 conjugate of embodiment 346 or 346.1, wherein the position of the structure of Formula (VIII) or Formula (IX), or a mixture of Formula (VIII) and Formula (IX), in the amino acid sequence of the IL-10 conjugate is selected from N82, K88, K99, N126, N129, and K130, or a pharmaceutically acceptable salt, solvate, or hydrate thereof.

Embodiment 349. The IL-10 conjugate of any one of embodiments 346 to 348, wherein the ratio of the amount of the structure of Formula (VIII) to the amount of the structure of Formula (IX) comprising the total amount of the IL-10 conjugate is about 1:1.

Embodiment 350. The IL-10 conjugate of any one of embodiments 346 to 348, wherein the ratio of the amount of the structure of Formula (VIII) to the amount of the structure of Formula (IX) comprising the total amount of the IL-10 conjugate is greater than 1:1.

Embodiment 351. The IL-10 conjugate of any one of embodiments 346 to 348, wherein the ratio of the amount of the structure of Formula (VIII) to the amount of the structure of Formula (IX) comprising the total amount of the IL-10 conjugate is less than 1:1.

Embodiment 352. The IL-10 conjugate of any one of embodiments 346 to 351, wherein n is an integer such that the molecular weight of the PEG group is from about 5,000 Daltons to about 40,000 Daltons.

Embodiment 353. The IL-10 conjugate of embodiment 352, wherein n is an integer such that the molecular weight of the PEG group is from about 5,000 Daltons to about 30,000 Daltons.

Embodiment 354. The IL-10 conjugate of embodiment 352, wherein n is an integer such that the molecular weight of the PEG group is from about 5,000 Daltons to about 25,000 Daltons.

Embodiment 355. The IL-10 conjugate of embodiment 352, wherein n is an integer such that the molecular weight of the PEG group is from about 7,500 Daltons to about 30,000 Daltons.

Embodiment 356. The IL-10 conjugate of embodiment 352, wherein n is an integer such that the molecular weight of the PEG group is from about 10,000 Daltons to about 20,000 Daltons.

Embodiment 357. The IL-10 conjugate of embodiment 346 or 346.1, wherein the position of the structure Formula (VIII) or Formula (IX), or a mixture of (VIII) and Formula (IX), in the amino acid sequence of the IL-10 conjugate is selected from N82, K88, A89, N129, and K130, and wherein n is an integer such that the molecular weight of the PEG group is from about 7,500 Daltons to about 30,000 Daltons.

Embodiment 358. The IL-10 conjugate of embodiment 357, wherein n is an integer such that the molecular weight of the PEG group is from about 10,000 Daltons to about 20,000 Daltons.

Embodiment 359. The IL-10 conjugate of embodiment 358, wherein the position of the structure Formula (VIII) or Formula (IX), or a mixture of Formula (VIII) and Formula (IX), in the amino acid sequence of the IL-10 conjugate is N82.

Embodiment 360. The IL-10 conjugate of embodiment 358, wherein the position of the structure Formula (VIII) or Formula (IX), or a mixture of Formula (VIII) and Formula (IX), in the amino acid sequence of the IL-10 conjugate is K88.

Embodiment 361. The IL-10 conjugate of embodiment 358, wherein the position of the structure Formula (VIII) or Formula (IX), or a mixture of Formula (VIII) and Formula (IX), in the amino acid sequence of the IL-10 conjugate is A89.

Embodiment 362. The IL-10 conjugate of embodiment 358, wherein the position of the structure Formula (VIII) or Formula (IX), or a mixture of Formula (VIII) and Formula (IX), in the amino acid sequence of the IL-10 conjugate is N129.

Embodiment 363. The IL-10 conjugate of embodiment 358, wherein the position of the structure Formula (VIII) or Formula (IX), or a mixture of Formula (VIII) and Formula (IX), in the amino acid sequence of the IL-10 conjugate is K130.

Embodiment 364. A method of treating cancer in a subject, comprising administering to a subject in need thereof an effective amount of an IL-10 conjugate of any one of embodiments 1 to 363.

Embodiment 365. The method of embodiment 364, wherein the cancer is a solid tumor or a liquid tumor.

Embodiment 366. The method of embodiment 365, wherein the cancer is a solid tumor.

Embodiment 367. The method of embodiment 366, wherein the solid tumor is a metastatic cancer.

Embodiment 368. The method of embodiment 366, wherein the solid tumor is a relapsed or refractory cancer from a prior treatment.

Embodiment 369. The method of any one of embodiments 364 to 368, wherein the cancer in the subject is selected from renal cell carcinoma, bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, squamous cell carcinoma, pancreatic cancer, and prostate cancer.

Embodiment 370. The method of embodiment 364, wherein the cancer in the subject is selected from renal cell carcinoma (RCC), non-small cell lung cancer (NSCLC), head and neck squamous cell cancer (HNSCC), classical Hodgkin lymphoma (cHL), primary mediastinal large B-cell lymphoma (PMBCL), urothelial carcinoma, microsatellite unstable cancer, microsatellite stable cancer, microsatellite-stable colorectal cancer, gastric cancer, cervical cancer, hepatocellular carcinoma (HCC), Merkel cell carcinoma (MCC), melanoma, small cell lung cancer (SCLC), esophageal, glioblastoma, mesothelioma, breast cancer, triple-negative breast cancer, prostate cancer, bladder cancer, ovarian cancer, tumors of moderate to low mutational burden, cutaneous squamous cell carcinoma (CSCC), squamous cell skin cancer (SCSC), tumors of low- to non-expressing PD-L1, tumors disseminated systemically to the liver and CNS beyond their primary anatomic originating site, and diffuse large B-cell lymphoma.

Embodiment 371. The method of embodiment 364, wherein the cancer in the subject is a hematologic malignancy.

Embodiment 372. The method of embodiment 371, wherein the hematologic malignancy comprises a leukemia, a lymphoma, or a myeloma.

Embodiment 373. The method of embodiment 371, wherein the hematologic malignancy is a T-cell malignancy.

Embodiment 374. The method of embodiment 371, wherein the hematological malignancy is a B-cell malignancy.

Embodiment 375. The method of embodiment 371, wherein the hematologic malignancy is a metastatic hematologic malignancy.

Embodiment 376. The method of embodiment 371, wherein the hematologic malignancy is a relapsed hematologic malignancy.

Embodiment 377. The method of embodiment 371, wherein the hematologic malignancy is a refractory hematologic malignancy.

Embodiment 378. The method of embodiment 371, wherein cancer is chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

Embodiment 379. The method of any one of embodiments 364 to 378, wherein the method further comprises administering to the subject in need thereof an effective amount of one or more additional agents.

Embodiment 380. The method of embodiment 379, wherein the one or more additional agents is one or more immune checkpoint inhibitors selected from the group consisting of PD-1 inhibitors, PD-L1 inhibitors, PD-L2 inhibitors, CTLA-4 inhibitors, OX40 agonists, and 4-1BB agonists.

Embodiment 381. The method of embodiment 380, wherein the one or more immune checkpoint inhibitors is selected from PD-1 inhibitors.

Embodiment 382. The method of embodiment 381, wherein the one or more PD-1 inhibitors is selected from pembrolizumab, nivolumab, cemiplimab, lambrolizumab, AMP-224, sintilimab, toripalimab, camrelizumab, tislelizumab, dostarlimab (GSK), PDR001 (Novartis), MGA012 (Macrogenics/Incyte), GLS-010 (Arcus/Wuxi), AGEN2024 (Agenus), cetrelimab (Janssen), ABBV-181 (Abbvie), AMG-404 (Amgen). BI-754091 (Boehringer Ingelheim), CC-90006 (Celgene), JTX-4014 (Jounce), PF-06801591 (Pfizer), and genolimzumab (Apollomics/Genor BioPharma).

Embodiment 383. The method of embodiment 380, wherein the one or more immune checkpoint inhibitors is selected from PD-L1 inhibitors.

Embodiment 384. The method of embodiment 383, wherein the PD-L1 inhibitors is selected from atezolizumab, avelumab, durvalumab, ASC22 (Alphamab/Ascletis), CX-072 (Cytomx), CS1001 (Cstone), cosibelimab (Checkpoint Therapeutics), INCB86550 (Incyte), and TG-1501 (TG Therapeutics).

Embodiment 385. The method of embodiment 380, wherein the one or more immune checkpoint inhibitors is selected from CTLA-4 inhibitors.

Embodiment 386. The method of embodiment 385, wherein the CTLA-4 inhibitors is selected from tremelimumab, ipilimumab, and AGEN-1884 (Agenus).

Embodiment 387. The method of embodiment 379, wherein the one or more additional agents comprises folinic acid, 5-fluorouracil, and oxaliplatin.

Embodiment 388. The method of embodiment 387, wherein the cancer is pancreatic cancer.

Embodiment 389. The method of embodiment 388, wherein the pancreatic cancer is pancreatic ductal adenocarcinoma (PDAC).

Embodiment 390. A method of treating a fibrotic disorder in a subject, comprising administering to a subject in need thereof an effective amount of an IL-10 conjugate of any one of embodiments 1 to 363.

Embodiment 391. The method of embodiment 390, wherein the fibrotic disorder in the subject is selected from liver fibrosis, idiopathic pulmonary fibrosis, and periportal fibrosis.

Embodiment 392. The method of embodiment 391, wherein the fibrotic disorder in the subject is liver fibrosis.

Embodiment 393. The method of embodiment 391, wherein the fibrotic disorder in the subject is idiopathic pulmonary fibrosis.

Embodiment 394. The method of embodiment 391, wherein the fibrotic disorder in the subject is periportal fibrosis.

Embodiment 395. A method of treating non-alcoholic steatohepatitis (NASH) in a subject, comprising administering to a subject in need thereof an effective amount of an IL-10 conjugate of any one of embodiments 1 to 363.

Embodiment 396. A method of treating nonalcoholic fatty liver disease (NAFLD) in a subject, comprising administering to a subject in need thereof an effective amount of an IL-10 conjugate of any one of embodiments 1 to 363.

Embodiment 397. The method of any one of embodiments 364 to 396, wherein administration of the effective amount of the IL-10 to the subject in need thereof does not cause a Grade 3 or Grade 4 adverse event in the subject.

Embodiment 398. The method of embodiment 397, wherein the Grade 3 or Grade 4 adverse event is selected from anemia, leukopenia, thrombocytopenia, increased ALT, anorexia, arthralgia, back pain, chills, diarrhea, dyslipidemia, fatigue, fever, flu-like symptoms, hypoalbuminemia, increased lipase, injection site reaction, myalgia, nausea, night sweats, pruritis, rash, erythematous rash, maculopapular rash, transaminitis, vomiting, and weakness.

Embodiment 399. The method of embodiment 398, wherein the Grade 3 or Grade 4 adverse event is selected from anemia, leukopenia, thrombocytopenia, erythematous rash, and maculopapular rash.

Embodiment 400. The method of embodiment 399, wherein the Grade 3 or Grade 4 adverse event is selected from anemia, thrombocytopenia, erythematous rash, and maculopapular rash.

Embodiment 401. The method of embodiment 400, wherein the Grade 3 or Grade 4 adverse event is selected from anemia and thrombocytopenia.

Embodiment 402. The method of embodiment 401, wherein the Grade 3 or Grade 4 adverse event is anemia.

Embodiment 403. The method of embodiment 401, wherein the Grade 3 or Grade 4 adverse event thrombocytopenia.

Embodiment 404. The method of any one of embodiments 364 to 403, wherein administration of the effective amount of the IL-10 conjugate to a group of subjects does not cause one or more Grade 4 adverse events in greater than 1% of the subjects following administration of the IL-10 conjugate to the subjects.

Embodiment 405. The method of any one of embodiments 364 to 404, wherein the IL-10 conjugate is administered to the subject in need thereof once per day, twice per day, three times per day, once per week, once every two weeks, once every three weeks, once every 4 weeks, once every 5 weeks, once every 6 weeks, once every 7 weeks, or once every 8 weeks.

Embodiment 406. The method of embodiment 405, wherein the IL-10 conjugate is administered to the subject in need thereof once per day, twice per day, three times per day, once per week, once every two weeks, once every three weeks, or once every 4 weeks.

Embodiment 407. The method of embodiment 406, wherein the IL-10 conjugate is administered to the subject in need thereof once per day, twice per day, once per week, once every two weeks, once every three weeks, or once every 4 weeks.

Embodiment 408. The method of embodiment 407, wherein the IL-10 conjugate is administered to the subject in need thereof once per day.

Embodiment 409. The method of embodiment 407, wherein the IL-10 conjugate is administered to the subject in need thereof once per week.

Embodiment 410. The method of embodiment 407, wherein the IL-10 conjugate is administered to the subject in need thereof once every two weeks.

Embodiment 411. The method of embodiment 407, wherein the IL-10 conjugate is administered to the subject in need thereof once every three weeks.

Embodiment 412. The method of embodiment 407, wherein the IL-10 conjugate is administered to the subject in need thereof once every four weeks.

Embodiment 413. The IL-10 conjugate of any one of embodiments 1-363, or the method of any one of embodiments 364-412, wherein the IL-2 conjugate is a pharmaceutically acceptable salt, solvate, or hydrate.

Embodiment 414. An IL-10 conjugate for use in the method of any one of embodiments 364-412.

Embodiment 415. The IL-10 conjugate of any one of embodiments 1-363, or the method of any one of embodiments 364-412, wherein q is 1 in the IL-10 conjugate.

Embodiment 416. The IL-10 conjugate of any one of embodiments 1-363, or the method of any one of embodiments 364-412, wherein q is 2 in the IL-10 conjugate.

Embodiment 417. The IL-10 conjugate of any one of embodiments 1-363, or the method of any one of embodiments 364-412, wherein q is 3 in the IL-10 conjugate.

Embodiment 418. Use of the IL-10 conjugate of any one of embodiments 1-363 or 415-417 for the manufacture of a medicament for treating cancer, a fibrotic disorder, NASH, or NAFLD according to the method of any one of embodiments 364-412.

### EXAMPLES

These examples are provided for illustrative purposes only and not to limit the scope of the claims provided herein.

### Example 1

Biochemical Interactions of PEGylated IL-10 with Human IL-10 Receptor

The kinetic and equilibrium dissociation constants of PEGylated IL-10 compound interaction with human IL-10 receptor are measured using Surface Plasmon Resonance (SPR) at Biosensor Tools LLC. For these studies, human IgG1 Fc-fused IL-10R extracellular domain is captured on the surface of a Protein A-coated CM4 biosensor chip. The surface is probed in duplicate, with two-fold dilution series starting at 2 μM of either native IL-10 (also referred to herein as “natural IL-10” or “wild-type IL-10”) or IL-10 muteins using a Biacore 2000 or similar SPR instrument. Test samples are injected for 60 s or more to allow measurement of association until a plateau is reached, followed by buffer only (wash) for 30 s or more to measure dissociation. Response units (RU, Y-axis) are plotted versus time (s, X-axis).

Ex-Vivo Immune Response Profiling of an IL-10 Mutein in Primary Human Leukocyte Reduction System (LRS)-Derived PBMC Samples

To determine how the differential receptor specificity of an IL-10 mutein affects activation of primary immune cell subpopulations, concentration-response profiling of lymphocyte activation in human LRS-derived peripheral blood mononuclear cell (PBMC) samples is performed using multi-color flow cytometry. These studies are performed at PrimityBio LLC (Fremont, Calif.). Fresh LRS-derived samples are treated with either native IL-10 or an IL-10 mutein in 5-fold dilution series starting with a top concentration of 30 μg/mL. After a 45 min incubation, samples are fixed and stained with antibodies to detect the phosphorylated form of the transcription factor STAT3 (pSTAT3), a marker of upstream engagement and activation of IL-10 receptor signaling complexes, and a panel of surface markers (Table 3) to follow pSTAT3 formation in specific T cell and natural killer (NK) cell subpopulations.

TABLE 3
Staining panel for flow cytometry study of LRS-derived PBMC samples.

| Cell Type | Marker Profile |
|---|---|
| Effector T cells (Teff) | CD3+, CD4+, CD8+, CD127+ |
| NK cells | CD3−, CD16+ |
| Regulatory T cells (Treg) | CD3+, CD4+, CD8−, IL-2Rα+, CD127− |

### Example 2

IL-10 Pegylated Compounds are Produced as Homogeneous Dipegylated Dimers

Samples of IL-10 conjugates corresponding to SEQ ID NOS: 43, 46, 47, 49, 50, and 59 tagged with an N-terminal [His] as defined above (corresponding to Compound A, Compound B, Compound C, Compound D, Compound E, and Compound F, respectively; for Compound F, here and throughout, the PEG was 10 kDa) were prepared by methods described herein. Samples of IL-10 conjugates corresponding to SEQ ID NOS: 43 and 49 in which the N-terminal [His] was removed (corresponding to Compound G and H, respectively) were prepared by methods described herein. Compounds A-H comprise [AzK_L1_PEG] and, as such, comprise a structure of Formula (IV) or Formula (V), or Formula (XII) or Formula (XIII) wherein substituent q is present, and q is 3.

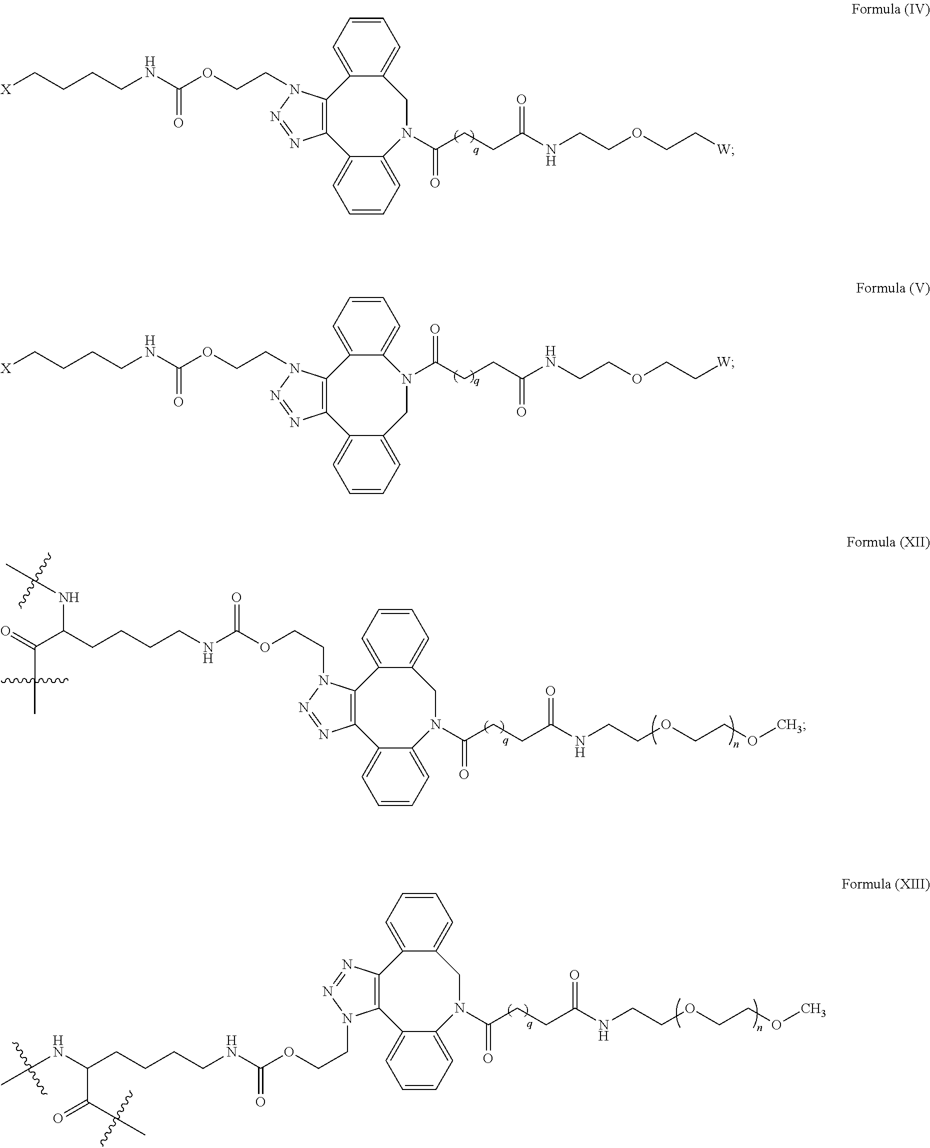

A summary of the structural features of Compounds A-H is provided in Table 3-A.

TABLE 3-A
Summary of Structural Features of Compounds A-H.

| | | Amino Acid | | [His] tag |
|---|---|---|---|---|
| | | Residue | | present at |
| | | Substituted | | N-terminus or |
| | | With | PEG | immediately |
| | Relevant | Unnatural | Molecular | following |
| | SEQ | Amino | Weight | initial |
| Compound | ID NO: | Acid | (kDa) | methionine |
| A | 43 | N82 | 20 | Yes |
| B | 46 | K99 | 20 | Yes |
| C | 47 | K125 | 20 | Yes |
| D | 49 | N129 | 20 | Yes |
| E | 50 | K130 | 20 | Yes |
| F | 59 | N82 | 10 | Yes |
| G | 43 | N82 | 20 | No |
| H | 49 | N129 | 20 | No |

[His] indicates a sequence comprising a His tag and TEV recognition site as defined above.

Briefly, the IL-10 conjugates were expressed as inclusion bodies in E. coli using methods disclosed herein wherein expression plasmids encoding the protein with the desired amino acid sequence were prepared that contained (a) an unnatural base pair comprising a first unnatural nucleotide and a second unnatural nucleotide to provide a codon at the desired position at which an unnatural amino acid N6-((2-azidoethoxy)-carbonyl)-L-lysine (AzK) was incorporated and a matching anticodon in a tRNA, (b) a plasmid encoding a tRNA derived from M. mazei Pyl and which comprises an unnatural nucleotide to provide a matching anticodon in place of its native sequence, (c) a plasmid encoding a M. barkeri derived pyrrolysyl-tRNA synthetase (Mb PylRS), and (d) N6-((2-azidoethoxy)-carbonyl)-L-lysine (AzK). The double-stranded oligonucleotide that encodes the amino acid sequence of the desired IL-10 variant contained a codon AXC at, for example, position 82, 88, 89, 99, 125, 126, 129, or 130 of the sequence that encodes the protein having SEQ ID NO: 1, wherein X is an unnatural nucleotide as disclosed herein. In some embodiments, the cell further comprises a plasmid, which may be the protein expression plasmid or another plasmid, that encodes an orthogonal tRNA gene from M. mazei that comprises an AXC-matching anticodon GYT in place of its native sequence, wherein Y is an unnatural nucleotide as disclosed herein and that may be the same or different as the unnatural nucleotide in the codon. X and Y were selected from unnatural nucleotides dTPT3TP, dNaMTP, and dCNMOTP as disclosed herein. The expressed protein was purified and re-folded using standard procedures before site-specifically pegylating the AzK-containing IL-10 product using DBCO-mediated copper-free click chemistry to attach stable, covalent mPEG moieties to the AzK as outlined in Scheme 6b wherein q is 3.

In some embodiments, the Natural His-TEV-IL-10 has the nucleotide sequence of SEQ ID NO: 75:

ATGCATCATCACCATCATCATGGTAGCAGCGAAAATCTGTATTTTCAGAG
CCCTGGTCAGGGCACCCAGAGCGAAAATTCATGTACCCATTTTCCGGGTA
ATCTGCCGAATATGCTGCGCGATCTGCGTGATGCATTTAGCCGTGTTAAA
ACCTTTTTCCAGATGAAAGATCAGCTGGATAATCTGCTGCTGAAAGAAAG
CCTGCTGGAAGATTTCAAAGGTTATCTGGGTTGTCAGGCACTGAGCGAAA
TGATTCAGTTTTATCTGGAAGAAGTTATGCCGCAGGCAGAAAATCAGGAT
CCGGATATTAAAGCACATGTTAATAGCCTGGGCGAAAATCTGAAAACCCT
GCGTCTGCGCCTGCGTCGTTGTCATCGTTTTCTGCCGTGTGAAAACAAAA
GCAAAGCAGTTGAACAGGTGAAAAACGCCTTTAACAAACTGCAAGAGAAA
GGCATCTATAAAGCCATGAGCGAATTCGACATCTTCATCAACTATATCGA
AGCCTACATGACCATGAAAATCCGCAATTAA

In some embodiments the Natural IL-10 has the nucleotide sequence of SE ID NO: 76:

ATGAGCCCTGGTCAGGGAACCCAATCCGAAAATTCATGTACCCATTTTCC
GGGTAATCTGCCGAATATGCTGCGCGATCTGCGTGATGCATTTAGCCGTG
TTAAAACCTTTTTCCAGATGAAAGATCAGCTGGATAATCTGCTGCTGAAA
GAAAGCCTGCTGGAAGATTTCAAAGGTTATCTGGGTTGTCAGGCACTGAG
CGAAATGATTCAGTTTTATCTGGAAGAAGTTATGCCGCAGGCAGAAAATC
AGGATCCGGATATTAAAGCACATGTTAATAGCCTGGGCGAAAATCTGAAA
ACCCTGCGTCTGCGCCTGCGTCGTTGTCATCGTTTTCTGCCGTGTGAAAA
CAAAAGCAAAGCAGTTGAACAGGTGAAAAACGCCTTTAACAAACTGCAAG
AGAAAGGCATCTATAAAGCCATGAGCGAATTCGACATCTTCATCACTATA
TCGAAGCCTACATGACCATGAAAATCCGCAATTAA

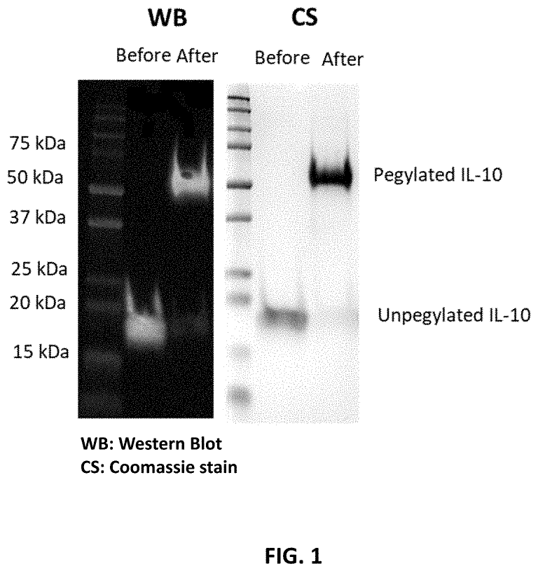

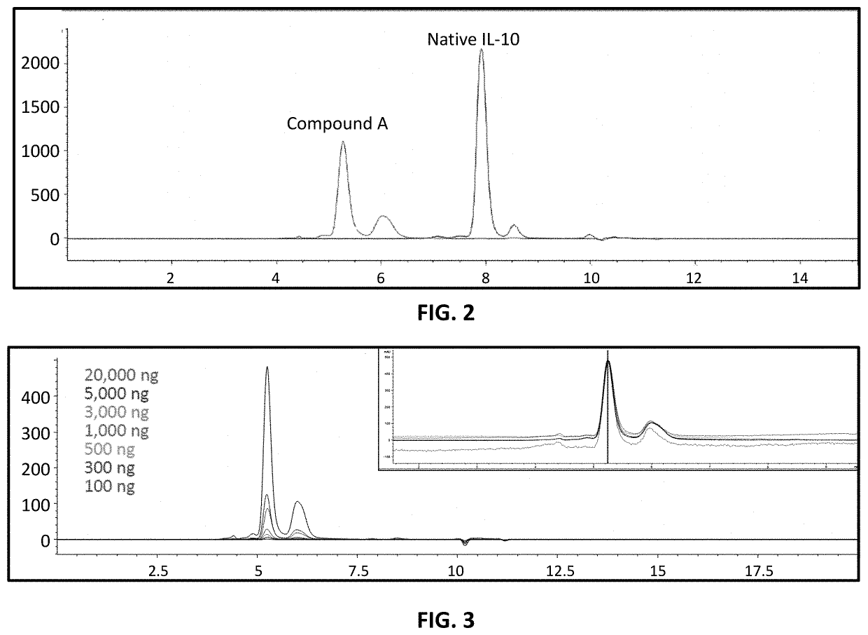

The conjugates following bioconjugation to the respective PEG-containing DBCO reagent were incubated with Laemmli sample buffer under reducing conditions for 5 min at 95° C. After cooling samples to room temperature, the samples were loaded onto SDS-PAGE gels for electrophoretic separation of proteins. The gel was incubated with water-soluble Coomassie stain or transferred to nitrocellulose membrane for detection of the respective compounds by Western Blot with an anti-IL-10 antibody. FIG. 1 illustrates a representative SDS-PAGE and Western Blot analysis of Compound A under reducing conditions shows homogeneous pegylation of IL-10 monomers. Molar mass determination of the conjugates was performed by size exclusion chromatography-multiangle light scattering (SEC-MALS) and were consistent with Compounds A to F being dipegylated dimers (1:1 protein:PEG ratio). Further analysis of compound dilutions showed no subunit dissociation in the range of concentrations tested. FIG. 2 illustrates a representative molar mass determination of Compound A by SEC-MALS. FIG. 3 illustrates a representative analysis of dimer stability of Compound A at low concentrations by size exclusion chromatography (SEC).

Removal of the [His] tag during preparation of Compound A, Compound B, Compound C, Compound D, Compound E, and Compound F can be accomplished by use of tobacco etch virus (TEV) protease according to methods known to those having ordinary skill in the art. Generally, TEV protease recognizes a linear epitope of the general form E-Xaa-Xaa-Y-Xaa-Q-(G/S), with cleavage occurring between Q and G or Q and S. Cleavage of the protein tag by TEV may be performed intracellularly during expression, or during purification, of the IL-10 conjugates described herein. For example, removal of the [His] tag from the unpegylated precursor of Compound A was accomplished using TEV protease at room temperature overnight. Detection was performed by SDS-PAGE under reducing conditions followed by Western Blot analysis using an antibody against IL-10. Further methods for the removal of the [His] tag using TEV protease are provided in Raran-Kurussi et al. (2017) Removal of Affinity Tags with TEV Protease. In: Burgess-Brown N. (eds) Heterologous Gene Expression in E. coli. Methods in Molecular Biology, vol 1586. Humana Press, New York, N.Y.; Phan et al. (2002). Structural basis for the substrate specificity of tobacco etch virus protease. J. Biol. Chem. 277: 50564-50572; and Kapust et al. (2000). Controlled intracellular processing of fusion proteins by TEV protease. Protein Expr. Purif. 19: 312-318.

### Example 3

Bioactivity of IL-10 Conjugates

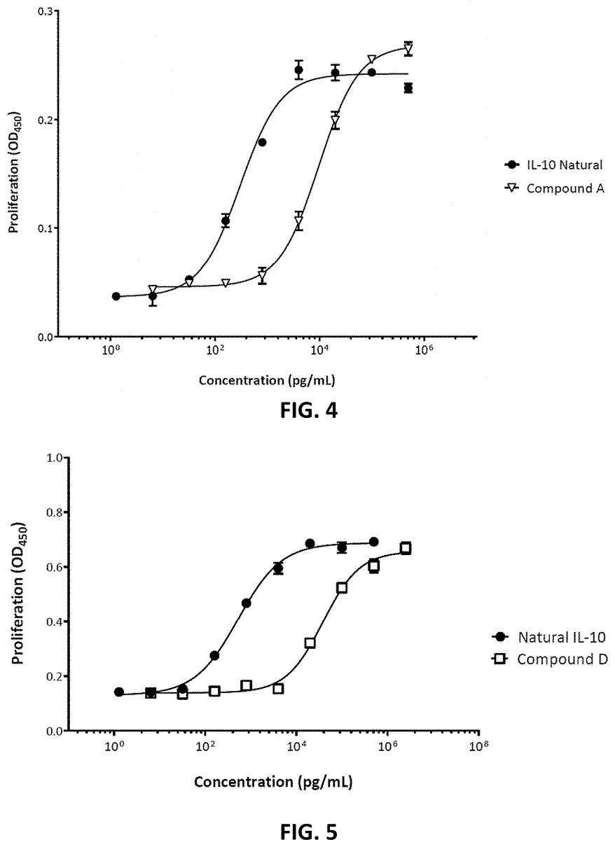

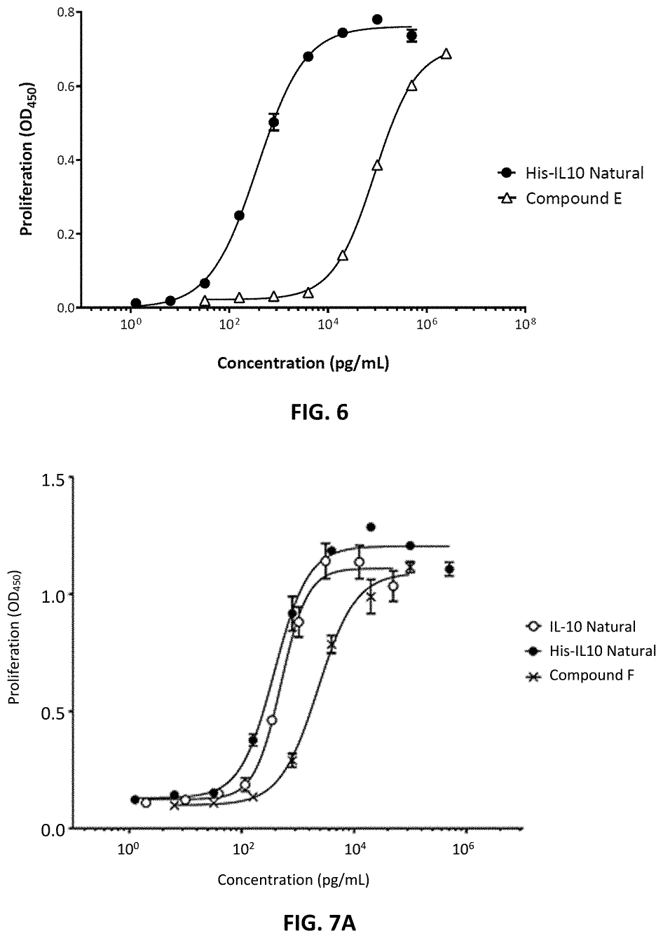

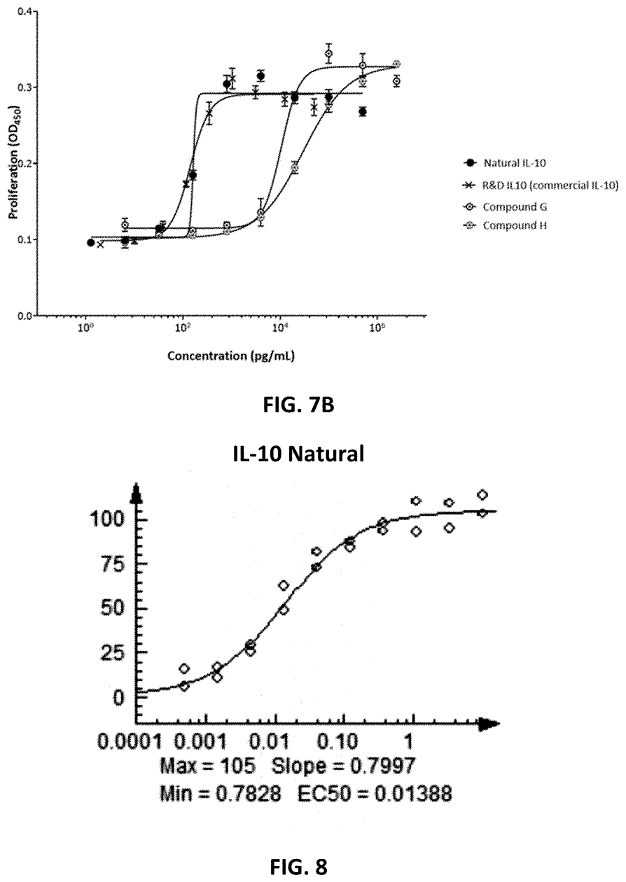

The bioactivity of, Compound A, Compound B, Compound C, Compound D, Compound E, Compound F, Compound G, and Compound H, having the structural features indicated in Table 3-A, were determined using two orthogonal assays: MC/9 cell proliferation assay and PathHunter® Cytokine Receptor Assay (DiscoverX). MC/9 cells depend on cytokine for growth. MC/9 cell cultures were prepared in the presence of IL-2, which was removed prior to stimulation with IL-10. The MC/9 cell proliferation assay measured the proliferation of MC/9 cells treated with increasing concentrations of IL-10 and pegylated compounds after 4 h of IL-2 starvation at 37° C. After 72 hours treatment with hIL-10, the Cell Proliferation Reagent WST-1 (Sigma, 11644807001) was added and cells were incubated for another 3 h at 37° C. before measuring the absorbance of the sample at OD450 against a background control. FIGS. 4 to 7b illustrate traces concentration of IL-10 conjugates (pg/mL) versus proliferation (OD₄₅₀) for Compounds A, D, E, F, G, and H, respectively, in the MC/9 proliferation assay. The data illustrate the [His] N-terminal tag was well tolerated with no significant difference in potency between natural IL-10 and natural [His]-IL-10. Table 4 illustrates the potency (EC₅₀) for different IL-10 compounds in the MC/9 proliferation assay.

TABLE 4
Potency of Exemplary IL-10 Conjugates.

| Compound | EC₅₀ (ng/mL) |
|---|---|
| Compound A | 9.69 |
| Compound B | 129 |
| Compound C | 440 |
| Compound D | 38.2 |
| Compound E | 89.8 |
| Compound F | 7.29 |
| Compound G | 10.5 |
| Compound H | 29.8 |

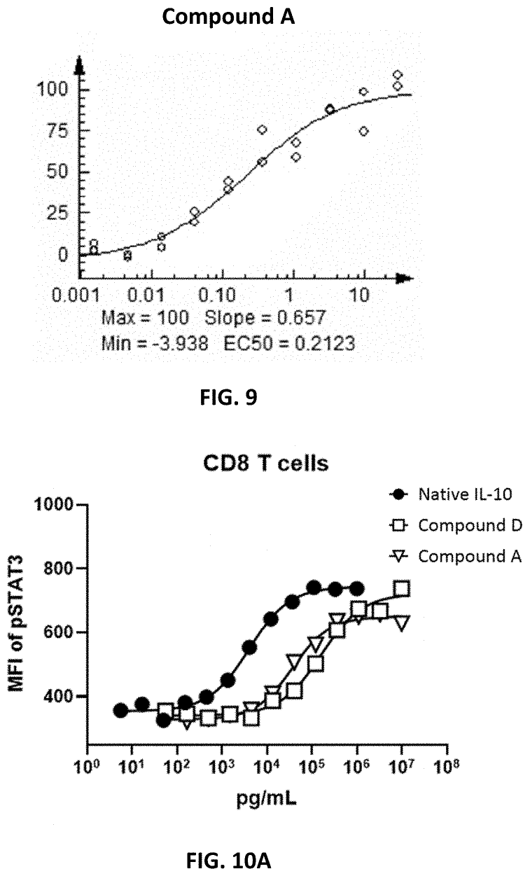

The bioactivity Compound A, Compound B, Compound C, Compound D, Compound E, and Compound F were also measured using the PathHunter® Cytokine Receptor Assay (DiscoverX/Eurofins), which measured the interaction of the 2 chains of the IL-10 receptor upon cytokine engagement. In this assay, one receptor chain was tagged with a small peptide epitope ProLink (PK) and the other chain was tagged with Enzyme Acceptor (EA). The binding of IL-10 or the IL-10 conjugates to the receptor induces dimerization, thus complementation of the PK and EA fragments generating an active unit of P-galactosidase which was detected by chemiluminescence. FIG. 8 illustrates the measurement of bioactivity of wild-type IL-10 in the PathHunter® assay. FIG. 9 illustrates the measurement of bioactivity of Compound A in the PathHunter® assay. Table 5 illustrates the bioactivity of Compound A in the PathHunter® IL-10 R1/R2 dimerization assay versus wild-type IL-10.

TABLE 5
Bioactivity of Exemplary IL-10 Conjugates.

| Compound | EC₅₀ (μM) |
|---|---|
| Wild-type IL-10 | 0.0139 |
| Compound A | 0.2123 |

### Example 4

Profiling of IL-10 Conjugates in Mouse Spleen

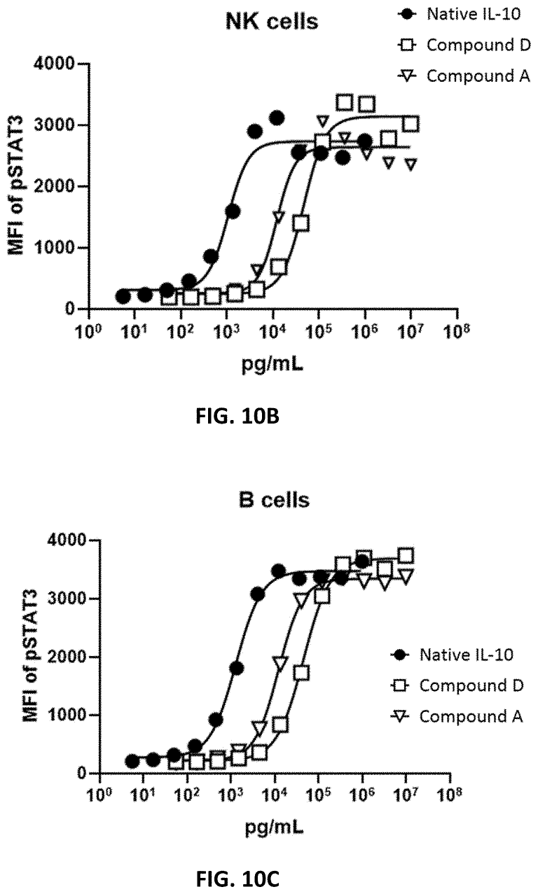

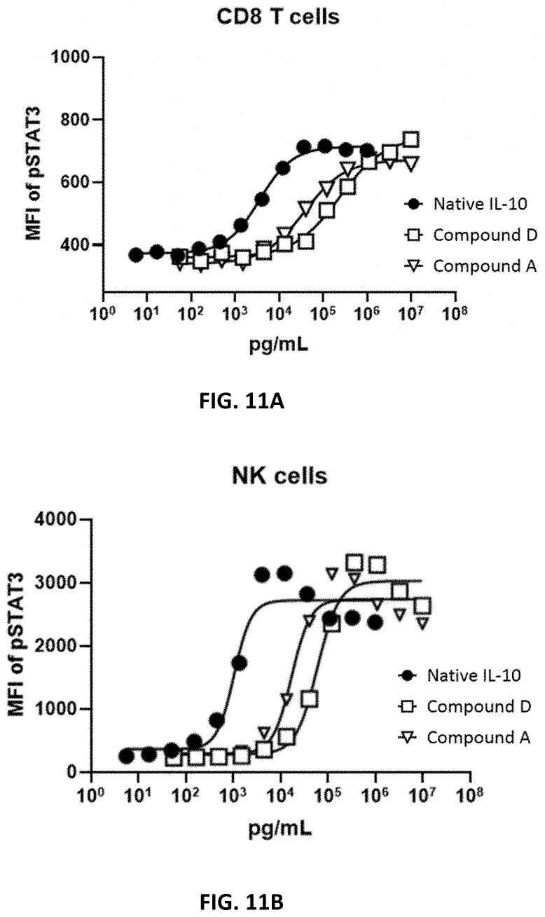

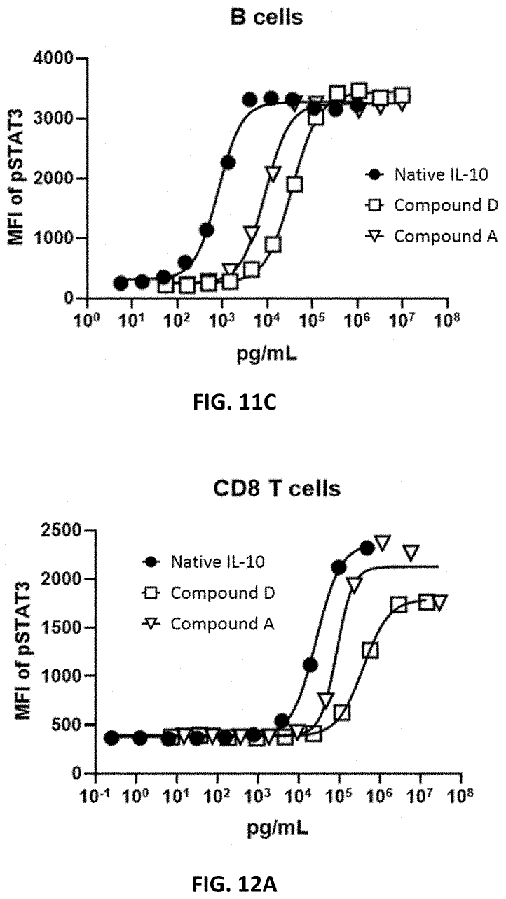

This study evaluated the STAT3 phosphorylation of CD8, NK and B cells in response to stimulation with natural [His]-IL-10, Compound A and Compound D. Treatment was performed on C57BL/6 and Balb/c splenocytes. The dose curve consisted of 12 dose points, 3-fold down from top dose with a 1 μg/mL top dose for HisIL-10 natural and 10 μg/mL for Compound A and Compound D. Mouse spleen splenocytes were prepared by slicing the spleen into small pieces followed by pressing and washing with PBS through a strainer. The cell suspension was centrifuged, and the supernatant was removed by aspiration. A 1× working solution of RBC lysis buffer (BioLegend 420301) was used to resuspend cells. After 4 min at room temperature the reaction was stopped by addition of 4- to 8-fold dilution with PBS and passed through a 70 μm strainer. Cells were centrifuged again and washed in complete splenocyte RPMI medium (RPMI, Gibco 22-400-089 with 10% fetal bovine serum and 1% penicillin/streptomycin (P/S)). Finally, cells were resuspended in complete RPMI medium diluted to 5.5×10⁶ cells/mL, and 90 μL of cells were added to each well of a 96 well u-bottom plate. Cells were incubated for 20 min or longer at 37° C. prior to stimulation. Cells were stimulated for 45 min at 37° C. followed by fixation with 200 μL of warmed fixation buffer (BD 554655) and incubated for 10 min in a 37° C. water bath. Cells were centrifuged and washed with Stain Buffer (BD 554657) twice. The cells were incubated the different antibodies described in Table 6. Table 7 illustrates the potency of wild-type [His]-IL-10, Compound A, and Compound D in Balb/c mouse splenocytes determined by STAT3 phosphorylation. Table 8 illustrates the potency of wild-type [His]-IL-10, Compound A, and Compound D in B57BL/6 mouse splenocytes determined by STAT3 phosphorylation. FIGS. 10A-C illustrate pSTAT3 profiling in Balb/c mouse splenocytes for wild-type IL-10 (closed circles), Compound A (open triangles), and Compound D (open squares) in CD8+ T cells, NK cells, and B cells, respectively. FIGS. 11A-C illustrates pSTAT3 profiling in B57BL/6 mouse splenocytes for wild-type IL-10 (closed circles), Compound A (open triangles), and Compound D (open squares) in CD8+ T cells, NK cells, and B cells, respectively.

TABLE 6
Antibodies used for profiling IL-10 conjugates in mouse spleen.

| Target | Clone | Fluor | 1X Dilution |
|---|---|---|---|
| FcX Block | 93 | N/A | 1:50 |
| CD49b | DX5 | eF506 | 1:50 |
| NKp46 (Balb/c) | 29A1.4 PK136 | BV605 SB600 | 1:50 |
| or NK1.1 (B6) | | | |
| CD62L | MEL-14 | BV421 | 1:200 |
| CD25 | PC61 | FITC | 1:100 |
| CD3 | 17A2 | APC/Cy7 | 1:400 |

TABLE 7
Potency of wild-type His-IL-10, Compound A, and Compound D
in Balb/c mouse splenocytes determined by STAT3 phosphorylation.

| | CD8 T cells | NK cells | B cells |
|---|---|---|---|
| Compound | EC₅₀ (ng/mL) | EC₅₀ (ng/mL) | EC₅₀ (ng/mL) |
| Wild-type His-IL-10 | 4.07 | 1.08 | 1.30 |
| Compound A | 37.5 | 12.3 | 12.5 |
| Compound D | 165 | 47.8 | 46.1 |

TABLE 8
Potency of wild-type His-IL-10, Compound A, and Compound D
in B57BL/6 mouse splenocytes determined by STAT3 phosphorylation.

| | CD8 T cells | NK cells | B cells |
|---|---|---|---|
| Compound | EC₅₀ (ng/mL) | EC₅₀ (ng/mL) | EC₅₀ (ng/mL) |
| Wild-type His-IL-10 | 3.65 | 1.06 | 0.83 |
| Compound A | 38.2 | 17.3 | 9.18 |
| Compound D | 244 | 60.9 | 36.9 |

### Example 5

Profiling of IL-10 Conjugates in a Human Leukoreduction System (LRS)

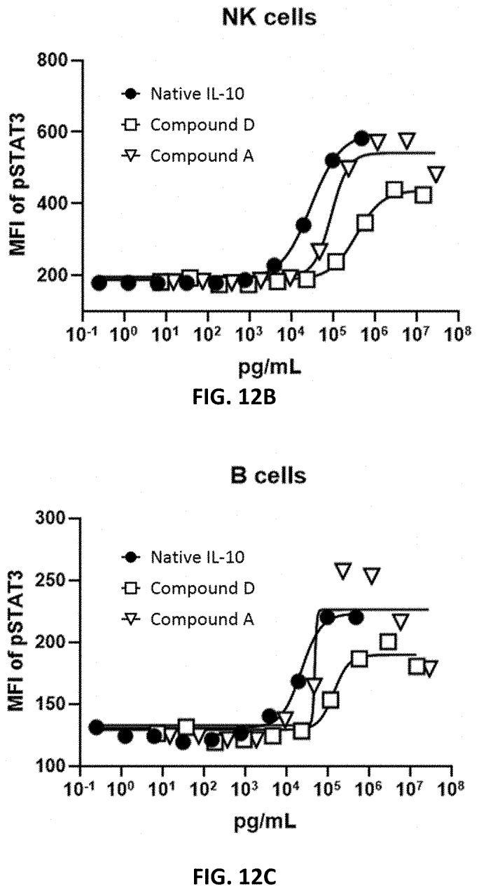

This study evaluated the STAT3 phosphorylation of B cells, NK and CD8+ T cell subsets in response to IL-10 stimulation for wild-type His-IL-10, Compound A, and Compound D. Treatment was done on 1 LRS donor. The top concentration was 0.5 μg/mL for wild-type His-IL-10 and 30 μg/mL for Compound A and Compound D. LRS blood was diluted in PBS and 90 μL of cells were added to each well of a sterile 96-well u-bottom plate. Cells were incubated with dilutions of the compounds for 45 min at 37° C. followed by fixation with Lyse/fix buffer. After 10 min at 37° C., cells were washed with staining buffer followed by incubation with the corresponding antibody solutions as indicated in Table 9. Cells were incubated for 20 min protected from light, followed by two washes with staining buffer. Cells were then permeabilized using ice-cold Perm Buffer III (BD Biosciences) for 30 min protected from light. Cells were incubated with the respective intracellular antibody cocktail as set forth in Table 10 for 1 h in the dark. Finally, cells were washed with staining buffer and prepared for flow cytometric analysis. FIGS. 12A-C illustrate the concentration of wild-type His-IL-10, Compound A, and Compound D versus MFI of pSTAT3 in CD8+ T cells, NK cells, and B cells, respectively.

TABLE 9
Antibody cocktail for staining of membrane markers.

| Target | Clone | Fluor | 1X Dilution |
|---|---|---|---|
| CD127 | eBioRDR5 | eFluor506 | 1:50 |
| CD19 | SJ25C1 | BV785 | 1:100 |
| CD3 | UCHT1 | APC/Cy7 | 1:500 |

TABLE 10
Antibody cocktail for staining of intracellular markers.

| Target | Clone | Fluor | 1X Dilution |
|---|---|---|---|
| CD4 | RPA-T4 | PE/Cy7 | 1:200 |
| CD8 | RPA-T8 | PerCP/Cy5.5 | 1:100 |
| CD25 | M-A251 | PE | 1:500 |
| CD45RA | HI100 | A488 | 1:500 |
| CD14 | M5E2 | BV605 | 1:50 |
| CD56 | HCD56 | BV421 | 1:100 |
| pSTAT3 | 4/P-Stat3 | AF647 | 1:5 |

### Example 6

Measurement of IL-10⁺ CMV Memory Recall Response

This study measured how the IL-10 conjugate Compound A altered the functional memory response of CD8+ T cells to CMV peptide versus wild-type His-IL-10. Donor CD8+ T cells were cultured with peptide loaded non-CD8 cells and multiple concentrations of wild-type His-IL-10 or Compound A for 5 days. After incubation, cells were stained for IFN gamma and PD1. From cryopreserved CMV⁺ PBMC, CD8 T cells were purified by positive selection using a CD8+ T cell isolation kit according to manufacturer's guidelines (Miltenyi Biotech, Auburn, Calif.). CD8 cells were adjusted to 4×10⁶ cells/mL. Non-CD8 cell concentration was adjusted based on measured % CD14+ monocytes by FACS.

$$\frac{2 \times {10}^{5} CD 8 per well \times 0.15}{\% CD 14 monocytes} = Total No . non CD 8 per well \times 10 = Conc . nonCD 8 cells$$

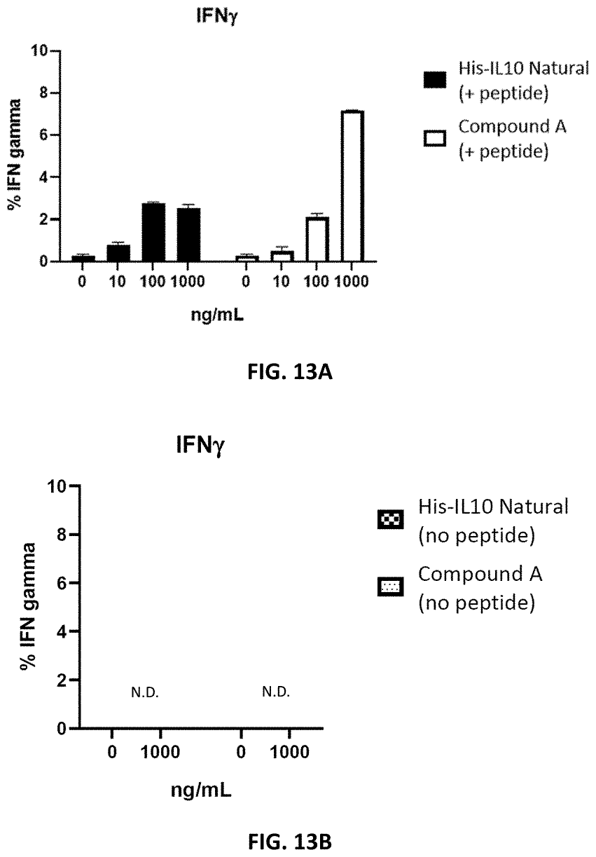

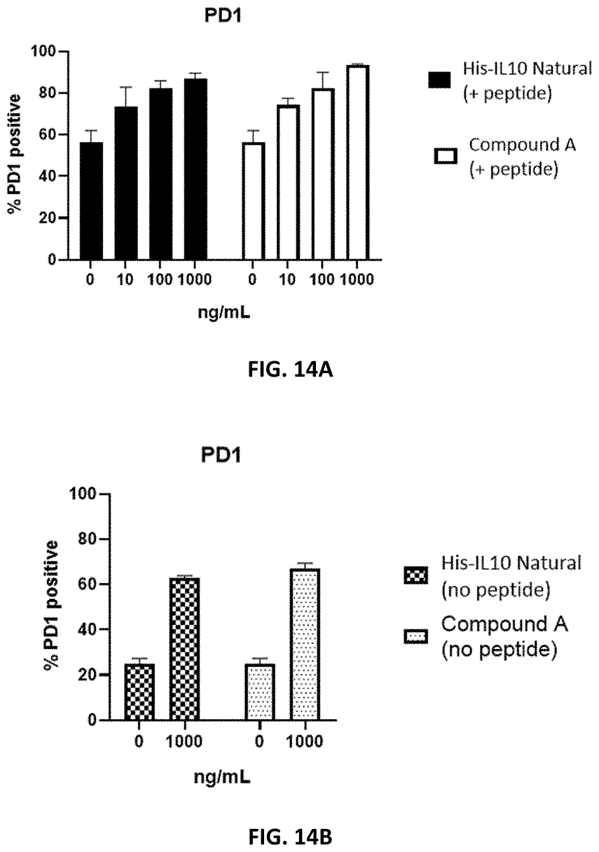

100 μL/well of non-CD8 cells were transferred to another tube for bulk CMV loading. CMV peptide was added at 2× final concentration, incubated for 2 h at room temperature with frequent mixing and then fractions were combined after normalizing CD8 T cell frequency to the frequency of CD14⁺ monocytes. After centrifugation, the cells were resuspended in complete media, transferred to wells on a 96-well pate and incubated with different compounds in complete media. The dose curve consisted of 3 dose points, 10-fold down from top dose. After 5 days of incubation at 37° C., cells were permeabilized and stained with the corresponding antibody cocktail for 30 min at room temperature protected from light. An additional aliquot of extra CD8 and non-CD8 cells was used to confirm the purity of each fraction. FIGS. 13A-B illustrate IFNγ release upon antigen-specific TCR activation by wild-type His-IL-10 or Compound A. FIGS. 14A-B illustrate the upregulation of PD-1 following treatment with His-IL-10 or Compound A and demonstrates that such upregulation is independent of TCR activation.

### Example 7

A Phase 1 Clinical Trial of IL-10 in Participants with Cancer

A clinical trial is performed to investigate the efficacy and safety of administering any one of the modified IL-10 polypeptides or IL-10 conjugates described herein in participants with cancer. In some instances, the study is multicenter, randomized, double-masked, and vehicle-controlled. The study aims to administer 1 μg/kg, 2 μg/kg, 2.5 μg/kg, 5 μg/kg, 10 μg/kg, 15 μg/kg, 20 μg/kg, 25 μg/kg, 30 μg/kg, 50 μg/kg, 100 μg/kg, 200 μg/kg, or more of the modified IL-10 polypeptide or IL-10 conjugate to participants with cancer, including melanoma, prostate cancer, ovarian, cancer, renal cell carcinoma, colorectal carcinoma, pancreatic carcinoma, non-small cell lung carcinoma, solid tumors, and breast cancer.

### Objectives and Outcome Measures

The objective and outcome measures of administering modified IL-10 polypeptides or IL-10 conjugates to participants with cancer is determined based on primary outcome (i.e. safety) and secondary outcome (i.e. efficacy). Primary outcome is determined by measurements of occurrence and severity of adverse events, such as Grade 3 and Grade 4 adverse events. Additional primary outcome is determined from pharmacokinetic (PK) parameters, including minimum and maximum serum drug concentration (Cₘᵢₙ and Cₘₐₓ), area under the curve of serum concentration over time (AUC), and half-life. Secondary outcome is based on change in tumor burden as determined by volumetric computer tomography (CT) or magnetic resonance imaging (MRI).

### Inclusion Criteria

A participant is eligible with histologically or cytologically confirmed advanced malignant solid tumor, limited to melanoma, castrate resistant prostate cancer (CRPC), ovarian cancer (OVCA), renal cell carcinoma, colorectal carcinoma (CRC), pancreatic carcinoma or non-small cell lung carcinoma (NSCLC) that is refractory to, intolerant of, for which no standard of therapy is available or where the participant refuses existing therapies. Participant must be at least 18 years or age and has adequate organ function.

### Exclusion Criteria

Participant is not eligible if pregnant or lactating, has been diagnosed with a neurological disorder, has suffered myocardial infarction within the last 6 months, unstable angina, requiring medication to control cardia arrhythmia, has undergone surgery within the last 28 days, is suffering from any type of infection, has a history of bleeding diathesis within the last 6 months, or has tested positive for HIV, hepatitis C, or hepatitis B.

### Study Design

Participants are randomly assigned into 1 of 3 treatment arms, with participants allocated in a 1:1:1 ratio. During phase A of the study, the first cohort of the participants receives daily subcutaneous (SC) injections of 1 μg/kg of one of the modified IL-10 polypeptides or IL-10 conjugates as described herein for 12 months. The second cohort of the participants receives daily SC injections of 10 μg/kg of one of the modified IL-10 polypeptides or IL-10 conjugates as described herein for 12 months. The third cohort of the participants receives daily SC injections of 50 μg/kg of one of the modified IL-10 polypeptides or IL-10 conjugates as described herein for 12 months. The escalation of dosages aim to determine the occurrence and severity of adverse events in the participants. During phase B of the study, the three cohorts receive daily SC injection of one of the modified IL-10 polypeptides or IL-10 conjugates and at least one additional immune check point inhibitors selected from a PD-1 inhibitor, PD-L1 inhibitor, PD-L2 inhibitor, CTLA-4 inhibitor, OX40 agonist, and 4-1BB agonist.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby. The disclosures of all patent and scientific literature cited herein are expressly incorporated herein in their entirety by reference. To the extent that any incorporated material is inconsistent with the express content of this disclosure, the express content controls.

What is claimed is:

1. An IL-10 conjugate comprising the amino acid sequence of SEQ ID NO: 1 in which at least one amino acid residue in the IL-10 conjugate is replaced by the structure of Formula (I): 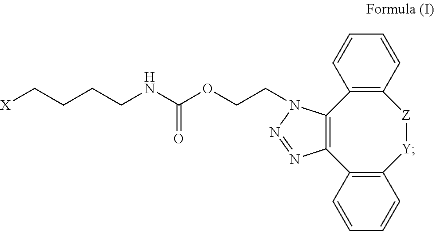
wherein:
Z is CH₂ and Y is 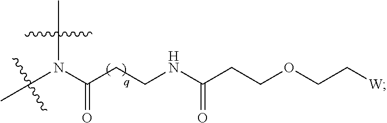
Y is CH₂ and Z is 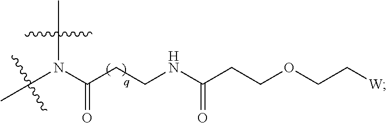
Z is CH₂ and Y is 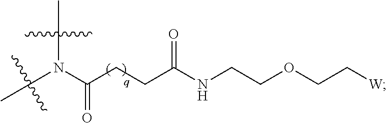
or
Y is CH₂ and Z is 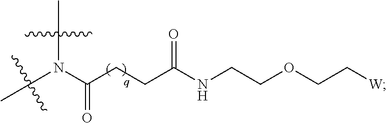
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa;
q is 1, 2, or 3;
X has the structure: 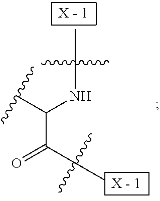 [SMILES: *NC(*)C(*)=O]
X−1 indicates the point of attachment to the preceding amino acid residue; and
X+1 indicates the point of attachment to the following amino acid residue.

2. The IL-10 conjugate of claim 1, wherein Z is CH₂ and Y is

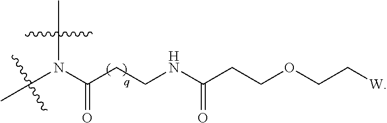

3. The IL-10 conjugate of claim 1, wherein Y is CH₂ and Z is

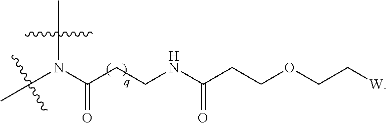

4. The IL-10 conjugate of claim 1, wherein Z is CH₂ and Y is

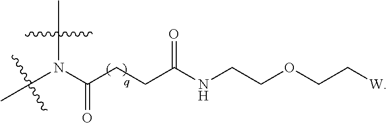

5. The IL-10 conjugate of claim 1, wherein Y is CH₂ and Z is

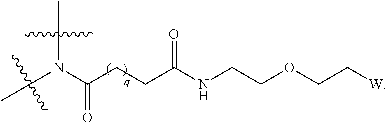

6. The IL-10 conjugate of any one of claims 1-5, wherein the PEG group has an average molecular weight selected from 5 kDa, 10 kDa, 20 kDa and 30 kDa.

7. The IL-10 conjugate of claim 6, wherein the PEG group has an average molecular weight selected from 10 kDa and 20 kDa.

8. The IL-10 conjugate of any one of claims 1-7, wherein the position of the structure of Formula (I) is selected from N82, K88, A89, K99, K125, N126, N129, and K130.

9. The IL-10 conjugate of claim 8, wherein the position of the structure of Formula (I) is selected from N82 and N129.

10. The IL-10 conjugate of claim 1, wherein the structure of Formula (I) has the structure of Formula (X) or Formula (XI), or is a mixture of Formula (X) and Formula (XI): 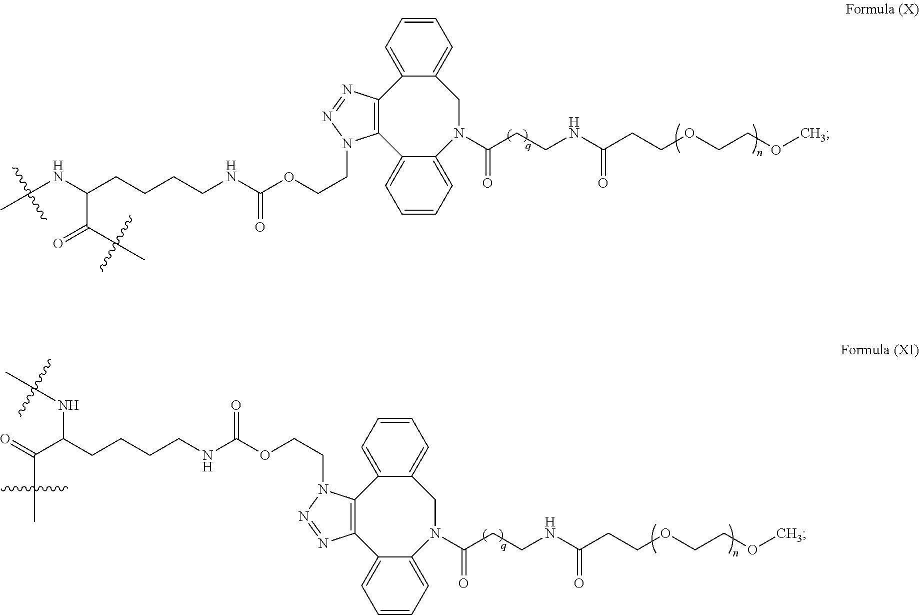
wherein:
q is 1, 2, or 3;
n is an integer in the range from about 2 to about 5000; and
the wavy lines indicate covalent bonds to amino acid residues within SEQ ID NO: 1 that are not replaced.

11. The IL-10 conjugate of claim 10, wherein the position of the structure of Formula (X) or Formula (XI) in SEQ ID NO: 1 is selected from N82, K88, A89, K99, K125, N126, N129, and K130.

12. The IL-10 conjugate of claim 11, wherein the position of the structure of Formula (X) or Formula (XI) in SEQ ID NO: 1 is selected from N82 and N129.

13. The IL-10 conjugate of any one of claims 10-12, wherein n is an integer such that —(OCH₂CH₂)ₙ—OCH₃ has a molecular weight of about 10 kDa or 20 kDa.

14. The IL-10 conjugate of claim 1, wherein the structure of Formula (I) has the structure of Formula (XII) or Formula (XIII), or is a mixture of Formula (XII) and Formula (XIII): 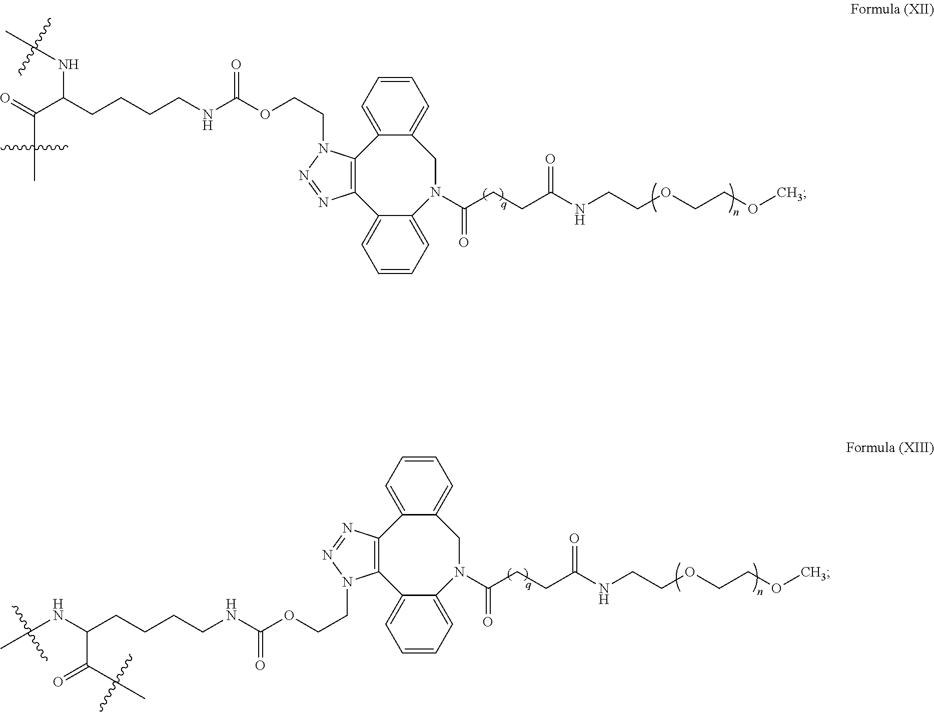
wherein:
q is 1, 2, or 3;
n is an integer in the range from about 2 to about 5000; and
the wavy lines indicate covalent bonds to amino acid residues within SEQ ID NO: 1 that are not replaced.

15. The IL-10 conjugate of claim 14, wherein the position of the structure of Formula (XII) or Formula (XIII) in SEQ ID NO: 1 is selected from N82, K88, A89, K99, K125, N126, N129, and K130.

16. The IL-10 conjugate of claim 14, wherein the position of the structure of Formula (XII) or Formula (XIII) in SEQ ID NO: 1 is selected from N82 and N129.

17. The IL-10 conjugate of any one of claims 14-16, wherein n is an integer such that —(OCH₂CH₂)ₙ—OCH₃ has a molecular weight of about 10 kDa or 20 kDa.

18. The IL-10 conjugate of any one of claims 1-17, wherein q is 1.

19. The IL-10 conjugate of any one of claims 1-17, wherein q is 2.

20. The IL-10 conjugate of any one of claims 1-17, wherein q is 3.

21. The IL-10 conjugate of any one of claims 1-20, wherein the IL-10 conjugate is a pharmaceutically acceptable salt, solvate, or hydrate.

22. A method of treating cancer in a subject in need thereof, comprising administering to the subject an effective amount of the IL-10 conjugate of any one of claims 1-21.

23. The method of claim 22, wherein the cancer is selected from renal cell carcinoma (RCC), non-small cell lung cancer (NSCLC), head and neck squamous cell cancer (HNSCC), classical Hodgkin lymphoma (cHL), primary mediastinal large B-cell lymphoma (PMBCL), urothelial carcinoma, microsatellite unstable cancer, microsatellite stable cancer, microsatellite-stable colorectal cancer, gastric cancer, cervical cancer, hepatocellular carcinoma (HCC), Merkel cell carcinoma (MCC), melanoma, small cell lung cancer (SCLC), esophageal, glioblastoma, mesothelioma, breast cancer, triple-negative breast cancer, prostate cancer, bladder cancer, ovarian cancer, tumors of moderate to low mutational burden, cutaneous squamous cell carcinoma (CSCC), squamous cell skin cancer (SCSC), tumors of low- to non-expressing PD-L1, tumors disseminated systemically to the liver and CNS beyond their primary anatomic originating site, and diffuse large B-cell lymphoma.

24. The method of claim 22 or 23, wherein the IL-10 conjugate is administered to the subject once per day, twice per day, three times per day, once per week, once every two weeks, once every three weeks, once every 4 weeks, once every 5 weeks, once every 6 weeks, once every 7 weeks, or once every 8 weeks.

25. The method of any one of claims 22-24, wherein the IL-10 conjugate is administered to the subject by intravenous administration.

26. A method of making an IL-10 conjugate, comprising:
reacting an IL-10 polypeptide comprising an unnatural amino acid of formula 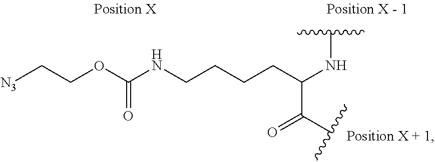 [SMILES: *NC(CCCCNC(=O)OCCN=[N+]=[N-])C(*)=O]
wherein the IL-10 polypeptide comprises the amino acid sequence of SEQ ID NO: 1 in which at least one amino acid residue in the IL-10 polypeptide is replaced by the unnatural amino acid, Position X−1 indicates the point of attachment to the preceding amino acid residue, Position X+1 indicates the point of attachment to the following amino acid residue, and Position X indicates the position of the amino acid for which the unnatural amino acid substitutes,
with an mPEG-DBCO of formula 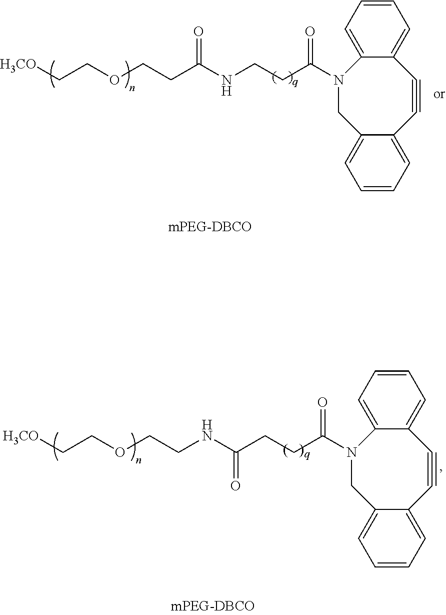
wherein q is 1, 2, or 3, and n is such that the mPEG-DBCO comprises a PEG having a molecular weight of about 5 kDa, 10 kDa, 15 kDa, 20 kDa, 25 kDa, 30 kDa, 35 kDa, 40 kDa, 45 kDa, 50 kDa, or 60 kDa,
thereby producing the IL-10 conjugate.

27. The method of claim 26, wherein q is 1.

28. The method of claim 26, wherein q is 2.

29. The method of claim 26, wherein q is 3.


SEQUENCE LISTING (79 sequences)

SEQ ID NO: 1 - protein, 161 aa, Homo sapiens
  misc_feature: IL-10 (homo sapiens) (mature form)
  misc_feature (1)..(1): Met may or may not be present; Alternatively, the Met residue can substitute and replace the Ser at the N terminus
MSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 178 aa, Homo sapiens
  misc_feature: IL-10 (homo sapiens) (precursor)
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 3 - protein, 161 aa, Artificial Sequence
  Synthetic IL-10_N82X
  misc_feature (1)..(1): Met may or may not be present; Alternatively, the Met residue can substitute and replace the Ser at the N terminus
  misc_feature (83)..(83): Xaa is an unnatural amino acid
MSPGQGTQSENSCTHFPGNLPNMLRDLRDAFSRVKTFFQMKDQLDNLLLKESLLEDFKGYLGCQALSEMIQFYLEEVMPQAEXQDPDIKAHVNSLGENLKTLRLRLRRCHRFLPCENKSKAVEQVKNAFNKLQEKGIYKAMSEFDIFINYIEAYMTMKIRN

SEQ ID NO: 4 - protein, 161 aa, Artificial Sequence
  Synthetic IL-10_K88X
  misc_feature (1)..(1): Met may or may not be present; Alternatively, the Met residue can substitute and replace the Ser at the N terminus
  misc_feature (89)..(89): Xaa is an unnatural amino acid
MSPGQGTQSENSCTHFPGNLPNMLRDLRDAFSRVKTFFQMKDQLDNLLLKESLLEDFKGYLGCQALSEMIQFYLEEVMPQAENQDPDIXAHVNSLGENLKTLRLRLRRCHRFLPCENKSKAVEQVKNAFNKLQEKGIYKAMSEFDIFINYIEAYMTMKIRN

SEQ ID NO: 5 - protein, 161 aa, Artificial Sequence
  Synthetic IL-10_A89X
  misc_feature (1)..(1): Met may or may not be present; Alternatively, the Met residue can substitute and replace the Ser at the N terminus
  misc_feature (90)..(90): Xaa is an unnatural amino acid
MSPGQGTQSENSCTHFPGNLPNMLRDLRDAFSRVKTFFQMKDQLDNLLLKESLLEDFKGYLGCQALSEMIQFYLEEVMPQAENQDPDIKXHVNSLGENLKTLRLRLRRCHRFLPCENKSKAVEQVKNAFNKLQEKGIYKAMSEFDIFINYIEAYMTMKIRN

SEQ ID NO: 6 - protein, 161 aa, Artificial Sequence
  Synthetic IL-10_K99X
  misc_feature (1)..(1): Met may or may not be present; Alternatively, the Met residue can substitute and replace the Ser at the N terminus
  misc_feature (100)..(100): Xaa is an unnatural amino acid
MSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLXTLRLRLRRCHRFLPCENKSKAVEQVKNAFNKLQEKGIYKAMSEFDIFINYIEAYMTMKIRN

SEQ ID NO: 7 - protein, 161 aa, Artificial Sequence
  Synthetic IL-10_K125X
  misc_feature (1)..(1): Met may or may not be present; Alternatively, the Met residue can substitute and replace the Ser at the N terminus
  misc_feature (126)..(126): Xaa is an unnatural amino acid
MSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVXNAFNKLQEKGIYKAMSEFDIFINYIEAYMTMKIRN

SEQ ID NO: 8 - protein, 161 aa, Artificial Sequence
  Synthetic IL-10_N126X
  misc_feature (1)..(1): Met may or may not be present; Alternatively, the Met residue can substitute and replace the Ser at the N terminus
  misc_feature (127)..(127): Xaa is an unnatural amino acid
MSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKXAFNKLQEKGIYKAMSEFDIFINYIEAYMTMKIRN

SEQ ID NO: 9 - protein, 161 aa, Artificial Sequence
  Synthetic IL-10_N129X
  misc_feature (1)..(1): Met may or may not be present; Alternatively, the Met residue can substitute and replace the Ser at the N terminus
  misc_feature (130)..(130): Xaa is an unnatural amino acid
MSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFXKLQEKGIYKAMSEFDIFINYIEAYMTMKIRN

SEQ ID NO: 10 - protein, 161 aa, Artificial Sequence
  Synthetic IL-10_K130X
  misc_feature (1)..(1): Met may or may not be present; Alternatively, the Met residue can substitute and replace the Ser at the N terminus
  misc_feature (131)..(131): Xaa is an unnatural amino acid
MSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNXLQEKGIYKAMSEFDIFINYIEAYMTMKIRN

SEQ ID NO: 11 - protein, 161 aa, Artificial Sequence; identical to SEQ ID NO: 3
  Synthetic IL-10_N82[AzK]
  misc_feature (1)..(1): Met may or may not be present; Alternatively, the Met residue can substitute and replace the Ser at the N terminus
  misc_feature (83)..(83): Xaa is [AzK]

SEQ ID NO: 12 - protein, 161 aa, Artificial Sequence; identical to SEQ ID NO: 4
  Synthetic IL-10_K88[AzK]
  misc_feature (1)..(1): Met may or may not be present; Alternatively, the Met residue can substitute and replace the Ser at the N terminus
  misc_feature (89)..(89): Xaa is [AzK]

SEQ ID NO: 13 - protein, 161 aa, Artificial Sequence; identical to SEQ ID NO: 5
  Synthetic IL-10_A89[AzK]
  misc_feature (1)..(1): Met may or may not be present; Alternatively, the Met residue can substitute and replace the Ser at the N terminus
  misc_feature (90)..(90): Xaa is [AzK]

SEQ ID NO: 14 - protein, 161 aa, Artificial Sequence; identical to SEQ ID NO: 6
  Synthetic IL-10_K99[AzK]
  misc_feature (1)..(1): Met may or may not be present; Alternatively, the Met residue can substitute and replace the Ser at the N terminus
  misc_feature (100)..(100): Xaa is [AzK]

SEQ ID NO: 15 - protein, 161 aa, Artificial Sequence; identical to SEQ ID NO: 7
  Synthetic IL-10_K125[AzK]
  misc_feature (1)..(1): Met may or may not be present; Alternatively, the Met residue can substitute and replace the Ser at the N terminus
  misc_feature (126)..(126): Xaa is [AzK]

SEQ ID NO: 16 - protein, 161 aa, Artificial Sequence; identical to SEQ ID NO: 8
  Synthetic IL-10_N126[AzK]
  misc_feature (1)..(1): Met may or may not be present; Alternatively, the Met residue can substitute and replace the Ser at the N terminus
  misc_feature (127)..(127): Xaa is [AzK]

SEQ ID NO: 17 - protein, 161 aa, Artificial Sequence; identical to SEQ ID NO: 9
  Synthetic IL-10_N129[AzK]
  misc_feature (1)..(1): Met may or may not be present; Alternatively, the Met residue can substitute and replace the Ser at the N terminus
  misc_feature (130)..(130): Xaa is [AzK]

SEQ ID NO: 18 - protein, 161 aa, Artificial Sequence; identical to SEQ ID NO: 10
  Synthetic IL-10_K130[AzK]
  misc_feature (1)..(1): Met may or may not be present; Alternatively, the Met residue can substitute and replace the Ser at the N terminus
  misc_feature (131)..(131): Xaa is [AzK]

SEQ ID NO: 19 - protein, 161 aa, Artificial Sequence; identical to SEQ ID NO: 3
  Synthetic IL-10_N82[AzK_PEG]
  misc_feature (1)..(1): Met may or may not be present; Alternatively, the Met residue can substitute and replace the Ser at the N terminus
  misc_feature (83)..(83): Xaa is [Azk_PEG]

SEQ ID NO: 20 - protein, 161 aa, Artificial Sequence; identical to SEQ ID NO: 4
  Synthetic IL-10_K88[AzK_PEG]
  misc_feature (1)..(1): Met may or may not be present; Alternatively, the Met residue can substitute and replace the Ser at the N terminus
  misc_feature (89)..(89): Xaa is [Azk_PEG]

SEQ ID NO: 21 - protein, 161 aa, Artificial Sequence; identical to SEQ ID NO: 5
  Synthetic IL-10_A89[AzK_PEG]
  misc_feature (1)..(1): Met may or may not be present; Alternatively, the Met residue can substitute and replace the Ser at the N terminus
  misc_feature (90)..(90): Xaa is [Azk_PEG]

SEQ ID NO: 22 - protein, 161 aa, Artificial Sequence; identical to SEQ ID NO: 6
  Synthetic IL-10_K99[AzK_PEG]
  misc_feature (1)..(1): Met may or may not be present; Alternatively, the Met residue can substitute and replace the Ser at the N terminus
  misc_feature (100)..(100): Xaa is [Azk_PEG]

SEQ ID NO: 23 - protein, 161 aa, Artificial Sequence; identical to SEQ ID NO: 7
  Synthetic IL-10_K125[AzK_PEG]
  misc_feature (1)..(1): Met may or may not be present; Alternatively, the Met residue can substitute and replace the Ser at the N terminus
  misc_feature (126)..(126): Xaa is [Azk_PEG]

SEQ ID NO: 24 - protein, 161 aa, Artificial Sequence; identical to SEQ ID NO: 8
  Synthetic IL-10_N126[AzK_PEG]
  misc_feature (1)..(1): Met may or may not be present; Alternatively, the Met residue can substitute and replace the Ser at the N terminus
  misc_feature (127)..(127): Xaa is [Azk_PEG]

SEQ ID NO: 25 - protein, 161 aa, Artificial Sequence; identical to SEQ ID NO: 9
  Synthetic IL-10_N129[AzK_PEG]
  misc_feature (1)..(1): Met may or may not be present; Alternatively, the Met residue can substitute and replace the Ser at the N terminus
  misc_feature (130)..(130): Xaa is [Azk_PEG]

SEQ ID NO: 26 - protein, 161 aa, Artificial Sequence; identical to SEQ ID NO: 10
  Synthetic IL-10_K130[AzK_PEG]
  misc_feature (1)..(1): Met may or may not be present; Alternatively, the Met residue can substitute and replace the Ser at the N terminus
  misc_feature (131)..(131): Xaa is [Azk_PEG]

SEQ ID NO: 27 - protein, 161 aa, Artificial Sequence; identical to SEQ ID NO: 3
  Synthetic IL-10_N82[AzK_PEG20kDa]
  misc_feature (1)..(1): Met may or may not be present; Alternatively, the Met residue can substitute and replace the Ser at the N terminus
  misc_feature (83)..(83): Xaa is [AzK_PEG20kDa]

SEQ ID NO: 28 - protein, 161 aa, Artificial Sequence; identical to SEQ ID NO: 4
  Synthetic IL-10_K88[AzK_PEG20kDa]
  misc_feature (1)..(1): Met may or may not be present; Alternatively, the Met residue can substitute and replace the Ser at the N terminus
  misc_feature (89)..(89): Xaa is [AzK_PEG20kDa]

SEQ ID NO: 29 - protein, 161 aa, Artificial Sequence; identical to SEQ ID NO: 5
  Synthetic IL-10_A89[AzK_PEG20kDa]
  misc_feature (1)..(1): Met may or may not be present; Alternatively, the Met residue can substitute and replace the Ser at the N terminus
  misc_feature (90)..(90): Xaa is [AzK_PEG20kDa]

SEQ ID NO: 30 - protein, 161 aa, Artificial Sequence; identical to SEQ ID NO: 6
  Synthetic IL-10_K99[AzK_PEG20kDa]
  misc_feature (1)..(1): Met may or may not be present; Alternatively, the Met residue can substitute and replace the Ser at the N terminus
  misc_feature (100)..(100): Xaa is [AzK_PEG20kDa]

SEQ ID NO: 31 - protein, 161 aa, Artificial Sequence; identical to SEQ ID NO: 7
  Synthetic IL-10_K125[AzK_PEG20kDa]
  misc_feature (1)..(1): Met may or may not be present; Alternatively, the Met residue can substitute and replace the Ser at the N terminus
  misc_feature (126)..(126): Xaa is [AzK_PEG20kDa]

SEQ ID NO: 32 - protein, 161 aa, Artificial Sequence; identical to SEQ ID NO: 8
  Synthetic IL-10_N126[AzK_PEG20kDa]
  misc_feature (1)..(1): Met may or may not be present; Alternatively, the Met residue can substitute and replace the Ser at the N terminus
  misc_feature (127)..(127): Xaa is [AzK_PEG20kDa]

SEQ ID NO: 33 - protein, 161 aa, Artificial Sequence; identical to SEQ ID NO: 9
  Synthetic IL-10_N129[AzK_PEG20kDa]
  misc_feature (1)..(1): Met may or may not be present; Alternatively, the Met residue can substitute and replace the Ser at the N terminus
  misc_feature (130)..(130): Xaa is [AzK_PEG20kDa]

SEQ ID NO: 34 - protein, 161 aa, Artificial Sequence; identical to SEQ ID NO: 10
  Synthetic IL-10_K130[AzK_PEG20kDa]
  misc_feature (1)..(1): Met may or may not be present; Alternatively, the Met residue can substitute and replace the Ser at the N terminus
  misc_feature (131)..(131): Xaa is [AzK_PEG20kDa]

SEQ ID NO: 35 - protein, 161 aa, Artificial Sequence; identical to SEQ ID NO: 3
  Synthetic IL-10_N82[AzK_PEG30kDa]
  misc_feature (1)..(1): Met may or may not be present; Alternatively, the Met residue can substitute and replace the Ser at the N terminus
  misc_feature (83)..(83): Xaa is [AzK_PEG30kDa]

SEQ ID NO: 36 - protein, 161 aa, Artificial Sequence; identical to SEQ ID NO: 4
  Synthetic IL-10_K88[AzK_PEG30kDa]
  misc_feature (1)..(1): Met may or may not be present; Alternatively, the Met residue can substitute and replace the Ser at the N terminus
  misc_feature (89)..(89): Xaa is [AzK_PEG30kDa]

SEQ ID NO: 37 - protein, 161 aa, Artificial Sequence; identical to SEQ ID NO: 5
  Synthetic IL-10_A89[AzK_PEG30kDa]
  misc_feature (1)..(1): Met may or may not be present; Alternatively, the Met residue can substitute and replace the Ser at the N terminus
  misc_feature (90)..(90): Xaa is [AzK_PEG30kDa]

SEQ ID NO: 38 - protein, 161 aa, Artificial Sequence; identical to SEQ ID NO: 6
  Synthetic IL-10_K99[AzK_PEG30kDa]
  misc_feature (1)..(1): Met may or may not be present; Alternatively, the Met residue can substitute and replace the Ser at the N terminus
  misc_feature (100)..(100): Xaa is [AzK_PEG30kDa]

SEQ ID NO: 39 - protein, 161 aa, Artificial Sequence; identical to SEQ ID NO: 7
  Synthetic IL-10_K125[AzK_PEG30kDa]
  misc_feature (1)..(1): Met may or may not be present; Alternatively, the Met residue can substitute and replace the Ser at the N terminus
  misc_feature (126)..(126): Xaa is [AzK_PEG30kDa]

SEQ ID NO: 40 - protein, 161 aa, Artificial Sequence; identical to SEQ ID NO: 8
  Synthetic IL-10_N126[AzK_PEG30kDa]
  misc_feature (1)..(1): Met may or may not be present; Alternatively, the Met residue can substitute and replace the Ser at the N terminus
  misc_feature (127)..(127): Xaa is [AzK_PEG30kDa]

SEQ ID NO: 41 - protein, 161 aa, Artificial Sequence; identical to SEQ ID NO: 9
  Synthetic IL-10_N129[AzK_PEG30kDa]
  misc_feature (1)..(1): Met may or may not be present; Alternatively, the Met residue can substitute and replace the Ser at the N terminus
  misc_feature (130)..(130): Xaa is [AzK_PEG30kDa]

SEQ ID NO: 42 - protein, 161 aa, Artificial Sequence; identical to SEQ ID NO: 10
  Synthetic IL-10_K130[AzK_PEG30kDa]
  misc_feature (1)..(1): Met may or may not be present; Alternatively, the Met residue can substitute and replace the Ser at the N terminus
  misc_feature (131)..(131): Xaa is [AzK_PEG30kDa]

SEQ ID NO: 43 - protein, 161 aa, Artificial Sequence; identical to SEQ ID NO: 3
  Synthetic IL-10_N82[AzK_L1_PEG20kDa]
  misc_feature (1)..(1): Met may or may not be present; Alternatively, the Met residue can substitute and replace the Ser at the N terminus
  misc_feature (83)..(83): Xaa is [AzK_L1_PEG20kDa]

SEQ ID NO: 44 - protein, 161 aa, Artificial Sequence; identical to SEQ ID NO: 4
  Synthetic IL-10_K88[AzK__L1_PEG20kDa]
  misc_feature (1)..(1): Met may or may not be present; Alternatively, the Met residue can substitute and replace the Ser at the N terminus
  misc_feature (89)..(89): Xaa is [AzK_L1_PEG20kDa]

SEQ ID NO: 45 - protein, 161 aa, Artificial Sequence; identical to SEQ ID NO: 5
  Synthetic IL-10_A89[AzK_L1_PEG20kDa]
  misc_feature (1)..(1): Met may or may not be present; Alternatively, the Met residue can substitute and replace the Ser at the N terminus
  misc_feature (90)..(90): Xaa is [AzK_L1_PEG20kDa]

SEQ ID NO: 46 - protein, 161 aa, Artificial Sequence; identical to SEQ ID NO: 6
  Synthetic IL-10_K99[AzK_L1_PEG20kDa]
  misc_feature (1)..(1): Met may or may not be present; Alternatively, the Met residue can substitute and replace the Ser at the N terminus
  misc_feature (100)..(100): Xaa is [AzK_L1_PEG20kDa]

SEQ ID NO: 47 - protein, 161 aa, Artificial Sequence; identical to SEQ ID NO: 7
  Synthetic IL-10_K125[AzK_L1_PEG20kDa]
  misc_feature (1)..(1): Met may or may not be present; Alternatively, the Met residue can substitute and replace the Ser at the N terminus
  misc_feature (126)..(126): Xaa is [AzK_L1_PEG20kDa]

SEQ ID NO: 48 - protein, 161 aa, Artificial Sequence; identical to SEQ ID NO: 8
  Synthetic IL-10_N126[AzK_L1_PEG20kDa]
  misc_feature (1)..(1): Met may or may not be present; Alternatively, the Met residue can substitute and replace the Ser at the N terminus
  misc_feature (127)..(127): Xaa is [AzK_L1_PEG20kDa]

SEQ ID NO: 49 - protein, 161 aa, Artificial Sequence; identical to SEQ ID NO: 9
  Synthetic IL-10_N129[AzK_L1_PEG20kDa]
  misc_feature (1)..(1): Met may or may not be present; Alternatively, the Met residue can substitute and replace the Ser at the N terminus
  misc_feature (130)..(130): Xaa is [AzK_L1_PEG20kDa]

SEQ ID NO: 50 - protein, 161 aa, Artificial Sequence; identical to SEQ ID NO: 10
  Synthetic IL-10_K130[AzK_L1_PEG20kDa]
  misc_feature (1)..(1): Met may or may not be present; Alternatively, the Met residue can substitute and replace the Ser at the N terminus
  misc_feature (131)..(131): Xaa is [AzK_L1_PEG20kDa]

SEQ ID NO: 51 - protein, 161 aa, Artificial Sequence; identical to SEQ ID NO: 3
  Synthetic IL-10_N82[AzK_L1_PEG30kDa]
  misc_feature (1)..(1): Met may or may not be present; Alternatively, the Met residue can substitute and replace the Ser at the N terminus
  misc_feature (83)..(83): Xaa is [AzK_L1_PEG30kDa]

SEQ ID NO: 52 - protein, 161 aa, Artificial Sequence; identical to SEQ ID NO: 4
  Synthetic IL-10_K88[AzK__L1_PEG30kDa]
  misc_feature (1)..(1): Met may or may not be present; Alternatively, the Met residue can substitute and replace the Ser at the N terminus
  misc_feature (89)..(89): Xaa is [AzK_L1_PEG30kDa]

SEQ ID NO: 53 - protein, 161 aa, Artificial Sequence; identical to SEQ ID NO: 5
  Synthetic IL-10_A89[AzK_L1_PEG30kDa]
  misc_feature (1)..(1): Met may or may not be present; Alternatively, the Met residue can substitute and replace the Ser at the N terminus
  misc_feature (90)..(90): Xaa is [AzK_L1_PEG30kDa]

SEQ ID NO: 54 - protein, 161 aa, Artificial Sequence; identical to SEQ ID NO: 6
  Synthetic IL-10_K99[AzK_L1_PEG30kDa]
  misc_feature (1)..(1): Met may or may not be present; Alternatively, the Met residue can substitute and replace the Ser at the N terminus
  misc_feature (100)..(100): Xaa is [AzK_L1_PEG30kDa]

SEQ ID NO: 55 - protein, 161 aa, Artificial Sequence; identical to SEQ ID NO: 7
  Synthetic IL-10_K125[AzK_L1_PEG30kDa]
  misc_feature (1)..(1): Met may or may not be present; Alternatively, the Met residue can substitute and replace the Ser at the N terminus
  misc_feature (126)..(126): Xaa is [AzK_L1_PEG30kDa]

SEQ ID NO: 56 - protein, 161 aa, Artificial Sequence; identical to SEQ ID NO: 8
  Synthetic IL-10_N126[AzK_L1_PEG30kDa]
  misc_feature (1)..(1): Met may or may not be present; Alternatively, the Met residue can substitute and replace the Ser at the N terminus
  misc_feature (127)..(127): Xaa is [AzK_L1_PEG30kDa]

SEQ ID NO: 57 - protein, 161 aa, Artificial Sequence; identical to SEQ ID NO: 9
  Synthetic IL-10_N129[AzK_L1_PEG30kDa]
  misc_feature (1)..(1): Met may or may not be present; Alternatively, the Met residue can substitute and replace the Ser at the N terminus
  misc_feature (130)..(130): Xaa is [AzK_L1_PEG30kDa]

SEQ ID NO: 58 - protein, 161 aa, Artificial Sequence; identical to SEQ ID NO: 10
  Synthetic IL-10_K130[AzK_L1_PEG30kDa]
  misc_feature (1)..(1): Met may or may not be present; Alternatively, the Met residue can substitute and replace the Ser at the N terminus
  misc_feature (131)..(131): Xaa is [AzK_L1_PEG30kDa]

SEQ ID NO: 59 - protein, 161 aa, Artificial Sequence; identical to SEQ ID NO: 3
  Synthetic IL-10_N82[AzK_L1_PEG]
  misc_feature (1)..(1): Met may or may not be present; Alternatively, the Met residue can substitute and replace the Ser at the N terminus
  misc_feature (83)..(83): Xaa is [AzK_L1_PEG]

SEQ ID NO: 60 - protein, 161 aa, Artificial Sequence; identical to SEQ ID NO: 4
  Synthetic IL-10_K88[AzK_L1_PEG]
  misc_feature (1)..(1): Met may or may not be present; Alternatively, the Met residue can substitute and replace the Ser at the N terminus
  misc_feature (89)..(89): Xaa is [AzK_L1_PEG]

SEQ ID NO: 61 - protein, 161 aa, Artificial Sequence; identical to SEQ ID NO: 5
  Synthetic IL-10_A89[AzK_L1_PEG]
  misc_feature (1)..(1): Met may or may not be present; Alternatively, the Met residue can substitute and replace the Ser at the N terminus
  misc_feature (90)..(90): Xaa is [AzK_L1_PEG]

SEQ ID NO: 62 - protein, 161 aa, Artificial Sequence; identical to SEQ ID NO: 6
  Synthetic IL-10_K99[AzK_L1_PEG]
  misc_feature (1)..(1): Met may or may not be present; Alternatively, the Met residue can substitute and replace the Ser at the N terminus
  misc_feature (100)..(100): Xaa is [AzK_L1_PEG]

SEQ ID NO: 63 - protein, 161 aa, Artificial Sequence; identical to SEQ ID NO: 7
  Synthetic IL-10_K125[AzK_L1_PEG]
  misc_feature (1)..(1): Met may or may not be present; Alternatively, the Met residue can substitute and replace the Ser at the N terminus
  misc_feature (126)..(126): Xaa is [AzK_L1_PEG]

SEQ ID NO: 64 - protein, 161 aa, Artificial Sequence; identical to SEQ ID NO: 8
  Synthetic IL-10_N126[AzK_L1_PEG]
  misc_feature (1)..(1): Met may or may not be present; Alternatively, the Met residue can substitute and replace the Ser at the N terminus
  misc_feature (127)..(127): Xaa is [AzK_L1_PEG]

SEQ ID NO: 65 - protein, 161 aa, Artificial Sequence; identical to SEQ ID NO: 9
  Synthetic IL-10_N129[AzK_L1_PEG]
  misc_feature (1)..(1): Met may or may not be present; Alternatively, the Met residue can substitute and replace the Ser at the N terminus
  misc_feature (130)..(130): Xaa is [AzK_L1_PEG]

SEQ ID NO: 66 - protein, 161 aa, Artificial Sequence; identical to SEQ ID NO: 10
  Synthetic IL-10_K130[AzK_L1_PEG]
  misc_feature (1)..(1): Met may or may not be present; Alternatively, the Met residue can substitute and replace the Ser at the N terminus
  misc_feature (131)..(131): Xaa is [AzK_L1_PEG]

SEQ ID NO: 67 - protein, 176 aa, Artificial Sequence
  Synthetic His-IL-10
  misc_feature (1)..(1): Met may or may not be present; Alternatively, the Met residue can substitute and replace the Ser at the N terminus
MHHHHHHGSSENLYFQSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 68 - protein, 176 aa, Artificial Sequence
  Synthetic His-IL-10_N82[AzK_PEG20kDa]
  misc_feature (1)..(1): Met may or may not be present; Alternatively, the Met residue can substitute and replace the Ser at the N terminus
  misc_feature (98)..(98): Xaa is [AzK_PEG20kDa]
MHHHHHHGSSENLYFQSPGQGTQSENSCTHFPGNLPNMLRDLRDAFSRVKTFFQMKDQLDNLLLKESLLEDFKGYLGCQALSEMIQFYLEEVMPQAEXQDPDIKAHVNSLGENLKTLRLRLRRCHRFLPCENKSKAVEQVKNAFNKLQEKGIYKAMSEFDIFINYIEAYMTMKIRN

SEQ ID NO: 69 - protein, 176 aa, Artificial Sequence
  Synthetic His-IL-10_K99[AzK_PEG20kDa]
  misc_feature (1)..(1): Met may or may not be present; Alternatively, the Met residue can substitute and replace the Ser at the N terminus
  misc_feature (115)..(115): Xaa is [AzK_PEG20kDa]
MHHHHHHGSSENLYFQSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLXTLRLRLRRCHRFLPCENKSKAVEQVKNAFNKLQEKGIYKAMSEFDIFINYIEAYMTMKIRN

SEQ ID NO: 70 - protein, 176 aa, Artificial Sequence
  Synthetic His-IL-10_K125[AzK_PEG20kDa]
  misc_feature (1)..(1): Met may or may not be present; Alternatively, the Met residue can substitute and replace the Ser at the N terminus
  misc_feature (141)..(141): Xaa is [AzK_PEG20kDa]
MHHHHHHGSSENLYFQSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVXNAFNKLQEKGIYKAMSEFDIFINYIEAYMTMKIRN

SEQ ID NO: 71 - protein, 176 aa, Artificial Sequence
  Synthetic His-IL-10_N129[AzK_PEG20kDa]
  misc_feature (1)..(1): Met may or may not be present; Alternatively, the Met residue can substitute and replace the Ser at the N terminus
  misc_feature (145)..(145): Xaa is [AzK_PEG20kDa]
MHHHHHHGSSENLYFQSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFXKLQEKGIYKAMSEFDIFINYIEAYMTMKIRN

SEQ ID NO: 72 - protein, 176 aa, Artificial Sequence
  Synthetic His-IL-10_K130[AzK_PEG20kDa]
  misc_feature (1)..(1): Met may or may not be present; Alternatively, the Met residue can substitute and replace the Ser at the N terminus
  misc_feature (146)..(146): Xaa is [AzK_PEG20kDa]
MHHHHHHGSSENLYFQSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNXLQEKGIYKAMSEFDIFINYIEAYMTMKIRN

SEQ ID NO: 73 - protein, 176 aa, Artificial Sequence; identical to SEQ ID NO: 68
  Synthetic His-IL-10_N82[AzK_PEG10kDa]
  misc_feature (98)..(98): Xaa is [AzK_PEG10kDa]

SEQ ID NO: 74 - protein, 575 aa, Phaeodactylum tricornutum
  misc_feature: untruncated PtNTT2; Phaeodactylum tricornutum CCAP 1055/1
MRPYPTIALISVFLSAATRISATSSHQASALPVKKGTHVPDSPKLSKLYIMAKTKSVSSSFDPPRGGSTVAPTTPLATGGALRKVRQAVFPIYGNQEVTKFLLIGSIKFFIILALTLTRDTKDTLIVTQCGAEAIAFLKIYGVLPAATAFIALYSKMSNAMGKKMLFYSTCIPFFTFFGLFDVFIYPNAERLHPSLEAVQAILPGGAASGGMAVLAKIATHWTSALFYVMAEIYSSVSVGLLFWQFANDVVNVDQAKRFYPLFAQMSGLAPVLAGQYVVRFASKAVNFEASMHRLTAAVTFAGIMICIFYQLSSSYVERTESAKPAADNEQSIKPKKKKPKMSMVESGKFLASSQYLRLIAMLVLGYGLSINFTEIMWKSLVKKQYPDPLDYQRFMGNFSSAVGLSTCIVIFFGVHVIRLLGWKVGALATPGIMAILALPFFACILLGLDSPARLEIAVIFGTIQSLLSKTSKYALFDPTTQMAYIPLDDESKVKGKAAIDVLGSRIGKSGGSLIQQGLVFVFGNIINAAPVVGVVYYSVLVAWMSAAGRLSGLFQAQTEMDKADKMEAKTNKEK

SEQ ID NO: 75 - DNA, 531 bp, Artificial Sequence
  Synthetic Natural His-TEV-IL-10
atgcatcatcaccatcatcatggtagcagcgaaaatctgtattttcagagccctggtcagggcacccagagcgaaaattcatgtacccattttccgggtaatctgccgaatatgctgcgcgatctgcgtgatgcatttagccgtgttaaaacctttttccagatgaaagatcagctggataatctgctgctgaaagaaagcctgctggaagatttcaaaggttatctgggttgtcaggcactgagcgaaatgattcagttttatctggaagaagttatgccgcaggcagaaaatcaggatccggatattaaagcacatgttaatagcctgggcgaaaatctgaaaaccctgcgtctgcgcctgcgtcgttgtcatcgttttctgccgtgtgaaaacaaaagcaaagcagttgaacaggtgaaaaacgcctttaacaaactgcaagagaaaggcatctataaagccatgagcgaattcgacatcttcatcaactatatcgaagcctacatgaccatgaaaatccgcaattaa

SEQ ID NO: 76 - DNA, 486 bp, Artificial Sequence
  Synthetic Natural IL-10
atgagccctggtcagggaacccaatccgaaaattcatgtacccattttccgggtaatctgccgaatatgctgcgcgatctgcgtgatgcatttagccgtgttaaaacctttttccagatgaaagatcagctggataatctgctgctgaaagaaagcctgctggaagatttcaaaggttatctgggttgtcaggcactgagcgaaatgattcagttttatctggaagaagttatgccgcaggcagaaaatcaggatccggatattaaagcacatgttaatagcctgggcgaaaatctgaaaaccctgcgtctgcgcctgcgtcgttgtcatcgttttctgccgtgtgaaaacaaaagcaaagcagttgaacaggtgaaaaacgcctttaacaaactgcaagagaaaggcatctataaagccatgagcgaattcgacatcttcatcaactatatcgaagcctacatgaccatgaaaatccgcaattaa

SEQ ID NO: 77 - protein, 5 aa, Artificial Sequence
  Repeat motif for elastin-like polypeptide conjugation moiety
  MISC_FEATURE (4)..(4): X is any amino acid except proline
VPGXG

SEQ ID NO: 78 - protein, 30 aa, Artificial Sequence
  Exemplary Cytoplasmic Transduction Peptide
  MISC_FEATURE (6)..(6): O-glycosylation site
  MISC_FEATURE (12)..(12): O-glycosylation site
  MISC_FEATURE (17)..(17): O-glycosylation site
  MISC_FEATURE (23)..(23): O-glycosylation site
FQSSSSKAPPPSLPSPSRLPGPSDTPILPQ

SEQ ID NO: 79 - protein, 31 aa, Artificial Sequence
  Exemplary Cytoplasmic Transduction Peptide
  MISC_FEATURE (7)..(7): O-glycosylation site
  MISC_FEATURE (13)..(3): O-glycosylation site
  MISC_FEATURE (18)..(18): O-glycosylation site
  MISC_FEATURE (24)..(24): O-glycosylation site
FQDSSSSKAPPPSLPSPSRLPGPSDTPILPQ